(19) United States
(12) Patent Application Publication
(10) Pub. No.: US 2004/0266748 A1
(43) Pub. Date: Dec. 30, 2004

(54) Photosensitizing carbamate derivatives

(76) Inventors: Byron C Robinson, Santa Barbara, CA (US); Avinash Phadke, Bandford, CT (US)

Correspondence Address: FINNEGAN, HENDERSON, FARABOW, GARRETT & DUNNER, LLP, 1300 I STREET, NW, WASHINGTON, DC, 20005, US

(21) Appl. No.: 10/491,528

(22) PCT Filed: Oct. 2, 2002
(86) PCT No.: PCT/US02/29832

Related U.S. Application Data
(60) Provisional application No. 60/326,427, filed on Oct. 3, 2001.

(51) Int. Cl. C07D 487/22; A61K 31/555; A61K 31/409; A61K 31/4178; A61K 31/4439

(52) U.S. Cl. 514/185; 514/397; 514/410; 540/145; 514/338

(57) ABSTRACT

Carbamate compounds an compositions useful in photodynamic therapy for treating opthalmic, cardiovascular, and skin diseases.

### FIELD OF THE INVENTION

The present invention is directed to carbamate derivatives useful as photoactive compounds in photodynamic therapy and processes for producing such compounds.

### BACKGROUND OF THE INVENTION

Photodynamic therapy is a procedure that uses photoactive (light-activated) drugs to target and destroy diseased cells. Photoactive drugs transform light energy into chemical energy in a manner similar to the action of chlorophyll in green plants. The photoactive drugs are inactive until irradiated with light of a specific wavelength thereby enabling physicians to target specific groups of cells and control the timing and selectivity of treatment. The result of this process is that diseased cells or target cells and tissues are destroyed with minimal damage to surrounding normal tissues.

Photodynamic therapy begins with the administration to a patient of a preferred amount of a photoactive compound that is selectively taken up and/or retained by the biologic target, i.e., tissue or cells. After the photoactive compound is taken up by the target tissue, light of the appropriate wavelength to be absorbed by the photoactive compound is delivered to the targeted area. This activating light excites the photoactive compound to a higher energy state. The extra energy of the excited photoactive compound can then be used to generate a biological response in the target area by interaction with oxygen. As a result of the irradiation, the photoactive compound exhibits cytotoxic activity, i.e., it destroys cells. Additionally, by localizing in the irradiated area, it is possible to contain the cytotoxicity to a specific target area. For a more detailed description of photodynamic therapy, see U.S. Pat. Nos. 5,225,433, 5,198,460, 5,171,749, 4,649,151, 5,399,583, 5,459,159, and 5,489,590, the disclosures of which are hereby incorporated herein by reference.

One important factor in the effectiveness of photodynamic therapy for some disease indications is the depth of tissue penetration by the activating light. It would therefore be desirable to find photoactive compounds that absorb at wavelengths in which light penetration through the tissue is deep. Thus, there is a need for photoactive compounds useful for photodynamic therapy that possess long wavelength absorptions in the 600-800 nm range, a region where light penetration through tissues is optimal.

There is also a need for compounds useful in photodiagnosis. Photodiagnosis is a technique for detecting the existence, position, and/or size of a tumor. For photodiagnosis, light of wavelength between 360 and 800 nm is suitable for activating tetrapyrrole compounds. Of course, each compound has a specific optimal wavelength of activation. A long wavelength ultraviolet lamp is particularly suitable for photodiagnosis.

A large number of naturally occurring and synthetic dyes are currently being evaluated as potential photoactive compounds in the field of photodynamic therapy. Perhaps the most widely studied class of photoactive dyes in this field are the tetrapyrrolic macrocyclic compounds generally called porphyrins.

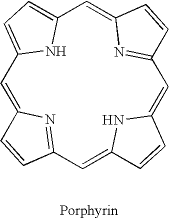

In general, porphyrins typically have a low energy absorption, called band I (or Qy) absorption at ˜620-650nm, with molar extinction coefficients on the order of 100-10,000M⁻¹cm⁻¹. Because of this fact, porphyrins have largely been criticized as having less than optimal wavelength and light absorption properties for use in photodynamic therapy of solid tumors. Compounds such as chlorins (dihydroporphyrins) and bacteriochlorins (tetrahydroporphyrins), where one or two pyrrole rings have been reduced, exhibit low energy band I absorptions that have high molar extinction co-efficients. Such compounds are useful in photodynamic therapy indications that require a large depth of light penetration through tissues.

Many examples of pheophorbides and bacteriopheophorbides are found in nature in plants, algae and bacteria. These sources enable large quantities of these compounds to be isolated and subsequently modified to produce compounds of interest to photodynamic therapy. Four useful intermediates derived from naturally occurring pheophorbides are shown below. These derivatives have been largely functionalized to produce new compounds with different photophysical, pharmacokinetic toxicity and distribution profiles.

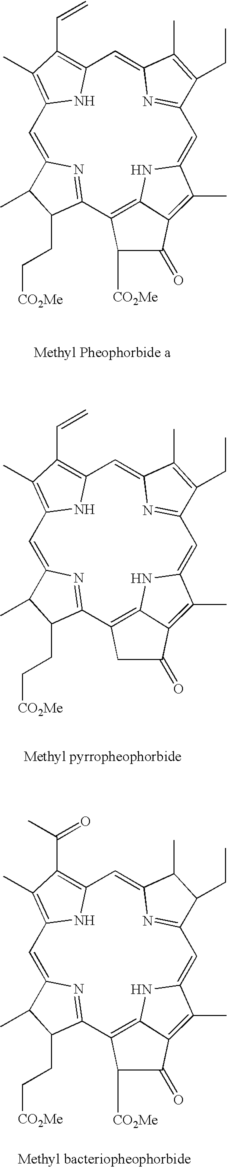

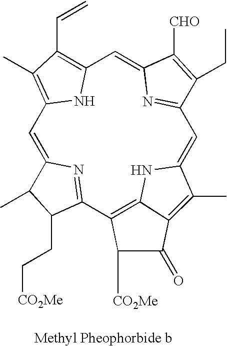

### SUMMARY OF THE INVENTION

To achieve the advantages in accordance with the purpose of the invention, as embodied and broadly described therein, provided are compounds of the following formulae that are useful in the field of photodynamic therapy. Formula I:

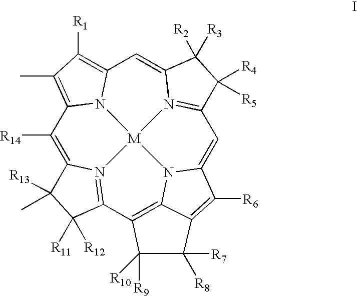

In formula I,

R₁, R₂, R₃, R₄, R₅, R₆, R₇, R₈, R₉, R₁₀, R₁₁, R₁₂, R₁₃, and R₁₄ are independently selected from the group consisting of:

H, halogen, methyl, ethyl, substituted or unsubstituted C1-C20 alkyl, heteroalkyl, haloalkyl, heterohaloalkyl, cycloalkyl, aryl, substituted aryl, alkenyl, substituted alkenyl, alkynyl, substituted alkynyl, amide, ester, ether, polyether, alkoxy, aryloxy, haloalkoxy, amino, alkylcarbonyloxy, alkoxycarbonyl, aryloxycarbonyl, azo, arylcarbonyloxy, alkoxycarbonyloxy, aryloxycarbonyloxy, sulfinyl, sulfonyl, silil, carbamoyl, heterocyclic, nitro, nitroso, formyloxy, isocyano, cyanate, isocyanate, thiocyanate, isothiocyanate, N(alkyl)₂, N(aryl)₂, CH═CH(aryl), CH═CHCH₂N(CH₃)₂, CH═CHCH₂N⁺(CH₃)₃A, CH═N(alkyl)₂⁺A, or N(alkyl)₃⁺A, CN, OH, CHO, COCH₃, CO(alkyl), CO₂H, CO₂Na, CO₂K, CH(CH₃)OH, CH(CH₃)O-alkyl, CH(CH₃)O-alkoxy, CH(CH₃)O-aryl, CH(CH₃)NH-alkyl, CH(CH₃)NH-cycloalkyl, CH(CH₃)NH-heteroalkyl, CH(CH₃)NH-heteroalkoxy, CH(CH₃)-(amino acid), CH(CH₃)-(amino acid ester), CH(CH₃)-(amino acid amide), C(X)₂C(X)₃, (where X is H or halogen), CH═NR₁₅ (where R₁₅ is OH, O-alkyl, O-ether, O-alkylamino, NHCOCH₂N(CH₃)₂, NHCOCH₂N(CH₃)₃⁺A, NHCOCH₂-(pyridinium)⁺A, (CH₂)ₙO-alkoxy, or (CH₂)ₙO-alkyl), where n is an integer ranging from 0 to 8 and A is a charge balancing ion;

CO₂R₁₆, where R₁₆ is selected from H, a physiologically acceptable counter ion, a C1-C20 straight or branched chain alkyl, haloalkyl, heteroalkyl, haloheteroalkyl, aryl, heteroaryl, heterocycle, a mono-, di-, or polyhydroxyalkyl residue, a mono-, di-, or polyhydroxyaryl residue, or a functional group of less than about 100,000 daltons;

(CH₂)ₙOH, or (CH₂)ₙOR₁₇, where R1₇ is selected from alkyl, haloalkyl, heteroalkyl, haloheteroalkyl, aryl, heteroaryl, heterocycle, a protecting group, a mono-, di-, or polyhydroxyalkyl residue, a mono-, di-, or polyhydroxyaryl residue, or a functional group of less than about 100,000 daltons, and n is an integer ranging from 0 to 4;

(CH₂)ₙCO₂R₁₈, (CHX)ₙCO₂R₁₈, or (CX₂)ₙCO₂R_{18,} where X is selected from OH, OR₁₉, or a halogen, and R₁₈ and R₁₉ are independently selected from H, a physiologically acceptable counter ion, acetyl, a straight or branched chain C1-C20 alkyl, haloalkyl, heteroalkyl, haloheteroalkyl, aryl, heteroaryl, heterocycle, a mono-, di-, or polyhydroxyalkyl residue, a mono-, di-, or polyhydroxyaryl residue, or a functional group of less than about 100,000 daltons, and n is an integer ranging from 0 to 4;

CONH(R₂₀), CONHNH(R₂₀), CO(R₂₀), CON(R₂₀)₂, CON(R₂₀)(R₂₁) (CH₂)ₙCONH(R₂₀), (CH₂)ₙCON(R₂₀)₂, (CH₂)ₙCOR₂₀, (CH₂)ₙCON(R₂₀)(R₂₁), (CX₂)ₙCONH(R₂₀), (CX₂)ₙCON(R₂₀)₂, (CX₂)ₙCON(R₂₀)(R₂₁), (CX₂)ₙCOR₂₀, (CH₂)ₙCONHNH(R₂₀), (CX₂)ₙCONHNH(R₂₀), (CHX)ₙCONH(R₂₀), (CHX)ₙCONHNH(R₂₀), (CHX)ₙCO(R₂₀), (CHX)ₙCON(R₂₀)₂, or (CHX)ₙCON(R₂₀)(R₂₁), where X is selected from OH, OR₂₂, SR₂₂, or a halogen, and R₂₀, R₂₁ and R₂₂ are independently selected from H, NH₂, acetyl, a straight or branched chain C1-C20 alkyl, haloalkyl, haloheteroalkyl, heteroalkyl, aryl, heteroaryl, heterocycle, a mono-, di-, or polyhydroxyalkyl residue, a mono-, di-, or polyhydroxyaryl residue, an amino acid ester, an amino acid amide, a mono-, di-, or polyetheralkyl residue, a mono-, di-, or polyetheraryl residue, or a functional group of less than about 100,000 daltons, and n is an integer ranging from 1 to 4;

S(R₂₃), CH(CH₃)S(R₂₃), (CH₂)ₙS(R₂₃), (CH₂)ₙNH(R₂₃), (CH₂)ₙNHNH(R₂₃), (CH₂)ₙN(R₂₃)₂, (CH₂)ₙN(R₂₃)(R₂₄), (CH₂)ₙN(R₂₃)(R₂₄)(R₂₅)⁺A, CH═N(R₂₃), or CH═NN(R₂₃)(R₂₄), where R₂₃, R₂₄ and R₂₅ are independently selected from H, OH, O-alkyl, NH₂, acetyl, a straight or branched, chain C1-C20 alkyl, haloalkyl, heteroalkyl, haloheteroalkyl, aryl, heteroaryl, heterocycle, amino acids (provided —NH(R₂₃) or —N(R₂₃)(R₂₄) is pat of the amino acid), a mono-, di-, or polyhydroxyalkyl residue, a mono-, di, or polyhydroxyaryl residue, a mono-, di-, or polyetheralkyl residue, a mono-, di-, or polyetheraryl residue, or a functional group of less than about 100,000 daltons, and where R₂₃, R₂₄ and R₂₅ together may possess the atoms necessary to constitute an aromatic ring system, n is an integer ranging from 0 to 4, and A is a physiologically acceptable counter ion;

(CH₂)ₙOPO(OR₂₆)₂, or (CH₂)ₙPO(OR₂₆)₂, where R₂₆ is selected from H, a physiologically acceptable counter ion, a straight or branched chain C1-C20 alkyl, haloalkyl, heteroalkyl, haloheteroalkyl, aryl, heteroaryl, heterocycle, a mono-, di-, or polyhydroxyalkyl residue, a mono-, di-, or polyhydroxyaryl residue, a mono-, di-, or polyetheralkyl residue, a mono-, di-, or polyetheraryl residue, or a functional group of less than about 100,000 daltons, and n is an integer ranging from 0 to 4;

(CH₂)ₙNHCOR₂₇, or (CH₂)ₙNHNHCOR₂₇, where R₂₇ is selected from a straight or branched chain C1-C20 alkyl, haloalkyl, heteroalkyl, haloheteroalkyl, aryl, heteroaryl, heterocycle, or a functional group of less than about 100,000 daltons, and n is an integer ranging from between 0 to 4;

SO₃R₂₈, SO₂NHR₂₈, SO₂N(R₂₈)₂, SO₂NHNHR₂₈, SO₂R₂₈, SO₃R₂₈, (CH₂)ₙSO₂NHR₂₈, (CH₂)ₙSO₂N(R₂₈)₂, (CH₂)ₙSO₂NHNHR₂₈, or (CH₂)ₙSO₂R₂₈, where R₂₈ is selected from H, OH, a physiologically acceptable counter ion, a straight or branched chain C1-C20 alkyl, haloalkyl, heteroalkyl, haloheteroalkyl, aryl, heteroaryl, heterocycle, a mono-, di-, or polyhydroxyalkyl residue, a mono-, di-, or polyhydroxyaryl residue, a mono-, di-, or polyetheralkyl residue, a mono-, di-, or polyetheraryl residue, or a functional group of less than about 100,000 daltons, and NHR₂₈ can also be an amino acid, an amino acid salt, an amino acid ester residue, or an amino acid amide residue, and n is an integer ranging from 0 to 4;

aryl or substituted aryl, which may optionally bear one or more substituents with a

molecular weight of less than or equal to about 100,000 daltons;

wherein:

R₃ and R₄ may form a bond;

R₁₂ and R₁₃ may form a bond;

R₇ and R₈ may form a ═O; and

R₉ and R₁₀ may form a ═O;

with the proviso that at least one of R₁ through R₂₈ is a functional group that possesses in part or whole of its structure, a carbamate functionality of the formulae —OCON(R₂₉)₂, —OCON═C(R₂₉)₂, —OCONR₂₉R₃₀, or —OCON═C(R₂₉)(R₃₀), where R₂₉ and R₃₀ are independently selected from H, C1-C20 alkyl, C1-C20 cycloalkyl, aryl, NH₂, N(CH₃)₂, (CH₂)ₙOH, (CH₂)ₙO-alkyl, (CH₂)ₙOCOCH₃, (CH₂)ₙO(CH₂)ₘOH, (CH₂)ₙO(CH₂)ₘOCOCH₃, (CH₂)ₙO(CH₂)ₘO-alkyl, (CH₂)ₙN((CH₂)ₘOH)₂, (CH₂)ₙN((CH₂)ₘO-alkyl)₂, (CH₂)ₙN((CH₂)ₘO-alkylether)₂, ((CH₂)ₙO)ₘ((CH₂)_{Q})OH, (CH₂)ₙO(CH₂)ₘNH₂, (CH₂)ₙO(CH₂)ₘN(CH₃)₂, (CH₂)ₙO(CH₂)ₘN(CH₃)₃⁺A, ( CH₂)ₙN((CH₂)ₘNH₂(CH₂)ₙN(CH₂)ₘN(CH₃)₂, (CH₂)ₙO-haloalkyl, (CH₂)ₙN(CH₂)ₘN(CH₃)₃⁺A)₂, ((CH₂)ₙO)ₘ(CH₂O)_{Q}COCH₃, an alkylphosphate residue, an alkylsulfonic acid residue, an alkylsulfonic ester residue, alkylsulfonic amide residue, an alkylmorpholino residue, an alkylheterocyclic residue, an alkylthiol residue, a mono-, di-, or polyhydroxyalkyl residue, a mono-, di-, or polyhydroxyaryl residue, a mono-, di-, or polyetheralkyl residue, a mono-, di-, or polyetheraryl residue, and Q, n and m are integers ranging from 0 to 10,000, and A is a physiologically acceptable counter ion.

In formula I, M can be selected from 2H, a metal cation, and photoactive metal ions preferably selected from Ga³⁺, Pt²⁺, Pd²⁺, Sn⁴⁺, In³⁺, Ge⁴⁺, Si⁴⁺, Al³⁺, Zn²⁺, Mg²⁺, wherein optionally associated with the metal ion is the appropriate number of physiologically acceptable charge balancing counter ions.

In accordance with the invention, a pharmaceutically acceptable salt, prodrug, solvate, or metabolite of the compound of formula I is also within the scope of the invention.

Formula II:

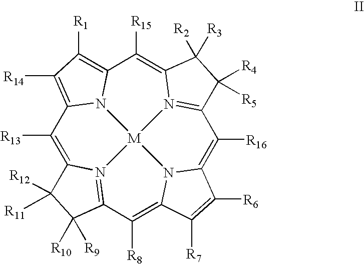

In formula II,

R₁, R₂, R₃, R₄, R₅, R₆, R₇, R₈, R₉, R₁₀, R₁₁, R₁₂, R₁₃, R₁₄, R₁₅, and R₁₆ are independently selected from the group consisting of:

H, halogen, methyl, ethyl, substituted or unsubstituted C1-C20 alkyl, heteroalkyl, haloalkyl, heterohaloalkyl, cycloalkyl, aryl, substituted aryl, alkenyl, substituted alkenyl, alkynyl, substituted alkynyl, amide, ester, ether, polyether, alkoxy, aryloxy, haloalkoxy, amino, alkylcarbonyloxy, alkoxycarbonyl, aryloxycarbonyl, azo, arylcarbonyloxy, alkoxycarbonyloxy, aryloxycarbonyloxy, sulfinyl, sulfonyl, silil, carbamoyl, heterocyclic, nitro, nitroso, formyloxy, isocyano, cyanate, isocyanate, thiocyanate, isothiocyanate, N(alkyl)₂, N(aryl)₂, CH═CH(aryl), CH═CHCH₂N(CH₃)₂, CH═CHCH₂N⁺(CH₃)₃A, CH═N(alkyl)₂⁺A, or N(alkyl)₃⁺A, CN, OH, CHO, COCH₃, CO(alkyl), CO₂H, CO₂Na, CO₂K, CH(CH₃)OH, CH(CH₃)O-alkyl, CH(CH₃)O-alkoxy, CH(CH₃)O-aryl, CH(CH₃)NH-alkyl, CH(CH₃)NH-cycloalkyl, CH(CH₃)NH-heteroalkyl, CH(CH₃)NH-heteroalkoxy, CH(CH₃)-(amino acid), CH(CH₃)-(amino acid ester), CH(CH₃)-(amino acid amide), C(X)₂C(X)₃, (where X is H or halogen), CH═NR₁₇ (where R₁₇ is OH, O-alkyl, O-ether, O-alkylamino, NHCOCH₂N(CH₃)₂, NHCOCH₂N(CH₃)₃⁺A, NHCOCH₂-(pyridinium)⁺A, (CH₂)ₙO-alkoxy, or (CH₂)ₙO-alkyl), where n is an integer ranging from 0 to 8 and A is a charge balancing ion;

CO₂R₁₈, where R₁₆ is selected from H, a physiologically acceptable counter ion, a C1-C20 straight or branched chain alkyl, haloalkyl, heteroalkyl, haloheteroalkyl, aryl, heteroaryl, heterocycle, a mono-, di-, or polyhydroxyalkyl residue, a mono-, di-, or polyhydroxyaryl residue, or a functional group of less than about 100,000 daltons;

(CH₂)ₙOH, or (CH₂)ₙOR₁₉, where R₁₉ is selected from alkyl, haloalkyl, heteroalkyl, haloheteroalkyl, aryl, heteroaryl, heterocycle, a protecting group, a mono-, di-, or polyhydroxyalkyl residue, a mono-, di-, or polyhydroxyaryl residue, or a functional group of less than about 100,000 daltons, and n is an integer ranging from 0 to 4;

(CH₂)ₙCO₂R₂₀, (CHX)ₙCO₂R₂₀, or (CX₂)ₙCO₂R₂₀, where X is selected from OH, OR₂₁, or a halogen, and R₂₀ and R₂₁ are independently selected from H, a physiologically acceptable counter ion, acetyl, a straight or branched chain C1-C20 alkyl, haloalkyl, heteroalkyl, haloheteroalkyl, aryl, heteroaryl, heterocycle, a mono-, di-, or polyhydroxyalkyl residue, a mono-, di-, or polyhydroxyaryl residue, or a functional group of less than about 100,000 daltons, and n is an integer ranging from 0 to 4;

CONH(R₂₂), CONHNH(R₂₂), CO(R₂₂), CON(R₂₂)₂, CON(R₂₂)(R₂₃), (CH₂)ₙCONH(R₂₂), (CH₂)ₙCON(R₂₂)₂, (CH₂)ₙCOR₂₂, (CH₂)ₙCON(R₂₂)(R₂₃), (CX₂)ₙCONH(R₂₂), (CX₂)ₙCON(R₂₂)₂, (CX₂)ₙCON(R₂₂)(R₂₃), (CX₂)ₙCOR₂₂, (CH₂)ₙCONHNH(R₂₂), (CX₂)ₙCONHNH(R₂₂), (CHX)ₙCONH(R₂₂), (CHX)ₙCONHNH(R₂₂), (CHX)ₙCO(R₂₂), (CHX)ₙCON(R₂₂)₂, or (CHX)ₙCON(R₂₂)(R₂₃), where X is selected from OH, OR₂₄, SR₂₄, or a halogen, and R₂₂ , R₂₃ and R₂₄ are independently selected from H, NH₂, acetyl, a straight or branched chain C1-C20 alkyl, haloalkyl, haloheteroalkyl, heteroalkyl, aryl, heteroaryl,.theterocycle, a mono-, di-, or polyhydroxyalkyl residue, a mono-, di-, or polyhydroxyaryl residue, an amino acid ester, an amino acid amide, a mono-, di-, or polyetheralkyl residue, a mono-, di-, or polyetheraryl residue, or a functional group of less than about 100,000 daltons, and n is an integer ranging from 1 to 4;

S(R₂₅), CH(CH₃)S(R₂₅), (CH₂)ₙS(R₂₅), (CH₂)ₙNH(R₂₅), (CH₂)ₙNHNH(R₂₅), (CH₂)ₙN(R₂₅)₂, (CH₂)ₙN(R₂₅)(R₂₆), (CH₂)ₙN(R₂₅)(R₂₆)(R₂₇)⁺A, CH═N(R₂₅), or CH═NN(R₂₅)(R₂₆), where R₂₄, R₂₆ and R₂₇ are independently selected from H, OH, O-alkyl, NH₂, acetyl, a straight or branched chain C1-C20 alkyl, haloalkyl, heteroalkyl, haloheteroalkyl, aryl, heteroaryl, heterocycle, amino acids (provided —NH(R₂₅) or —N(R₂₅)(R₂₆) is part of the amino acid), a mono-, di-, or polyhydroxyalkyl residue, a mono-, di-, or polyhydroxyaryl residue, a mono-, di-, or polyetheralkyl residue, a mono-, di-, or polyetheraryl residue, or a functional group of less than about 100,000 daltons, where R₂₅, R₂₆ and R₂₇ together may possess the atoms necessary to constitute an aromatic ring system, n is an integer ranging from 0 to 4, and A is a physiologically acceptable counter ion;

(CH₂)ₙOPO(OR₂₈)₂ or (CH₂)ₙPO(OR₂₈)₂, where R₂₈ is selected from H, a physiologically acceptable counter ion, a straight or branched chain C1-C20 alkyl, haloalkyl, heteroalkyl, haloheteroalkyl, aryl, heteroaryl, heterocycle, a mono-, di-, or polyhydroxyalkyl residue, a mono-, di-, or polyhydroxyaryl residue, a mono-, di-, or polyetheralkyl residue, a mono-, di-, or polyetheraryl residue, or a functional group of less than about 100,000 daltons, and n is an integer ranging from 0 to 4;

(CH₂)ₙNHCOR₂₉, or (CH₂)ₙNHNHCOR₂₉, where R₂₉ is selected from a straight or branched chain C1-C20 alkyl, haloalkyl, heteroalkyl, haloheteroalkyl, aryl, heteroaryl, heterocycle, or a functional group of less than about 100,000 daltons, and n is an integer ranging from 0 to 4;

SO₃R₃₀, SO₂NHR₃₀, SO₂N(R₃₀)₂, SO₂NHNHR₃₀, SO₂R₃₀, SO₃R₃₀, (CH₂)ₙSO₂NHR₃₀, (CH₂)ₙSO₂N(R₃₀)₂, (CH₂)ₙSO₂NHNHR₃₀, or (CH₂)ₙSO₂R₃₀, where R₃₀ is selected from H, OH, a physiologically acceptable counter ion, a straight or branched chain C1-C20 alkyl, haloalkyl, heteroalkyl, haloheteroalkyl, aryl, heteroaryl, heterocycle, a mono-, di-, or polyhydroxyalkyl residue, a mono-, di-, or polyhydroxyaryl residue, a mono-, di-, or polyetheralkyl residue, a mono-, di-, or polyetheraryl residue, or a functional group of less than about 100,000 daltons, and NHR₂₈ can also be an amino acid, an amino acid salt, an amino acid ester residue or an amino acid amide residue, and n is an integer ranging from 0 to 4;

aryl or substituted aryl, which may optionally bear one or more substituents with a molecular weight of less than or equal to about 1100,000 daltons; and

wherein:

R₃ and R4 may form a bond; and

R₁₀ and R₁₁ may form a bond;

with the proviso that at least one of R₁ through R₃₀ is a functional group that possesses in part or whole of its structure, a carbamate functionality of the formulae —OCON(R₂₉)₂, —OCON═C(R₂₉)₂, —OCONR₂₉R₃₀, or —OCON═C(R₂₉)(R₃₀), where R₂₉ and R₃₀ are independently selected from H, C1-C20 alkyl, C1-C20 cycloalkyl, aryl, NH₂, N(CH₃)₂, (CH₂)ₙOH, (CH₂)ₙO-alkyl, (CH₂)ₙOCOCH₃, (CH₂)ₙO(CH₂)ₘOH, (CH₂)ₙO(CH₂)ₘOCOCH₃, (CH₂)ₙO(CH₂)ₘO-alkyl, (CH₂)ₙN((CH₂)ₘOH)₂, (CH₂)ₙN((CH₂)ₘO-alkyl)₂, (CH₂)ₙN((CH₂)ₘO-alkylether)₂, ((CH₂)ₙO)ₘ(CH₂)_{Q}OH, (CH₂)ₙO(CH₂)ₘNH₂, (CH₂)ₙO(CH₂)ₘN(CH₃)₂, (CH₂)ₙO(CH₂)ₘN(CH₃)₃⁺A, (CH₂)ₙN((CH₂)ₘNH₂)₂, (CH₂)ₙN(CH₂)ₘN(CH₃)₂, (CH₂)ₙO-haloalkyl, (CH₂)ₙN((CH₂)ₘN(CH₃)₃⁺A)₂, ((CH₂)ₙO)ₘ(CH₂O)_{Q}COCH₃, an alkylphosphate residue, an alkylsulfonic acid residue, an alkylsulfonic ester or alkylsulfonic amide reside, an alkylmorpholino residue, an alkylheterocyclic residue, an alkylthiol residue, a mono-, di-, or polyhydroxyalkyl residue, a mono-, di-, or polyhydroxyaryl residue, a mono-, di-, or polyetheralkyl residue, a mono-, di-, or polyetheraryl residue, Q, n and m are integers ranging from 0 to 10,000, and A is a physiologically acceptable counter ion.

In formula II, M can be selected from 2H, a metal cation, and photoactive metal ions preferably selected from Ga³⁺, Pt²⁺, Pd²⁺, Sn⁴⁺, In³⁺, Ge⁴⁺, Si⁴⁺, Al³⁺, Zn²⁺, Mg²⁺, wherein optionally associated with the metal ion is the appropriate number of physiologically acceptable charge balancing counter ions.

In accordance with the invention, a pharmaceutically acceptable salt, prodrug, solvate, or metabolite of the compounds of formula II is also within the scope of the invention.

Formula IIIA and IIIB:

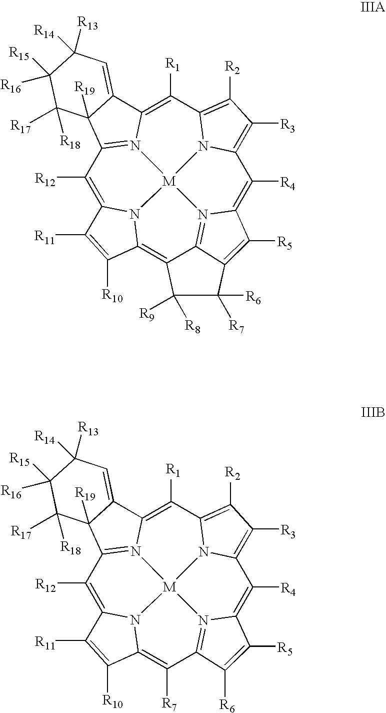

wherein:

R₁, R₂, R₃, R₄, R₅, R₆, R₇, R₈, R₉, R₁₀, R₁₁, R₁₂, R₁₃, R₁₄, R₁₅, R₁₆, R₁₇, R₁₈, and R₁₉, are independently selected from the group consisting of:

H, halogen, methyl, ethyl, substituted or unsubstituted C1-C20 alkyl, heteroalkyl, haloalkyl, heterohaloalkyl, cycloalkyl, aryl, substituted aryl, alkenyl, substituted alkenyl, alkynyl, substituted alkynyl, amide, ester, ether, polyether, alkoxy, aryloxy, haloalkoxy, amino, alkylcarbonyloxy, alkoxycarbonyl, aryloxycarbonyl, azo, arylcarbonyloxy, alkoxycarbonyloxy, aryloxycarbonyloxy, sulfinyl, sulfonyl, silil, carbamoyl, heterocyclic, nitro, nitroso, formyloxy, isocyano, cyanate, isocyanate, thiocyanate, isothiocyanate, N(alkyl)₂, N(aryl)₂, CH═CH(aryl), CH═CHCH₂N(CH₃)₂, CH═CHCH₂N⁺(CH₃)₃A, CH═N(alkyl)₂⁺A, N(alkyl)₃⁺A, CN, OH, CHO, COCH₃, CO(alkyl), CO₂H, CO₂Na, CO₂K, CH(CH₃)OH, CH(CH₃)O-alkyl, CH(CH₃)O-alkoxy, CH(CH₃)Q-aryl, CH(CH₃)NH-alkyl, CH(CH₃)NH-cycloalkyl, CH(CH₃)NH-heteroalkyl, CH(CH₃)NH-heteroalkoxy, CH(CH₃)-(amino acid), CH(CH₃)-(amino acid ester), CH(CH₃)-(amino acid amide), C(X)₂C(X)₃, (where X is H or halogen), CH═NR₂₀ (where R₂₀ is OH, O-alkyl, O-ether, O-alkylamino, NHCOCH₂N(CH₃)₂, NHCOCH₂N(CH₃)₃⁺A, NHCOCH₂-(pyridinium)⁺A, (CH₂)ₙO-alkoxy, or (CH₂)ₙO-alkyl), where n is an integer ranging from 0 to 8 and A is a charge balancing ion;

CO₂R₂₁, where R₂₁ is selected from H, a physiologically acceptable counter ion, a C1-C20 straight or branched chain alkyl, haloalkyl, heteroalkyl, haloheteroalkyl, aryl, heteroaryl, heterocycle, a mono-, di-, or polyhydroxyalkyl residue, a mono-, di-, or polyhydroxyaryl residue, or a functional group of less than about 100,000 daltons;

(CH₂)ₙOH, or (CH₂)ₙOR₂₂, where R₂₂ is selected from alkyl, haloalkyl, heteroalkyl, haloheteroalkyl, aryl, heteroaryl, heterocycle, a protecting group, a mono-, di-, or polyhydroxyalkyl residue, a mono-, di-, or polyhydroxyaryl residue, or a functional group of less than about 100,000 daltons, and n is an integer ranging from 0 to 4;

(CH₂)ₙCO₂R₂₃, (CHX)ₙCO₂R₂₃, or (CX₂)ₙCO₂R₂₃, where X is selected from OH, OR₂₄, or a halogen, and R₂₃ and R₂₄ are independently selected from H, a physiologically acceptable counter ion, acetyl, a straight or branched chain C1-C20 alkyl, haloalkyl, heteroalkyl, haloheteroalkyl, aryl, heteroaryl, heterocycle, a mono-, di-, or polyhydroxyalkyl residue, a mono-, di-, or polyhydroxyaryl residue, or a functional group of less than about 100,000 daltons, and n is an integer ranging from 0 to 4;

CONH(R₂₅), CONHNH(R₂₅), CO(R₂₅), CON(R₂₅)₂, CON(R₂₅)(R₂₆), (CH₂)ₙCONH(R₂₅), (CH₂)ₙCON(R₂₅)₂, (CH₂)ₙCOR₂₅, (CH₂)ₙCON(R₂₅)(R₂₆), (CX₂)ₙCONH(R₂₅), (CX₂)ₙCON(R₂₅)₂, (CX₂)ₙCON(R₂₅)(R₂₆), (CX₂)ₙCOR₂₅, (CH₂)ₙCONHNH(R₂₅), (CX₂)ₙCONHNH(R₂₅), (CHX)ₙCONH(R₂₅), (CHX)ₙCONHNH(R₂₅), (CHX)ₙCO(R₂₅), (CHX)ₙCON(R₂₅)₂, or (CHX)ₙCON(R₂₅)(R₂₆), where X is selected from OH, OR₂₇, SR₂₇, or a halogen, and R₂₅ , R₂₆ and R₂₇ are independently selected from H, NH₂, acetyl, a straight or branched chain C1-C20 alkyl, halo alkyl, haloheteroalkyl, heteroalkyl, aryl, heteroaryl, heterocycle, a mono-, di-, or polyhydroxyalkyl residue, a mono-, di-, or polyhydroxylaryl residue, an amino acid ester, an amino acid amide, a mono-, di-, or polyetheralkyl residue, a mono-, di-, or polyetheraryl residue, or a functional group of less than about 100,000 daltons, and n is an integer ranging from 1 to 4;

S(R₂₈), CH(CH₃)S(R₂₈), (CH₂)ₙS(R₂₈), (CH₂)ₙNH(R₂₈), (CH₂)ₙNHNH(R₂₈), (CH₂)ₙN(R₂₈)₂, (CH₂)ₙN(R₂₈)(R₂₉), (CH₂)ₙN(R₂₈)(R₂₉)(R₃₀)⁺A, CH═N(R₂₈), or CH═NN(R₂₈)(R₂₉), where R₂₈, R₂₉ and R₃₀ are independently selected from H, OH, O-alkyl, NH₂, acetyl, a straight or branched chain C1-C20 alkyl, haloalkyl, heteroalkyl, haloheteroalkyl, aryl, heteroaryl, heterocycle, amino acids (provided —NH(R₂₈) or —N(R₂₈)(R₂₉) is part of the amino acid), a mono-, di-, or polyhydroxyalkyl residue, a mono-, di-, or polyhydroxyaryl residue, a mono-, di-, or polyetheralkyl residue, a mono-, di-, or polyetheraryl residue, or a functional group of less than about 100,000 daltons, where R₂₈, R₂₉ and R₃₀ together may possess the atoms necessary to constitute an aromatic ring system, n is an integer ranging from 0 to 4, and A is a physiologically acceptable counter ion;

(CH₂)ₙO PO(OR₃₁)₂, or (CH₂)ₙPO(OR₃₁)₂, where R₃₁ is selected from H, a physiologically acceptable counter ion, a straight or branched chain C1-C20 alkyl, haloalkyl, heteroalkyl, haloheteroalkyl, aryl, heteroaryl, heterocycle, a mono-, di-, or polyhydroxyalkyl residue, a mono-, di-, or polyhydroxyaryl residue, a mono-, di-, or polyetheralkyl residue, a mono-, di-, or polyetheraryl residue, or a functional group of less than about 100,000 daltons, and n is an integer ranging from 0 to 4;

(CH₂)ₙNHCOR₃₂, or (CH₂)ₙNHNHCOR₃₂, where R₃₂ is selected from a straight or branched chain C1-C20 alkyl, haloalkyl, heteroalkyl, haloheteroalkyl, aryl, heteroaryl, heterocycle, or a functional group of less than about 100,000 daltons, and n is an integer ranging from 0 to 4;

SO₃R₃₄, SO₂NHR₃₄, SO₂N(R₃₄)₂, SO₂NHNHR₃₄, SO₂R₃₄, SO₃R₃₄, (CH₂)ₙSO₂NHR₃₄, (CH₂)ₙSO₂N(R₃₄)₂, (CH₂)ₙSO₂NHNHR₃₄, or (CH₂)ₙSO₂R₃₄, where R₃₄ is selected from H, OH, a physiologically acceptable counter ion, a straight or branched chain C1-C20 alkyl, haloalkyl, heteroalkyl, haloheteroalkyl, aryl, heteroaryl,, heterocycle, a mono-, di-, or polyhydroxyalkyl residue, a mono-, di-, or polyhydroxyaryl residue, a mono-, di-, or polyetheralkyl residue, a mono-, di-, or polyetheraryl residue, or a functional group of less than about 100,000 daltons, NHR₃₄ can also be an amino acid, an amino acid salt, an amino acid ester residue, an amino acid amide residue, and n is an integer ranging from 1 to 4;

aryl or substituted aryl, which may optionally bear one or more substituents with a molecular weight of less than or equal to about 100,000 daltons;

wherein:

R₁₄ and R₁₅ may form a bond; and

R₆ and R₇ may form a ═O;

with the proviso that at least one of R₁ through R₃₄ is a functional group that possesses in part or whole of its structure, a carbamate functionality of the formulae —OCON(R₃₅)₂, —OCON═C(R₃₅)₂, —OCONR₃₅R₃₆, or —OCON═C(R₃₅)(R₃₆), where R₃₅ and R₃₆ are independently selected from H, C1-C20 alkyl, C1-C20 cycloalkyl, aryl, NH₂, N(CH₃)₂, (CH₂)ₙOH, (CH₂)ₙO-alkyl, (CH₂)ₙOCOCH₃, (CH₂)ₙO(CH₂)ₘOH, (CH₂)ₙO(CH₂)ₘOCOCH₃, (CH₂)ₙO(CH₂)ₘO-alkyl, (CH₂)ₙN((CH₂)ₘOH)₂, (CH₂)ₙN((CH₂)ₘO-alkyl)₂, (CH₂)ₙN((CH₂)ₘO-alkylether)₂, ((CH₂)ₙO)ₘ(CH₂)_{Q}OH, (CH₂)ₙO(CH₂)ₘNH₂, (CH₂)ₙO(CH₂)ₘN(CH₃)₂, (CH₂)ₙO(CH₂)ₘN(CH₃)₃⁺A, (CH₂)ₙN((CH₂)ₘNH₂)₂, (CH₂)ₙN(CH₂)ₘN(CH₃)₂, (CH₂)ₙO-haloalkyl, (CH₂)ₙN((CH₂)ₘN(CH₃)₃⁺A)₂, ((CH₂)ₙO)ₘ,((CH₂O)_{Q}COCH₃, an alkylphosphate residue, an alkylsulfonic acid residue, an alkylsulfonic ester or alkylsulfonic amide reside, an alkylmorpholino residue, an alkylheterocyclic residue, an alkylthiol residue, a mono-, di-, or polyhydroxyalkyl residue, a mono-, di-, or polyhydroxyaryl residue, a mono-, di-, or polyetheralkyl residue, a mono-, di-, or polyetheraryl residue, Q, n and m are integers ranging from 0 to 10,000, and A is a physiologically acceptable counter ion;

In Formulae IIIA and IIB, M can be selected from 2H, a metal cation, or photoactive metal ions preferably selected from Ga³⁺, Pt²⁺, Pd²⁺, Sn⁴⁺, In³⁺, Ge⁴⁺, Si⁴⁺, Al³⁺, Zn²⁺, Mg²⁺, wherein optionally associated with the metal ion is the appropriate number of physiologically acceptable charge balancing counter ions.

In accordance with the invention, a pharmaceutically acceptable salt, prodrug, solvate, or metabolite of the compounds of formulae IIA and IIB is within the scope of the invention.

Formulae IVA and IVB:

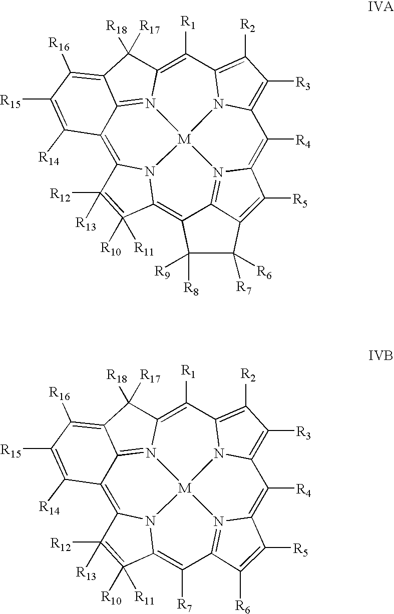

wherein:

R₁, R₂, R₃, R₄, R₅, R₆, R₇, R₈, R₉, R₁₀, R₁₁, R₁₂, R₁₃, R₁₄, R₁₅, R₁₆, R₁₇, and R₁₈, are independently selected from the group consisting of:

H, halogen, methyl, ethyl, substituted or unsubstituted C1-C20 alkyl, heteroalkyl, haloalkyl, heterohaloalkyl, cycloalkyl, aryl, substituted aryl, alkenyl, substituted alkenyl, alkynyl, substituted alkynyl, amide, ester, ether, polyether, alkoxy, aryloxy, haloalkoxy, amino, alkylcarbonyloxy, alkoxycarbonyl, aryloxycarbonyl, azo, arylcarbonyloxy, alkoxycarbonyloxy, aryloxycarbonyloxy, sulfinyl, sulfonyl, silil, carbamoyl, heterocyclic, nitro, nitroso, formyloxy, isocyano, cyanate, isocyanate, thiocyanate, isothiocyanate, N(alkyl)₂, N(aryl)₂, CH═CH(aryl), CH═CHCH₂N(CH₃)₂, CH═CHCH₂N⁺(CH₃)₃A, CH═N(alkyl)₂⁺A, N(alkyl)₃⁺A, CN, OH, CHO, COCH₃, CO(alkyl), CO₂H, CO₂Na, CO₂K, CH(CH₃)OH, CH(CH₃)O-alkyl, CH(CH₃)O-alkoxy, CH(CH₃)O-aryl, CH(CH₃)NH-alkyl, CH(CH₃)NH-cycloalkyl, CH(CH₃)N H-heteroalkyl, CH(CH₃)NH-heteroalkoxy, CH(CH₃)-(amino acid ), CH(CH₃)-(amino acid ester), CH(CH₃)-(amino acid amide), C(X)₂C(X)₃, (where X is H or halogen), CH═NR₁₉ (where R₁₉ is OH, O-alkyl, O-ether, O-alkylamino, NHCOCH₂N(CH₃)₂, NHCOCH₂N(CH₃)₃⁺A, NHCOCH₂-(pyridinium)⁺A, (CH₂)ₙO-alkoxy, or (CH₂)ₙO-alkyl), where n is an integer ranging from 0 to 8, and A is a charge balancing ion;

CO₂R₂₀, where R₂₀ is selected from H, a physiologically acceptable counter ion, a C1-C20 straight or branched chain alkyl, haloalkyl, heteroalkyl, haloheteroalkyl, aryl, heteroaryl, heterocycle, a mono-, di-, or polyhydroxyalkyl residue, a mono-, di-, or polyhydroxyaryl residue, or a functional group of less than about 100,000 daltons;

(CH₂)ₙOH, or (CH₂)ₙOR₂₁, where R₂₁ is selected from alkyl, haloalkyl, heteroalkyl, haloheteroalkyl, aryl, heteroaryl, heterocycle, a protecting group, a mono-, di-, or polyhydroxyalkyl residue, a mono-, di-, or polyhydroxyaryl residue, or a functional group of less than about 100,000 daltons, and n is an integer ranging from 0 to 4;

(CH₂)ₙCO₂R₂₂, (CHX)ₙCO₂R₂₂, or (CX₂)ₙCO₂R₂₂, where X is selected from OH, OR₂₃, or a halogen, and R₂₂ and R₂₃ are independently selected from H, a physiologically acceptable counter ion, acetyl, a straight or branched chain C1-C20 alkyl, haloalkyl, heteroalkyl, haloheteroalkyl, aryl, heteroaryl, heterocycle, a mono-, di-, or polyhydroxyalkyl residue, a mono-, di-, or polyhydroxyaryl residue, or a functional group of less than about 100,000 daltons, and n is an integer ranging from 0 to 4;

CONH(R₂₄), CONHNH(R₂₄), CO(R₂₄), CON(R₂₄)₂, CON(R₂₄)(R₂₅), (CH₂)ₙCONH(R₂₄), (CH₂)ₙCON(R₂₄)₂, (CH₂)ₙCOR₂₄, (CH₂)ₙCON(R₂₄)(R₂₅), (CX₂)ₙCONH(R₂₄), (CX₂)ₙCON(R₂₄)₂, (CX₂)ₙCON(R₂₄)(R₂₅), (CX₂)ₙCOR₂₄, (CH₂)ₙCONHNH(R₂₄), (CX₂)ₙCONHNH(R₂₄), (CHX)ₙCONH(R₂₄), (CHX)ₙCONHNH(R₂₄), (CHX)ₙCO(R₂₄), (CHX)ₙCON(R₂₄)₂, or (CHX)ₙCON(R₂₄)(R₂₅), where X is selected from OH, OR₂₆, SR₂₆, or a halogen, and R₂₄ , R₂₅ and R₂₆ are independently selected from H, NH₂, acetyl, a straight or branched chain C1-C20 alkyl, haloalkyl, haloheteroalkyl, heteroalkyl, aryl, heteroaryl, heterocycle, a mono-, di-, or polyhydroxyalkyl residue, a mono-, di-, or polyhydroxyaryl residue, an amino acid ester, an amino acid amide, a mono-, di-, or polyetheralkyl residue, a mono-, di-, or polyetheraryl residue, or a functional group of less than about 100,000 daltons, and n is an integer ranging from 1 to 4;

S(R₂₇), CH(CH₃)S(R₂₇), (CH₂)ₙS(R₂₇), (CH₂)ₙNH(R₂₇), (CH₂)ₙNHNH(R₂₇), (CH₂)ₙN(R₂₇)₂, (CH₂)ₙN(R₂₇)(R₂₈), (CH₂)ₙN(R₂₇)(R₂₈)(R₂₉)⁺A, CH═N(R₂₇), or CH═NN(R₂₇)(R₂₈), where R₂₇, R₂₈ and R₂₉ are independently selected from H, OH, O-alkyl, NH₂, acetyl, a straight or branched chain C1-C20 alkyl, haloalkyl, heteroalkyl, haloheteroalkyl, aryl, heteroaryl, heterocycle, amino acids (provided —NH(R₂₇) or —N(R₂₇)(R₂₈) is part of the amino acid), a mono-, di-, or polyhydroxyalkyl residue, a mono-, di-, or polyhydroxyaryl residue, a mono-, di-, or polyetheralkyl residue, a mono-, di-, or polyetheraryl residue, or a functional group of less than about 100,000 daltons, where R₂₇, R₂₈ and R₂₉ together may possess the atoms necessary to constitute an aromatic ring system, n is an integer ranging from 0 to 4, and A is a physiologically acceptable counter ion;

(CH₂)ₙOPO(OR₃₀)₂ or (CH₂)ₙPO(OR₃₀)₂, where R₃₀ is selected from H, a physiologically acceptable counter ion, a straight or branched chain C1-C20 alkyl, haloalkyl, heteroalkyl, haloheteroalkyl, aryl, heteroaryl, heterocycle, a mono-, di-, or polyhydroxyalkyl residue, a mono-, di-, or polyhydroxyaryl residue, a mono-, di-, or polyetheralkyl residue, a mono-, di-, or polyetheraryl residue, or a functional group of less than about 100,000 daltons, and n is an integer ranging from 0 to 4;

(CH₂)ₙNHCOR₃₁, or (CH₂)ₙNHNHCOR₃₁, where R₃₁ is selected from a straight or branched chain Cl-C20 alkyl, haloalkyl, heteroalkyl, haloheteroalkyl, aryl, heteroaryl, heterocycle, or a functional group of less than about 100,000 daltons, and n is an integer ranging from 0 to 4;

SO₃R₃₂, SO₂NHR₃₂, SO₂N(R₃₂)₂, SO₂NHNHR₃₃, SO₂R₃₃, SO₃R₃₃, (CH₂)ₙSO₂NHR₃₃, (CH₂)ₙSO₂N(R₃₃)₂, (CH₂)ₙSO₂NHNHR₃₃, or (CH₂)ₙSO₂R₃₃, where R₃₃ is selected from H, OH, a physiologically acceptable counter ion, a straight or branched chain C1-C20 alkyl, A haloalkyl, heteroalkyl, haloheteroalkyl, aryl, heteroaryl, heterocycle, a mono-, di-, or polyhydroxyalkyl residue, a mono-, di-, or polyhydroxyaryl residue, a mono-, di-, or polyetheralkyl residue, a mono-, di-, or polyetheraryl residue, or a functional group of less than about 100,000 daltons, NHR₃₃ can also be an amino acid, an amino acid salt, an amino acid ester residue, an amino acid amide residue, and n is an integer ranging from 1 to 4;

aryl or substituted aryl, which may optionally bear one or more substituents with a molecular weight of less than or equal to about 100,000 daltons;

wherein:

R₁₀ and R₁₃ may form a bond;

R₆ and R₇ may form a ═O; and

R₈ and R₉ may form a ═O;

with the proviso that at least one of R₁ through R₃₃ is a functional group that possesses in part or whole of its structure, a carbamate functionality of the formulae —OCON(R₃₄)₂, —OCON═C(R₃₄)₂, —OCONR₃₄R₃₅ or —OCON═C(R₃₄)(R₃₅), where R₃₄ and R₃₅ are independently selected from H, C1-C20 alkyl, C1-C20 cyclioalkyl, aryl, NH₂, N(CH₃)₂, (CH₂)ₙOH, (CH₂)ₙO-alkyl, (CH₂)ₙOCOCH₃, (CH₂)ₙO(CH₂)ₘOH, (CH₂)ₙO(CH₂)ₘOCOCH₃, (CH₂)ₙO(CH₂)ₘO-alkyl, (CH₂)ₙN((CH₂)ₘOH)₂, (CH₂)ₙN((CH₂)ₘO-alkyl)₂, (CH₂)ₙN((CH₂)ₘO-alkylether)₂, ((CH₂)ₙO)ₘ(CH₂)_{Q}OH, (CH₂)ₙO(CH₂)ₘNH₂, (CH₂)ₙO(CH₂)ₘN(CH₃)₂, (CH₂)ₙO(CH₂)ₘN(CH₃)₃⁺A, (CH₂)ₙN((CH₂)ₘNH₂)₂, (CH₂)ₙN(CH₂)ₘN(CH₃)₂, (CH₂)ₙO-haloalkyl, (CH₂)ₙN((CH₂)ₘN(CH₃)₃⁺A)₂, ((CH₂)ₙO)ₘ(CH₂O)_{Q}COCH₃, an alkylphosphate residue, an alkylsulfonic acid residue, an alkylsulfonic ester or alkylsulfonic amide reside, an alkylmorpholino residue, an alkylheterocyclic residue, an alkylthiol residue, a mono-, di-, or polyhydroxyalkyl residue, a mono-, di-, or polyhydroxyaryl residue, a mono-, di-, or polyetheralkyl residue, a mono-, di-, or polyetheraryl residue, Q, n and m are integers between 0 and 10,000, and A is physiologically acceptable counter ion.

In formulae IVA and IVB, M can be selected from 2H, a metal cation, or photoactive metal ions preferably selected from Ga³⁺, Pt²⁺, Pd²⁺, Sn⁴⁺, In³⁺, Ge⁴⁺, Si⁴+, Al³⁺, Zn²+, Mg²⁺, wherein optionally associated with the metal ion is the appropriate number of physiologically acceptable charge balancing counter ions.

In accordance with the invention, a pharmaceutically acceptable salt, prodrug, solvate, or metabolite of the compounds of formula IVA and IVB is also within the scope of the invention.

Formula V:

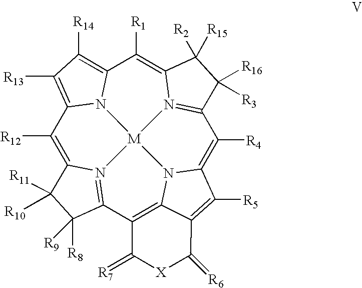

wherein:

R₁, R₂, R₃, R₄, R₅, R₆, R₇, R₈, R₉, R₁₀, R₁₁, R₁₂, R₁₃, R₁₄, R₁₅, R₁₆, are independently selected from the group consisting of:

H, halogen, methyl, ethyl, substituted or unsubstituted C1-C20 alkyl, heteroalkyl, haloalkyl, heterohaloalkyl, cycloalkyl, aryl, substituted aryl, alkenyl, substituted alkenyl, alkynyl, substituted alkynyl, amide, ester, ether, polyether, alkoxy, aryloxy, haloalkoxy, amino, alkylcarbonyloxy, alkoxycarbonyl, aryloxycarbonyl, azo, arylcarbonyloxy, alkoxycarbonyloxy, aryloxycarbonyloxy, sulfinyl, sulfonyl, silil, carbamoyl, heterocyclic, nitro, nitroso, formyloxy, isocyano, cyanate, isocyanate, thiocyanate, isothiocyanate, N(alkyl)₂, N(aryl)₂, CH═CH(aryl), CH═CHCH₂N(CH₃)₂, CH═CHCH₂N⁺(CH₃)₃A, CH═N(alkyl)₂⁺A, N(alkyl)₃⁺A CN, OH, CHO, COCH₃, CO(alkyl), CO₂H, CO₂Na, CO₂K, CH(CH₃)OH, CH(CH₃)O-alkyl, CH(CH₃)O-alkoxy, CH(CH₃)O-aryl, CH(CH₃)NH-alkyl, CH(CH₃)NH-cycloalkyl, CH(CH₃)NH-heteroalkyl, CH(CH₃)NH-heteroalkoxy, CH(CH₃)-(amino acid), CH(CH₃)-(amino acid ester), CH(CH₃)-(amino acid amide), C(X)₂C(X)₃, (where X is H or halogen), CH═NR₁₇ (where R₁₇ is OH, O-alkyl, O-ether, O-alkylamino, NHCOCH₂N(CH₃)₂, NHCOCH₂N(CH₃)₃⁺A, NHCOCH₂-(pyridinium)⁺A, (CH₂)ₙO-alkoxy, or (CH₂)ₙO-alkyl), where n is an integer ranging from 0 to 8, and A is a charge balancing ion;

CO₂R₁₈, where R₁₈ is selected from H, a physiologically acceptable counter ion, a C1-C20 straight or branched chain alkyl, haloalkyl, heteroalkyl, haloheteroalkyl, aryl, heteroaryl, heterocycle, a mono-, di-, or polyhydroxyalkyl residue, a mono-, di-, or polyhydroxyaryl residue, or a functional group of less than about 100,000 daltons;

(CH₂)ₙOH, or (CH₂)ₙOR₁₉, where R₁₉ is selected from alkyl, haloalkyl, heteroalkyl, haloheteroalkyl, aryl, heteroaryl, heterocycle, a protecting group, a mono-, di-, or polyhydroxyalkyl residue, a mono-, di-, or polyhydroxyaryl residue, or a functional group of less than about 100,000 daltons, and n is an integer ranging from 0 to 4;

(CH₂)ₙCO₂R₂₀, (CHX)ₙCO₂R₂₀, or (CX₂)ₙCO₂R₂₀, where X is selected from OH, OR₂₁, or a halogen, and R₂₀ and R₂₁ are independently selected from H, a physiologically acceptable counter ion, acetyl, a straight or branched chain C1-C20 alkyl, haloalkyl, heteroalkyl, haloheteroalkyl, aryl, heteroaryl, heterocycle, a mono-, di-, or polyhydroxyalkyl residue, a mono-, di-, or polyhydroxyaryl residue, or a functional group of less than about 100,000 daltons, and n is an integer ranging from 1 to 4;

CONH(R₂₂), CONHNH(R₂₂), CO(R₂₂), CON(R₂₂)₂, CON(R₂₂)(R₂₃), (CH₂)ₙCONH(R₂₂), (CH₂)ₙCON(R₂₂)₂, (CH₂)ₙCOR₂₂, (CH₂)ₙCON(R₂₂)(R₂₃), (CX₂)ₙCONH(R₂₂), (CX₂)ₙCON(R₂₂)₂, (CX₂)ₙCON(R₂₂)(R₂₃), (CX₂)ₙCOR₂₂, (CH₂)ₙCONHNH(R₂₂), (CX₂)ₙCONHNH(R₂₂), (CHX)ₙCONH(R₂₂), (CHX)ₙCONHNH(R₂₂), (CHX)ₙCO(R₂₂), (CHX)ₙCON(R₂₂)₂, or (CHX)ₙCON(R₂₂)(R₂₃), where X is selected from OH, OR₂₄, SR₂₄, or a halogen, and R₂₂, R₂₃ and R₂₄ are independently selected from H, NH₂, acetyl, a straight or branched chain C1-C20 alkyl, haloalkyl, haloheteroalkyl, heteroalkyl, aryl, heteroaryl, heterocycle, a mono-, di-, or polyhydroxyalkyl residue, a mono-, di-, or polyhydroxyaryl residue, an amino acid ester, an amino acid amide, a mono-, di-, or polyetheralkyl residue, a mono-, di-, or polyetheraryl residue, or a functional group of less than about 100,000 daltons, and n is an integer ranging from 1 to 4;

S(R₂₅), CH(CH₃)S(R₂₅), (CH₂)ₙS(R₂₅), (CH₂)ₙNH(R₂₅), (CH₂)ₙNHNH(R₂₅), (CH₂)ₙN(R₂₅)₂, (CH₂)ₙN(R₂₅)(R₂₆), (CH₂)ₙN(R₂₅)(R₂₆)(R₂₇)⁺A, CH═N(R₂₅), or CH═NN(R₂₅)(R₂₆), where R₂₅, R₂₆ and R₂₇ are independently selected from H, OH, O-alkyl, NH₂, acetyl, a straight or branched chain C1-C20 alkyl, haloalkyl, heteroalkyl, haloheteroalkyl, aryl, heteroaryl, heterocycle, amino acids (provided —NH(R₂₅) or —N(R₂₅)(R₂₆) is part of the amino acid), a mono-, di-, or polyhydroxyalkyl residue, a mono-, di-, or polyhydroxyaryl residue, a mono-, di-, or polyetheralkyl residue, a mono-, di-, or polyetheraryl residue, or a functional group of less than about 100,000 daltons, where R₂₅, R₂₆ and R₂₇ may together possess the atoms necessary to constitute an aromatic ring system, n is an integer ranging from 0 to 4, and A is a physiologically acceptable counter ion;

(CH₂)ₙOPO(OR₂₈)₂, or (CH₂)ₙPO(OR₂₈)₂, where R₂₈ is selected from H, a physiologically acceptable counter ion, a straight or branched chain C1-C20 alkyl, haloalkyl, heteroalkyl, haloheteroalkyl, aryl, heteroaryl, heterocycle, a mono-, di-, or polyhydroxyalkyl residue, a mono-, di-, or polyhydroxyaryl residue, a mono-, di-, or polyetheralkyl residue, a mono-, di-, or polyetheraryl residue, or a functional group of less than about 100,000 daltons, and n is an integer ranging from 0 to 4;

(CH₂)ₙNHCOR₂₉, or (CH₂)ₙNHNHCOR₂₉, where R₂₉ is selected from a straight or branched chain C1-C20 alkyl, haloalkyl, heteroalkyl, haloheteroalkyl, aryl, heteroaryl, heterocycle, or a functional group of less than about 100,000 daltons, and n is an integer ranging from 0 to 4;

SO₃R₃₀, SO₂NHR₃₀, SO₂N(R₃₀)₂, SO₂NHNHR₃₀, SO₂R₃₀, SO₃R₃₀, (CH₂)ₙSO₂NHR₃₀, (CH₂)ₙSO₂N(R₃₀)₂, (CH₂)ₙSO₂NHNHR₃₀, or (CH₂)ₙSO₂R₃₀, where R₃₀ is selected from H, OH, a physiologically acceptable counter ion, a straight or branched chain C1-C20 alkyl, haloalkyl, heteroalkyl, haloheteroalkyl, aryl, heteroaryl, heterocycle, a mono-, di-, or polyhydroxyalkyl residue, a mono-, di-, or polyhydroxyaryl residue, a mono-, di-, or polyetheralkyl residue, a mono-, di-, or polyetheraryl residue, or a functional group of less than about 100,000 daltons, NHR₃₀ can also be an amino acid, an amino acid salt, an amino acid ester residue, or an amino acid amide residue, and n is an integer ranging from 0 to 4;

aryl or substituted aryl, which may optionally bear one or more substituents with a molecular weight of less than or equal to about 100,000 daltons;

wherein:

R₁₅ and R₁₆ may form a bond;

R₉ and R₁₀ may form a bond;

R₂ and R₆ may independently be O or N(R₃₁), where R₃₁ is alkyl;

X is O or N(R₃₂), where R₃₂ is selected from alkyl, an amino acid, an amino acid ester, an amino acid amide, (CH₂)ₙOH, (CH₂)n_{O}-alkyl, (CH₂)ₙOCOCH₃, (CH₂)ₙO(CH₂)ₘOH, (CH₂)ₙO(CH₂)ₘOCOCH₃, (CH₂)ₙO(CH₂)ₘO-alkyl, (CH₂)ₙN((CH₂)ₘOH)₂, (CH₂)ₙN((CH₂)ₘO-alkyl)₂, (CH₂)ₙN((CH₂)ₘO-alkylether)₂, ((CH₂)ₙO)ₘ(CH₂)_{Q}OH, (CH₂)ₙO(CH₂)ₘNH₂, (CH₂)ₙO(CH₂)ₘN(CH₃)₂, (CH₂)ₙO(CH₂)ₘN(CH₃)₃⁺A, (CH₂)ₙN((CH₂)ₘNH₂)₂, (CH₂)ₙN(CH₂)ₘN(CH₃)₂, (CH₂)ₙO-haloalkyl, (CH₂)ₙN(CH₂)ₘN(CH₃)₃⁺A, ((CH₂)ₙO)ₘ(CH₂O)_{Q}COCH₃, a mono-, di-, or polyhydroxyalkyl residue, a mono-, di-, or polyhydroxyaryl residue, a mono-, di-, or polyetheralkyl residue, a mono-, di-, or polyetheraryl residue, and Q, n and m are integers ranging from 0 to 10,000; or a functional group that possesses a carbamate moiety functionality of the formulae —OCON(R₃₃)₂, —OCON═C(R₃₃)₂, —OCONR₃₃R₃₄ or —OCON═C(R₃₃)(R₃₄), where R₃₃ and R34 are as described below, or a functional group having a molecular weight less than or equal to 100,000 daltons;

with the proviso that at least one of R₁ through R₃₀ is a functional group that possesses in part or whole of its structure, a carbamate functionality of the formulae —OCON(R₃₃)₂, —OCON═C(R₃₃)₂, —OCONR₃₃R₃₄ and —OCON═C(R₃₃)(R₃₄), where R₃₃ and R₃₄ are independently selected from H, C1-C20 alkyl, C1-C20 cycloalkyl, aryl, NH₂, N(CH₃)₂, (CH₂)ₙOH, (CH₂)ₙO-alkyl, (CH₂)ₙOCOCH₃, (CH₂)ₙO(CH₂)ₘOH, (CH₂)ₙO(CH₂)ₘOCOCH₃, (CH₂)ₙO(CH₂)ₘO-alkyl, (CH₂)ₙN((CH₂)ₘOH)₂, (CH₂)ₙN((CH₂)ₘO-alkyl)₂, (CH₂)ₙN((CH₂)ₘO-alkylether)₂, ((CH₂)ₙO)ₘ(CH₂)_{Q}OH, (CH₂)ₙO(CH₂)ₘNH₂, (CH₂)ₙO(CH₂)ₘN(CH₃)₂, (CH₂)ₙO(CH₂)ₘN(CH₃)₃⁺A, (CH₂)ₙN((CH₂)ₘNH₂)₂, (CH₂)ₙN(CH₂)ₘN(CH₃)₂, (CH₂)ₙO-haloalkyl, (CH₂)ₙN((CH₂)ₘN(CH₃)₃⁺A)₂, ((CH₂)ₙO)ₘ(CH₂O)_{Q}COCH₃, an alkylphosphate residue, an alkylsulfonic acid residue, an alkylsulfonic ester or alkylsulfonic amide reside, an alkylmorpholino residue, an alkylheterocyclic residue, an alkylthiol residue, a mono-, di-, or polyhydroxyalkyl residue, a mono-, di-, or polyhydroxyaryl residue, a mono-, di-, or polyetheralkyl residue, a mono-, di-, or polyetheraryl residue, and Q, n and m are integers ranging from 0 to 1 (10,000;

In formula V, M can be selected from 2H, a metal cation, or photoactive metal ions preferably selected from Ga³⁺, Pt²⁺, Pd²⁺, Sn⁴⁺, In³⁺, Ge⁴⁺, Si⁴⁺, Al³⁺, Zn²⁺, or Mg²⁺, wherein optionally associated with the metal ion is the appropriate number of physiologically acceptable charge balancing counter ions.

In accordance with the invention, a pharmaceutically acceptable salt, prodrug, solvate, or metabolite of the compounds of formula V is within the scope of the invention.

The invention further provides processes for preparing photosensitizers comprising contacting a tetrapyrrolic precursor containing a hydroxyl group in a solvent with carbonyldiimidazole followed by an amine compound in the presence of solvent to form a compound of formulae I, II, IIIA and IIIB, IVA and IVB or V.

The metal cation of formulae I, II, IIIA, IIIB, IVA, IVB and V may include one of the following: Ag, Al, Au, Cd, Ce, Co, Cr, Cu, Dy, Er, Eu, Fe, Ga, Gd, Ge, Hf, Ho, In, Ir, La, Lu, Mg, Mn, Mg, Mo, Nd, Ni, Pb, Pd, Pr, Pt, Rh, Ru, Sb, Sc, Si, Sm, Tb, Tc, Th, Ti, Tm, U, V, Y, Yb, W, Zn, and Zr, and may be radioactive for scintillation imaging.

Additional advantages of the invention will be set forth in the detailed description that follows, and in part will be obvious from the description or may be learned by practice of the invention. The advantages of the invention can be realized and attained by means of the elements and combinations particularly pointed out in the appended claims.

### DETAILED DESCRIPTION OF THE INVENTION

In accordance with the invention, as embodied and broadly described herein, compounds are provided that are particularly useful as photoactive compounds in photodynamic therapy. The present invention is directed to compounds of formulae I, II, IIIA, IIIB, IVA, IVB and V as described above.

When a human or animal with a disease site is treated with doses of a compound of the present invention and when appropriate light rays or electromagnetic waves are applied, the compound emits light (i.e., it fluoresces). Thereby, the existence, position and size of the tumor can be detected. This is called photodiagnosis.

When the disease site is irradiated with light of a proper wavelength and intensity, the compound is activated to exert a cell killing effect against the tumor. This is called phototherapy.

Compounds intended for photodiagnosis and phototherapy ideally should have the following properties:

(a) non-toxic at normal therapeutic dosage unless and until activated by light;

(b) selectively photoactive;

(c) emit characteristic and detectable fluorescence when light rays or electromagnetic waves are applied;

(d) activated to an extent sufficient to exert a cell killing effect against tumors when irradiated with light rays or when electromagnetic waves are applied; and

(e) easily metabolized or excreted after treatment.

The instant compounds can be used for diagnosis and the therapeutic treatment of a broad range of disease indications including tumors. Examples of tumors include, but are not limited to, gastric cancer, enteric cancer, lung cancer, breast cancer, uterine cancer, esophageal cancer, ovarian cancer, pancreatic cancer, pharyngeal cancer, sarcomas, hepatic cancer, cancer of the urinary bladder, cancer of the upper jaw, cancer of the bile duct, cancer of the tongue, cerebral tumor, skin cancer, malignant goiter, prostatic cancer, cancer of the parotid gland, Hodgkin's disease, multiple myeloma, renal cancer, leukemia, and malignant lymphocytoma. For diagnosis, the sole requirement is that the tumor be capable of selectively fluorescing when exposed to proper light. For treatment, the tumor must be penetrable by the activation energy. For diagnosis, light of shorter wavelength is typically used whereas for therapeutic purposes light of longer wavelength is generally used to permit ready penetration of the tumor tissue. It is necessary that the light rays have sufficient intensity to cause the compounds to fluoresce for diagnosis and to exert a cell killing effect for therapy.

The compounds of the present invention are also useful for the treatment of ophthalmologic disorders such as age-related macular degeneration and choroidal neovascularization; dermatological disorders such as psoriasis; gynecological disorders such as dysfunctional uterine bleeding; urological disorders such as condyloma virus; cardiovascular disorders such as restenosis and atherosclerotic plaques; and for hair removal. One may envisage that normal or diseased tissue on any part of the body may be treated with photodynamic therapy. Thus, normal or abnormal conditions of the hematological system, the lymphatic reticuloendothelial system, the nervous system, the endocrine and exocrine system, the skeletomuscular system including bone, connective tissue, cartilage and skeletal muscle, the pulmonary system, the gastrointestinal system including the liver, the reproductive system, the skin, the immune system, the cardiovascular system, the urinary system, the ocular system and the auditory or olfactory system may be treated.

The source of irradiation for photodiagnosis and phototherapy is not limited, but a laser beam is preferable because intensive light rays in a desired wavelength range can be selectively applied. For example, in photodiagnosis, a compound of the invention can be administered to a human or animal body, and after a certain period of time, light rays can be applied to the part to be examined. When an endoscope can be used for the affected part, such as lungs, gullet, stomach, womb, urinary bladder or rectum, the compounds can be irradiated using the endoscope, and the tumor portion selectively fluoresces. This portion is observed visually, or observed through an adapted fiber scope by eye or on a CRT screen.

In phototherapy, after administration of the dosage, the irradiation can be carried out, for example, by laser light from the tip of quartz fibers. In addition to the irradiation of the surface of the tumor, the internal part of the tumor can be irradiated by inserting the tip of quartz fibers into the tumor. The irradiation can be visually observed or imaged on a CRT screen.

In accordance with the invention, as embodied and broadly described herein, the present inventors discovered that tetrapyrrolic macrocycles containing hydroxyl groups could be converted into a new class of photodynamically active compounds. Not only are these compounds excellent photosensitizers when activated at their absorption wavelengths at early treatment timepoints, but surprisingly they are metabolized in a matter of hours in blood plasma to photoinactive tetrapyrroles. As a result, it has been possible to produce photodynamically active tetrapyrroles that display no normal skin toxicities in rats past 6 hrs, at drug doses up to 4 mg/Kg. Early time point treatments (within 30 min) produce excellent chorriocapillaris closure in the rabbit model (28 day shut down study) that is superior to the currently approved drug Visudyne® (QLT Inc). These results will be described in the experimental section. Thus, the compounds of the invention are particularly valuable, as they potentially make it possible to inject a human patient with the drugs of the invention, treat within a 1 hr timeframe and have little or no skin phototoxicity or occular phototoxicity after a 6 hr time point or earlier (depending on the drug). This would be a distinct advantage clinically and also from a patient care perspective.

### Synthesis of Carbamate Tetrapyrroles

Accordingly, in the one embodiment the present invention relates to processes for producing tetrapyrroles of the formulae I, II, IIIA, IIIB, IVA, IVB, and V. The processes involve contacting the corresponding alcohol substituted tetrapyrrole in a suitable solvent with a coupling reagent like carbonyl diimidazole or p-nitrophenylcarbonate and 4-dimethylaminopyridine, then adding an amine, for a period of time and at a temperature sufficient to form compounds of the formulae I, II, IIIA, IIIB, IVA, IVB and V. The only limitation to the choice of tetrapyrrolic compound used is that it must possess at least one hydroxyl group with which to form the carbamate moiety. Particularly preferred compounds are those derived from chlorophyll or hemoglobin. The following describes the peripheral functional group modification of chemical precursors to compounds of formulae I-V, which may be modified to produce analogs possessing hydroxyl groups.

### Pheophorbides (FIG. 1)

Methyl pheophorbide a is an abundant starting material for the synthesis of derivatized pheophorbides as well as the synthesis of carbamate pheophorbide derivatives. Pheophorbides may be converted to pyrropheophorbides via demethoxycarbonylation of the 10′-ester group. Methyl pheophorbide b, like methyl pheophorbide a except it possesses a formyl group in the 3 position, may also be used according to the invention. FIG. 1 shows the positions for chemical reactivity of methyl pheophorbide a or b according to classical pheophorbide chemistry.

### Chlorin e6 Derivatives (FIG. 1)

Trimethyl ester chlorin e6 is an easily prepared tetrapyrrolic macrocycle derived from methyl pheophorbide. Similar chlorin e6 analog may be synthesized from functionalized pheophorbides. As with pheophorbides, chlorin e6 derivatives possess several functionalities that may be modified chemically to give hydroxy-bearing substituents.

[image]

### Purpurin 18 and purpurin 18 imides

Purpurin 18 is an easily prepared tetrapyrrolic macrocycle derived from methyl pheophorbide. Peripheral groups around the macrocycle have been extensively modified. The synthesis of purpurin 18 imides follows the anhydride ring opening of purpurin 18 by amines, followed by base treatment to form the imide ring. As with pheophorbides, purpurin 18 and purpurin 18 imides possess several functionalities that may be modified chemically to give hydroxy-bearing substituents.

### Benzoporphyrin Derivatives

Benzoporphyrins are commonly prepared from either protoporphyrin IX dimethyl esters or from chlorophyll analogs such as methyl pyrropheophorbide. As with pheophorbides, benzoporphyrin derivatives possess several functionalities that may be modified chemically to give hydroxy-bearing substituents.

### Benzochlorin Derivatives

Benzochlorins are commonly prepared from chlorophyll analogs such as methyl pyrropheophorbide or chlorin e6 (M. Graca H. Vincente, K. M. Smith, J. Org. Chem., 1991, 56, 4407-4418), but are also synthesized from porphyrin analogs (U.S. Pat. Nos. 5,789,586, 5,552,134, and 5,512,559). Such derivatives can be made with functionality that either possesses hydroxyl groups or can be modified chemically to give hydroxy-bearing substituents.

### Porphyrins

The most ubiquitous tetrapyrrolic class found in nature is the porphyrins. Many analogs are derived from Hemin (a hemoglobin extract), for example, hematoporphyrin and protoporphyrin, and may be further functionalized accordingly to produce hydroxylated tetrapyrroles. Alternatively, they may be made synthetically to possess the desired functionality (for example see “Porphyrins and Metalloporphyrins” Ed. K. Smith, Elsevier, 1975, N.Y., “The Porphyrins”, Ed. D. Dolphin, Vol I-V, Academic Press, 1978, and “The Porphyrin Handbook”, Ed. K. Kadish, K. M. Smith, R. Guilard, Academic Press, 1999). In any case, porphyrin derivatives that possess hydroxyl groups are synthetically easy to prepare and abundant in the literature.

### Modification of Peripheral Groups to Give Tetrapyrroles Possessing Hydroxyl Groups

Clearly, it is well recognized in the art that synthetic tetrapyrroles may be produced that possess one or more hydroxyl groups. The following section outlines chemistries that have been used to modify functional groups on tetrapyrroles to produce alcohol-containing moieties.

### Vinyl Group Modification

A large number of tetrapyrrolic macrocycles possess vinyl groups. Vinyl groups (—CH═CH₂) may be treated with 33% HBr/AcOH, which converts the vinyl group to a reactive 1′-bromo ethyl group. The bromine in this intermediate may be replaced via the addition of either water or dialcohols to give the 1-hydroxymethyl tetrapyrroles (—CH(OH)CH₃) or functionalized ether derivatives that may possess an alcohol group (—CH(O—R—OH)CH₃, depending on the alcohol used). Reaction of vinyl groups with TI(NO₂)₃ in methanol, followed by acid hydrolysis yields —CH₂CHO, which on reduction with sodium borohydride, for example, yields the 2-(2-hydroxyethyl) group (CH₂CH₂OH). Oxidation of 1-hydroxymethyl groups with, for example, acetic anhydride/dimethylsulfoxide produces acetyl groups. Vinyl groups may also be treated with either KMnO₄ oxidation, OsO₄/morpholine N-oxide/NalO₄, or more simply by ozonolysis to produce formyl groups.

### Acetyl, Formyl and Ester Groups

Functional groups possessing a ketone moiety (for example formyl, acetyl and esters) may be reduced to give moieties possessing an hydroxyl group. Ester functionalities on tetrapyrroles may be modified to produce alcohol esters, for example, ethylene glycol esters, using standard esterification techniques well known to those skilled in the art. The formation of amides possessing an alcohol moiety is possible (—CONH—R—OH and the like) by reacting the acid moiety with coupling reagents like chloroethylformate, 1,3-dicyclohexylcarbodiimide or carbonyl diimidazole, followed by the aminoalcohol. Alternatively, methyl esters may be reacted with aminoalcohols directly to produce the amide alcohol derivatives. In this way a vast variety of carboxylic amide tetrapyrroles possessing hydroxyl groups may be generated. Schemes 1-6 highlight the types of peripheral modifications that are recognized in the art to produce tetrapyrroles possessing hydroxy groups. Schemes 1-6 only show mono or di-hydroxylated compounds. It should be recognized that poly-hydroxylated molecules can also be made.

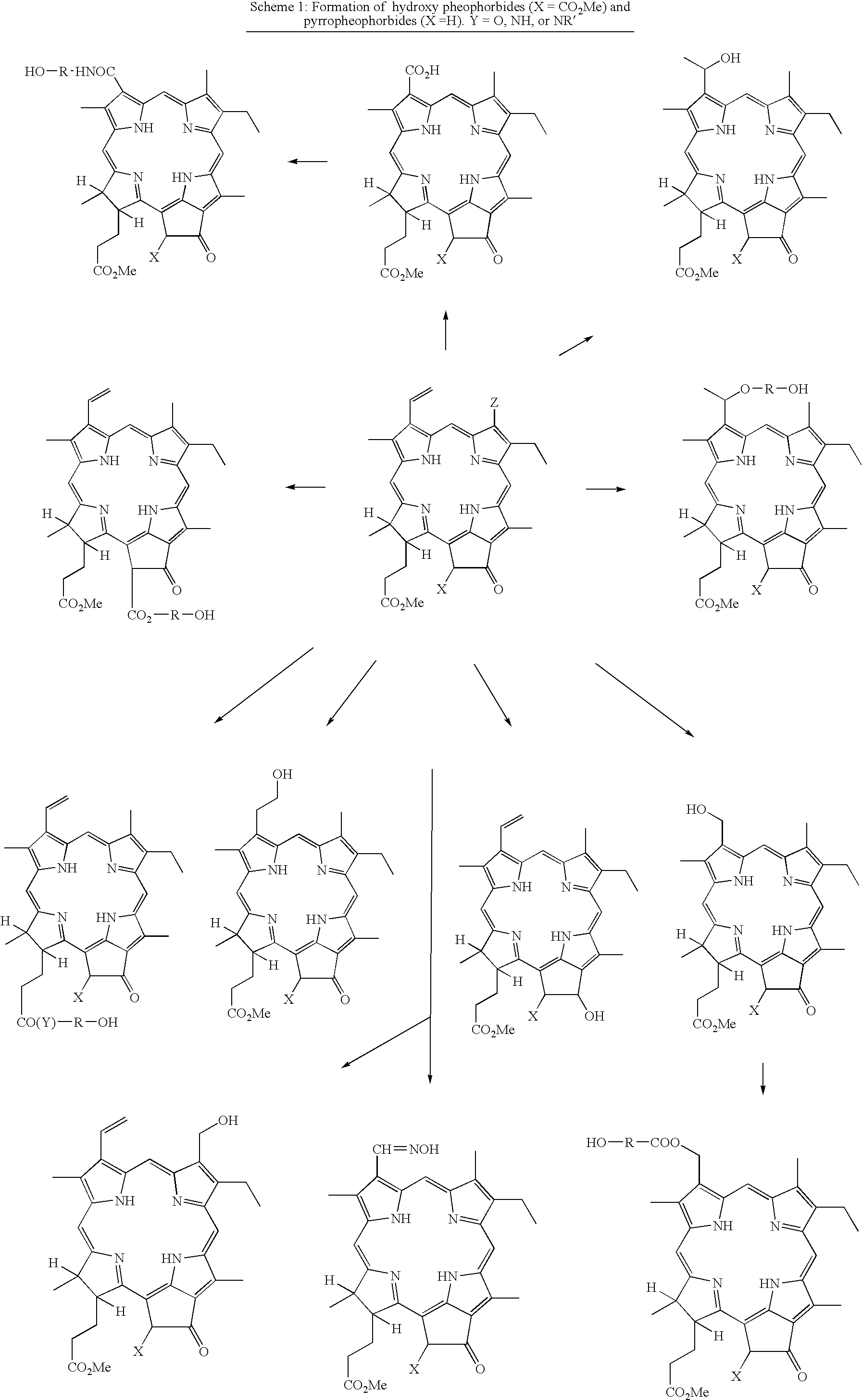

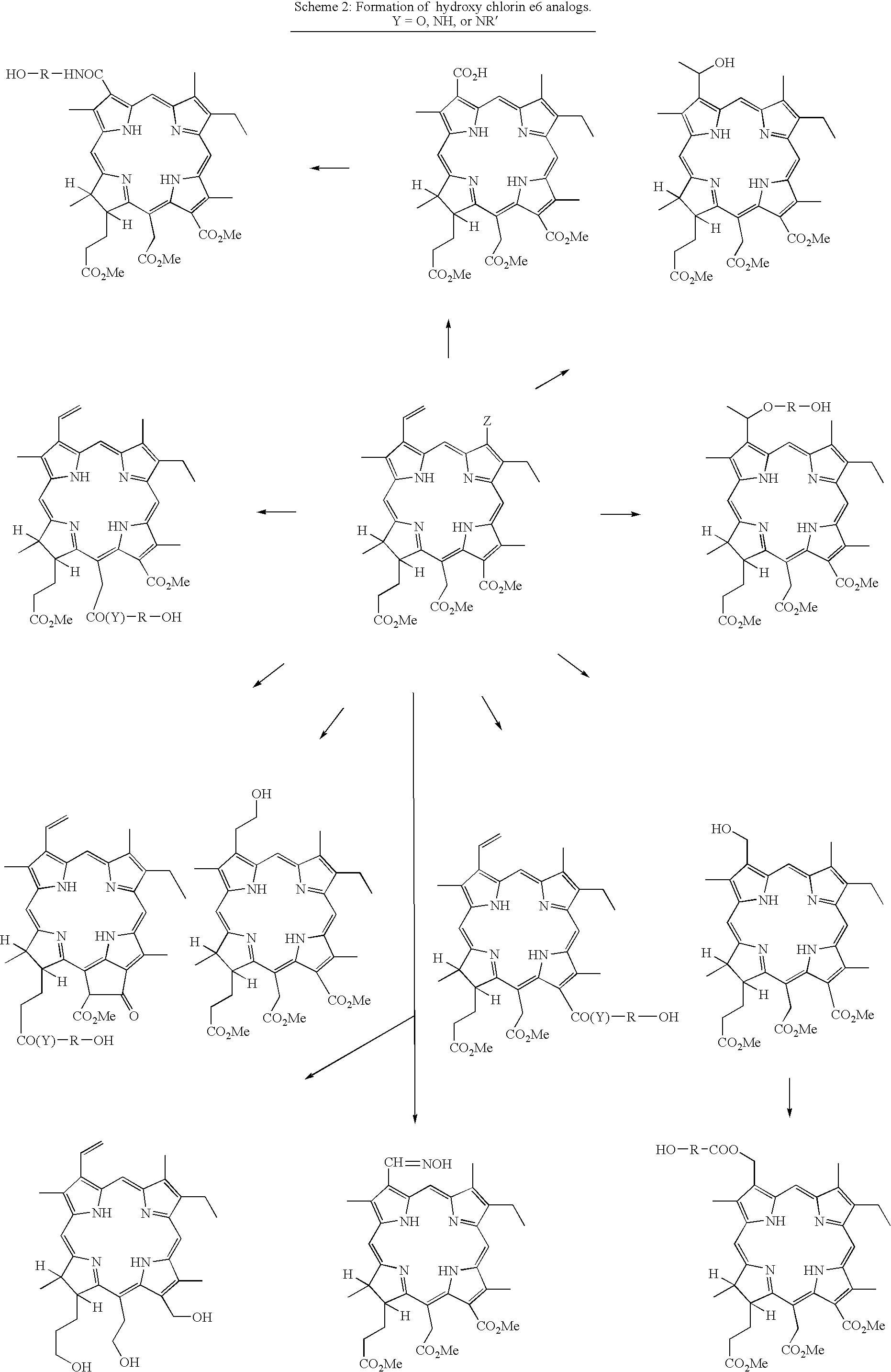

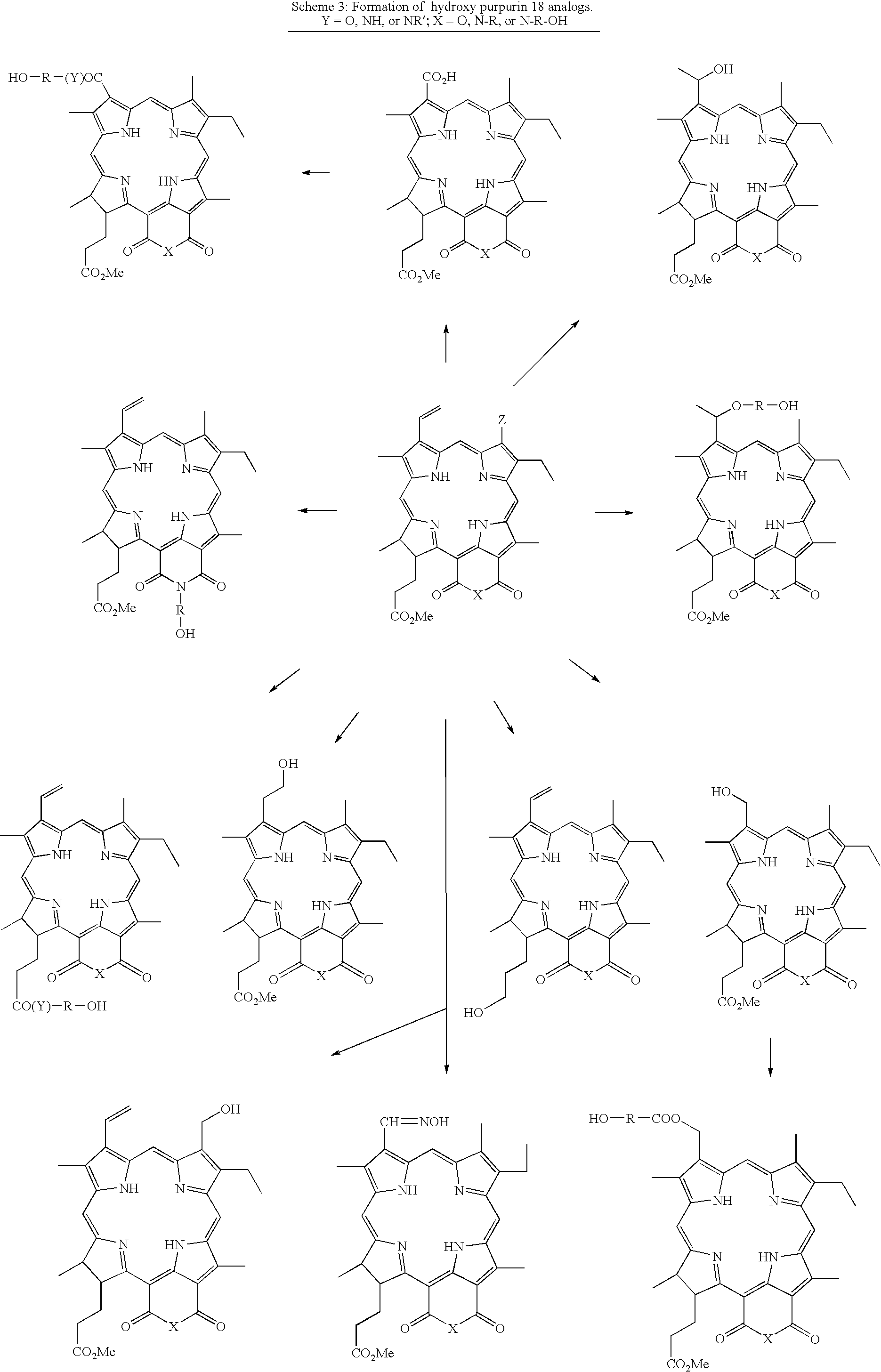

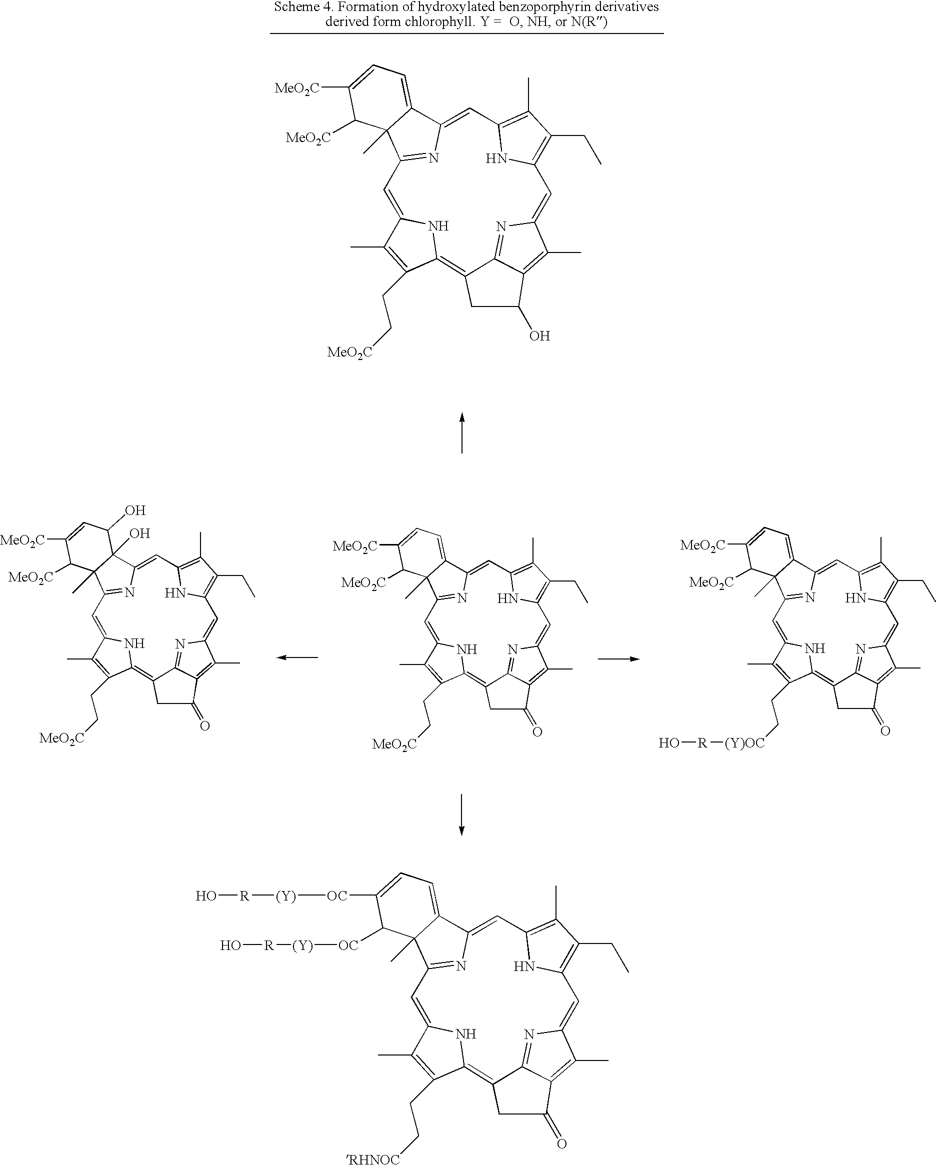

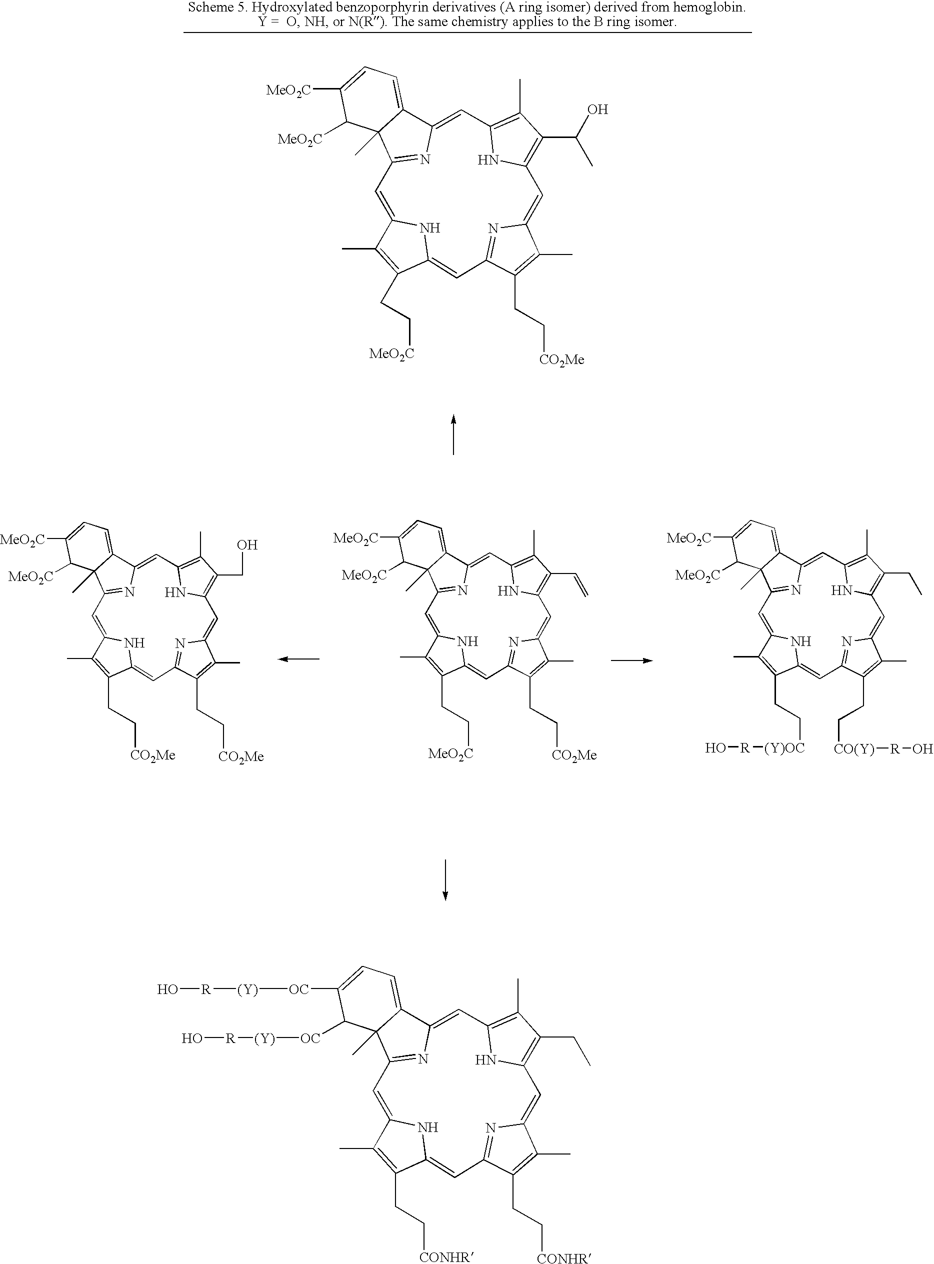

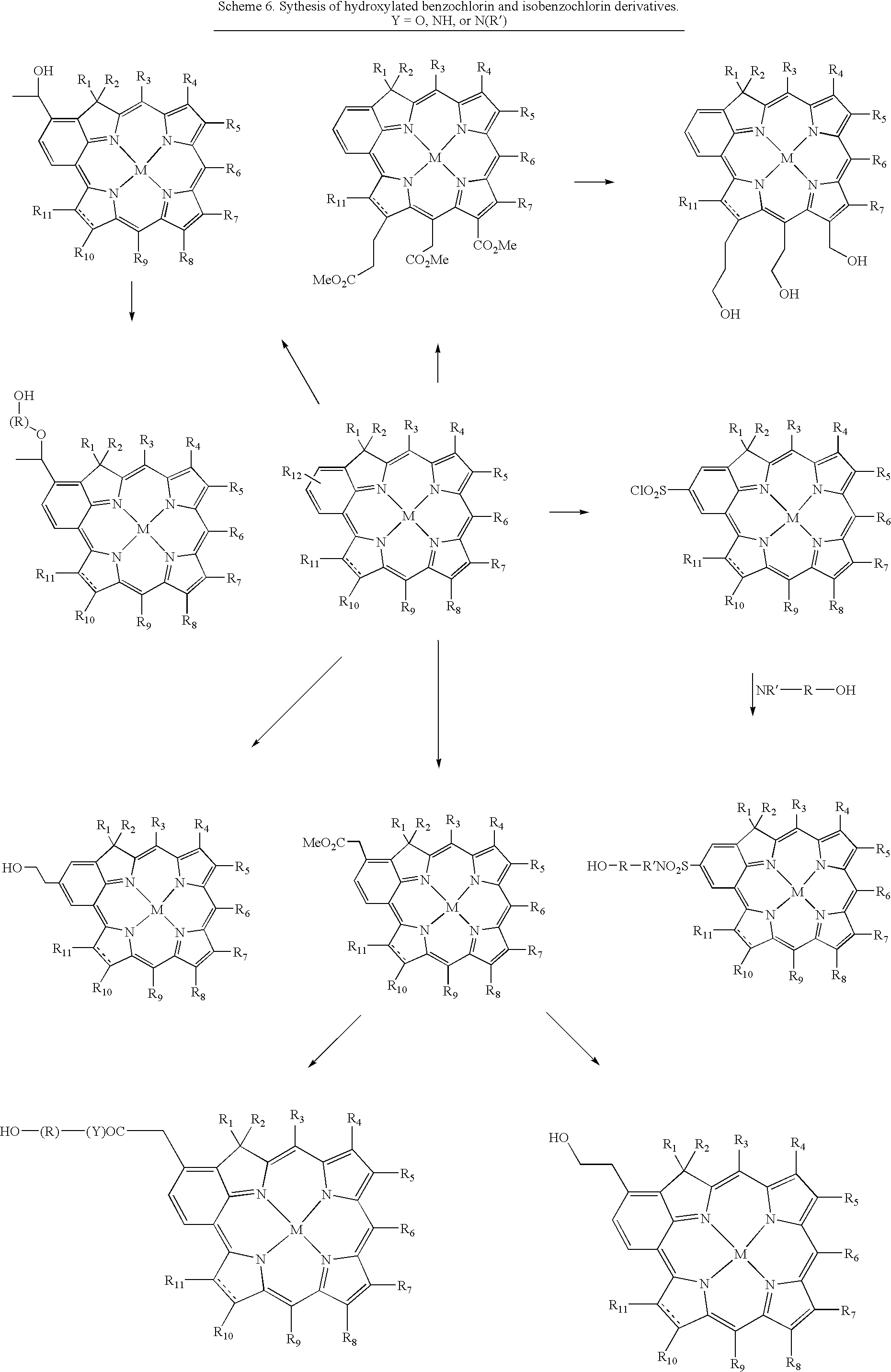

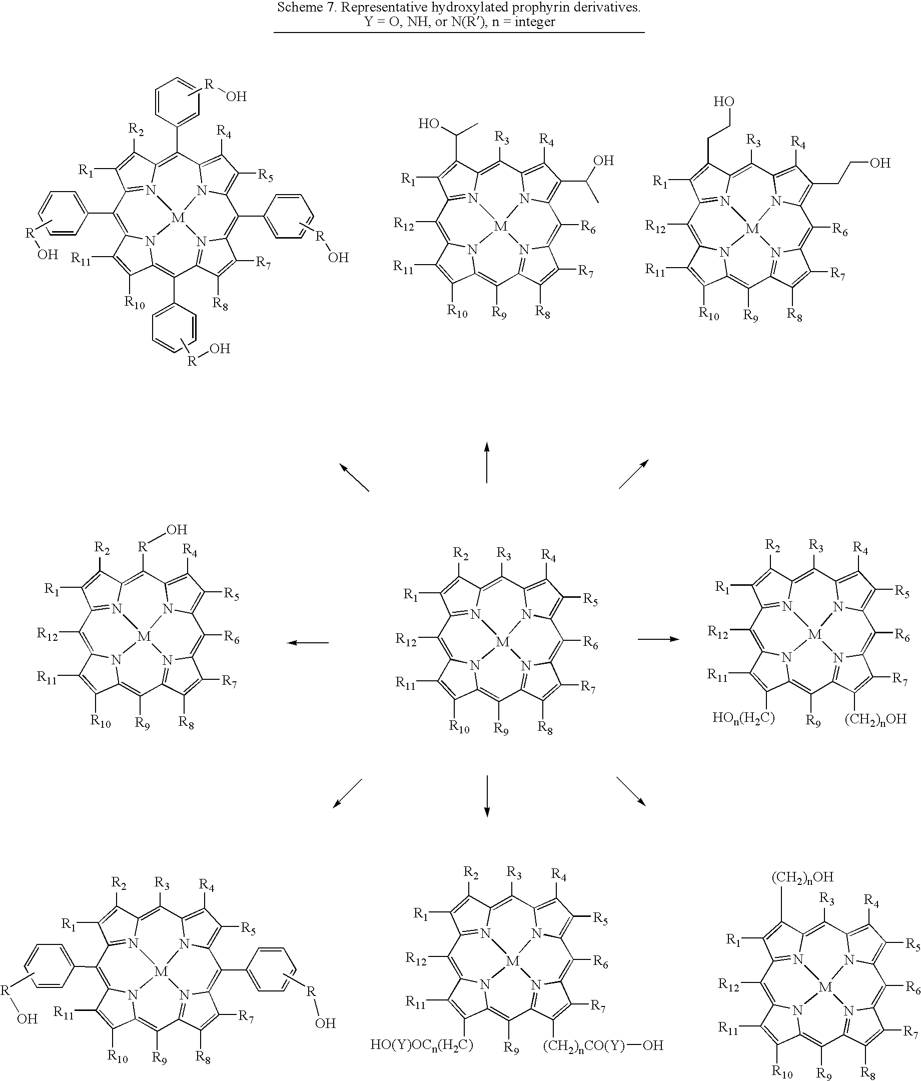

Schemes 1-7 represent chemical modifications that can be made on tetrapyrrolic compounds to produce hydroxylated tetrapyrroles. One or more of these modifications can be carried out on a single molecule if desired. These hydroxylated molecules may then be reacted to form carbamates. The invention thus provides carbamate photosensitizers that are particularly effective in photodynamic therapy. The invention also enables production of compounds that are rapidly metabolized in vivo. Specifically, the invention enables generation of carbamate photosensitizers that are photodynamically or diagnostically active. That is, the carbamate photosensitizers of the invention are capable of inducing a therapeutically acceptable or diagnostic effect at the disease site following light administration, yet metabolize rapidly in blood plasma or cellular components to produce metabolites that are significantly less photodynamically active than the carbamate photosensitizer. Thus, the invention makes it possible to select molecules with hydroxyl groups that are poor photosensitizers in vivo and generate active compounds via functionalization through the carbamate moiety.

The scope of the invention is not limited to tetrapyrrolic molecules. Indeed, any photosensitizer that possesses a hydroxyl group may be converted to a carbamate via the invention. Photosensitizers amenable to the modifications described in the specification or capable of being modified by chemistry well known to those skilled in the art include but are not limited to angelicins, some biological macromolecules such as lipofuscin, photosystem II reaction centers, and D1-D2-cyt b-559 photosystem II reaction centers, chalcogenapyrillium dyes, chlorins, chlorophylls, coumarins, cyanines, ceratin DNA and related compounds such as adenosine, cytosine, 2′-deoxyguanosine-5′-monophosphate, deoxyribonucleic acid, guanine, 4-thiouridine, 2′-thyrnidine 5′-monophosphate, thymidylyl(3′-5′)-2′-deoxyadenosine, thymidylyl(3′-5′)-2′-deoxyguanosine, thymine, uracil, certain drugs such as adriamycin, afloqualone, amodiaquine dihydrochloride, chloroquine diphosphate, chlorpromazine hydrochloride, daunomycin, daunomycinone, 5-iminodaunomycin, doxycycline, furosemide, gilvocarcin M, gilvocarcin V, hydroxychloroquine sulfate, lumidoxycycline, mefloquine hydrochloride, mequitazine, merbromin (Mercurochrome), primaquine diphosphate, quinacrine dihydrochloride, quinine sulfate, tetracycline hydrochloride, certain flavins and related compounds such as alloxazine, flavin mononucleotide, 3-hydroxyflavone, limichrome, limiflavin, 6-methylalloxazine, 7-methylalloxazine, 8-methylalloxazine, 9-methylalloxazine, 1-methyl limichrome, methyl-2-methoxybenzoate, 5-nitrosalicyclic acid, proflavine, riboflavin, fullerenes, metalloporphyrins, phthalocyanines, metallophthalocyanines, texaphyrins, methylene blue derivatives, naphthalimides, naphthalocyanines, certain natural compounds such as bis(4-hydroxy-3-methoxyphenyl)-1,6-heptadiene-3,5-dione, 4-(4-hydroxy-3-methoxyphenyl)-3-buten-2-one, N-formylkynurenine, kynurenic acid, kynurenine, 3-hydroxykynurenine, DL-3-hydroxykynurenine, sanguinarine, berberine, carmane, 5,7,9(11 ),22-ergostatetraene-3 β-ol, nile blue derivatives, NSAIDs (nonsteroidal antiinflammatory drugs), perylenequinones, phenols, pheophorbides, pheophytins, photosensitizer dimers and conjugates, phthalocyanines, sapphyrins, pentaphyrins, porphycenes, porphyrins, psoralens, purpurins. quinones, retinoids, rhodamines, thiophenes, verdins, xanthene dyes (Redmond and Gamlin, Photochem Photobiol, 70(4):391-475 (1 999)).

Exemplary angelicins include but are not limited to the following and derivatives thereof: 3-aceto-angelicin; angelicin; 3,4′-dimethyangelicin; 4,4′-dimethyl angelicin; 4,5-dimethyl angelicin; 6,4′-dimethyl angelicin, 6,4′-dimethyl angelicin; 4,4′,5′-trimethyl angelicin; 4,4′,5′-trimethyl-l′-thioangelicin; 4,6,4′-trimethyl-l′-thioangelicin; 4,6,4′-trimethyl angelicin; 4,6,5′-trimethyl-l′-thioangelicin; 6,4,4′-trimethyl angelicin; 6,4′,5′-trimethyl angelicin; 4,6,4′,5′-tetramethyl-l′-thloangelicin; and 4,6,4′,5′-tetramethyl angelicin.

Exemplary chalcogenapyrillium dyes include but are not limited to the following and derivatives thereof: pyrilium perchlorate, 4,4′-(1,3-propenyl)-bis[2,6-di(l,1-dimethylethyl)]-; pyrilium perchlorate, 2,6-bis(l,1 dimethyl-ethyl)-4-[1-[2,6-bis(l,1-dimethyl-ethyl)selenopyran-4-ylidene]-3-propenyl-; pyrilium hexofluoro phosphate, 2,6-bis-(1,1-dimethyl-ethyl)-selenopyran-4-ylidene; 3-propenyl-; pyrilium hexofluoro phosphate, 2,6-bis(1,1-dimethyl-ethyl)-selenopyran-4-ylidene]-3-propenyl-; pyrilium perchlorate, 2,6-bis(1,1-dimethyl-ethyl)-4-[1-[2,6-bis(1,1-dimethyl-ethyl)telluropyran-4-ylidene]-3-propenyl-; pyrilium hexofluoro phosphate, 2,6-bis(l,1-dimethyl-ethyl)-4-[1-[2,6-bis(1,1-dimethyl-ethyl)telluropyran-4-ylidene]-3-propenyl-; pyrilium perchlorate, 2,6-bis(1,1-dimethyl-ethyl)-4-[1-[2,6-bis(1,1 -dimethyl-ethyl)thiapyran-4-ylidene]-3-propenyl]-; selenopyrilium hexofluoro phosphate, 2,6-bis(l,1-dimethyl-ethyl)-4-[1-[2,6-bis(1,1-dimethyl-ethyl)selenopyran-4-ylidene]-3-propenyl]-; selenopyrilium, 2,6-bis(l,1-dimethylethyl)-4-[1-[2,6-bis(l,1-dimethylethyl)selenopyran-4-ylidene]-3-propenyl]-; selenopyrilium percheorate, 2,6bis(l,l-dimethyl-ethyl)-4-[1-[2,6-bis(1,1-dimethyl-ethyl)4-[1-[2,6-bis(1,1-dimethylethyl)telluropyran4-ylidene]-3-propenyl]-; selenopyrilium hexofluoro phosphate, 2,6-bis(1,1-dimethyl-ethyl)4-[1-[2,6-bis(l,1-dimethyl-ethyl)telluropyran-4-ylidene]-3-propenyl];selenopyriliumhexofluorophosphate,2,6-bis(l,l-dimethyl-ethyl)-4-(2˜[2,6-bis(1,1-dimethyl-ethyl)selenopyran-4-ylidene]-4-(2-butenyl)]-; selenopynlium hexofluorophosphate;2,6-bis(l,1-dimethyl-ethyl)-4-[2-[2,6-bis)l,1-dimethyl-ethyl)selenopyran-4-ylidene]-4-(2-pentenyl)]-; telluropyrilium tetrafluoroborate; 2,6-bis(1,1-dimethylethyl)-4-[1-[2,6-bis(1,1-dimethyl-ethyl)-telluropyran-4-ylidene]-3-propenyl]-; telluropyrilium hexofluoro phosphate, 2,6-bis(1,1 -dimethyl-ethyl)-4-[1-[2,6-bis(1,1 -dimethyl-ethyl)telluropyran4-ylidene]-3-propenyl]-; telluropyrilium hexofluoro phosphate, 2,6-bis(1,1-dimethyl-ethyl)-4-[1-[2,6-bis(1,1-dimethyl-ethyl)telluropyran-4-ylidene]ethyl-; telluropyrilium hexofluoro phosphate, 2,6-bis(1,1-dimethyl-ethyl)-4-[1-[2,6-bis(1,1-dimethyl-ethyl)-telluropyran-4-ylidene]methyl-; thiopyrilium hexofluoro phosphate, 2,6-bis(1,1-dimethyl-ethyl)-4-[1-[2,6-bis(l,l-dimethyl-ethyl)thiopyran-4-ylidene]-3-propenyl]-; thiopyrilium hexofluorophosphate,2,6-bis(l,1-dimethyl-ethyl)-4-[1-[2,6-bis(l,1-dimethyl-ethyl)selenopyran-4-ylidene]3-propenyl]-; and thiopyrilium hexofluoro phosphate, 2,6-bis(1,1-dimethyl-ethyl)-4[1-[2,6-bis(l,1-dimethyl-ethyl)telluropyran-4-ylidene]-3-propenyl]-.

Exemplary chlorin dyes include but are not limited to the following and derivatives thereof: 5-azachlorin dimethyl ester derivatives; 5,10,15,20-tetrakis-(m-hydroxyphenyl)bacteriochlorin; benzoporphyrin derivative monoacid ring A; benzoporphyrin derivative monoacid ring-A; porphine-2.18-dipropanoic acid, 7-[2-dimethyl-amino)-2-oxoethyl]-8-ethylidene-7,8-dihydro-3,7,12,17-tetramethyl, dimethylester; porphine-2,18-dipropanoic acid, 7-[2-dimethylamino)-2-oxoethyl]-8-ethylidene -7,8-dihydro-3,7,12,17-tetramethyl, dimethylester Z; porphine-2,18-dipropanoic acid, 7-[2-dimethyl-amino)-2-oxoethyl]-8-ethyl-7,8-dihydro-3,7,12,17-tetramethyl, dimethylester Z; porphine-2,18-dipropanoic acid, 7-[2-dimethylamino)-2-oxoethyl]-8-n-heptyl-7,8-dihydro-3,7,12,17-tetramethyl, dimethylester; tin (II) porphine-2,18-dipropanoic acid, 7-[2-(dimethylamino-2-oxoethyl]-8-n-heptyl-7,8-dihydro-3,7,12,17-tetramethyl, dimethylester; chlorin e6; chlorin e6 dimethyl ester; chlorin e6 Ka; chlorin e6 monomethyl ester; chlorin e6 Na; chlorin p6; chlorin p6-trimethylester; chlorin derivative zinc (II) porphine-2,18-dipropanoic acid, 7-[2-(dimethylamino)-2-oxoethyl]-8-n-heptyl-7,8-dihydro-3 7,12,17-tetramethyl, dimethylester; 13′-deoxy-20-formyl-vic-dihydroxy-bacteriochlorin di-tert-butyl aspartate; 13′-deoxy-20-formyl-4-keto-bacteriochlorin di-tert-butyl aspartate; di-L-aspartyl chlorin e6; mesochlorin; 5,10,15,20-tetrakis-(m-hydroxyphenyl)chlorin; meta-(tetrahydroxyphenyl)chlorin; methyl-13′-deoxy-20-formyl-4-keto-bacteriochlorin; mono-L-aspartyl chlorin e6; photoprotoporphyrin IX dimethyl ester; phycocyanobilin dimethyl ester; protochlorophyllide a; tin (II) chlorin e6; tin chlorin e6; tin L-aspartyl chlorin e6; tin octaethyl-benzochlorin; tin (IV) chlorin; zinc chlorin e6; and Zinc L-aspartyl chlorin e6.

Exemplary chlorophyll derived photosensitizers include but are not limited to the following or derivatives thereof: chlorophyll a, chlorophyll b; oil soluble chlorophyll; bacteriochlorophyll a; bacteriochlorophyll b; bacteriochlorophyll c; bacteriochlorophyll d; protochlorophyll; protochlorophyll a; amphiphilic chlorophyll derivative 1; and amphiphilic chlorophyll derivative 2.

Exemplary coumarins include but are not limited to the following or derivatives thereof: 3-benzoyl-7-methoxycoumarin; 7-diethylamino-3-thenoylcoumarin; 5,7-dimethoxy-3-(1-naphthoyl)coumarin; 6-methylcoumarin; 2H-selenolo[3,2-g][1]benzopyran-2-one; 2H-selenolo[3,2-g][1]benzothiopyran-2-one;7H-selenolo[3,2-g][1]benzoseleno-pyran-7-one; 7H-selenopyrano[3,2-f][1]benzofuran-7-one; 7H-selenopyrano[3,2-f][1]benzo-thiophene-7-one; 2H-thienol[3,2-g][1]benzopyran-2-one; 7H-thienol[3,2-g][1]benzothiopyran-7-one; 7H-thiopyrano[3,2-fl [1]benzofuran-7-one; coal tar mixture; khellin; RG 708; RG277; and visnagin.

Exemplary cyanines include but are not limited to the following or derivatives thereof: benzoselenazole dye; benzoxazole dye; 1,1′-diethy˜toxacarbocyanine; 1,1′-diethyloxadicarbocyanine; 1,1′-diethylthiacarbocyanine; 3,3′-dialkylthiacarbocyanines (n=2-18); 3,3′-diethylthiacarbocyanine iodide; 3,3′-dihexylselenacarbocyanine; kryptocyanine; MC540 benzoxazole derivative; MC540 quinoline derivative; merocyanine 540; and meso-ethyl, 3,3′-dihexylselenacarbocyanine.

Exemplary fullerenes include but are not limited to the following and derivatives thereof: C60; C70; C76; dihydro-fullerene; 1,9-(4-hydroxycyclohexano)-buckminster-fullerene; [1-methyl-succinate-4-methyl-cyclohexadiene-2,3]-buckminster-fullerene; and tetrahydro fullerene.

Exemplary metalloporphyrins or texaphyrins include but are not limited to the following and derivatives thereof: cadmium (II) chlorotexaphyrin nitrate; LuTex; Antrin; cadmium (II) meso-diphenyl tetrabenzoporphyrin; cadmium meso-tetra-(4-N-methylpyridyl)-porphine; cadmium (II) texaphyrin; cadmium (II) texaphyrin nitrate; cobalt meso-tetra-(4-N-methylpyridyl)porphine; cobalt (II) meso(4-sulfonatophenyl)porphine; copper hematoporphyrin; copper meso-tetra-(4-N-methylpyridyl)-porphine; copper (II) meso(4-sulfonatophenyl)porphine; Europium (III) dimethyltexaphyrin dihydroxide; gallium tetraphenylporphyrin; iron meso-tetra(4-N-methylpyridyl)porphine; lutetium (III) tetra(N-methyl-3-pyridyl )-porphyrin chloride; magnesium (II) meso-diphenyl-tetrabenzoporphyrin; magnesium tetrabenzoporphyrin; magnesium tetraphenylporphyrin; magnesium (II) meso(4-sulfonatophenyl)-porphine; magnesium (II) texaphyrin hydroxide metalloporphyrin; magnesium meso-tetra-(4-N-methylpyridyl)porphine; manganese meso-tetra-(4-N-methyl pyridyl)porphine; nickel meso-tetra(4-N-methylpyridyl)porphine; nickel (II) meso-tetra(4-sulfonatophenyl)porphine; palladium (II) meso-tetra-(4-N-methylpyridyl)-porphine; palladium meso-tetra-(4-N-methylpyridyl)-porphine; palladium tetraphenylporphyrin; palladium (II) meso(4-sulfonatophenyl)-porphine; platinum (II) meso(4-sulfonatophenyl)-porphine; samarium (II) dimethyltexaphyrin dihydroxide; silver (II) meso(4-sulfonatophenyl)porphine; tin (IV) protoporphyrin; tin (IV) meso-tetra-(4-N-methylpyridyl)-porphine; tin meso-tetra(4-sulfonatophenyl)-porphine; tin (IV) tetrakis (4-sulfonatophenyl)porphyrin dichloride; zinc (II) 15-aza-3,7,12,18-tetramethyl-porphyrinato-1,3,17-diyl-dipropionic acid-dimethylester; zinc (II) chlorotexaphyrin chloride; zinc coproporphyrin III; zinc (II) 2,11,20,30-tetra-(1,1-dimethyl-ethyl)tetranaphtho(2,3b:2′,3′-g:2″3″-I:2′″3′″-q)porphyrazine; zinc (II) 2-(3-pyridyloxy)benzo[b]-10,19,28-tri(1,1-dimethylethyl)trinaphtho[2′,3′-g:2″3″1::2′″,3′″-q]porphyrazine; zinc (II) 2,18-bis-(3-pyridyloxy)dibenzo[b,l]-10,26-di(1,1-dimethyl-ethyl)dinaphtho[2′,3′-g:2′″,3′″-q]porphyrazine; zinc (II) 2,9-bis-(3-pyridyloxy)dibenzo[b,g]-17,26-di(1,1-dimethyl-ethyl)dinaphtho[2″,3″-1:2′″,3′″-q]porphyrazine; zinc (II) 2,9,16-tris-(3-pyridyloxy)tribenzo[b,g,l]-24-(1,1-dimethyl-ethyl)naphtho[2′″,3′″-q]porphyrazine; zinc (II) 2,3-bis-(3-pyridyloxy)benzo[b]-10,19,28-tri(1,1-dimethyl-ethyl)trinaphtho[2′,3′-g:2″,3″1:2′″,3′″-q]porphyrazine; zinc (II) 2,3,18,19-tetrakis-(3-pyridyloxy)dibenzo[b,l]-10.26-di(1,1-dimethyl-ethyl)trinaphtho[2′,3′-g:2′″,3′″-q]porphyrazine; zinc (II) 2,3,9,10-tetrakis-(3-pyridyloxy)dibenzo[b,g]-17,26-di(l,1-dimethyl-ethyl)dinaphtho[2″,3″-1:2′″,3′″-q]porphyrazine; zinc (II) 2,3,9,10,16,17-hexakis(3-pyridyloxy)-tribenzo(b,g,l]-24-(1,1-dimethyl-ethyl)naphtho[2′″,3′″-q]porphyrazine; zinc (II) 2-(3-N-methyl)pyridyloxy)benzo[b]-10,19,28-tri(1,1-dimethyl-ethyl)trinaphtho[2′,3′-g:2″,3″1:2′″,3′″-q]porphyrazine monoiodide; zinc (II) 2,18-bis-(3-(N-methyl)pyridyloxy)dibenzo[b,l]-10,26-di(1,1-dimethviethyl)dinaphtho[2′,3′-g:2′″,3′″-q]porphyrazine diiodide; zinc (II) 2,9-bis-(3-(N-methyl)pyridyloxy)dibenzo[b,g]-17,26-di(1,1-dimethylethyl)dinaphtho[2″,3″-1:2′″,3′″-q]porphyrazine diiodide; zinc (II) 2,9,16-tris-(3-(N-methylpyridyloxy)tribenzo[b,g,l]-24-(1,1-dimethylethyl)naphtho[2′″,3′″-q]porphyrazine triiodide; zinc (II) 2,3-bis-(3-(N-methyl)pyridyloxy)benzo[b]-10,19,28-tri(1,1-dimethylethyl)trinaphtho[2′,3′-g:2″,3″-l:2′″,3′″-q]porphyrazine diiodide; zinc (II) 2,3,18,19-tetrakis-(3-(N-methyl)pyridyloxy)dibenzo[b,l]-10,26-di(1,1-dimethyl)dinaphtho[2′,3′-g:2′″,3′″-q]porphyrazine tetraiodide; zinc (II) 2,3,9,10-tetrakis-(3-(N-methyl)pyridyloxy)dibenzo[g,g]-17,26-di(1,1-dimethylethyl)dinaphtho[2″,3″-l:2′″,3′″-q]porphyrazine tetraiodide; zinc (II) 2,3,9,10,16,17-hexakis-(3-(N-methy;)pyridyloxy)tribenzo(b,g,1]-24-(1,1-dimethylethyl)naphthol[2′″,3′″-q]porphyrazine hexaiodide; zinc (II) meso-diphenyl tetrabenzoporphyrin; zinc (II) meso-triphenyl tetrabenzoporphyrin; zinc (II) meso-tetrakis-(2,6-dichloro-3-sulfonatophenyl)porphyrin; zinc (H) meso-tetra-(4-Nmethylpyridyl)-porphine; zinc (II) 5,10,15,20-meso-tetra(4-octylphenylpropynyl)-porphine; zinc porphyrin c; zinc protoporphyrin; zinc protoporphyrin IX; zinc (II) meso-triphenyl-tetrabenzoporphyrin; zinc tetrabenzoporphyrin; zinc (II) tetrabenzoporphyrin; zinc tetranaphthaloporphyrin; zinc tetraphenylporphyrin; zinc (II) 5,10,15,20-tetraphenylporphyrin; zinc (II) meso-(4-sulfonatophenyl)-porphine; and zinc (II) texaphyrin chloride, gallium deuteroporphyrin, gallium deuteroporphyrin dimethyl ester.

Exemplary metallophthalocyanines include but are not limited to the following and derivatives thereof: aluminum mono-(6-carboxypentyl-amino-sulfonyl)-trisulfo-phthalocyanine; aluminum di-(6-carboxy-pentylamino-sulfonyl)-trisulfophthalocyanine; aluminum (III) octa-n-butoxy phthalocyanine; aluminum phthalocyanine; aluminum (III) phthalocyanine disulfonate; aluminum phthalocyanine disulfonate; aluminum phthalocyanine disulfonate (cis isomer); aluminum phthalocyanine disulfonate (clinical prep.); aluminum phthalocyanine phthalimido-methyl sulfonate; aluminum phthalocyanine sulfonate; aluminum phthalocyanine trisulfonate; aluminum (III) phthalocyanine trisulfonate; aluminum (III) phthalocyanine tetrasulfonate; aluminum phthalocyanine tetrasulfonate; chloroaluminum phthalocyanine; chloroaluminum phthalocyanine sulfonate; chloroaluminum phthalocyanine disulfonate; chloroaluminum phthalocyanine tetrasulfonate; chloroaluminum-t-butyl-phthalocyanine; cobalt phthalocyanine sulfonate; copper phthalocyanine sulfonate; copper (II) tetra-carboxy-phthalocvanine; copper (II)-phthalocyanine; copper i-butyl-phthalocyanine; copper phthalocyanine sulfonate; copper (II) tetrakis-methylene-thio[(dimethylamino)methylidyne]lphthalocyanine tetrachloride; dichlorosilicon phthalocyanine; gallium (III) octa-n-butoxy phthalocyanine; gallium (II) phthalocyanine disulfonate; gallium phthalocyanine disulfonate; gallium phthalocyanine tetrasulfonate-chloride; gallium(II) phthalocyanine tetrasulfonate; gallium phthalocyanine trisulfonatechloride; gallium (II) phthalocyanine trisulfonate; GaPcS₁tBu₃; GaPcS₂tBu₂; GaPcS₃tBu; germanium (IV) octa-n-butoxy phthalocyanine; germanium phthalocyanine derivative; silicon phthalocyanine derivative; germanium (IV) phthalocyanine octakis-alkoxy-derivatives; iron phthalocyanine sulfonate; lead (II) 2,3,9,10,16,17,23,24-octakis-(3,6-dioxaheptyloxy)phthalocyanine; magnesium t-butylphthalocyanine; nickel (II) 2,3,9,10,16,17,23,24-octakis(3,6-dioxaheptyloxy)phthalocyanine; palladium (II) octa-n-butoxy phthalocyanine; palladium (II) tetra(t-butyl)-phthalocyanine; (diol)(t-butyl)₃-phthalocyanato palladium(II); ruthenium(II) dipotassium(bis(triphenyl-phosphine-monosulphonate)phthalocyanine; silicon phthalocyanine bis(tri-ii-hexyl-siloxy)-; silicon phthalocyanine bis(tri-phenyl-siloxy)-; HOSiPcOSi(CH₃)₂(CH₂)₃N(CH₃)₂; HOSiPcOSi(CH₃)₂(CH₂)₃N(CH₂CH₃)₂; SiPc[OSi(CH₃)₂(CH₂)₃N(CH₃)₂]₂; SiPc[OSi(CH₃)₂(CH₂)₃N(CH₂CH₃)(CH₂)₂N(CH₃)₂]₂; tin (IV) octa-n-butoxy phthalocyanine; vanadium phthalocyanine sulfonate; zinc (II) octa-n-butoxy phthalocyanine; zinc (II) 2,3,9,10,16,17,23,24-octakis(2-ethoxy-ethoxy) phthalocyanine; zinc (II) 2,3,9,10,16,17,23,24-octakis-(3,6-dioxaheptyloxy) phthalocyanine; zinc (II) 1,4,8,11,15,18,22,25-octa-n-butoxy-phthalocyanine; Zn(II)phthalocyanine-octabutoxy; Zn(II)-phthalocyanine; zinc phthalocyanine; perdeuterated zinc phthalocyanine, zinc (II) phthalocyanine disulfonate; zinc phthalocyanine disulfonate; zinc phthalocyanine sulfonate; zinc phthalocyanine tetrabromo-; zinc (II) phthalocyanine tetra-t-butyl-; zinc (II) phthalocyanine tetra-(t-butyl)-; zinc phthalocyanine tetracarboxy-; zinc phthalocvanine tetrachloro-; zinc phthalocyanine tetrahydroxyl; zinc phthalocyanine tetraiodo-; zinc (II) tetrakis-(1,1-dimethyl-2-phthalimido)ethyl phthalocyanine: zinc (II) tetrakis-(1,1-dimethyl-2-amino)-ethyl-phthalocvanine: zinc (II) phthalocyanine tetrakis(1,1-dimethyl-2-trimethyl ammonium)ethyl tetraiodide; zinc phthalocyanine tetrasulphonate; zinc phthalocyanine tetrasulfonate; zinc (II) phthalocyanine tetrasulfonate; zinc (II) phthalocyanine trisulfonate; zinc phthalocyanine trisulfonate; zinc (II) (t-butyl)₃-phthalocyanine diol; zinc tetradibenzobarreleno-octabutoxyphthalocyanine; zinc (II) 2,9,16,23,-tetrakis-(3-(N-methyl)pyridyloxy)phthalocyanine tetraiodide; and zinc (II) 2,3,9,10,16,17,23,24-octakis-(3-(N-methyl)pyridyloxy)phthalocyanine complex octaiodide; and zinc (II) 2.3,9,10,16,17,23,24-octakis-(3-pyridyloxy)phthalocyanine.

Exemplary methylene blue derivatives include but are not limited to the following and derivatives thereof: 1-methyl methylene blue; 1,9-dimethyl methylene blue; methylene blue; methylene blue; methylene violet; bromomethylene violet; 4-iodomethylene violet; 1,9-dimethyl-3-dimethyl-amino-7-diethyl-amino-phenothiazine; and 1,9-dimethyl-3-diethylamino-7-dibutyl-amino-phenothiazine.

Exemplary naphthalimide blue derivatives include but are not limited to the following and derivatives thereof: NN′-bis-(hydroperoxy-2-methoxyethyl)-1,4,5,8-naphthaldiimide; N-(hydroperoxy-2-methoxyethyl)-l,8-naphthalimide; 1,8-naphthalimide; N,N′-bis(2,2-dimethoxyethyl)-1,4,5,8-naphthaldiimide; and N,N′-bis(2,2-dimethylpropyl)-1,4,5,8-naphthaldiimide.

Exemplary naphthalocyanines include aluminum t-butyl-chloronaphthalocyanine; silicon bis(dimethyloctadecylsiloxy)-2,3-naphthalocyanine; silicon bis(dimethyloctadecylsiloxy)naphthalocyanine; silicon bis(dimethylhexylsiloxy)-2,3-naphthalocyanine; silicon bis(dimethylhexylsiloxy)naphthalocyanine; silicon bis(t-butyldimethylsiloxy)-2,3-naphthalocyanine; silicon bis(tert-butyldimethylsiloxy)naphthalocyanine; silicon bis(tri-n-hexylsiloxy)-2,3-naphthalocyanine; silicon bis(tri-n-hexylsiloxy) naphthalocyanine-, silicon naphthalocyanine; t-butylnaphthalocyanine; zinc (II) naphthalocyanine; zinc (II) tetraacetyl-amidonaphthalocyanine; zinc (II) tetraaminonaphthalocyanine; zinc (II) tetrabenzamidonaphthalocyanine; zinc (II) tetrahexylamidonaphthalocyanine; zinc (II) tetramethoxy-benzamidonaphthalocyanine; zinc (II) tetramethoxynaphthalocyanine; zinc naphthalocyanine tetrasulfonate; and zinc (II) tetradodecylamidonaphthalocyanine.

Exemplary nile blue derivatives include but are not limited to the following and derivatives thereof: benzo[a]phenothiazinium; 5-amino-9-diethylamino-; benzo[a]phenothiazinium; 5-amino-9-diethylamino-6-iodo-; benzo[a]phenothiazinium; 5-benzylamino-9-diethylamino-; benzo[a]phenoxazinium; 5-amino-6,8-dibromo-9-ethylamino-; benzo[a]phenoxazinium; 5-amino-6,8-diiodo-9-ethylamino-; benzo[a]phenoxazinium; 5-amino-6-bromo-9-diethylamino-; benzo[a]phenoxazinium; 5-amino-9-diethylamino-(nile blue A); benzo[a]phenoxazinium; 5-amino-9-diethylamino-2,6-diiodo-1-benzo[a]phenoxazinium; 5-amino-9-diethylamino-2,-iodo; benzo[a]phenoxazinium; 5-amino-9-diethylamino-6-iodo-; benzo[a]phenoxazinium; 5-benzylamino-9-diethylamino-(nile blue 2B); 5-ethylamino-9-diethylamino-benzo[a]-phenoselenazinium chloride; 5-ethylamino-9-diethyl-aminobenzo[a]-phenothiazinium chloride; and 5-ethylamino-9-diethyl-aminobenzo[a]-phenoxazinium chloride.

Exemplary NSAIDs (nonsteroidal anti-inflammatory drugs) include but are not limited to the following and derivatives thereof: benoxaprofen; carprofen; carprofen dechlorinated (2-(2-carbazolyl)propionic acid); carprofen (3-chlorocarbazole); chlorobenoxaprofen; 2,4-dichlorobenoxaprofen; cinoxacin; ciprofloxacin; decarboxy-ketoprofen; decarboxy-suprofen; decarboxy-benoxaprofen; decarboxy-tiaprofenic acid; enoxacin; fleroxacin; fleroxacin-N-oxide;flumequine; indoprofen; ketoprofen; lomelfloxacin; 2-methyl-4-oxo-2H-1,2-benzothiazine-1,1-dioxide; N-demethyl fleroxacin; nabumetone; nalidixic acid; naproxen; norfloxacin; ofloxacin; pefloxacin; pipemidic acid; piroxicarn; suprofen; and tiaprofenic acid.

Exemplary perylenequinones include but are not limited to the following and derivatives thereof: hypericins such as hypericin; hypericin monobasic sodium salt; di-aluminum hypericin; di-copper hypericin; gadolinium hypericin; terbium hypericin, hypocrellins such as acetoxy hypocrellin A; acetoxy-hypocrellin B; acetoxy iso-hypocrellin A; acetoxy iso-hypocrellin B; 3,10-bis-[2-(2-aminoethylamino)ethanol]hypocrellin B; 3,10-bis-[2-(2-aminoethyl)morpholine]hypocrellin B; 3,10-bis[4-(2-aminoethyl)morpholine]hypocrellin B; n-butylaminated hypocrellin B; 3,10-bis(butylamine)hypocrellin B; 4,9-bis(butylamine)hypocrellin B; carboxylic acid hypocrellin B; cystamine-hypocrellin B; 5-chloro hypocrellin A or 8-chloro hypocrellin A; 5-chloro hypocrellin B or 8-chloro hypocrellin B; 8-chloro hypocrellin B; 8-chloro hypocrellin A or 5-chloro hypocrellin A; 8-chloro hypocrellin B or 5-chloro hypocrellin B; deacetylated aldehyde hypocrellin B; deacetylated hypocrellin B; deacetylated hypocrellin A; deacylated, aldehyde hypocrellin B; demethylated hypocrellin B; 5,8-dibromo hypocrellin A; 5,8-dibromo hypocrellin B; 5,8-dibromoiso-hypocrellin B; 5,8-dibromo[1,12-CBr═CMeCBr(COMe)]hypocrellin B; 5,8-dibromo[1,12-CHBrC(═CH₂)CBr(COMe))hypocrellin B; 5,8-dibromo[1-CH₂COMe, 12-COCOCH₂Br-]hypocrellin B; 5,8-dichloro hypocrellin A; 5,8 dichloro hypocrellin B; 5,8-dichlorodeacetylated hypocrellin B; 5,8-diiodo hypocrellin A; 5,8-diiodo hypocrellin B; 5,8-diiodo[1,12-CH═CMeCH(COCH₂|₂)-]hypocrellin B; 5,8-diiodo[1,12-CH₂C(CH₂I)═C(COMe)-]hypocrellin B; 2-(N.N-diethylamino)ethylaminated hypocrellin B; 3,10-bis[2-(NN-diethylamino)-ethylamine]hypocrellin B; 4,9-bis(2-(NN-diethyl-amino)-ethylamine]iso-hypocrellin B; dihydro-1,4-thiazine carboxylic acid hypocrellin B; dihydro-1,4-thiazine hypocrellin B; 2-(NN-dimethylamino)propylamine hypocrellin B; dimethyl-1,3,5,8,10,12-hexamethoxy-4,9-perylenequinone-6,7diacetate; dimethyl-5,8-dihydroxy-1,3,10,13-tetramethoxy-4,9-peryienequinone-6,7-diacetate; 2,11-dione hypocrellin A; ethanolamine hypocrellin B; ethanolamine iso-hypocrellin B; ethylenediamine hypocrellin B; 1,1-hydroxy hypocrellin B or 2-hydroxy hypocrellin B; hypocrellin A; hypocrellin B; 5-iodo-[1,12-CH₂C(CH₂I)═C(COMe)-]hypocrellin B; 8-iodo[1,12-CH₂C(CH₂I)═C(COMe)-]hypocrellin B; 9-methylamino iso-hypocrellin B; 3,10-bis[2-(N,N-methylamino)propylamine]hypocrellin B; 4,9-bis(methylamine iso-hypocrellin B; 14-methylamine iso-hypocrellin B; 4-methylamine iso-hypocrellin B; methoxy hypocrellin A; methoxy hypocrellin B; methoxy iso-hypocrellin A; methoxy iso-hypocrellin B; methylamine hypocrcllin B; 2-morpholino ethylaminated hypocrellin B; pentaacetoxy hypocrellin A; PQP derivative; tetraacetoxy hypocrellin B; 5,8,15-tribromo-hypocrellin B; calphostin C; cercosporins such as acetoxy cercosporin; acetoxy iso-cercosporin; aminocercosporin; cercosporin; cercosporin+iso-cercosporin (1/1 molar); diaminocercosporin; dimethylcercosporin; 5,8-dithiophenol cercosporin; iso-cercosporin; methoxycercosporin; methoxy iso-cercosporin; methylcercosporin; noranhydrocercosporin; cisinochrome A; cisinochrome B; phleichrome; and rubellin A.

Exemplary phenols include but are not limited to the following and deriavtives thereof: 2-benzylphenol; 2,2′-dihydroxybiphenyl; 2,5-dihydroxybiphenyl; 2-hydroxybiphenyl; 2-methoxybiphenyl; and 4-hydroxybiphenyl.

Exemplary pheophorbides include but are not limited to the following and derivatives thereof: pheophorbide a; methyl -13′-deoxy-20-formyl-7,8-vic-dihydro-bacterio-meso-pheophorbide a; methyl-2-(1-dodecyloxyethyl)-2-devinyl-pyropheophorbide a; methyl-2-(1-heptyloxyethyl)-2-devinylpyropheophorbide a; methyl-2-( 1-hexyl-oxyethyl )-2-devinyl-pyropheophorbide a; methyl-2-(1-methoxy-ethyl)-2-devinyl-pyropheophorbide a; methyl-2-(1-pentyloxyethyl)-2-devinyl-pyropheophorbide a; magnesium methyl bacteriopheophorbide d; methyl-bacteriopheophorbide d; and pheophorbide.

Exemplary pheophytins include but are not limited to the following and derivatives thereof: bacteriopheophytin a; bacteriopheophytin b; bacteriopheophytin c; bacteriopheophytin d; 10-hydroxy pheophytin a; pheophytin; pheophytin a; and protopheophytin.

Exemplary photosensitizer dimers and conjugates include but are not limited to the following and derivatives thereof: aluminum mono-(6-carboxy-pentyl-amino-sulfonyl)-trisulfophthalocyanine bovine serum albumin conjugate; dihematoporphyrin ether (ester); dihematoporphyrin ether; dihematoporphyrin ether (ester)-chlorin; hematoporphyrin-chlorin ester; hematoporphyrin-low-density lipoprotein conjugate; hematoporphyrin-high density lipoprotein conjugate; porphine-2,7,18-tripropanoic acid, -13,13′-(1,3-propanediyl)bis[3,8,12,17-tetramethyl]-; porphine-2,7,18-tripropanoic acid, 13,13′-(1,11-undecanediyl)bis[3,8,12,17-tetramethyl]-; porphine-2,7,18-tripropanoic acid, 13,13′-(1,6-hexanediyl)bis[3,8,12,17-tetramethylj-; SnCe6-MAb conjugate 1.7:1; SnCe6-MAb conjugate 1.7:1; SnCe6-MAb conjugate 6.8: 1; SnCe6-MAb conjugate 11.2:1; SnCe6-MAb conjugate 18.9:1; SnCe6-dextran conjugate 0.9:1; SnCe6-dextran conjugate 3.5:1; SnCe6-dextran conjugate 5.5:1; SnCe6-dextran conjugate 9.9:1; α-terthienyl-bovine serum albumin conjugate (12:1); α-terthienyl-bovine serum albumin conjugate (4:1); and tetraphenylporphine linked to 7-chloroquinoline.

Exemplary phthalocyanines include but are not limited to the following and derivatives thereof: (diol)(t-butyl)₃-phthalocyanine; (t-butyl)4-phthalocyanine; cis-octabutoxy-dibenzo-dinaphtho-porphyrazine; trans-octabutoxydibenzo-dinaphtho-porphyrazine; 2,3,9,10,16,17,23,24-octakis2-ethoxyethoxy)phthalocyanine; 2,3,9,10,16,17,23,24-octakis(3,6-dioxaheptyloxy)phthalocyanine; octa-n-butoxy phthalocyanine; phthalocyanine; phthalocyanine sulfonate; phthalocvanine tetrasulphonate; phthalocyanine tetrasulfonate; t-butyl-phthalocyanine; tetra-t-butyl phthalocyanine; and tetradibenzobarreleno-octabutoxy-phthalocyanine.

Exemplary porphycenes include but are not limited to the following or derivatives thereof: 2,3-(2′-carboxy-2′-methoxycarbonylbenzo)-7,12,17-tris(2-methoxyethyl)porphycene; 2-(2-hydroxyethyl)-7,12,17-tri(2-methoxyethyl)porphycene; 2-(2-hydroxyethyl)-7,12,17-tri-n-propyl-porphycene; 2-(2-methoxyethyl)-7,12,17-tri-n-propyl-porphycene; 2,7,12,17-tetrakis(2-methoxyethyl)porphycene; 2,7,12,17-tetrakis(2-methoxyethyl)-9-hydroxy-porphycene; 2,7,12,17-tetrakis(2-methoxyethyl)-9-methoxy-porphycene; 2,7,12,17-tetrakis(2-methoxyethyl)-9-n-hexyloxy-porphycene; 2,7,12,17-tetrakis(2-methoxyethyl)-9-acetoxy-porphycene; 2,7,12.17-tetrakis(2-methoxyethyl)-9-caproyloxy-porphycene; 2,7,12,17-tetrakis(2-methoxyethyl)-9-pelargonyloxy-porphycene; 2,7,12,17-tetrakis(2-methoxyethyl)-9-stearoyloxy-porphycene; 2,7,12,17-tetrakis(2-methoxyethyl)-9-(N-t-butoxycarbonylglycinoxyl porphycene; 2,7,12,17-tetrakis(2-methoxyethyl)-9-[4-((β-apo-7-carotenyl)benzoyloxyl-porphycene; 2,7,12,17-tetrakis(2-methoxyethyl)-9-amino-porphycene; 2,7,12,17-tetrakis(2-methoxyethyl)-9-acetamido-porphycene; 2,7,12,17-tetrakis(2-methoxyethyl)-9-glutaramido-porphycene; 2,7,12,17-tetrakis(2-methoxyethyl)-9-(methyl-glutaramido)-porphycene; 2,7,12,17-tetrakis(2-methoxyethyl)-9-(glutarimido)-porphycene; 2,7,12,17-tetrakis(2-methoxyethyl)-3-(N,N-dimethylaininomethyl)-porphycene; 2,7,12,17-tetrakis(2-methoxyethyl)-3-(N,N-dimethylaminomethyl)-porphycene hydrochloride; 2,7,12,17-tetrakis(2-ethoxyethyl)-porphycene; 2,7,12,17-tetra-n-propyl-porphycene; 2,7,12,17-tetra-n-propyl-9-hydroxy-porphycene; 2,7,12,17-tetra-n-propyl-9-methoxy-porphycene; 2,7,12,17-tetra-propyl-9-acetoxy porphycene; 2,7,12,17-tetra-n-propyl-9-(t-butyl glutaroxy)porphycene; 2,7,12,17-tetra-n-propyl-9-(N-ti-butoxycarbonylglycinoxy)-porphycene; 2,7,12,17-tetra-n-propyl-9-(4-N-t-butoxy-carbonyl-butyroxy)-porphycene; 2,7,12,17-tetra-n-propyl-9-amino-porphycene; 2,7,12,17-tetra-n-propyl-9-acetamidoporphycene; 2,7,12,17-tetra-n-propyl-9-glutaramido-porphycene; 2,7,12,17-tetra-n˜propyl-9-(methyl glutaramido)-porphycene; 2,7,12,17-tetra-n-propyl-3-(NN-dimethylaminomethyl)porphycene; 2,7,12,17-tetra-n-propyl-9,10-benzo porphycene; 2,7,12,17-tetra-n-propyl-9.-p-benzoyl carboxylporphycene; 2,7,12,17-tetra-n-propyl-porphycene; 2,7,12,17-tetra-t-butyl-3,6;13,16-dibenzo-porphycene; 2,7-bis-(2-hydroxyethyl)-12,17-di-n-propyl-porphycene; 2,7-bis(2-methoxyethyl)-12,17-di-n-propyl-porphycene; and porphycene.

Exemplary porphyrins include but are not limited to the following and derivatives thereof: 5-azaprotoporphyrin dimethylester; bis-porphyrin; coproporphyrin III; coproporphyrin III tetramethylester; deuteroporphyrin; deuteroporphyrin IX dimethylester; diformyldeuteroporphyrin IX dimethyl ester, dodecaphenylporphyrin; hematoporphyrin; hematoporphyrin IX; hematoporphyrin monomer; hematoporphyrin dimer; hematoporphyrin derivative; hematoporphyrin IX dimethylester; haematoporphyrin IX dimethylester; mesoporphyrin dimethylester; mesoporphyrin IX dimethylester; monoformyl-monovinyl-deuteroporphyrin IX dimethylester; monohydroxyethylvinyl deuteroporphyrin; 5,10,15,20-tetra(o-hydroxyphenyl)porphyrin; 5,10,15,20-tetra(m-hydroxyphenyl)porphyrin; 5,10,15,20-tetrakis-(m-hydroxyphenyl)porphyrin; 5,10,15,20-tetra(p-hydroxyphenyl)porphyrin; 5,10,15,20-tetrakis-(3-methoxyphenyl)porphyrin; 5,10,15,20-tetrakis-(3,4-dimethoxyphenyl)porphyrin; 5,10,15,20-tetrakis (3,5-dimethoxyphenyl)porphyrin; 5,10,15,20-tetrakis-(3,4,5-trimethoxyphenyl)porphyrin; 2,3,7,8,12,13,17,18-octaethyl-5,10,15,20-tetraphenylporphyrin; Photofrin; porphyrin c; protoporphyrin; protoporphyrin IX; protoporphyrin dimethylester; protoporphyrin IX dimethylester; protoporphyrin propylaminoethylformamide iodide; protoporphyrin N,N-dimethylaminopropylformamide; protoporphyrin propylaminopropylformainide iodide; protoporphyrin butylforinamide; protoporphyrin N˜-dimethylamino-formamide; protoporphyrin formamide; sapphyrin 13,12,13,22-tetraethyl-2,7,18,23 tetramethyl sapphyrin-8,17-dipropanol; sapphyrin 2 3,12,13,22-tetraethyl-2,7,18,23 tetramethyl sapphyrin-8-monoglycoside; sapphyrin 3; meso-tetra-(4-N-carboxyphenyl)-porphine; tetra-(3-methoxyphenyl)-porphine; tetra-(3-methoxy-2,4-difluorophenyl)-porphine; 5,10,15,20-tetrakis(4-N-methylpyridyl)porphine; meso-tetra-(4-N-methylpyridyl)porphine tetrachloride; meso-tetra(4-N-methylpyridyl)porphine; meso-tetra-(3-N-methylpyridyl)-porphine; meso-tetra-(2-N-methylpyridyl)porphine; tetra(4-NNN-trimethylanifinium)porphine; meso-tetra-(4-NNN″-trimethylamino-phenyl) porphine tetrachloride; tetranaphthaloporphyrin; 5,10,15,20-tetraphenylporphyrin; tetraphenylporphyrin; meso-tetra-(4-N-sulfonatophenyl)-porphine; tetraphenylporphine tetrasulfonate; meso-tetra-(4-sulfonatophenyl)porphine; tetra-(4-sulfonatophenyl)porphine; tetraphenylporphyrin sulfonate; meso-tetra-(4-sulfonatophenyl)porphine; tetrakis-(4-sulfonatophenyl)porphyrin; meso-tetra (4-sulfonatophenyl)porphine; meso-(4-sulfonatophenyl)porphine; meso-tetra-(4-sulfonatophenyl)porphine; tetrakis(4-sulfonatophenyl)porphyrin; meso-tetra-(4-N-trimethylanilinium)-porphine; uroporphyrin; uroporphyrin I; uroporphyrin IX; and uroporphyrin III.

Exemplary psoralens include but are not limited to the following and derivatives thereof: psoralen; 5-methoxypsoralen; 8-methoxypsoralen; 5,8-dimethoxypsoralen; 3-carbethoxypsoralen; 3-carbethoxy-pseudopsoralen; 8-hydroxypsoralen; pseudopsoralen; 4,5′,8-tn′methylpsoralen; allopsoralen; 3-aceto-allopsoralen; 4,7-dimethyl-allopsoralen; 4,7,4′-trimethyl-allopsoralen; 4,7,5′-trimethyl-allopsoralen; isopseudopsoralen; 3-acetoisopseudopsoralen; 4,5′-dimethyl-isopseudopsoralen; 5′,7-dimethylisopseudopsoralen; pseudoisopsoralen; 3-acetopseudoisopsoralen; 3,4′,5′-trimethylaza-psoralen; 4,4′,8-trimethy]-S′-amino-methylpsoralen; 4,4′,8-trimethyl-phthalamyl-psoralen; 4,5′,8-trimethyl-4′-aminomethyl psoralen; 4,5′,8-trimethyl-bromopsoralen; 5-nitro-8-methoxy-psoralen; 5′-acetyl-4,8-dimethyl-psoralen; 5′-aceto-8-methyl-psoralen; and 5′-aceto4,8-dimethyl-psoralen.

Exemplary purpurins include but are not limited to the following and derivatives thereof: octaethylpurpurin; octaethylpurpurinnzinc; oxidized octaethylpurpurin; reduced octaethylpurpurin; reduced octaethylpurpurin tin; purpurin 18; purpurin-18; purpurin18-methyl ester; purpurin; tin ethyl etiopurpurin 1; Zn(II) aetio-purpurin ethvl ester; and zinc etiopurpurin.

Exemplary quinones include but are not limited to the following and derivatives thereof: 1-amino-4,5-dimethoxy anthraquinone; 1 ,5-diamino-4,8-dimethoxy anthraquinone; 1,8-diamino-4,5-dimethoxy anthraquinone; 2,5-diamino-1,8-dihydroxy anthraquinone; 2,7-diamino-1,8-dihydroxy anthraquinone; 4,5-diamino-1,8-dihydroxy anthraquinone; mono-methylated 4,5- or 2,7-diamino-1,8-dihydroxy anthraquinone; anthralin (keto form); anthralin; anthralin anion; 1,8-dihydroxy anthraquinone; 1,8-dihydroxy anthraquinone (Chrysazin); 1,2-dihydroxy anthraquinonc; 1,2-dihydroxy anthraquinone (Alizarin); 1,4-dihydroxy anthraquinone (Quinizarin); 2,6-dihydroxy anthraquinone; 2,6-dihydroxy anthraquinone (Anthraflavin); 1-hydroxy anthraquinone (Erythroxy-anthraquinone); 2-hydroxyanthraquinone; 1,2,5,8-tetra-hydroxy anthraquinone (Quinalizarin); 3-methyl-1,6,8-trihydroxy anthraquinone (Emodin); anthraquinone; anthraquinonc-2-sulfonic acid; benzoquinone; tetramethyl benzoquinone; hydroquinone; chlorohydroquinone; resorcinol; and 4-chlororesorcinol.

Exemplary retinoids include but are not limited to the following and derivatives thereof: all-trans retinal; C₁₇ aldehyde; C22 aldehyde; 11-cis-retinal; 13-cis retinal; retinal; and retinal palmitate.

Exemplary rhodamines include but are not limited to the following and derivatives thereof: 4,5-dibromo-rhodamine methyl ester; 4,5-dibromo-rhodamine n-butyl ester; rhodamine 101 methyl ester; rhodamine 123; rhodamine 6G; rhodamine 6G hexyl ester; tetrabromo-rhodamine 123; and tetramethyl-rhodamine ethyl ester.

Exemplary thiophenes include but are not limited to the following and derivatives thereof: terthiophenes such as 2,2′:5′,2″-terthiophene; 2,2′:5′,2″-terthiophene-5-carboxamide; 2,2′:5′,2″-terthiophene-5-carboxylic acid; 2,2′:5′,2″-terthiophene-5-L-serine ethyl ester; 2,2′:5′,2″-terthiophene-5-N-isopropynyl-formamide; 5-acetoxymethyl-2,2′:5′,2″-terthiophene; 5-benzyl-2,2′:5′,2″-terthiophene-sulphide; 5-benzyl-2,2′:5′,2″-terthiophene-sulfoxide; 5-benzyl-2,2′:5′,2″-terthiophene-sulphone; 5-bromo-2,2′:5′,2″-terthiophene; 5-(butynyl-3′″-hydroxy)-2,2′:5′,2″-terthiophene; 5-carboxyl-5″-trimethylsilyl-2,2′:5′,2″-terthiophene; 5-cyano-2,2′:5′,2″-terthiophene; 5,5″-dibromo-2,2′:5′,2″-terthiophene; 5-(1′″,1′″-dibromoethenyl)-2,2′:5′,2″-terthiophene; 5,5″-dicyano-2,2′:5′,2″-terthlophene; 5,5″-diformyl-2,2′:5′,2″-terthiophene; 5-difluoromethyl-2,2′:5′,2″-terthiophene; 5,5″-diiodo-2,2′:5′,2′″-terthiophene; 3,3″-dimethyl-2,2′:5′,2″-terthiophene; 5,5″-dimethyl-2,2′:5′,2″-terthiophene; 5-(3′″,3′″-dimethylacryloyloxymethyl)-2,2′:5′,2″-terthiophene; 5,5″-di-{t-butyl)-2,2′:5′,2″-terthiophene; 5,5″-dithiomethyl-2,2′:5′,2″-terthiophene; 3′-ethoxy-2,2′:5′,2″-terthiophene; ethyl 2,2′:5′,2″-terthiophene-5-carboxylic acid; 5-formyl-2,2′:5′,2″-terthiophene; 5-hydroxyethyl-2.2′:5′,2″-terthiophene; 5-hydroxymethyl-2,2′:5′,2″-terthiophene; 5-iodo-2,2′:5′,2″-terthlophene-, 5-methoxy-2,2′:5′,2″-terthiophene; 3′-methoxy-2,2′:5′,2″-terthiophene; 5-methyl-2,2′:5′,2″-terthlophene; 5-(3′″-methyl-2′″-butenyl)-2,2′:5′,2″-terthiophene; methyl 2.2′:5′,2″-terthiophene-5-[3′″-acrylate]; methyl 2,2′:5′,2″-terthiophene-5-(3′″-propionate); N-allyl-2,2′:5′,2″-terthiophene-5-sulphonamide; N-benzyl-2,2′:5′,2″-terthiophene-5-sulphonamide; N-butyl-2,2′:5′,2″-terthiophene-5-sulphonamide; N,N-diethyl-2,2′:5′,2″-terthiophene-5-sulphonamide; 3,3′,4′,3″-tciramethyl-2,2′:5′,2″-terthiophene; 5-t-butyl-5″-trimethylsilyl-2,2′:5′,2″-terthiophene; 3′-thiomethyl-2,2′:5′,2″-terthiophene; 5-thiomethyl-2 ,2′:5′,2″-terthiophene; 5-trimethylsilyl-2,2′:5′,2″-terthiophene, bithiophenes such as 2,2′-bithiophene; 5-cyano-2,2′-bithiophene; 5-formyl-2,2′-bithiophene; 5-phenyl-2,2′-bithiophene; 5-(propynyl)-2,2′-bithiophene; 5-(hexynyl)-2,2′-bithiophene; 5-(octynyl)-2,2′-bithiophene; 5-(butynyl-4″-hydroxy)-2,2′-bithiophene; 5-(pentynyl-5″-hydroxy)-2,2′-bithiophene; 5-(3″,4″-dihydroxybutynyl)-2,2′-bithiophene derivative; 5-(ethoxybutynyl)-2,2′-bithiophene derivative, and misclaneous thiophenes such as 2,5-diphenylthiophene; 2,5-di(2-thienyl)furan; pyridine,2,6-bis(2-thienyl)-; pyridine, 2,6-bis(thienyl)-; thiophene, 2-(1-naphthalenyl)-; thiophene, 2-(2-naphthalenyl)-; thiophene, 2,2′-(1,2-phenylene)bis-; thiophene, 2,2′-(l,3-phenylene)bis-; thiophene, 2,2′-(1,4-phenylene)bis-; 2,2′:5′,2″,5″,2′″-quaterthiophene; α-quaterthienyl; α-tetrathiophene-, α-pentathiophene; α-hexathiophene; and α-heptathiophene.

Exemplary verdins include but are not limited to the following and derivatives thereof: copro (II) verdin trimethyl ester; deuteroverdin methyl ester; mesoverdin methyl ester; and zinc methyl pyroverdin.

Exemplary vitamins include but are not limited to the following and derivatives thereof: ergosterol (provitamin D2);β-dicyano-7-de(carboxymethyl)-7,8-didehydro-cobyrinate (Pyrocobester); pyrocobester; and vitamin D3.

Exemplary xanthene dyes include but are not limited to the following and derivatives thereof: Eosin B (4′,5′-dibromo,2′,7′-dinitro-fluorescein, dianion); eosin Y; eosin Y (2′,4′,5′,7′-tetrabromo-fluorescein, dianion); eosin (2′,4′,5′,7′-tetrabromo-fluorescein, dianion); eosin (2′,4′,5′,7′-tetrabromofluorescein, dianion)methyl ester; eosin (2′,4′,5′,7′-tetrabromo-fluorcscein, monoanion)p-isopropylbenzyl ester; eosin derivative (2′,7′-dibromo-fluorescein, dianion); eosin derivative (4′,5′-dibromo-fluorescein, dianion); eosin derivative (2′,7′-dichloro-fluorescein, dianion)-eosin derivative (4′,5′-dichloro-fluorescein, dianion);eosin derivative (2′,7′-diiodo-fluorescein, dianion); eosin derivative (4′,5′-diiodofluorescein, dianion); eosin derivative. (tribromo-fluorescein, dianion); eosin derivative (2′,4′,5′,7′-tetrachloro-fluorescein, dianion); eosin; eosin dicetylpyridinium chloride ion pair; erythrosin B (2′,4′,5′,7′-tetraiodo-fluorescein, dianion); erythrosin; erythrosin dianion; eosin B; fluorescein; fluorescein dianion; phloxin B (2′,4′,5′,7′-tetrabromo-3,4,5,6-tetrachloro-fluorescein, dianion); phloxin B (tetrachloro-tetrabromo-fluorescein); phloxine B; rose bengal (3,4,5,6-tetrachloro-21,41,51,71-tetraiodofluorescein, dianion); rose bengal; rose bengal dianion; rose bengal 0-methyl-methylester; rose bengal 6′-O-acetyl ethyl ester; rose bengal benzyl ester diphenyl-methyl-sulfonium salt; rose bengal benzyl ester triethylammonium salt; rose bengal benzyl ester 2,4,6,-triphenylpyrilium salt; rose bengal benzyl ester benzyltriphenylphosphonium salt; rose bengal benzyl ester benzyltriphenyl phosphonium salt; rose bengal benzyl ester diphenyl-iodonium salt; rose bengal benzyl ester diphenylmethylsulfonium salt; rose bengal benzyl ester diphenyl-methyl-sulfonium salt; rose bengal benzyl ester triethyl-ammonium salt; rose bengal benzyl ester triphenylpyrilium; rose bengal bis-(triethyl-ammonium)salt)(3,4,5,6-tetrachloro-2′,4′,5′,7′-tetraiodofluorescein bis(triethyl-ammonium salt); rose bengal bis(triethylammonium)salt rose bengal bis(benzyl-triphenyl-phosphonium)salt (3,4,5,6-tetrachloro-2′,4′,5′,7′-tetraiodofluorescein, bis(benzyl-triphenyl-phosphonium)salt); rose bengal bis(diphenyl-iodonium)salt(3,4,5,6-tetrachloro-2′,4′,5′,7′-tetraiodofluorescein bis(diphenyl-iodonium)salt); rose bengal di-acetyl-pyridinium chloride ion pair; rose bengal ethyl ester triethyl ammonium salt; rose bengal ethyl ester triethyl ammonium salt; rose bengal ethyl ester; rose bengal methyl ester; rose bengal octyl ester tri-n-butyl-ammonium salt RB; rose bengal, 6′-O-acetyl-, and ethyl ester.

Also suitable in the practice of the invention are the class of photosensitizers referred to as “green porphyrins” and derivatives thereof. A “green porphyrin” (Gp) is a porphyrin derivative obtained by reacting a porphyrin nucleus with an alkyne in a Diels-Alder type reaction to obtain a mono-hydrobenzoporphyrin. The resultant macropyrrolic compounds are called benzoporphyrin derivatives (BPDs), which are synthetic chlorin-like porphyrins with various structural analogs, as shown in U.S. Pat. Nos. 5,283,255, 4,920,143,4,883,790, and 5,171,749, the disclosures of which are hereby incorporated by reference herein. Examples of green porphyrin derivatives are also disclosed in U.S. Pat. Nos. 5,880,145 and 6,153,639, and WO 9,850,387, the disclosures of which are hereby incorporated by reference herein.

Typically, green porphyrins are selected from a group of tetrapyrrolic porphyrin derivatives obtained by Diels-Alder reactions of acetylene derivatives with protoporphyrins under conditions that promote reaction at only one of the two available conjugated, nonaromatic diene structures present in the protoporphyrin-IX ring systems (rings A and B). Metallated forms of a Gp, in which a metal cation replaces one or two hydrogens in the center of the ring system, may also be used in the practice of the invention. The preparation of the green porphyrin compounds useful in this invention is described in detail in U.S. Pat. No. 5,095,030, which is hereby incorporated by reference herein. Preferably, the BPD is a benzoporphyrin derivative di-acid (BPD-DA), mono-acid ring A (BPD-MA), mono-acid ring B (BPD-MB), or mixtures thereof. Examples of pyrrolic macrocycles directly applicable to the invention are shown below wherein A, B, C, D, and X can be hetero atoms or carbons.

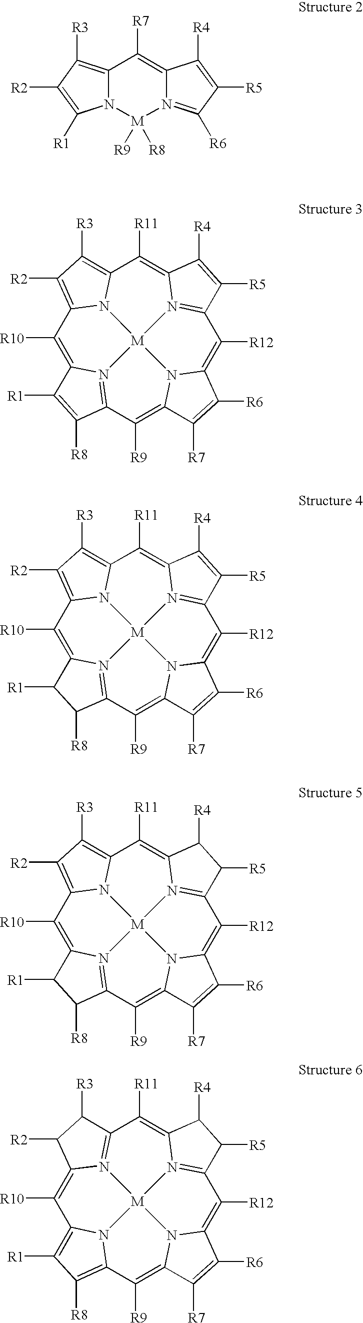

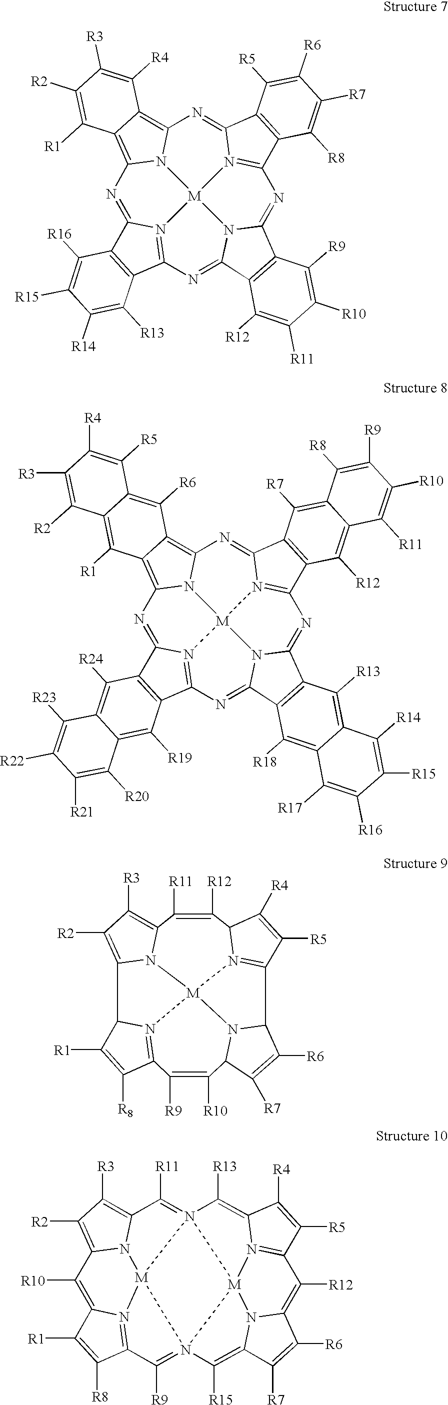

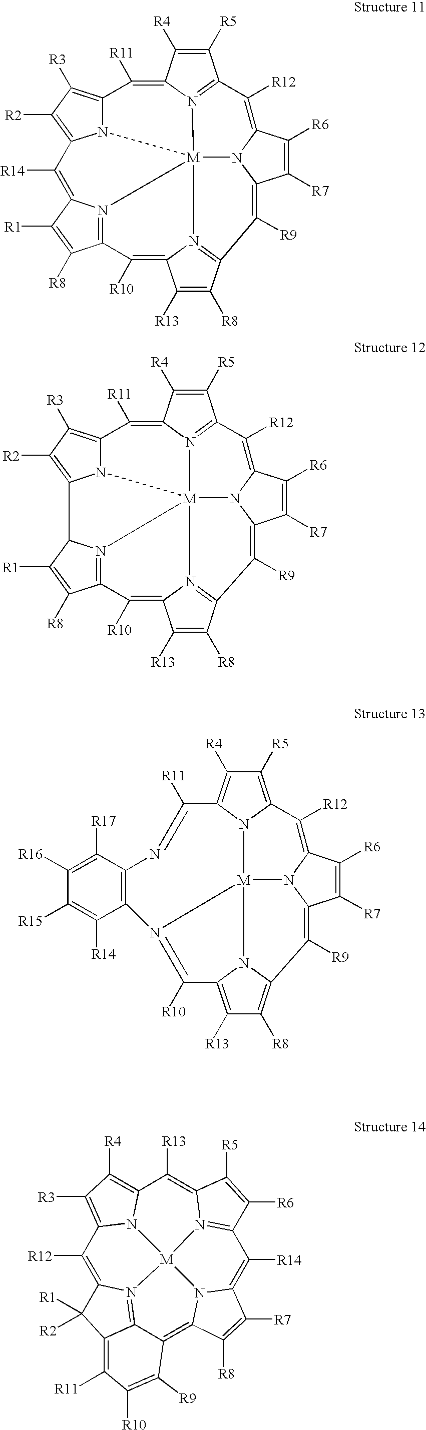

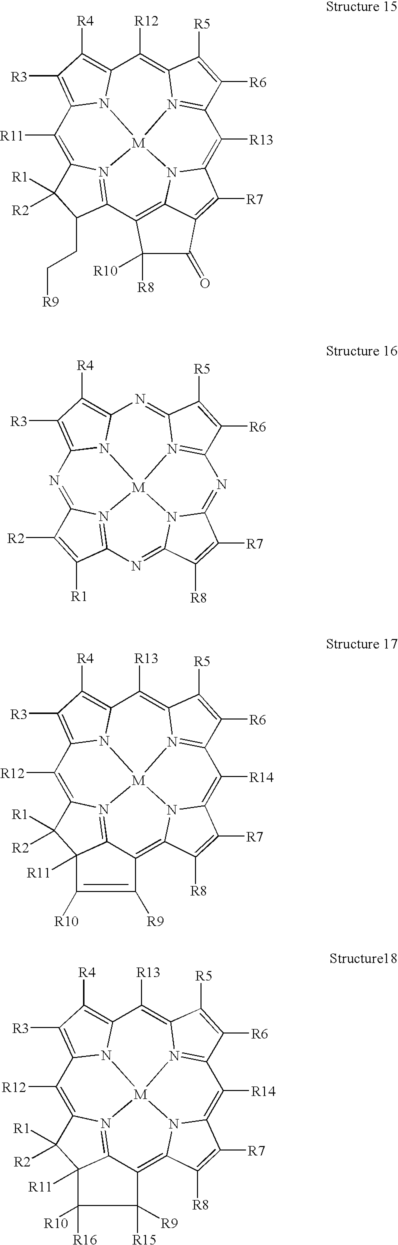

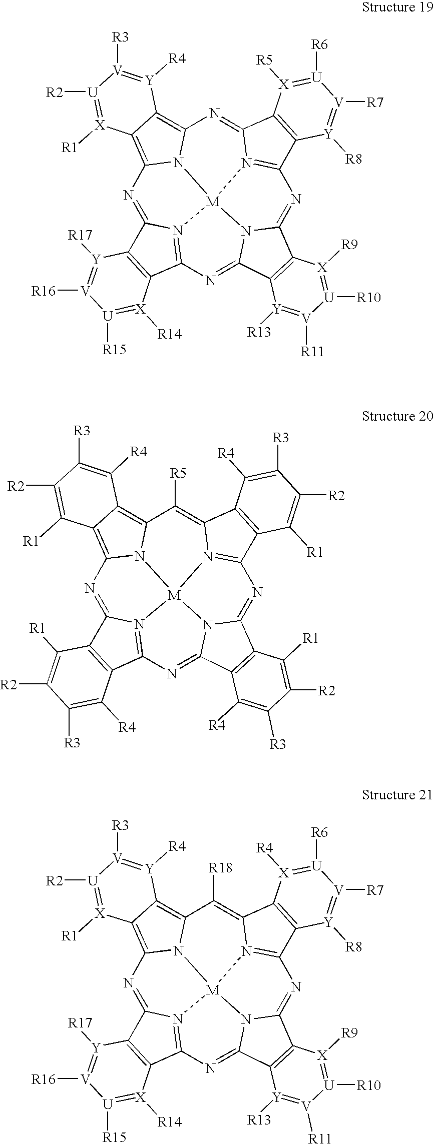

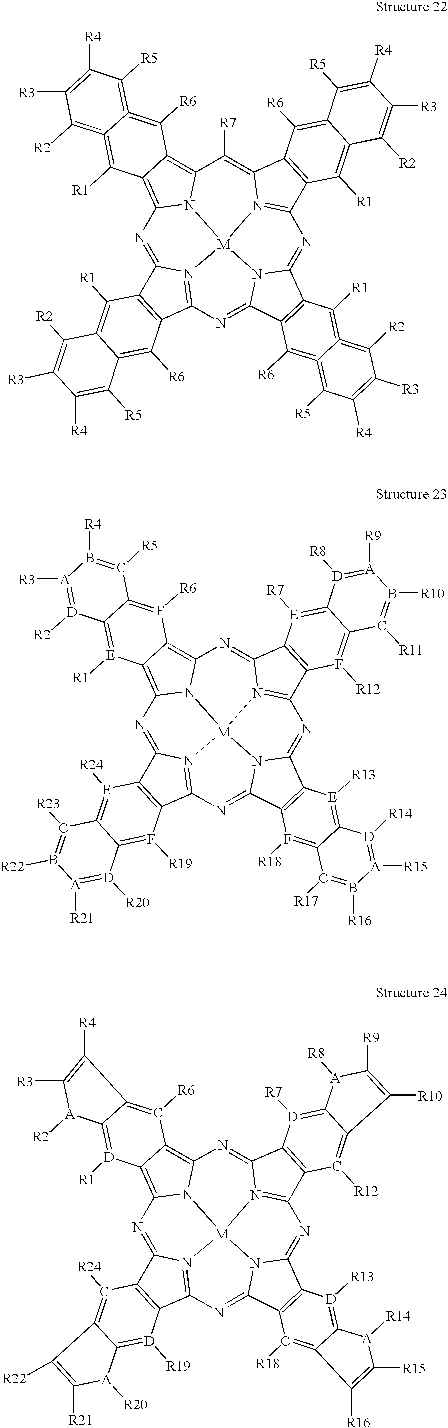

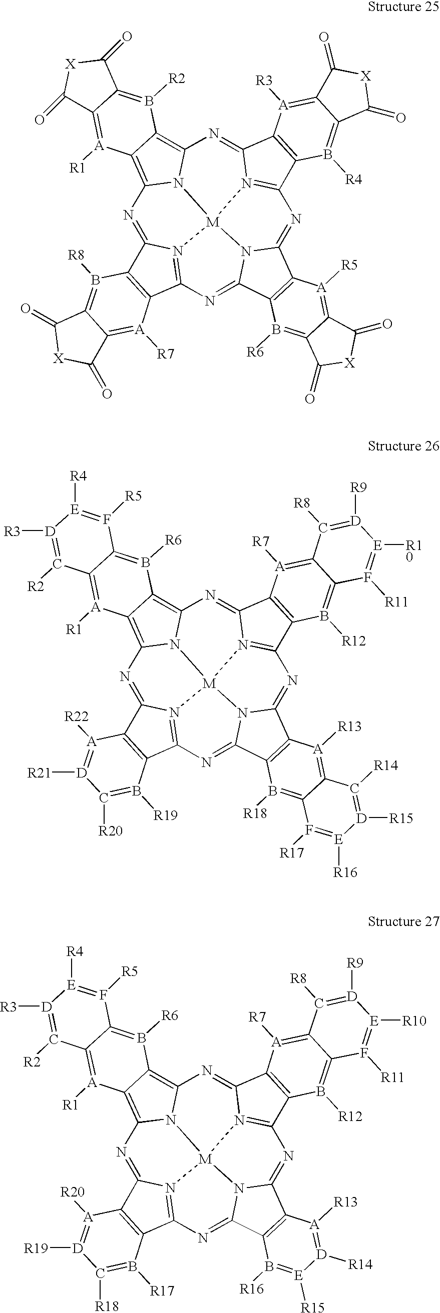

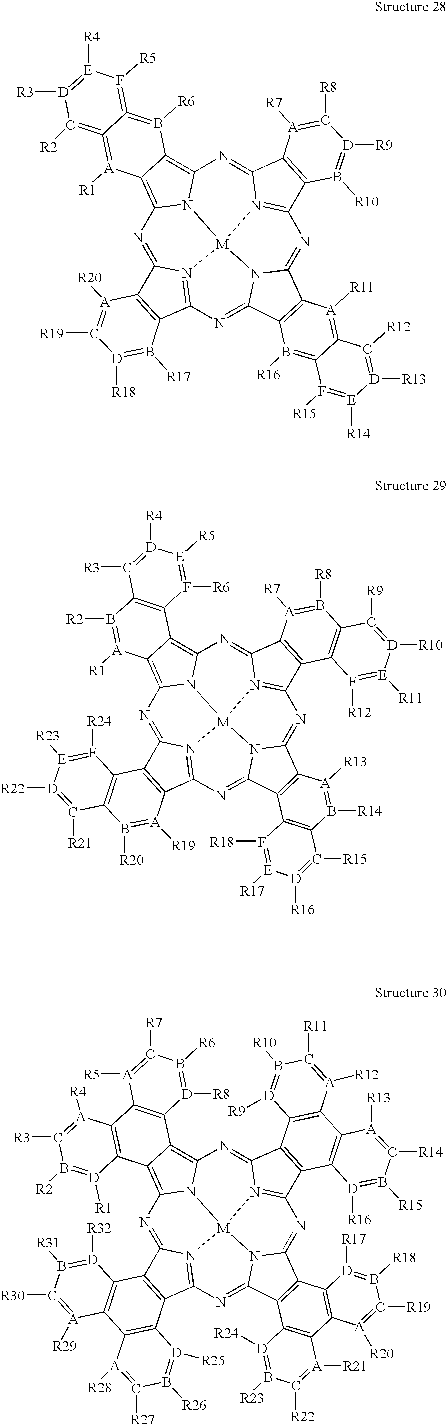

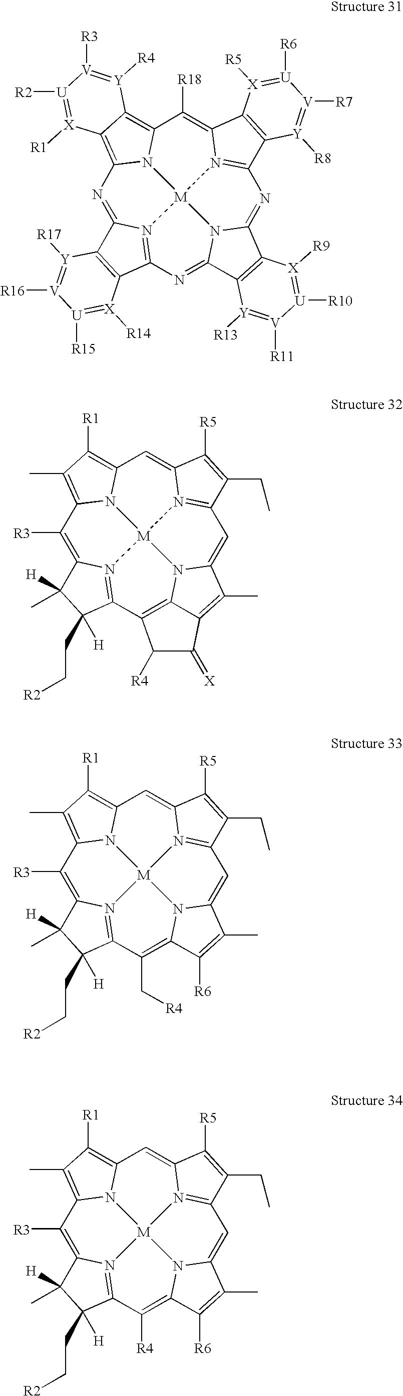

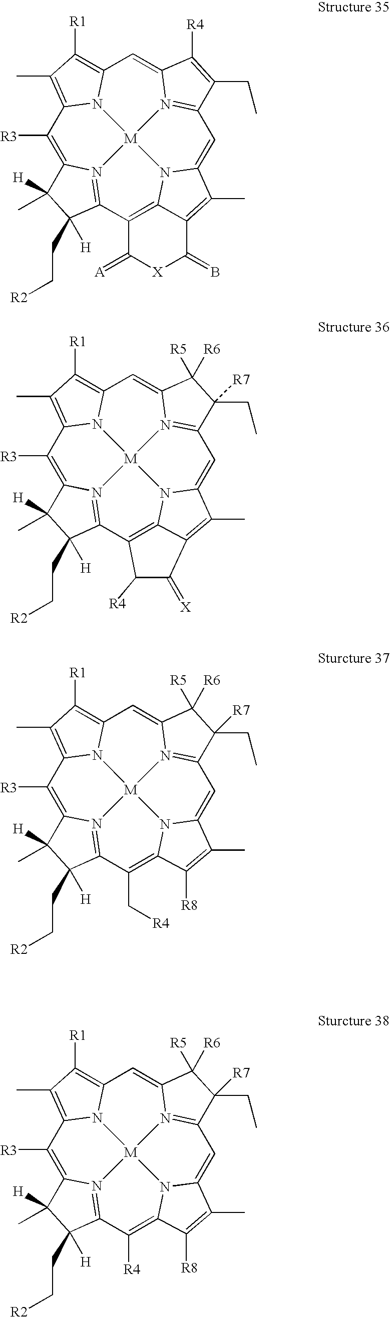

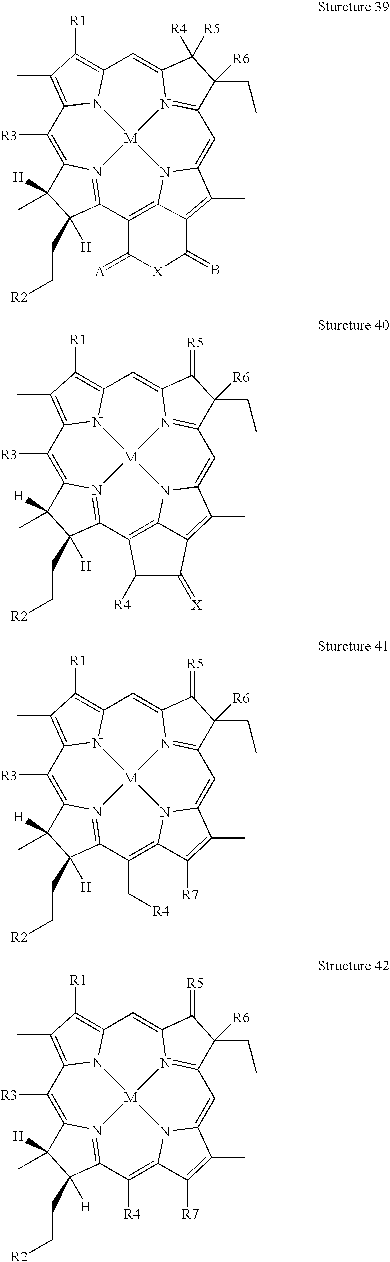

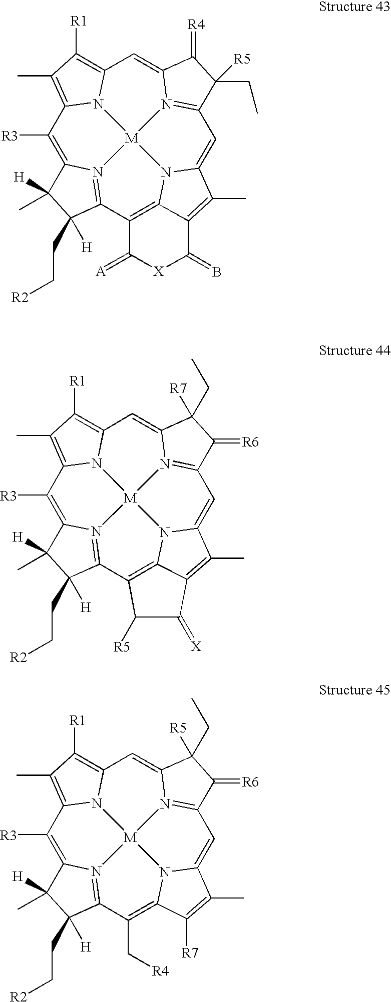

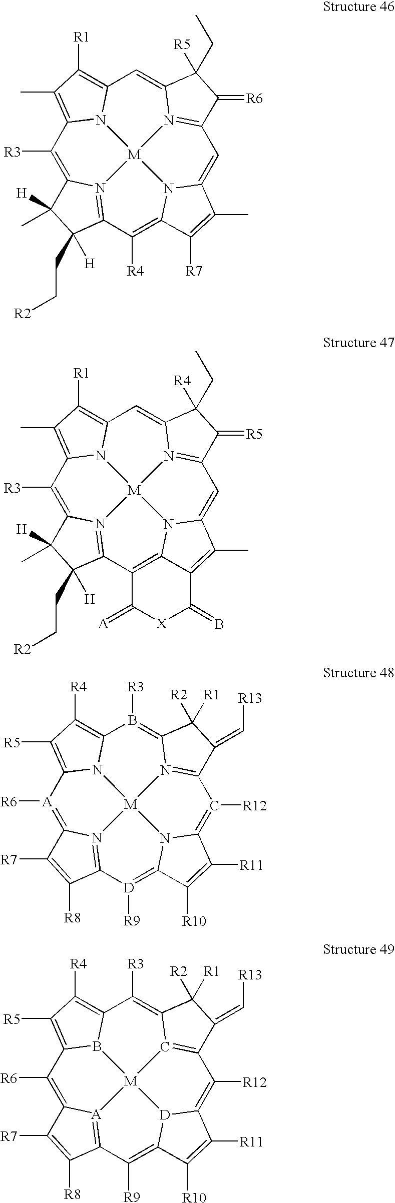

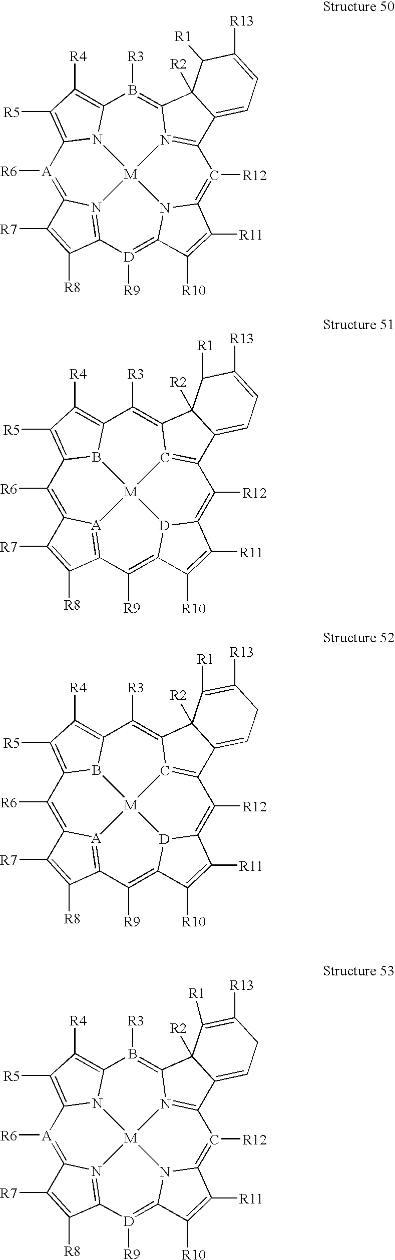

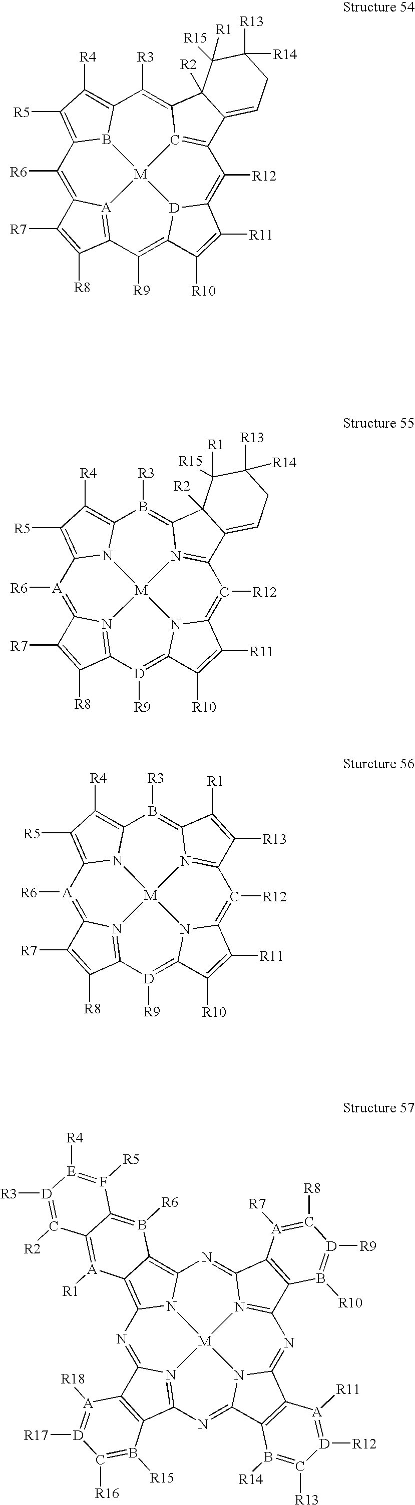

Examples and illustrations from the literature of types of photosensitizers disclosed in Structures 2 to 57 that may be used in photodynamic therapy or imaging and are applicable to the formation of carbamate analogs include:

Dipyrromethenes: (Structure 2).

Dipyrromethenes have been used widely as intermediates in the synthesis of porphyrins (for example, see “The Porphyrins” Ed. D.Dolphin, Academic Press, 1978, Volume II, 215-223; Volume I, Chapter IV, 101-234). References within these volumes provide actual experimental details. These compounds can be coordinated with metal salts to produce metallo complexes (for example, see A. W. Johnson, I. T. Kay, R. Price, K. B. Shaw, J. Chem. Soc, Perkin Trans I, 3416-3424, 1959; U.S. Pat. No. 5,189,029; U.S. Pat. No. 5,446,157). As shown in Structure 2, these molecules can be synthesized such that a wide variety of functionalities can be directly attached to the basic diyrromethene ring structure. Such functionality can be used to increase water solubility or lipophilicity, to conjugate to biomolecules such as antibodies or proteins, or to increase the wavelength of absorption of the molecules by increasing the conjugation of the macrocycle. As such, these molecules can be used for light activated photochemistry or diagnosis.

### Porphyrins: (Structure 3)

Routes to the synthesis of the ubiquitous tetrapyrrolic macrocycles that contain in their macrocyclic ring system 11 double bonds (excluding peripheral substituents), is outlined in detail in several publications including “Porphyrins and Metalloporphyrins” Ed. K. M. Smith, Elsevier Publishing Company, New York, 1975, Chapter 2, 29.55 and chapter 19, 778-785; and “The Porphyrins” Ed. D.Dolphin, Academic Press, 1978, Volume I. References within these volumes provide actual experimental details. A very large number of porphyrinic compounds have been synthesized. Because they are prevalent in nature, a large number of studies on the chemical modification of these compounds have been undertaken (“The Porphyrins” Ed. D.Dolphin, Academic Press, 1978, Volume I, 289-339:). A great deal of work has been undertaken on the synthesis of porphyrins from mono-pyrroles (“The Porphyrins” Ed. D.Dolphin, Academic Press, 1978, Volume I, chapter 3, 85-100, chapter 4, 101-234, chapter 5, 235-264, and chapter 6, 265-288). Examples of such work include the synthesis of mono, di, tri and tetraphenyl porphyrins (“The Porphyrins” Ed. D.Dolphin, Academic Press, 1978, Volume I, chapter 3, 88-90; Gunter, M. J., Mander, L. N., J. Org. Chem. 46, 4792-4795, 1981.). Such compounds can be widely functionalized as the aromatic rings may possess different substituents or have incorporated in them heteroatoms. Porphyrins also can be synthesized that possess annelated aromatic rings on the β-pyrrole positions (T. D. Lash, C. Wijesinghe, A. T. Osuma, J. R. Patel, Tetrahedron Letters, 38(12), 2031-2034.1997.), which can have the effect of extending conjugation and modifying the absorption and photophysical properties of the compounds. Porphyrin-type compounds have been synthesized from pyrroles and 5-membered ring heterocycles (such as thiophenes or furans for example), which incorporate one or more heteroatoms besides nitrogen within the central porphyrin “core” (“Porphyrins and Metalloporphyrins” Ed. K. M. Smith, Elsevier Publishing Company, New York, 1975, Chapter 18,729-732). Such compounds can be modified similarly to produce highly functionalized derivatives. In addition, porphyrin dimers, trimers or oligomers have been synthesized with great abandon. (See, H. Meier, Y. Kobuke, S. Kugimiya, J. Chem. Soc. Chem. Commun. 923,1989; G. M. Dubowchik, A. D. Hamilton, J. Chem. Soc. Chem. Commun, 904,,1985; R. K. Pandey, F-Y. Shaiu, C. J. Medforth, T. J. Dougherty, K. M. Smith, Tetrahedron Letters, 31,7399, 1990; D. R. Arnold, L. J. Nitschinsk, Tetrahedron Letters, 48, 8781,1992; J. L. Sessler, S. Piering, Tetrahedron Letters, 28, 6569,1987; A. Osaku, F. Kobayashi, K. Maruyama, Bull. Chem. Soc. Jpn, 64, 1213,1991).

### Chlorins: (Structures 4, 14, 15, 17, 18, 32-35, and 48-55)

Chlorins or hydroporphyrins are porphyrins that have only 10 double bonds in their macrocyclic ring system (excluding peripheral substituents). The “reduction” of the porphyrin macrocycle has pronounced effects on both the absorption profile of the macrocycle and the photophysical properties of the compound. Many naturally occuring chlorins may be extracted from plants, seaweeds or algae (e.g., see “Porphyrins and Metalloporphyrins” Ed. K. M. Smith, Elsevier Publishing Company, New York, 1975, Section H, 774-778) and simple chemical modifications to pheophorbides can give pyrropheophorbides, chlorin e6, purpurin 18 and other chlorin ring systems. Routes to the synthesis of chlorin macrocycles are outlined in “Porphyrins and Metalloporphyrins” Ed. K. M. Smith, Elsevier Publishing Company, New York, 1975, Chapter 2, 61-116, and Chapter 19, 774-778; and “The Porphyrins” Ed. D.Dolphin, Academic Press, 1978, Volume II, 1-37 and 131-143. References within these volumes provide actual experimental details. Considerable research has been directed toward the synthesis of chlorin derivatives from porphyrins. Catalytic hydrogenation and hydroboration (H. H. Inhoffen, J. W. Buchler, R. Thomas, Tetrahedon Letters, 1145, 1969), diimide reductions (H. W. Whitlock Jr., R Hanauer, R., Oester, M. Y., B. K. Bower, J. Am. Chem. Soc. 91, 7585,1969), osmium tetroxide (R. Bonnett, A. N. Nizhnick, M. C. Berenbaum, J. Chem. Soc. Chem. Comm., 1822, 1989) and hydrogen peroxide (C. K. Chang, Biochemistry, 19, 1971, 1971), alkali metals and electrochemical reduction (N. S. Hush, J. R. Rowlands, J. Am. Chem. Soc., 89, 2976, 1967), aromatic radicals (G. L. Closs, L. E. Closs, J. Am. Chem. Soc, 85, 818, 1963) have all been used to produce chlorins from porphyrins. The use of light as a reductive tool has also been extensively studied by several researchers. The reaction of singlet oxygen on vinyl porphyrin has been used extensively to produce chlorins (H. H. Inhoffen, H. Brockman, K. M. Bleisnerv, Ann. Chem. 730, 173, 1969; D. Brault, C. Vever-Bizet, Mougee, C., Bensasson, R., Photochem. Photobiol. 47, 151, 1988). The reduction of free base and metalloporphyrins with light and reducing agents (such as amines or ascorbates) (Y. Harel, J. Manassen, J. Am. Chem. Soc., 100, 6228, 1977; J. H. Fuhrhop, T. Lumbantobing, Tetrahedron Letters, 2815, 1970; D. G. Whitten, J. C., Yau, F. A. Carol, J. Am. Chem. Soc., 93, 2291, 1971) also produces chlorins. Cyclization of meso-acrylate containing porphyrins has been used extensively to produce purpurin derivatives (Structures 17 and 18) (A. R. Morgan, N. C. Tertel., J. Org. Chem., 51, 1347, 1986) while acid cyclization of meso-acrolein porphyrins has been used extensively to produce benzochlorins (Structure 14) (M. G. H. Vincente, I. N. Rezzano, K. M. Smith, Tetrahedron Letters, 31, 1365, 1990; M. J. Gunter, B. C. Robinson, Tetrahedron., 47, 7853, 1991). Diels-alder addition of dienophiles with vinyl-containing porphyrins has been used extensively to produce chlorins (Structures 50-55) (R. Grigg, A. W. Johnson, A. Sweeney, Chem. Commun. 697, 1968; H. J. Callot, A. W. Johnson, A. Sweeney, J. Chem. Soc. Perkin Trans. I, 1424, 1973). Acetamidoporphyrins can be cyclized to produce chlorins via an intramolecular Vilsmeier reaction (G. L. Collier, A. H. Jackson, G. W., Kenner, J. Chem. Soc., C., 564, 1969). Recently, chlorin analogs of purpurin 18 based on purpurin 18 have been produced that possess nitrogens on the cyclic “anhydride” ring system (Structure 35, A or B=NR).

### Bacteriochlorins and Isobacteriochlorins: (Structures 5, 6, 36 to 47)

Bacteriochlorins and isobacteriochlorins are tetrahydroporphyrins. These derivatives have only nine double bonds in their macrocyclic ring system (excluding peripheral groups). The “double” reduction of the porphyrin nucleus at the pyrrole positions has a pronounced effect on the absorption properties and photophysical properties. Typically, bacteriochlorins absorb in the 720-850 nm range while isobacteriochlorins absorb in the 500-650 nm range (“The Porphyrins” Ed. D.Dolphin, Academic Press, 1978, Volume III, Chapter 1; references within these volumes provide actual experimental details). Examples of-the synthesis of bacteriochlorins and isobacteriochlorins can be found in the following references: H. H. Inhoffen, P. Jager, R. Mahlhop and C. D. Mengler, Justus Liebigs Ann. Chem. 704, 188, 1967; H. Mittenzwei, S. Z. Hoppe-Seyler, Physiol. Chem. 275, 93, 1942; H. Brockmann Jr., G . Knobloch, Arch. Mikrobiol, 85, 123, 1972; J. J. Katz, H. H., Strain, A. L., Harkness, M. H. Studier, W. A., Svec, T. R. Janson, B. T. Cope, J. Am. Chem. Soc. 94, 7983, 1972; U.S. Pat. No. 5,648,485; U.S. Pat. No. 5,149,708; H. W. Whitlock, R. Hanauer, M. Y. Oester, B. K. Bower, J. Am. Chem Soc. 91, 7485, 1969; H. H. Inhoffen, H. Sheer, Tetrahedron Letters, 1115, 1972; H. H. Inhoffen, J. W. Buchler, R. Thomas, Tetrahedron Letters, 5145, 1969; and J. H. Fuhrhop, T. Lumbantobing, Tetrahedron Letters, 2815, 1970. In particular, osmium tetroxide has proved useful in the synthesis of β, β-dihydroxy-bacteriochlorins and isobacteriochlorins from chlorins (U.S. Pat. No. 5,591,847) and the acid rearrangement of these derivatives has produced numerous bacteriochlorin derivatives. The treatment of porphyrins and chlorins with hydrogen peroxide has been used to produce bacteriochlorins and isobacteriochlorins (H. H. Inhoffen, W. Nolte, Justus Liebigs Ann. Chem. 725, 167, 1969). Diels-alder addition of dienophiles with porphyrins containing two vinyl substituents has been used extensively to produce bacteriochlorins and isobacteriochlorins (R. Grigg, A. W. Johnson, A. Sweeney, Chem. Commun., 697, 1968; H. J. Callot, A. W. Johnson, A. Sweeney, J. Chem. Soc. Perkin Trans. I, 1424, 1973).

### Phthalocyanines and Naphthalocyanines: (Structures 7, 8, 19, 20-31)

Phthalocyanines and phthalocyanine analogs are perhaps some of the most widely studied photosensitizers in the field of photodynamic therapy and are also widely used as optical recording media. As such, the number of structurally different phthalocyanine derivatives is enormous. Not only can the peripheral functionality of these compounds be widely varied, which changes their electronic spectra and photophysics, but metallation of the macrocycle also results in photophysical changes. In addition, carbons in the aromatic rings may be substituted with heteroatoms (such as nitrogen and sulphur phosphorus) that markedly change the photophysical properties of the compounds. Examples of references that disclose the formation of such compounds include: “Phthalocyanines, Properties and Applications, Eds. C. C. Leznoff, A. B. P. Lever, VCH Publishers Inc., 1989; “The Phthalocyanines”, Eds. F. H. Moser, A. L. Thomas, CRC Press, Volumes I and II, 1983; “The Porphyrins” Ed. D.Dolphin, Academic Press, 1978, Volume I, Chapter 9, 374-380; A. K. Sobbi, D.Wohrle, D. Schlettwein, J. Chem. Soc. Perkin Trans. 2, 481-488, 1993; J. H. Weber, D. H. Busch, Inorg. Chem. 192, 713, 1988.; R. P. Linstead, F. T. Weiss, J. Chem. Soc., 2975, 1950; U.S. Pat. Nos. 5,166,197, 5,484,778, and 5,484,915. A great number of binuclear phthalocyanines/napthalocyanines have been synthesized that share a common benzene or naphthalene ring (J. Yang, M. R. Van De Mark, Tetrahedron Letters, 34, 5223, 1993; N. Kobayashi, H. Y. Higashi, T. Osa, Chemistry Letters, 1813, 1994).

### Azaporphyrins: (Structures 16, 56)

Porphyrins that possess at least one meso-nitrogen linking atom are called azaporphyrins. The number of meso-nitrogen linking atoms may be extended from one to four. Phthalocyanines and naphthalocyanine may be regarded as tetraazoporphyrins with extended conjugation due to annelated benzene and napthalene rings. The synthesis of mono, di, tri and tetraazoporphyrin analogs is discussed in “The Porphyrins” Ed. D.Dolphin, Academic Press, 1978, Volume I, Chapter 9, 365-388; “Phthalocyanine, Properties and Applications, Eds. C. C. Leznoff, A. B. P. Lever, VCH Publishers Inc., 1989; “The Phthalocyanines” , Eds. F. H. Moser, A. L. Thomas, CRC Press, Volumes I and II, 1983. References within these volumes provide actual experimental details. The synthesis of a series of tetrabenzotriazoporphyrins and tetranapthotriazoporphyrins has recently been published (Y-H Tse, A. Goel, M. Hue, A. B. P. Lever, C. C. Leznoff, Can. J. Chem. 71, 742, 1993). It can be envisaged that chemistry typical of phthalocyanine chemistry and porphyrin chemistry may be applied to these compounds, such that heteroatoms may be introduced into the annelated benzene or napthalene rings.

### Asymmetrical Benzonaphthoporphyrazines: (Structures 26-28, 57)

Asymmetrical tetraazoporphyrins that have both a benzene and a naphthalene annelated unit in the macrocyclic ring system are loosely called benzonaphthoporphyrazines. The synthesis of these derivatives is carried out using classical phthalocyanine syntheses however, using mixed aromatic dinitriles (U. Michelsen, H. Kliesch, G. Schnurpfeil, A. K. Sobbi, D. Wohrle, Photochem. Photobiol, 64, 694, 1996; Canadian Patent No. 2,130,853. References to the synthesis of these macrocycles can also be found in “Phthalocyanine, Properties and Applications, Eds. C. C. Leznoff, A. B. P. Lever, VCH Publishers Inc., 1989; “The Phthalocyanines”, Eds. F. H. Moser, A. L. Thomas, CRC Press, Volumes I and II, 1983.

### Texaphyrins: (Structure 13)

Texaphyrins are tripyrrol dimethene derived “expanded porphyrin” macrocycles that have a central core larger than that of a porphyrin. The reaction of diformyl tripyrranes with functionalized aromatic diamines in the presence of a metal gives rise to functionalized metallated texaphyrins (U.S. Pat. Nos. 5,252,720, 4,935,498; and 5,567,687).

### Pentaphyrins and Sapphyrins: (Structures 11, 12)

Sapphyrins and pentaphyrins are fully conjugated macrocycles that possess five pyrrole units. Structural analogs of the sapphyrins and pentaphyrins are outlined in “Porphyrins and Metalloporphyrins”, Ed. K. M. Smith, Elsevier, Chapter 18, 750-751; “The Porphyrins Ed. D. Dolphin, Academic Press, NY, Chapter 10, 351-356; Broadherst et al, J. Chem. Soc. Perkin Trans. I, 2111, 1972; U.S. Pat. No. 5,543,514.

### Porphycenes: (Structure 9)

Porphycenes are isomeric analogs of porphyrins that have eleven double bonds in their macrocyclic core and are derived by a mere reshuffling of the pyrrole and methine moieties. Routes to the synthesis of functionalized porphycenes are outlined in the following references: U.S. Pat. Nos. 5,409,900, 5,262,401, 5,244,671, 5,610,175, 5,637,608, and 5,179,120; D. Martire, N. Jux, P. F. Armendia, R. M. Negri, J. Lex, S. E. Braslavsky, K. Schaffner, E. Vogel. J. Am. Chem. Soc., 114, 1992; N. Jux, P. Koch, H. Schmickler, J.Lex, E.Vogel. Angew. Chem. Int. Ed. Engl. 29, 1385, 1990.

The present invention provides for the synthesis of photodynamically active compounds and the resulting compounds may be used in phototherapy for diagnosis or treatment. Additionally, the compounds may be useful in the field of scintillation imaging if made radioactive.

As an example of the invention, in reaction Scheme 8, a tetrapyrrole (pyr) possessing a hydroxyl group is converted into the photodynamically active compound of formula I. The reaction can be achieved with the proper choice of solvent and reaction conditions. Those solvents may include methylene chloride, chloroform, toluene, pyrrolidine, 1,2-dichloroethane, and mixtures thereof. Contacting the hydroxyl group with carbonyldiimidazole (or bis(p-nitrophenyl)carbonate) in the presence of a catalytic amount of 4-dimethylaminopyridine (DMAP) followed by an amine or imine at room temperature yields the compounds of the invention. Amines that can be used include, but are not limited to, alkylamines, aminoalcohols, aminoethers, diamines, and aminoacids. The following examples outline some of the photosensitizer classes and modifications that have been performed according to the invention.

The following reaction schemes are given to highlight some of the types of compounds that are capable of being synthesized within the scope of the invention and are not intended to limit the invention. It would be obvious to those skilled in the art as to the chemical modifications to the tetrapyrrolic ring structures (or other photosensitizers) and peripheral groups that may be undertaken in accordance with the invention.

### Pheophorbide Carbamate Derivatives.

In reaction Scheme 8, the compound of formula pyr is first treated with carbonyldiimidazole in the presence of DMAP in methylene chloride followed by an amine to give compounds (1), (2), (3), (4), (5), (6). This produces compounds that are functionalized with carbamates at the 2-position.

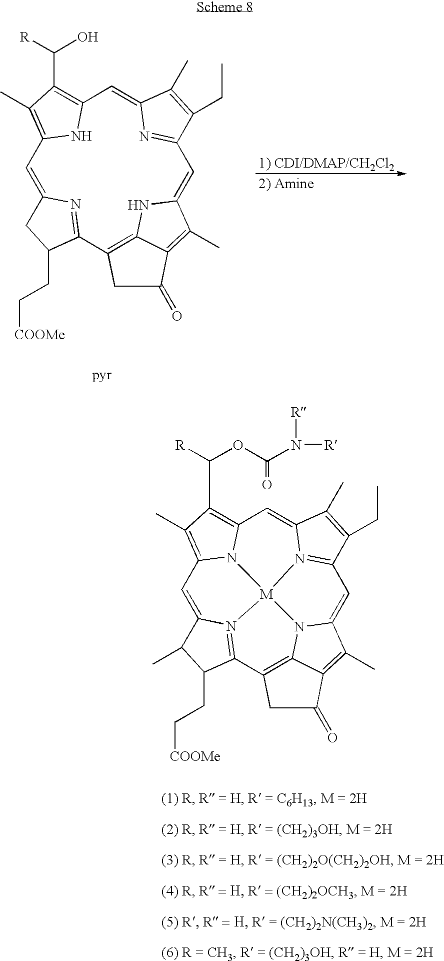

In reaction Scheme 9, compounds of the formula pyr are first treated with carbonyldiimidazole (CDI) in the presence of DMAP in methylene chloride followed by an imine to give compounds (7) and (8).

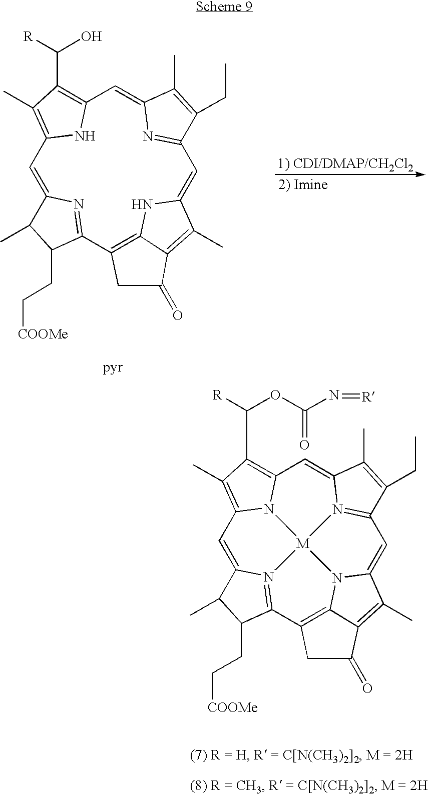

In Scheme 10, 9-desoxo-9-hydroxypyrropheophorbide methyl ester (Rpheo) is reacted with CDI/DMAP followed by an amine. This produces pyrropheophorbides that are functionalized with carbamates at the 9-position.

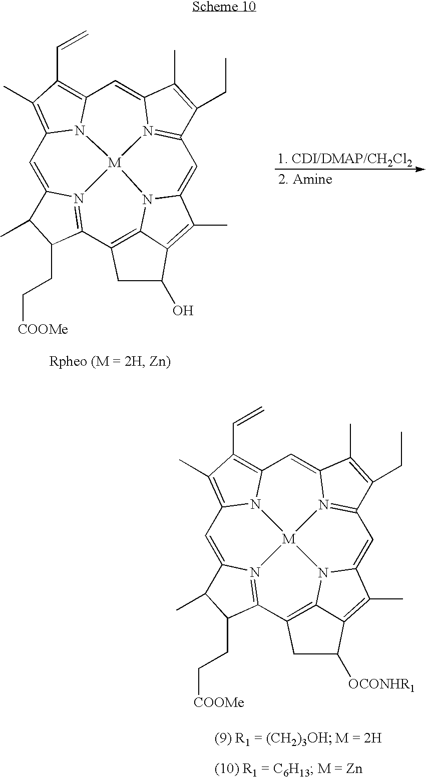

In Scheme 11, the propionic acid side chain of pyrropheophorbide is functionalized with either an alcoholic ester or amide, to give compounds like (11) and (12) (and the like). These may then be reacted according to the invention to produce pyrropheoporpbide carbamates functionalized on the propionic acid side chain.

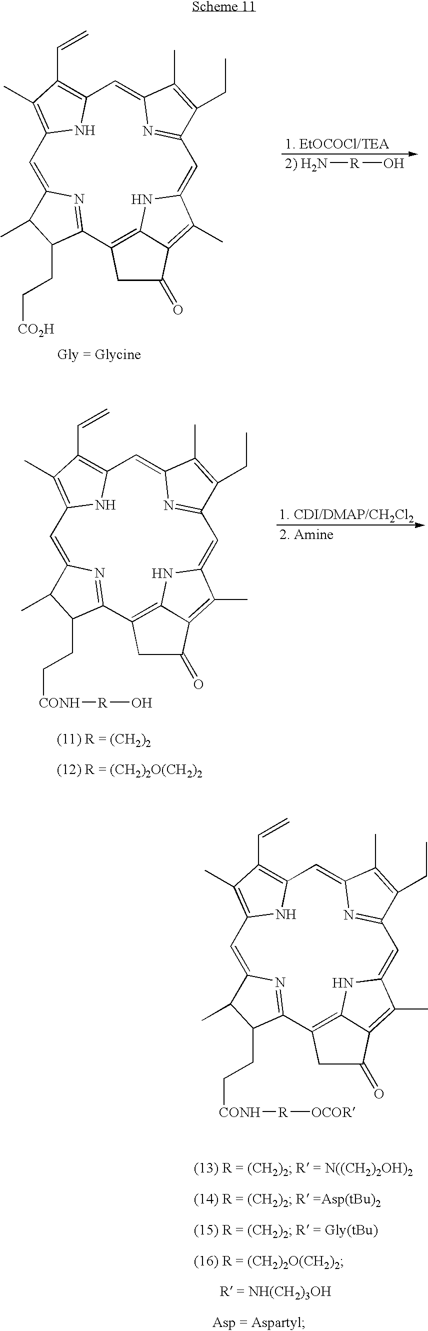

Scheme 12 outlines the synthesis of pyrropheophorbide carbamates functionalized at the 3-position. In this instance, pyrropheophorbide b is reduced with sodium borohydride to give the 3-methylalcohol derivative. This is then reacted according to the invention to give 3-functionalized pheophorbide carbamates.

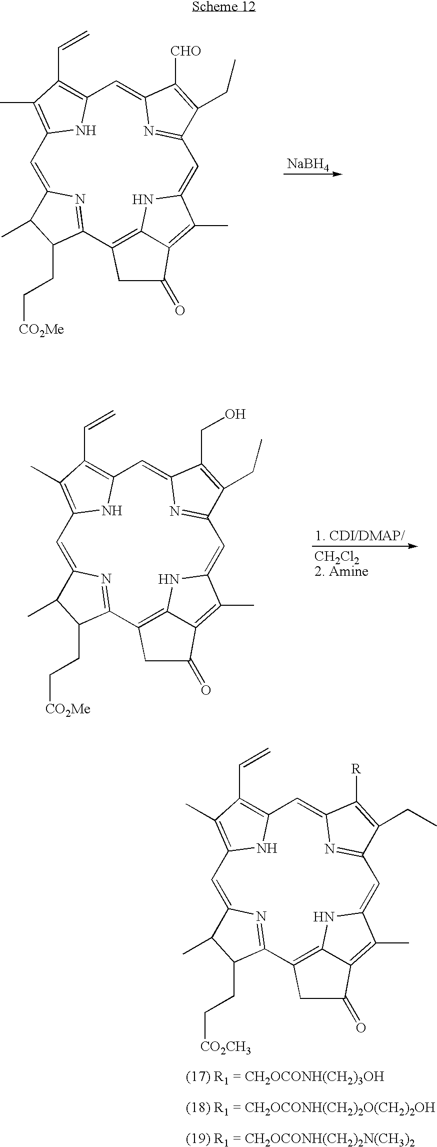

### Chlorin e6 Carbamates

Compounds of formula II are conveniently prepared as described above. As an example, in reaction Scheme 13, compounds of formula Ce6 are converted to compounds like (20)-(25) according to the invention. This produces chlorin e6 carbamates functionalized at the 2-position.

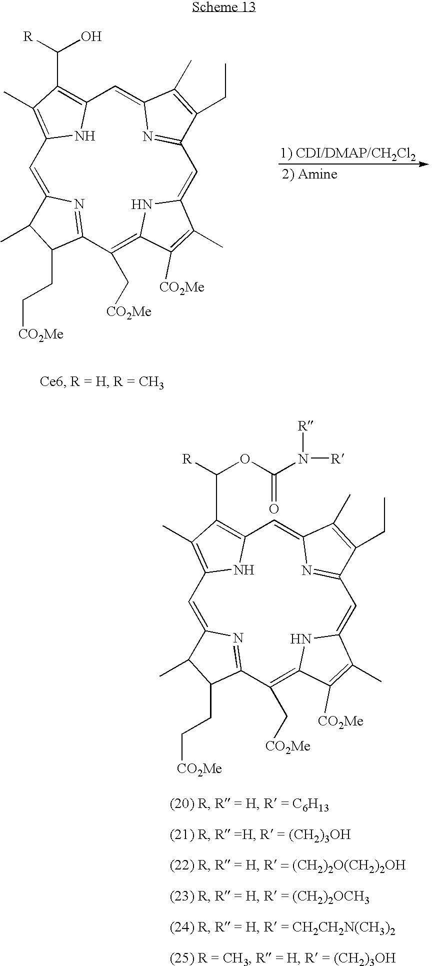

Reaction Scheme 14 outlines the synthesis of chlorin e6 carbamates derived from chlorin e6 6-amides. In this instance, pheophorbides have been ring opened with a hydroxylated amine to produce chlorin e6 6-amides possessing hydroxyl groups. These in turn may be reacted according to the invention to produce carbamate derivates such as (26) and the like.

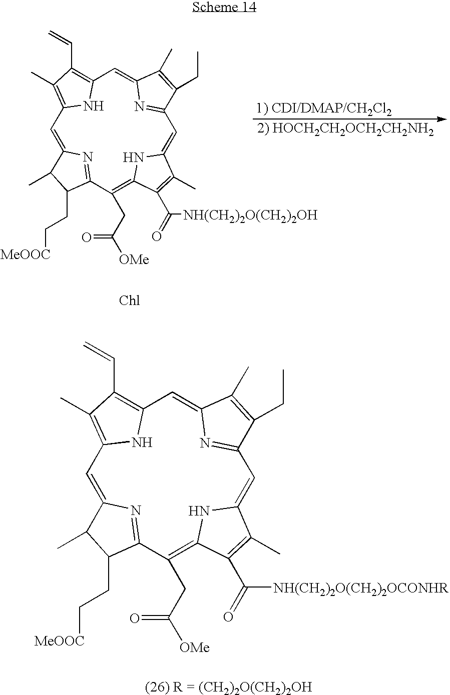

### Benzoporphyrin Carbamate Derivatives

Benzoporphyrin derivatives derived from pyrropheoporphyrin or protoporphyrin IX have been modified according to the invention to produce benzoporphyrin carbamates. In Scheme 15, the benzoporphyrin derivative B (derived via the reaction of the ethylene glycol ketone protected methyl pyrropheoporphyrin, Pandey et al, Tetrahedron, 52:15, 5349-5362, 1998), with dimethyl acetylenedicarboxylate, base cyclization and subsequent ketone deprotection) is reduced with sodium borohydride to give the 9-desoxo-9-hydroxy derivative Bp. Treatment of Bp with CDI/DMAP followed by an amine gives the desired carbamate analogs (27) and (28). This produces benzoporphyrin derivatives functionalized at the 9-position.

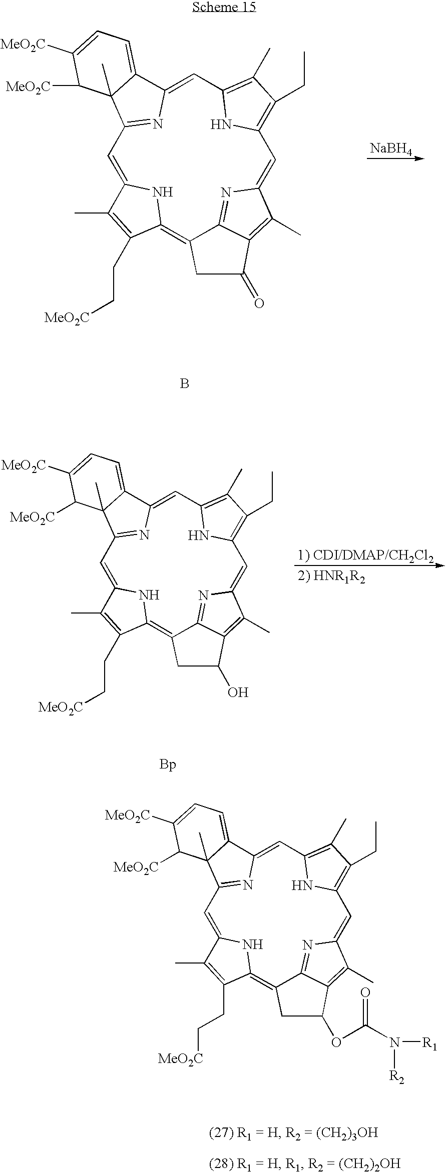

Alternatively, reduction of the acetyl benzoporphyrin derivative shown in Scheme 16 produces the 4-(1-hydroxyethyl) benzoporphyrin derivative, which may be modified according to the invention to give benzoporphyrin carbamate derivatives, examples of which are (29) and (30). Clearly, carbamates may be made from either alcohol esters or alcohol amides of the propionic acid group.

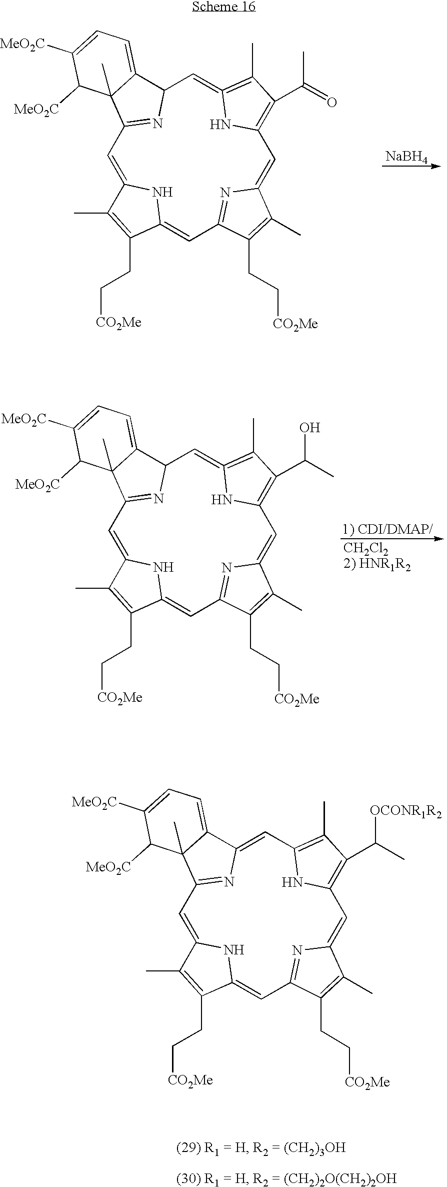

### Benzochlorin and Isobenzochlorin Carbamates

A large number of benzochlorins possessing hydroxyl groups have been synthesized in the literature. Scheme 17 outlines the synthesis of some isobacteriobenzochlorins according to the invention.

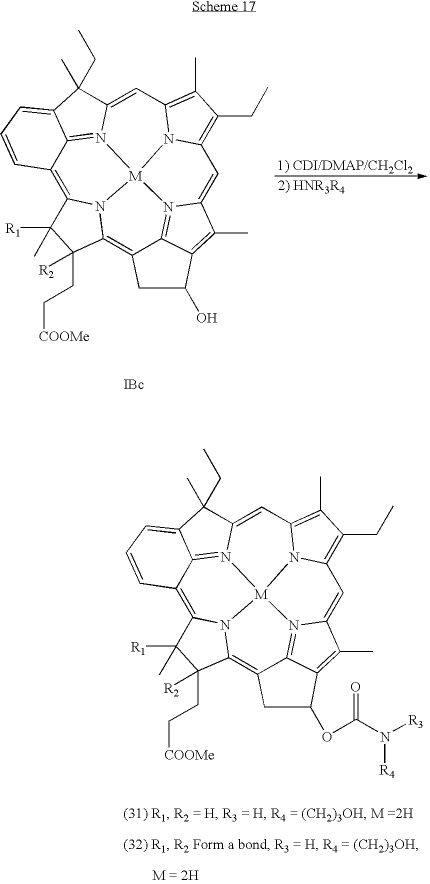

In this instance, a demetallated isobenzochlorin (Smith et al, J. Org. Chem., 56, 4407-4418, 1991) is reduced with sodium borohydride to give IBc. This is converted to the carbamates (31) and (32) according to the invention.

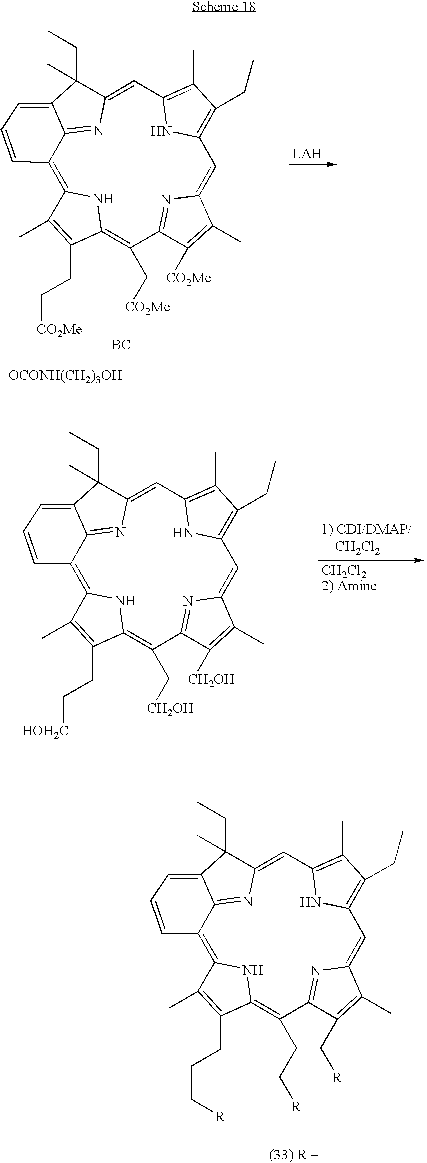

The chlorin e6 based benzochlorin BC was reduced with lithium aluminium hydride to give the benzochlorin triol, which was converted according to the invention to the benzochlorin tricarbamate.

Alternatively, the sulfonylamide benzochlorins (34) and (35) of Scheme 19 (produced in accordance with the teachings of U.S. Pat. No. 5,789,589) were converted to the carbamate benzochlorins (36) and (37) respectively.

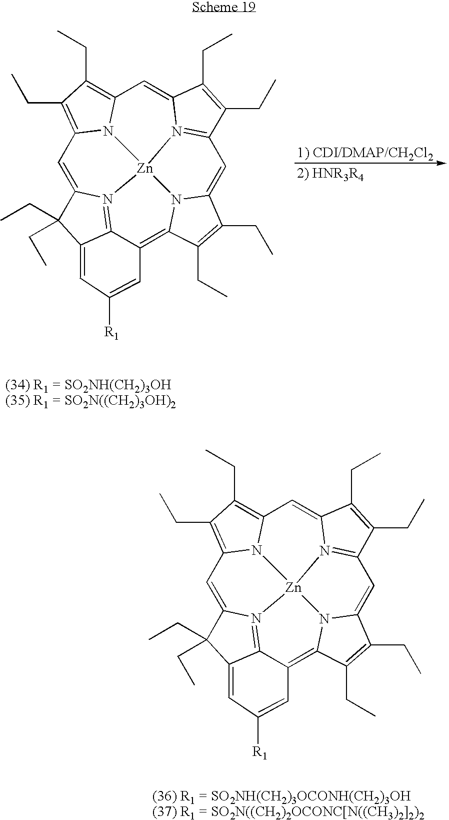

### Purpurin 18 Carbamates

Purpurin 18 and purpurin 18 imides and their bacteriopurpurin analogs are relatively straightforward to make synthetically (Zheng, G., et al, Bioorganic & Med. Chem. Letters, 10,123-127,2000; Kosyrev, A. N., et al., Tet. Lett., 37(36), 6431-6434, 1996).

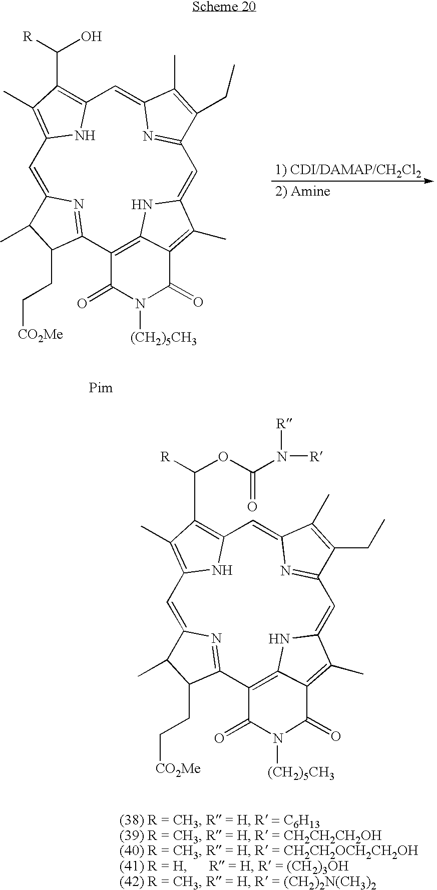

Scheme 20 outlines the synthesis of carbamate derivatives from the 2-(1-hydroxyethyl) purpurin hexylimide Pim. Clearly, other purpurin imide derivatives can be synthesized and modified according to the invention. Scheme 21 outlines the synthesis of a purpurin 18 imide propionic amide derivative that enables the formation of a carbamate on the propionic amide group. In this instance, the ester on the propionic acid group of the purpurin imide is hydrolyzed to form the acid derivative. This is then converted to an amide that is hydroxylated. These hydroxylated purpurin imides may then be reacted in accordance with the invention to produce carbamate derivatives.

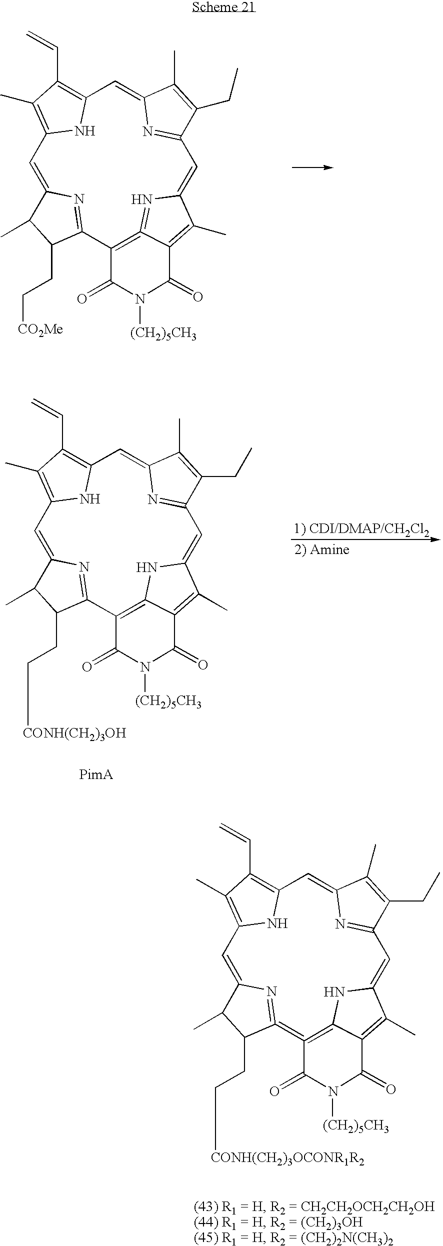

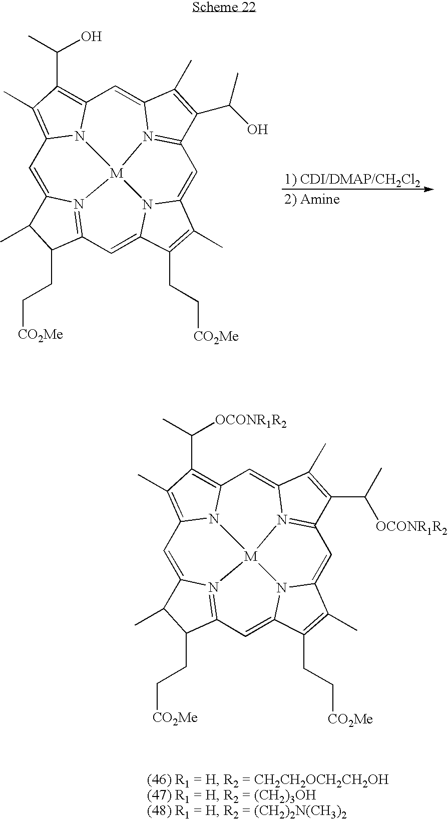

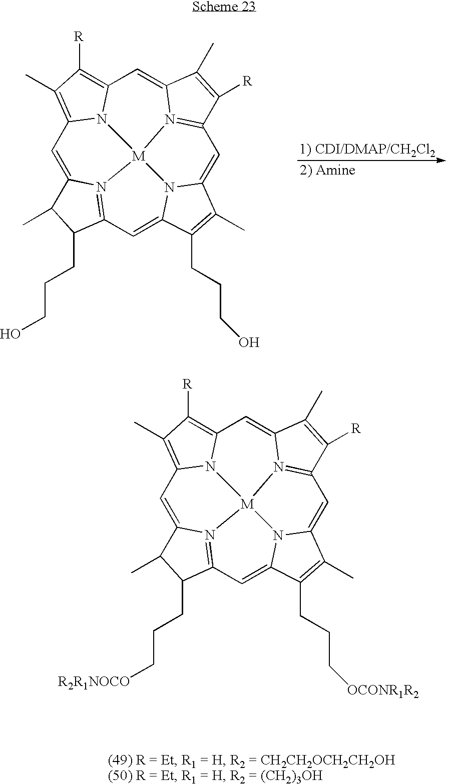

Schemes 22 and 23 outline the synthesis of carbamate derivatives from hematoporphyrin and the dipropylalcohol mesoporphyrin. Clearly, these porphyrinic ring systems allow other functionalization, which can be modified according to the invention.

### Metabolism of Tetrapyrrolic Carbamates

Carbamates are used extensively in herbicides. As such, human metabolites of such chemicals have been reported. Scheme 24 shows the metabolites of phenmedipham.

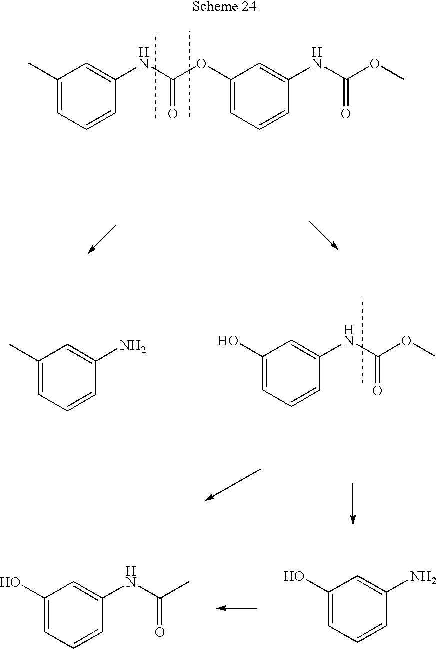

Ester and amide cleavage appear to be the major metabolic routes. As stated previously, one of the inventors' surprising and unexpected biological observations was that the carbamate analogs produce limited skin phototoxicity. Table 1 outlines the normal skin clearance of several carbamate photosensitizers as determined by irradiating hairless rats at the activation wavelength of the photosensitizer at 150 mW/cm2/125 J at different time points post injection. As can be clearly seen, carbamate analogs elicit skin responses at very early time points (1-6 hrs) and not past 6 hrs. Surprisingly, the skin responses observed for the carbamates do not correlate with the normal skin response of the parent hydroxylated tetrapyrrole (expected from ester metabolism). Examples in Table (1) include compounds (26) and (Chl) and (37) and (35). In these cases the hydroxylated parent tetrapyrroles (Chl) and (35), at drug doses of 0.5 μmol/Kg, elicit maximal normal skin responses at 24 and 48 hrs, respectively. By comparison, their carbamate analogs (26) and (37), at drug doses of 1.0 μmol/Kg and 1.5 μmol/Kg, respectively, elicit maximal skin responses at 6 hrs only. Clearly, if ester metabolism of the carbamate back to the parent hydroxylated macrocycle was rapid in blood plasma one would expect skin responses similar to parent hydroxylated macrocycle. This is not the case. It is known by the present inventors and others that metabolism of the propionic acid methyl ester functionality is generally slow in rat and human blood plasma (10-20% metabolism at 24 hrs).

TABLE 1
Normal skin clearance of carbamate photosensitizers

| Compound | Drug dose | Light dose | Skin Response |
|---|---|---|---|
| (7) | 0.5 μmol/Kg | 150 mW/cm²/125 J | 1 hr |
| | 1.0 μmol/Kg | | 1 hr |
| (2) | 0.5 μmol/Kg | 150 mW/cm²/125 J | 6 hr |
| | 1.0 μmol/Kg | | 6 hr |
| (26) | 0.5 μmol/Kg | 150 mW/cm²/125 J | 6 hr |
| | 1.0 μmol/Kg | | 6 hr |
| (Chl)* | 0.5 μmol/Kg | 150 mW/cm²/125 J | 24 hrs |
| (4) | 0.5 μmol/Kg | 150 mW/cm²/125 J | 6 hr |
| | 1.0 μmol/Kg | | 6 hr |
| (5) | 1.0 μmol/Kg | 150 mW/cm²/125 J | 6 hr |
| | 2.0 μmol/Kg | | 6 hr |
| (1) | 1.0 μmol/Kg | 150 mW/cm²/125 J | 6 hr |
| | 2.0 μmol/Kg | | 6 hr |
| (3) | 1.0 μmol/Kg | 150 mW/cm²/125 J | 6 hr |
| | 2.0 μmol/Kg | | 6 hr |
| | 4.0 μmol/Kg | | 6 hr |
| (6) | 0.5 μmol/Kg | 150 mW/cm²/125 J | 6 hr |
| | 0.75 μmol/Kg | | 6 hr |
| (37) | 1.5 μmol/Kg | 150 mW/cm²/125 J | 6 hr |
| (35)* | 0.5 μmol/Kg | 150 mW/cm²/125 J | 48 hr |
| Visudyne* | 1.4 μmol/Kg | 150 mW/cm²/125 J | 24 hr |
| | 2.8 μmol/Kg | | 24 hr |

*Not carbamate photosensitizers

In an attempt to determine what was happening, an HPLC evaluation of several carbamate analogs in rat whole blood plasma was performed. Surprisingly, at very short time points post administration (15 min), it was found that significant metabolism of the carbamate compounds occurred. In our HPLC evaluation of Scheme 25 (shown below), the major metabolite at early time points proved to be compound (52)—no trace of compound (51) could be detected. Over a period of 1-6 hrs, rapid metabolism of the parent carbamate macrocycle occurred in blood plasma. By 24 hrs, little or no parent carbamate macrocycle remained in the plasma.

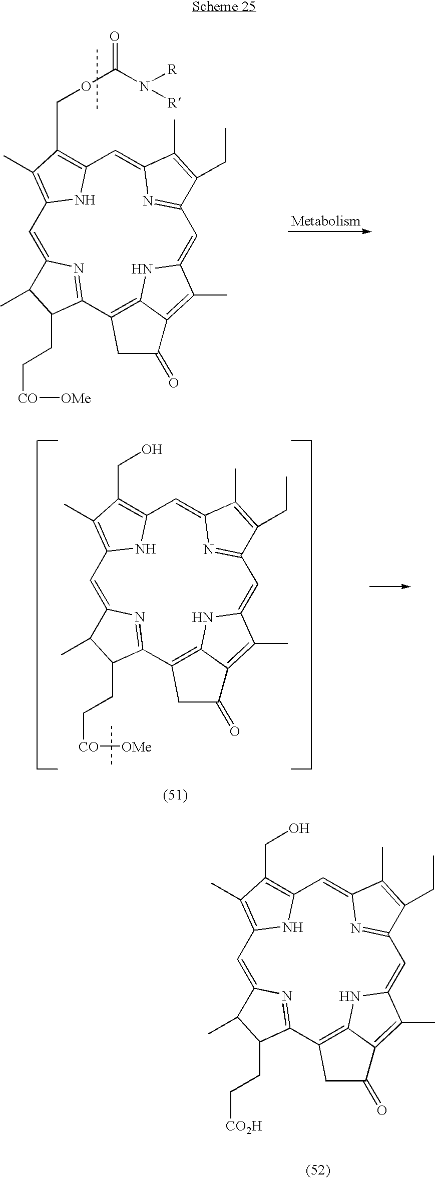

Clearly, the introduction of the carbamate moiety dramatically and unexpectedly enhanced the metabolism of the propionic ester functionality, thus producing (52) within minutes post injection. Compound (52) has been found to be a poor photodynamic agent. Thus, rapid metabolism in the body of carbamate derivatives effectively reduces skin phototoxicity by producing photodynamically less active compounds. Clearly, other compounds, such as (35) and (26) display a similar metabolism enhancement due to the carbamate moiety. Thus, we have found that the introduction of the carbamate moiety generates photoactive molecules (which can be used for therapy at short time points following drug administration), and enhances metabolism of the molecules to limit phototoxic side effects in the administered patient.

The scope of the invention is not limited to the disclosure herein. As shown, any porphyrinic molecule possessing a hydroxyl group may be modified according to the invention to form the desired carbamate derivative. We have shown that distinctly different ring systems show metabolic enhancement when functionalized with carbamates. A large number of porphyrins with widely differing functionality are described in the literature (for example, see “Porphyrins and Metalloporphyrins,” Ed. K. Smith, Elsevier, 1975, New York; “The Porphyrins”, Ed. D. Dolphin, Vol I-V, Academic Press, 1978; “The Porphyrin Handbook”, Ed. K. Kadish, K. M. Smith, R. Guilard, Academic Press, 1999, the disclosures of which are hereby incorporated by reference herein), and are relevant to this invention. They contain various and ranging substituents on the β-pyrrole positions or meso-positions of the porphyrin ring, either symmetrically or asymmetrically substituted on the ring. Examples of such functionality include functional groups having a molecular weight less than about 100,000 daltons and can be a biologically active group or an organic group. Examples include, but are not limited to: (1) hydrogen; (2) halogen, such as fluoro, chloro, iodo and bromo (3) lower alkyl, such as methyl, ethyl, n-propyl, butyl, hexyl, heptyl, octyl, isopropyl, t-butyl, n-pentyl and like groups; (4) lower alkoxy, such as methoxy, ethoxy, isopropoxy, n-butoxy, t-pentoxy and the like; (5) hydroxy; (6) carboxylic acid or acid salts, such as —CH₂COOH, —CH₂COONa, —CH₂CH₂COOH, —CH₂CH₂COONa, —CH₂CH₂CH(Br)COOH, —CH₂CH₂CH(CH₃)COOH, —CH₂CH(Br)COOH, —CH₂CH(CH₃)COOH, —CH(CI)CH₂CH(CH₃)COOH, —CH₂CH₂C(CH₃)₂COOH, —CH₂CH₂C(CH₃)₂COOK, —CH₂CH₂CH₂CH₂COOH, C(CH₃)₂COOH, CH(Cl)₂COOH and the like; (7) carboxylic acid esters, such as —CH₂CH₂COOCH₃, —CH₂CH₂COOCH₂CH₃, —CH₂CH(CH₃)COOCH₂CH₃, —CH₂CH₂CH₂COOCH₂CH₂CH₃, —CH₂CH(CH₃)COOCH₂CH₃, —CH₂CH₂COOCH₂CH₂OH, —CH₂CH₂COOCH₂CH₂N(CH₃)₂ and the like; (8) sulfonic acid or acid salts, for example, group I and group II salts, ammonium salts, and organic cation salts such as alkyl and quaternary ammonium salts; (9) sulfonylamides such as —SO₂NH(alkyl), —SO₂N(alkyl)₂, —SO₂NH(alkyl-OH), —SO₂N(alkyl-OH)₂, —SO₂NH(alkyl)-N(alkyl)₂, —SO₂N(alkyl-N(alkyl)₂)₂, SO₂(NH(alkyl)-N(alkyl)₃⁺Z⁻) and the like, wherein Z⁻ is a counterion, —SO₂NHCH₂CO₂H, substituted and unsubstituted benzene sulfonamides, sulfonylamides of aminoacids and the like; (10) sulfonic acid esters, such as SO₃(alkyl), SO₃(alkyl-OH), SO₃(alkyl-N(alkyl)₂), SO₃(alkyl-N(alkyl)₃⁺Z⁻) and the like, wherein Z⁻ is a counterion, SO₃CH₂CO₂H, and the like; (11) amino, such as unsubstituted or substituted primary amino, methylamino, ethylamino, n-propylamino, isopropylamino, butylamino, sec-butylamino, dimethylamino, trimethylamino, diethylamino, triethylamino, di-n-propylamino, methylethylamino, dimethyl-sec-butylamino, 2-aminoethoxy, ethylenediamino, cyclohexylamino, benzylamino, phenylethylamino, anilino, N-methylanilino, N,N-dimethylanilino, N-methyl-N-ethylanilino, 3,5-dibromo-4-anilino, p-toluidino, diphenylamino, 4,4′-dinitrodiphenylamino and the like; (12) cyano; (13) nitro; (14) a biologically active group; (15) amides, such as —CH₂CH₂CONHCH₃, —CH₂CH₂CONHCH₂CH₃, —CH₂CH₂CON(CH₃)₂, —CH₂CH₂CON(CH₂CH₃)₂, —CH₂CONHCH₃, —CH₂CONHCH₂CH₃, —CH₂CON(CH₃)₂, —CH₂CON(CH₂CH₃)₂, and amides of amino acids and the like; (16) iminium salts, for example CH═N(CH₃)₂⁺Z⁻ and the like, wherein Z⁻ is a counterion), (17) boron containing complexes, (18) carbon cage complexes (e.g., C60 and the like); (19) metal cluster complexes, for example derivatives of EDTA, crown ethers, cyclams, and cyclens; (20) other porphyrin, chlorin, bacteriochlorin, isobacteriochlorin, azoporphyrin, tetraazoporphyrin, phthalocyanine, naphthalocyanine, texaphyrins, tetrapyrrolic macrocycles or dye molecules and the like; (21) alkynyl, including alkyl, aryl, acid and heteroatom substituted alkylnes; (22) leaving or protecting groups; and (23) any other substituent that increases the hydrophilic, amphiphilic or lipophilic nature or stability of the compounds.

The term “biologically active group” can be any group that selectively promotes the accumulation, elimination, binding rate, or tightness of binding in a particular biological environment. For example, one category of biologically active groups is the substituents derived from sugars, specifically: (1) aldoses such as glyceraldehyde, erythrose, threose, ribose, arabinose, xylose, lyxose, allose, altrose, glucose, mannose, gulose, idose, galactose, and talose; (2) ketoses such as hydroxyacetone, erythrulose, rebulose, xylulose, psicose, fructose, sorbose, and tagatose; (3) pyranoses such as glucopyranose; (4) furanoses such as fructo-furanose; (5) O-acyl derivatives such as penta-O-acetyl-α-glucose; (6) O-methyl derivatives such as methyl α-glucoside, methyl β-glucoside, methyl α-glucopyranoside, and methyl-2,3,4,6-tetra-O-methyl-glucopyranoside; (7) phenylosazones such as glucose phenylosazone; (8) sugar alcohols such as sorbitol, mannitol, glycerol, and myo-inositol; (9) sugar acids such as gluconic acid, glucaric acid and glucuronic acid, δ-gluconolactone, δ-glucuronolactone, ascorbic acid, and dehydroascorbic acid; (10) phosphoric acid esters such as α-glucose 1-phosphoric acid, α-glucose 6-phosphoric acid, α-fructose 1,6-diphosphoric acid, and α-fructose 6-phosphoric acid; (11) deoxy sugars such as 2-deoxy-ribose, rhammose (deoxy-mannose), and fructose (6-deoxy-galactose); (12) amino sugars such as glucosamine, galactosamine, muramic acid, and neurarninic acid; (13) disaccharides such as maltose, sucrose and trehalose; (14) trisaccharides such as raffinose (fructose, glucose, galactose) and melezitose (glucose, fructose); (15) polysaccharides (glycans) such as glucans and mannans; and (16) storage polysaccharides such as α-amylose, amylopectin, dextrins, and dextrans.

Amino acid derivatives are also useful biologically active substituents, such as those derived from valine, leucine, isoleucine, threonine, methionine, phenylalanine, tryptophan, alanine, arginine, aspartic acid, cystine, cysteine, glutamic acid, glycine, histidine, proline, serine, tyrosine, asparagine and glutamine. Also useful are peptides, particularly those known to have affinity for specific receptors, for example, oxytocin, vasopressin, bradykinin, LHRH, thrombin and the like.

Another useful group of biologically active substituents are those derived from nucleosides, for example, ribonucleosides such as adenosine, guanosine, cytidine, and uridine, and 2′-deoxyribonucleosides such as 2′-deoxyadenosine, 2′-deoxyguanosine, 2′-deoxycytidine, and 2′-deoxythymidine.

Another category of biologically active groups that is particularly useful is any ligand that is specific for a particular biological receptor. The term “ligand specific for a receptor” refers to a moiety that binds a receptor at cell surfaces, and thus contains contours and charge patterns that are complementary to those of the biological receptor. The ligand is not the receptor itself, but a substance complementary to it. It is well understood that a wide variety of cell types have specific receptors designed to bind hormones, growth factors, or neurotransmitters. However, while these embodiments of ligands specific for receptors are known and understood, the phrase “ligand specific for a receptor” as used herein refers to any substance, natural or synthetic, that binds specifically to a receptor.

Examples of such ligands include: (1) the steroid hormones, such as progesterone, estrogens, androgens, and the adrenal cortical hormones; (2) growth factors, such as epidermal growth factor, nerve growth factor, fibroblast growth factor, and the like; (3) other protein hormones, such as human growth hormone, parathyroid hormone, and the like; (4) neurotransmitters, such as acetylcholine, serotonin, dopamine, and the like; and (5) antibodies. Any analog of these substances that also succeeds in binding to a biological receptor is also included.

Particularly useful examples of substituents tending to increase the amphiphilic nature of the compounds include: (1) short or long chain alcohols, for example, —C₁₂H₂₄—OH where —C₁₂H₂₄ is hydrophobic; (2) fatty acids and their salts, such as the sodium salt of the long-chain fatty acid oleic acid; (3) phosphoglycerides, such as phosphatidic acid, phosphatidyl ethanolamine, phosphatidyl choline, phosphatidyl serine, phosphatidyl inositol, phosphatidyl glycerol, phosphatidyl 3′-O-alanyl glycerol, cardiolipin, or phosphatidyl choline; (4) sphingolipids, such as sphingomyelin; and (5) glycolipids, such as glycosyldiacylglycerols, cerebrosides, sulfate esters of cerebrosides or gangliosides. It would be obvious to one skilled in the art what other groups, or combinations of the groups described, would be suitable in the invention.

The compounds of the present invention, or their pharmaceutically acceptable salts, solvates, prodrugs, or metabolites, can be administered to the host in a variety of forms adapted to the chosen route of administration, e.g., orally, intravenously, intramuscularly or subcutaneously.

The active compound may be orally administered, for example, with an inert diluent or with an assimilable edible carrier, or it may be enclosed in hard or soft shell gelatin capsules, or it may be compressed into tablets, or it may be incorporated directly with food. For oral therapeutic administration, the active compound may be incorporated with excipients and used in the form of ingestible tablets, buccal tablets, troches, capsules, elixirs, suspensions, syrups, wafers, and the like. Such compositions and preparations should contain at least about 0.1% of active compound. The percentage of the compositions and preparations may, of course, be varied and may, for example, conveniently be between about 2 to about 60% of the weight of the administered product. The amount of active compound in such therapeutically useful compositions is such that a suitable dosage will be obtained. Preferred compositions or preparations according to the present invention are prepared so that an oral dosage unit form contains between about 50 and 300 mg of active compound.

The tablets, troches, pills, capsules and the like may also contain the following: a binder such as gum tragacanth, acacia, corn starch or gelatin; excipients such as dicalcium phosphate; a disintegrating agent such as corn starch, potato starch, alginic acid and the like; a lubricant such as magnesium stearate; a sweetening agent such as sucrose, lactose or saccharin; or a flavoring agent such as peppermint, oil of wintergreen, or cherry flavoring. When the dosage unit form is a capsule, it may contain, in addition to materials of the above type, a liquid carrier. Various other materials may be present as coatings or to otherwise modify the physical form of the dosage unit. For instance, tablets, pills, or capsules may be coated with shellac, sugar or both. A syrup or elixir may contain the active compound, sucrose as a sweetening agent, methyl and propylparabens as preservatives, a dye or flavoring such as cherry or orange flavor. Of course, any material used in preparing any dosage unit form should be pharmaceutically pure and substantially non-toxic in the amounts employed. In addition, the active compound may be incorporated into sustained-release preparations and formulations.

The active compound may also be administered parenterally or intraperitoneally. Solutions of the active compound as a free base or pharmacologically acceptable salt can be prepared in water suitably mixed with a surfactant such as hydroxypropylcellulose. Dispersions can also be prepared in glycerol, liquid polyethylene glycols, and mixtures thereof and in oils. Under ordinary conditions of storage and use, these preparations contain a preservative to prevent the growth of microorganisms.

The pharmaceutical forms suitable for injectable use include sterile aqueous solutions or dispersions and sterile powders for the extemporanous preparation of sterile injectable solutions, dispersions, or liposomal or emulsion formulations. In all cases the form must be sterile and must be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating such as lecithin, by the maintenance of the required particle size in the case of dispersions and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compound in the required amount in the appropriate solvent with various other ingredients enumerated above, as required, followed by filtered sterilization. Generally, dispersions are prepared by incorporating the various sterilized active ingredients into a sterile vehicle that contains the basic dispersion medium and the required additional ingredients from those enumerated above. In the case of sterile powders for the preparation of sterile injectable solutions, the preferred methods of preparation are vacuum drying and freeze-drying, which yield a powder of the active ingredient plus any additional desired ingredient from previously sterile-filtered solutions thereof.

The compounds of the invention may also be applied directly to tumors in the host whether internal or external, in topical compositions. Exemplary compositions include solutions of the inventive compounds in solvents, particularly aqueous solvents, most preferably water. Alternatively, for topical application particularly to skin tumors, the present new compounds may be dispersed in the usual cream or salve formulations commonly used for this purpose (such as liposomes, ointments, gels, hydrogels, and oils) or may be provided in the form of spray solutions or suspensions that may include a propellant usually employed in aerosol preparations.

As used herein, “pharmaceutically acceptable carrier” includes any and all solvents, dispersion media, coatings, antibacterial and antifungal agents, isotonic and absorption delaying agents and the like. The use of such media and agents for pharmaceutical active substances is well known in the art. Except insofar as any conventional media or agent is incompatible with the active ingredient, its use in the therapeutic compositions is contemplated. Supplementary active ingredients can also be incorporated into the compositions.

It is especially advantageous to formulate parenteral compositions in dosage unit form for ease of administration and uniformity of dosage. Dosage unit form as used herein refers to physically discrete units suited as unitary dosages for the mammalian subjects to be treated, each unit containing a predetermined quantity of active material calculated to produce the desired therapeutic effect in association with the required pharmaceutical carrier. The specifications for the novel dosage unit forms of the invention are dictated by and directly dependent on (a) the unique characteristics of the active material and the particular therapeutic effect to be achieved, and (b) the limitations inherent in the art of compounding such an active material for the treatment of tumors in living subjects.

### Definitions

As used in the present application, the following definitions apply.

The term “alkyl” as used herein refers to substituted or unsubstituted, straight or branched chain groups, preferably having one to ten, more preferably having one to six, and most preferably having from one to four carbon atoms. The term “C₁-C₆ alkyl” represents a straight or branched alkyl chain having from one to six carbon atoms. Exemplary C₁-C₆ alkyl groups include methyl, ethyl, n-propyl, isopropyl, butyl, isobutyl, sec-butyl, t-butyl, pentyl, neo-pentyl, hexyl, isohexyl, and the like. The term “C₁-C₆ alkyl” includes within its definition the term “C₁-C₄ alkyl.” Such alkyl groups may themselves be ethers or thioethers, or aminoethers or dendrimers.

The term “cycloalkyl” represents a substituted or unsubstituted, saturated or partially saturated, mono- or poly-carbocyclic ring, preferably having 5-14 ring carbon atoms. Exemplary cycloalkyls include monocyclic rings having from 3-7, preferably 3-6, carbon atoms, such as cyclopropyl, cyclobutyl, cyclopentyl, cyclohexyl and the like. An exemplary cycloalkyl is a C₅-C₇ cycloalkyl, which is a saturated hydrocarbon ring structure containing from five to seven carbon atoms.

The term “aryl” as used herein refers to an aromatic, monovalent, monocyclic, bicyclic, or tricyclic radical containing 6, 10, 14, or 18 carbon ring atoms, which may be unsubstituted or substituted, and to which may be fused one or more cycloalkyl groups, heterocycloalkyl groups, or heteroaryl groups, which themselves may be unsubstituted or substituted by one or more suitable substituents. Illustrative examples of aryl groups include, but are not limited to, phenyl, napthyl, anthryl, phenanthryl, fluoren-2-yl, indan-5-yl, and the like.

The term “halogen” represents chlorine, fluorine, bromine or iodine. The term “halocarbon” represents one or more halogens bonded to a carbon bearing group.

The term “carbocycle” represents a substituted or unsubstituted aromatic or a saturated or a partially saturated 5-14 membered monocyclic or polycyclic ring, such as a 5- to 7-membered monocyclic or 7- to 10-membered bicyclic ring, wherein all the ring members are carbon atoms.

The term “electron withdrawing group” is intended to mean a chemical group containing an electronegative element such as halogen, sulfur, nitrogen or oxygen.

A “heterocycloalkyl group” is intended to mean a non-aromatic, monovalent, monocyclic, bicyclic, or tricyclic radical, which is saturated or unsaturated, containing 3 to 18 ring atoms, and which includes 1 to 5 heteroatoms selected from nitrogen, oxygen and sulfur, wherein the radical is unsubstituted or substituted, and to which may be fused one or more cycloalkyl groups, aryl groups, or heteroaryl groups, which themselves may be unsubstituted or substituted. Illustrative examples of heterocycloalkyl groups include, but are not limited to, azetidinyl, pyrrolidyl, piperidyl, piperazinyl, morpholinyl, tetrahydro-2H-1,4-thiazinyl, tetrahydrofuryl, dihydrofuryl, tetrahydropyranyl, dihydropyranyl, 1,3-dioxolanyl, 1,3-dioxanyl, 1,4-dioxanyl, 1,3-oxathiolanyl, 1,3-oxathianyl, 1,3-dithianyl, azabicylo[3.2.1]octyl, azabicylo[3.3.1 ]nonyl, azabicylo[4.3.0]nonyl, oxabicylo[2.2.1]heptyl, 1,5,9-triazacyclododecyl, and the like.

A “heteroaryl group” is intended to mean an aromatic, monovalent, monocyclic, bicyclic, or tricyclic radical containing 5 to 18 ring atoms, including 1 to 5 heteroatoms selected from nitrogen, oxygen and sulfur, which may be unsubstituted or substituted, and to which may be fused one or more cycloalkyl groups, heterocycloalkyl groups, or aryl groups, which themselves may be unsubstituted or substituted. Illustrative examples of heteroaryl groups include, but are not limited to, thienyl, pyrrolyl, imidazolyl, pyrazolyl, furyl, isothiazolyl, furazanyl, isoxazolyl, thiazolyl, pyridyl, pyrazinyl, pyrimidinyl, pyridazinyl, triazinyl, benzo[b]thienyl, naphtho[2,3-b]thianthrenyl, isobenzofuranyl, chromenyl, xanthenyl, phenoxathienyl, indolizinyl, isoindolyl, indolyl, indazolyl, purinyl, isoquinolyl, quinolyl, phthalazinyl, naphthyridinyl, quinoxyalinyl, quinzolinyl, benzothiazolyl, benzimidazolyl, tetrahydroquinolinyl, cinnolinyl, pteridinyl, carbazolyl, beta-carbolinyl, phenanthridinyl, acridinyl, perimidinyl, phenanthrolinyl, phenazinyl, isothiazolyl, phenothiazinyl, and phenoxazinyl.

The term “leaving group” as used herein refers to any group that departs from a molecule in a substitution reaction by breakage of a bond. Examples of leaving groups include, but are not limited to, halides, tosylates, arenesulfonates, alkylsulfonates, and triflates.

Suitable protecting groups are known to those skilled in the art. Examples of suitable protecting groups can be found in T. Green & P. Wuts, Protective Groups in Organic Synthesis (2d ed. 1991), which is hereby incorporated by reference herein.

Suitable salt anions include, but are not limited to, inorganics such as halogens, pseudohalogens, sulfates, hydrogen sulfates, nitrates, hydroxides, phosphates, hydrogen phosphates, dihydrogen phosphates, perchlorates, and related complex inorganic anions; and organics such as carboxylates, sulfonates, bicarbonates and carbonates.

Examples of substituents for alkyl and aryl groups include mercapto, thioether, nitro (NO₂), amino, aryloxyl, halogen, hydroxyl, alkoxyl, and acyl, as well as aryl, cycloalkyl and saturated and partially saturated heterocycles. Examples of substituents for cycloalkyl groups include those listed above for alkyl and aryl, as well as aryl and alkyl groups themselves.

Exemplary substituted aryls include a phenyl or naphthyl ring substituted with one or more substituents, preferably one to three substituents, independently selected from halo, hydroxy, morpholino(C₁-C₄)alkoxy carbonyl, pyridyl, (C₁-C₄)alkoxycarbonyl, halo (C₁-C₄)alkyl, C₁-C₄ alkyl, C₁-C₄ alkoxy, carboxy, C₁-C₄ alkocarbonyl, carbamoyl, N—(C₁-C₄)alkylcarbamoyl, amino, C₁-C₄ alkylamino, di(C₁-C₄)alkylamino or a group of the formula —(CH₂)ₐ—R₇ where a is 1, 2, 3 or 4; and R₇ is hydroxy, C₁-C₄ alkoxy, carboxy, C₁-C₄ alkoxycarbonyl, amino, carbamoyl, C₁-C₄ alkylamino or di(C₁-C₄)alkylamino.

Another substituted alkyl is halo(C₁-C₄)alkyl, which represents a straight or branched alkyl chain having from one to four carbon atoms with 1-3 halogen atoms attached to it. Exemplary halo(C₁-C₄)alkyl groups include chloromethyl, 2-bromoethyl, 1-chloroisopropyl, 3-fluoropropyl, 2,3-dibromobutyl, 3-chloroisobutyl, iodo-t-butyl, trifluoromethyl, and the like.

Another substituted alkyl is hydroxy (C₁-C₄)alkyl, which represents a straight or branched alkyl chain having from one to four carbon atoms with a hydroxy group attached to it. Exemplary hydroxy(C₁-C₄)alkyl groups include hydroxymethyl, 2-hydroxyethyl, 3-hydroxypropyl, 2-hydroxyisopropyl, 4-hydroxybutyl, and the like.

Yet another substituted alkyl is C₁-C₄ alkylthio(C₁-C₄)alkyl, which is a straight or branched C₁-C₄ alkyl group with a C₁-C₄ alkylthio group attached to it. Exemplary C₁-C₄ alkylthio(C₁-C₄)alkyl groups include methylthiomethyl, ethylthiomethyl, propylthiopropyl, sec-butylthiomethyl, and the like.

Yet another exemplary substituted alkyl is heterocycle(C₁-C₄)alkyl, which is a straight or branched alkyl chain having from one to four carbon atoms with a heterocycle attached to it. Exemplary heterocycle(C₁-C₄)alkyls include pyrrolylmethyl, quinolinylmethyl, 1-indolylethyl, 2-furylethyl, 3-thien-2-ylpropyl, 1-imidazolylisopropyl, 4-thiazolylbutyl and the like.

Yet another substituted alkyl is aryl(C₁-C₄)alkyl, which is a straight or branched alkyl chain having from one to four carbon atoms with an aryl group attached to it. Exemplary aryl(C₁-C₄)alkyl groups include phenylmethyl, 2-phenylethyl, 3-naphthyl-propyl, 1-naphthylisopropyl, 4-phenylbutyl and the like.

The heterocycloalkyls and the heteroaryls can, for example, be substituted with 1, 2 or 3 substituents independently selected from halo, halo(C₁-C₄)alkyl, C₁-C₄ alkyl, C₁-C₄ alkoxy, carboxy, C₁-C₄ alkoxycarbonyl, carbamoyl, —(C₁-C₄)alkylcarbamoyl, amino, C₁-C₄ alkylamino, di(C₁-C₄)alkylamino or a group having the structure —(CH₂)ₐ—R₇ where a is 1, 2, 3 or 4 and R₇ is hydroxy, C₁-C₄ alkoxy, carboxy, C₁-C₄ alkoxycarbonyl, amino, carbamoyl, C₁-C₄ alkylamino or di(C₁-C₄)alkylamino.

Examples of substituted heterocycloalkyls include, but are not limited to, 3-N-t-butyl carboxamide decahydroisoquinolinyl and 6-N-t-butyl carboxamide octahydro-thieno[3,2-c]pyridinyl. Examples of substituted heteroaryls include, but are not limited to, 3-methylimidazolyl, 3-methoxypyridyl, 4-chloroquinolinyl, 4-aminothiazolyl, 8-methylquinolinyl, 6-chloroquinoxalinyl, 3-ethylpyridyl, 6-methoxybenzimidazolyl, 4-hydroxyfuryl, 4-methylisoquinolinyl, 6,8-dibromoquinolinyl, 4,8-dimethylnaphthyl, 2-methyl-1,2,3,4-tetrahydroisoquinolinyl, N-methyl-quinolin-2-yl, 2-tibutoxycarbonyl-1,2,3,4-isoquinolin-7-yl and the like.

A “pharmaceutically acceptable solvate” is intended to mean a solvate that retains the biological effectiveness and properties of the biologically active components of the inventive compounds. Examples of pharmaceutically acceptable solvates include, but are not limited to, compounds prepared using water, isopropanol, ethanol, DMSO, and other excipients generally referred to as GRAS ingredients.

In the case of solid formulations, it is understood that the compounds of the invention may exist in different polymorph forms, such as stable and metastable crystalline forms and isotropic and amorphous forms, all of which are intended to be within the scope of the present invention.

A “pharmaceutically acceptable salt” is intended to mean those salts that retain the biological effectiveness and properties of the free acids and bases and that are not biologically or otherwise undesirable. Examples of pharmaceutically acceptable salts include, but are not limited to, sulfates, pyrosulfates, bisulfates, sulfites, bisulfites, phosphates, monohydrogen phosphates, dihydrogen phosphates, metaphosphates, pyrophosphates, chlorides, bromides, iodides, acetates, propionates, citrates, decanoates, caprylates, acrylates, formates, isobutyrates, caproates, heptanoates, propiolates, oxalates, malonates, succinates, suberates, sebacates, fumarates, maleates, butyne-1,4-dioates, hexyne-1,6-dioates, benzoates, chlorobenzoates, methylbenzoates, dinitrobenzoates, hydroxybenzoates, methoxybenzoates, phthalates, sulfonates, xylenesulfonates, phenylacetates, phenyl propionates, phenyl butyrates, citrates, lactates, hydroxybutyrates, glycolates, tartrates, methanesulfoantes, propanesulfonates, naphthalene-1-sulfonates, naphthalene-2-sulfonates, and mandelates.

If a compound of the present invention is a base, the desired salt may be prepared by any suitable method known to the art, including treatment of the free base with an inorganic acid, such as hydrochloric acid, hydrobromic acid, sulfuric acid, nitric acid, phosphoric acid and the like, or with an organic acid, such as acetic acid, maleic acid, succinic acid, mandelic acid, fumaric acid, malonic acid, pyruvic acid, oxalic acid, glycolic acid, salicylic acid, pyranosidyl acids such as glucuronic acid and galacturonic acid, alpha-hyrodoxy acids such as citric acid and tartaric acid, amino acids such as aspartic acid and glutamic acid, aromatic acids such as benzoic acid and cinnamic acid, sulfonic acids such as p-toluenesulfonic acid or ethanesulfonic acid, or the like.

If a compound of the present invention is an acid, the desired salt may be prepared by any suitable method known to the art, including treatment of the free acid with an inorganic or organic base, such as an amine (primary, secondary or tertiary), or an alkali metal or alkaline earth metal hydroxide or the like. Illustrative examples of suitable salts include organic salts derived from amino acids such as glycine and arginine; ammonia; primary, secondary and tertiary amines; cyclic amines such as piperidine, morpholine and piperazine; and inorganic salts derived from sodium, calcium, potassium, magnesium, manganese, iron, copper, zinc, aluminum, and lithium.

### EXAMPLES

In the following synthetic examples silica gel 60 (230-400 mesh) was used for column chromatography. Analytical thin layer chromatography was performed on Merck 60 F254 silica gel (precoated on aluminum). All compounds were analyzed by ¹H NMR, UV and characterized by mass spectrometry (MS). ¹H spectra were recorded using a Unity Inova Varian 500 MHz spectrometer. Electronic spectra were recorded on a Beckman DU 640 spectrophotometer. High resolution mass spectra were obtained on a VG 70SE double focussing mass spectrometer equipped with an oversize data system.

### Example 1

### Compound (1)

2-Desvinyl-2-hydroxymethyl pyropheophorbide methyl ester (pyr, R═H) (100 mg) was stirred with CDl (100 mg) in CH₂Cl₂ (25 ml) and in the presence of DMAP (25 mg) at room temperature until the reaction was complete (3 h). Hexyl amine (0.5 ml) was then added to the solution and stirred for 6 h at room temperature. The reaction mixture was diluted with CH₂Cl₂ (25 ml) and washed with 1N HCl (1×50 ml) followed by 10% aq. NaHCO₃ (1×50 ml) and water (1×50 ml), dried and evaporated to dryness. The residue was purified by column chromatography on silica gel. The product was eluted using 4% acetone/CH₂Cl₂ and then crystallized from CH₂Cl₂/Isopropyl ether/hexane. Yield of compound (1)=90 mg.

### Example 2

### Compound (2)

2-Desvinyl-2-hydroxymethyl pyropheophorbide methyl ester (pyr, R=H) (250 mg) was stirred with CDl (150 mg) in CH₂Cl₂ (50 ml) and in the presence of DMAP (25 mg) at room temperature until the reaction was complete (3 h). 3-Amino-1-propanol (1.5 ml) was then added to the solution and stirred overnight at room temperature. The reaction mixture was washed with water (2×50 ml), dried and evaporated to dryness. The residue was purified by column chromatography on silica gel. The product was isolated using 5% MeOH/CH₂Cl₂ and crystallized from CH₂Cl₂/MeOH/Ether. Yield of compound (2)=250 mg.

### Example 3

### Compound (3)

2-Desvinyl-2-hydroxymethyl pyropheophorbide methyl ester (pyr, R═H) (150 mg) was stirred with CDl (100 mg) in CH₂Cl₂ (50 ml) and in the presence of DMAP (25 mg) at room temperature until the reaction was complete (3 h). 2-(2-Aminoethoxy)ethanol (1.0 ml) was then added to the solution and stirred for 6 h at room temperature. The reaction mixture was washed with water (2×50 ml), dried and evaporated to dryness. The residue was purified by column chromatography on silica gel. The product was isolated using 10% MeOH/CH₂Cl₂ and crystallized from CH₂Cl₂/hexane. Yield of compound (3)=143 mg.

### Example 4

### Compound (4)

2-Desvinyl-2-hydroxymethyl pyropheophorbide methyl ester (pyr, R=H) (100 mg) was stirred with CDl (100 mg) in CH₂Cl₂ (10 ml) and in the presence of DMAP (10 mg) at room temperature until the reaction was complete (3 h). 2-Methoxy-ethylamine (0.5 ml) was then added to the solution and stirred for 4 h at room temperature. The reaction mixture was washed with water (2×50 ml), dried and evaporated to dryness. The residue was purified by column chromatography on silica gel. The product was isolated using 2% acetone/CH₂Cl₂ and crystallized from CH₂Cl₂/hexane. Yield of compound (4)=108 mg.

### Example 5

### Compound (5)

2-Desvinyl-2-hydroxymethyl pyropheophorbide methyl ester (pyr, R═H) (100 mg) was stirred with CDl (100 mg) in CH₂Cl₂ (20 ml) and in the presence of DMAP (25 mg) at room temperature until the reaction was complete (3 h). N,N-Dimethylethylenediamine (0.5 ml) was then added to the solution and stirred for 4 h at room temperature. The reaction mixture was washed with water (2×50 ml), dried and evaporated to dryness. The residue was purified by column chromatography on silica gel. The product was isolated using 5% MeOH/CH₂Cl₂ and crystallized from CH₂Cl₂/hexane. Yield of compound (5)=110 mg.

### Example 6

### Compound (6)

2-Desvinyl-1-hydroxy-1-ethyl pyropheophorbide methyl ester (pyr, R═CH₃) (125 mg) was stirred with CDl (125 mg) in CH₂Cl₂ (25 ml) and in the presence of DMAP (25 mg) at room temperature until the reaction was complete (3 h). 3-Amino-1-propanol (0.5 ml) was then added to the solution and stirred overnight at room temperature. The reaction mixture was washed with water (2×50 ml), dried and evaporated to dryness. The residue was purified by column chromatography on silica gel. The product was isolated using 7% MeOH/CH₂Cl₂ and crystallized from CH₂Cl₂/ether/hexane. Yield of compound (6)=90 mg.

### Example 7

### Compound (7)

2-Desvinyl-2-hydroxymethyl pyrropheophorbide methyl ester (pyr, R═H) (100 mg) was stirred with CDl (100 mg) in CH₂Cl₂ (20 ml) and in the presence of DMAP (25 mg) at room temperature until the reaction was complete (3 h). 1,1,3,3-Tetramethylguanidine (0.5 ml) was then added to the solution and stirred for 24 h at room temperature. The reaction mixture was washed with water (2×50 ml), dried and evaporated to dryness. The residue was purified by column chromatography on silica gel. The product was isolated using 5% MeOH/CH₂Cl₂ and crystallized from CH₂Cl₂ /hexane. Yield of compound (7)=67 mg.

### Example 8

### Compound (8)

2-Desvinyl-2-(1-hydroxyethyl)pyrropheophorbide methyl ester (pyr, R═H) (100 mg) was stirred with CDl (100 mg) in CH₂Cl₂ (20 ml) and in the presence of DMAP (25 mg) at room temperature until the reaction was complete (3 h). 1,1,3,3-Tetramethylguanidine (0.5 ml) was then added to the solution and stirred for 24 h at room temperature. The reaction mixture was washed with water (2×50 ml), dried and evaporated to dryness. The residue was purified by column chromatography on silica gel. The product was isolated using 5% MeOH/CH₂Cl₂ and crystallized from CH₂Cl₂/hexane. Yield of compound (8)=70 mg.

### Example 9

### Compound (9)

9-Desoxo-9-hydroxy pyrropheophorbide methyl ester (Rpheo, M=2H) (100 mg) was stirred with CDl (100 mg) in CH₂Cl₂ (20 ml) and in the presence of DMAP (25 mg) at room temperature until the reaction was complete (3 h). 3-Aminopropanol (0.5 ml) was then added to the solution and stirred for 24 h at room temperature. The reaction mixture was washed with water (2×50 ml), dried and evaporated to dryness. The residue was purified by column chromatography on silica gel. The product was isolated using 5% MeOH/CH₂Cl₂ and crystallized from CH₂Cl₂ /hexane. Yield of compound (9)=72 mg.

### Example 10

### Compound (10)

9-Desoxo-9-hydroxy pyrropheophorbide methyl ester (Rpheo, M=2H) (100 mg) was stirred with CDl (100 mg) in CH₂Cl₂ (20 ml) and in the presence of DMAP (25 mg) at room temperature until the reaction was complete (3 h). Hexylamine (0.5 ml) was then added to the solution and stirred for 24 h at room temperature. The reaction mixture was washed with water (2×50 ml), dried, and evaporated to dryness. The residue was purified by column chromatography on silica gel. The major fraction was isolated using 5% MeOH/ CH₂Cl₂ and evaporated to dryness. The free base hexyl carbamate (72 mg) was dissolved in chloroform (20 mL) and a solution of zinc chloride (50 mg) in methanol (2.0 mL) was added. The solution was warmed at 65° C. for 1 hr and then cooled to room temperature. The organic layer was washed well with water and collected and dried over sodium sulfate. The solution was filtered and evaporated to dryness. The product was crystallized from CH₂Cl₂/hexane. Yield of compound (10)=70 mg.

### Example 11

### Compound (11)

Pyrropheophorbide (300 mg) was dissolved in dichloromethane (50 mL) and tetrahydrofuran (50 mL) and triethylamine added (0.3 mL). The solution was cooled to O° C. in an ice bath. Ethyl chloroformate (0.3 mL) was added and the solution stirred for 1 hr at room temperature. 3-aminopropylalcohol (1 ml) was added and the reaction closely monitored by TLC (5% acetone/dichloromethane). When deemed complete the reaction was poured into water (100 mL) and the organic phase separated and rotoevaporated. The residue was chromatographed on silica using 2% methanol/dichloromethane as eluent and the major grey fraction collected. The organic layer was removed by rotoevaporation and the product precipitated from dichloromethane/methanol. Yield of compound (11)=289 mg.

### Example 12

### Compound (12)

Pyrropheophorbide (300 mg) was dissolved in dichloromethane (50 mL) and tetrahydrofuran (50 mL) and triethylamine added (0.3 mL). The solution was cooled to O° C. in an ice bath. Ethyl chloroformate (0.3 mL) was added and the solution stirred for 1 hr at room temperature. 2-(2-Aminoethoxy)ethanol (1.0 ml) was added and the reaction closely monitored by TLC (5% acetone/dichloromethane). When deemed complete the reaction was poured into water (100 mL) and the organic phase separated and rotoevaporated. The residue was chromatographed on silica using 2% methanol/dichloromethane as eluent and the major grey fraction collected. The organic layer was removed by rotoevaporation and the product precipitated from dichloromethane/hexane. Yield of title compound (12)=292 mg.

### Example 13

### Compound (13)

Compound (11) (100 mg) was stirred with CDl (100 mg) in CH₂Cl₂ (20 ml) and in the presence of DMAP (25 mg) at room temperature until the reaction was complete. Diethanolamine (0.5 ml) was then added to the solution and stirred for 24 h at room temperature. The reaction mixture was washed with water (2×50 ml), dried and evaporated to dryness. The residue was purified by column chromatography on silica gel. The product was isolated using 5% MeOH/CH₂Cl₂ and crystallized from CH₂Cl₂/hexane. Yield of compound (13)=72 mg.

### Example 14

### Compound (14)

Compound (11) (100 mg) was stirred with CDl (100 mg) in CH₂Cl₂ (20 ml) and in the presence of DMAP (25 mg) at room temperature until the reaction was complete. Aspartic acid di-t-Butyl ester (500 mg) was then added to the solution and stirred for 24 h at room temperature. The reaction mixture was washed with water (2×50 ml), dried and evaporated to dryness. The residue was purified by column chromatography on silica gel. The product was isolated using 5% MeOH/CH₂Cl₂ and crystallized from CH₂Cl₂/hexane. Yield of compound (14)=92 mg.

### Example 15

### Compound (15)

Compound (11) (100 mg) was stirred with CDl (100 mg) in CH₂Cl₂ (20 ml) and in the presence of DMAP (25 mg) at room temperature until the reaction was complete. Glycine t-butyl ester (500 mg) was then added to the solution and stirred for 24 h at room temperature. The reaction mixture was washed with water (2×50 ml), dried and evaporated to dryness. The residue was purified by column chromatography on silica gel. The required product was isolated using 5% MeOH/ CH₂Cl₂ and crystallized from CH₂Cl₂/hexane. Yield of compound (15)=90 mg.

### Example 16

### Compound (16)

Compound (12) (100 mg) was stirred with CDl (100 mg) in CH₂Cl₂ (20 ml) and in the presence of DMAP (25 mg) at room temperature until the reaction was complete. 3-Aminopropanol (0.5 mL) was then added to the solution and stirred for 24 h at room temperature. The reaction mixture was washed with water (2×50 ml), dried and evaporated to dryness. The residue was purified by column chromatography on silica gel. The product was isolated using 5% MeOH/CH₂Cl₂ and crystallized from CH₂Cl₂/hexane. Yield of compound (16)=90 mg.

### Example 17

### Compound (17)

Methyl 3-hydroxymethyl pyrropheophorbide (100 mg) was stirred with CDl (100 mg) in CH₂Cl₂ (20 ml) and in the presence of DMAP (25 mg) at room temperature until the reaction was complete. 3-Aminopropanol (0.5 mL) was then added to the solution and stirred for 24 h at room temperature. The reaction mixture was washed with water (2×50 ml), dried and evaporated to dryness. The residue was purified by column chromatography on silica gel. The product was isolated using 5% MeOH/CH₂Cl₂ and crystallized from CH₂Cl₂/hexane. Yield of compound (17)=90 mg.

### Example 18

### Compound (18)

Methyl 3-hydroxymethyl pyrropheophorbide (100 mg) was stirred with CDl (100 mg) in CH₂Cl₂ (20 ml) and in the presence of DMAP (25 mg) at room temperature until the reaction was complete. 2-(2-Aminoethoxy)ethanol (0.5 mL) was then added to the solution and stirred for 24 h at room temperature. The reaction mixture was washed with water (2×50 ml), dried and evaporated to dryness. The residue was purified by column chromatography on silica gel. The product was isolated using 5% MeOH/CH₂Cl₂ and crystallized from CH₂Cl₂ /hexane. Yield of compound (18)=92 mg.

### Example 19

### Compound (19)

Methyl 3-hydroxymethyl pyrropheophorbide (100 mg) was stirred with CDl (100 mg) in CH₂Cl₂ (20 ml) and in the presence of DMAP (25 mg) at room temperature until the reaction was complete. N,N-Dimethylaminoethylamine (0.5 mL) was then added to the solution and stirred for 24 h at room temperature. The reaction mixture was washed with water (2×50 ml), dried and evaporated to dryness. The residue was purified by column chromatography on silica gel. The product was isolated using 5% MeOH/CH₂Cl₂ and crystallized from CH₂Cl₂ /hexane. Yield of compound (19)=92 mg.

### Example 20

### Compound (20)

2-Desvinyl-1-hydroxymethyl chlorin e6 tri-methyl ester (Ce6, R═H) (100 mg) was stirred with CDl (100 mg) in CH₂Cl₂ (25 ml) and in the presence of DMAP (25 mg) at room temperature until the reaction was complete (3 h). Hexyl amine (0.5 ml) was then added to the solution and stirred for 6 h at room temperature. The reaction mixture was diluted with CH₂Cl₂ (25 ml) and washed with 1N HCl (1×50 ml) followed by 10% aq. NaHCO₃ (1×50 ml) and water (1×50 ml), dried and evaporated to dryness. The residue was purified by column chromatography on silica gel. The product was eluted using 4% acetone/ CH₂Cl₂ and was then crystallized from CH₂Cl₂/isopropyl ether/hexane. Yield of compound (20)=85 mg.

### Example 21

### Compound (21)

2-Desvinyl-2-hydroxymethyl chlorin e6 tri-methyl ester (Ce6, R═H) (150 mg) was stirred with CDl (150 mg) in CH₂Cl₂ (50 ml) and in the presence of DMAP (25 mg) at room temperature until the reaction was complete (3 h). 3-Amino-1-propanol (1.5 ml) was then added to the solution and stirred overnight at room temperature. The reaction mixture was washed with water (2×50 ml), dried and evaporated to dryness. The residue was purified by column chromatography on silica gel. The product was isolated using 5% MeOH/ CH₂Cl₂ and crystallized from CH₂Cl₂/MeOH/ether. Yield of compound (21)=150 mg.

### Example 22

### Compound (22)

2-Desvinyl-2-(1-hydroxymethyl) chlorin e6 tri-methyl ester (Ce6, R═H) (150 mg) was stirred with CDl (100 mg) in CH₂Cl₂ (50 ml) and in the presence of DMAP (25 mg) at room temperature until the reaction was complete (3 h). 2-(2-Aminoethoxy)ethanol (1.0 ml) was then added to the solution and stirred for 6 h at room temperature. The reaction mixture was washed with water (2×50 ml), dried and evaporated to dryness. The residue was purified by column chromatography on silica gel. The product was isolated using 10% MeOH/CH₂CH ₂ and crystallized from CH₂Cl₂/hexane. Yield of compound (22)=133 mg.

### Example 23

### Compound (23)

2-Desvinyl-2-(1-hydroxymethyl) chlorin e6 tri-methyl ester (Ce6, R═H) (100 mg) was stirred with CDl (50 mg) in CH₂Cl₂ (10 ml) and in the presence of DMAP (10 mg) at room temperature until the reaction was complete (3 h). 2-Methoxy-ethylamine (0.5 ml) was then added to the solution and stirred for 4 h at room temperature. The reaction mixture was washed with water (2×50 ml), dried and evaporated to dryness. The residue was purified by column chromatography on silica gel. The product was isolated using 2% acetone/CH₂Cl₂ and crystallized from CH₂Cl₂/hexane. Yield of compound (23)=110 mg.

### Example 24

### Compound (24)

2-Desvinyl-2-(1-hydroxymethyl) chlorin e6 tri-methyl ester (Ce6, R═H) (100 mg) was stirred with CDl (100 mg) in CH₂Cl₂ (20 ml) and in the presence of DMAP (25 mg) at room temperature until the reaction was complete (3 h). N,N-Dimethylethylenediamine (0.5 ml) was then added to the solution and stirred for 4 h at room temperature. The reaction mixture was washed with water (2×50 ml), dried and evaporated to dryness. The residue was purified by column chromatography on silica gel. The product was isolated using 5% MeOH/CH₂Cl₂ and crystallized from CH₂Cl₂/hexane. Yield of compound (24)=102 mg.

### Example 25

### Compound (25)

2-Desvinyl-2-(1-hydroxyethyl) chlorin e6 tri-methyl ester (Ce6, R═CH₃) (125 mg) was stirred with CDl (125 mg) in CH₂Cl₂ (25 ml) and in the presence of DMAP (25 mg) at room temperature until the reaction was complete (3 h). 3-Amino-1-propanol (0.5 ml) was then added to the solution and stirred overnight at room temperature. The reaction mixture was washed with water (2×50 ml), dried and evaporated to dryness. The residue was purified by column chromatography on silica gel. The product was isolated using 7% MeOH/CH₂Cl₂ and crystallized from CH₂Cl₂/ether/hexane. Yield of compound (25)=84 mg.

### Example 26

### Compound (26)

Compound Chl (150 mg) (derived from the ring opening reaction of methyl pheophorbide and 2-(2-aminoethoxy)ethanol) was stirred with CDl (100 mg) in CH₂Cl₂ (50 ml) and in the presence of DMAP (25 mg) at room temperature until the reaction was complete (3 h). 2-(2-Aminoethoxy)ethanol (1.0 ml) was then added to the solution and stirred for 6 h at room temperature. The reaction mixture was washed with water (2×50 ml), dried and evaporated to dryness. The residue was purified by column chromatography on silica gel. The product was isolated using 10% MeOH/CH₂Cl₂ and crystallized from CH₂Cl₂/hexane. Yield of compound (26)=133 mg.

### Example 27

### Compound (27)

9-Desoxo-9-hydroxy benzoporphyrin (Bp) (125 mg) was stirred with CDl (125 mg) in CH₂Cl₂ (25 ml) and in the presence of DMAP (25 mg) at room temperature until the reaction was complete (3 h). 3-Amino-1-propanol (0.5 ml) was then added to the solution and stirred overnight at room temperature. The reaction mixture was washed with water (2×50 ml), dried and evaporated to dryness. The residue was purified by column chromatography on silica gel. The product was isolated using 7% MeOH/CH₂Cl₂ and crystallized from CH₂Cl₂/ether/hexane. Yield of compound (27)=84 mg.

### Example 28

### Compound (28)

9-Desoxo-9-hydroxy benzoporphyrin (Bp) (125 mg) was stirred with CDl (125 mg) in CH₂Cl₂ (25 ml) and in the presence of DMAP (25 mg) at room temperature until the reaction was complete (3 h). 3-Amino-1-propanol (0.5 ml) was then added to the solution and stirred overnight at room temperature. The reaction mixture was washed with water (2×50 ml), dried and evaporated to dryness. The residue was purified by column chromatography on silica gel. The product was isolated using 7% MeOH/CH₂Cl₂ and crystallized from CH₂Cl₂/ether/hexane. Yield of compound (28)=84 mg.

### Example 29

### Compound (29)

4-(1-Hydroxyethyl)-benzoporphyrin tetramethyl ester (Ring A isomer) (Scheme 16) (125 mg) was stirred with CDl (125 mg) in CH₂Cl₂ (25 ml) and in the presence of DMAP (25 mg) at room temperature until the reaction was complete (3 h). 3-Amino-1-propanol (0.5 ml) was then added to the solution and stirred overnight at room temperature. The reaction mixture was washed with water (2×50 ml), dried and evaporated to dryness. The residue was purified by column chromatography on silica gel. The product was isolated using 7% MeOH/CH₂Cl₂ and crystallized from CH₂Cl₂/ether/hexane. Yield of compound (29)=90 mg.

### Example 30

### Compound (30)

4-(1-Hydroxyethyl)-benzoporphyrin tetramethyl ester (Ring A isomer) (Scheme 16) (125 mg) was stirred with CDl (125 mg) in CH₂Cl₂ (25 ml) and in the presence of DMAP (25 mg) at room temperature until the reaction was complete (3 h). 2-(2-aminoethoxy)ethanol (0.5 ml) was then added to the solution and stirred overnight at room temperature. The reaction mixture was washed with water (2×50 ml), dried and evaporated to dryness. The residue was purified by column chromatography on silica gel. The product was isolated using 7% MeOH/CH₂Cl₂ and crystallized from CH₂Cl₂/ether/hexane. Yield of compound (30)=100 mg.

### Example 31

### Compound (31)

IBc (150 mg, M=2H, R₁, R₂═H (Scheme 17)) was stirred with CDl (400 mg) in CH₂Cl₂ (50 ml) and in the presence of DMAP (70 mg) at room temperature until the reaction was complete. 3-Amino-1-propanol (1.5 ml) was then added to the solution and stirred overnight at room temperature. The reaction mixture was washed with water (2×50 ml), dried and evaporated to dryness. The residue was purified by column chromatography on silica gel. The product was isolated using 5% MeOH/CH₂Cl₂ and crystallized from CH₂Cl₂/MeOH/ether. Yield of compound (31)=127 mg.

### Example 32

### Compound (32)

IBc (150 mg, M=2H, R₁, R₂=bond (Scheme 17)) was stirred with CDl (400 mg) in CH₂Cl₂ (50 ml) and in the presence of DMAP (70 mg) at room temperature until the reaction was complete. 3-Amino-1-propanol (1.5 ml) was then added to the solution and stirred overnight at room temperature. The reaction mixture was washed with water (2×50 ml), dried and evaporated to dryness. The residue was purified by column chromatography on silica gel. The product was isolated using 5% MeOH/CH₂Cl₂ and crystallized from CH₂Cl₂/MeOH/ether. Yield of compound (32)=117 mg.

### Example 33

### Compound (33)

The benzochlorin triol (Scheme 18,150 mg) was stirred with CDl (400 mg) in CH₂Cl₂ (50 ml) and in the presence of DMAP (70 mg) at room temperature until the reaction was complete. 3-Amino-1-propanol (1.5 ml) was then added to the solution and stirred overnight at room temperature. The reaction mixture was washed with water (2×50 ml), dried and evaporated to dryness. The residue was purified by column chromatography on silica gel. The product was isolated using 5% MeOH/CH₂Cl₂ and crystallized from CH₂Cl₂/MeOH/ether. Yield of compound (33)=110 mg.

### Example 34

### Compound (36)

Sulfonyl chloride octaethylbenzochlorin (150 mg) was dissolved in dichloromethane (20 mL) and 3-aminopropanol (0.3 mL) was added. The solution was stirred for 1 hr and methanol (20 mL) was added. The dichloromethane was removed by rotary evaporation and the precipitated benzochlorin filtered. The solid was dissolved in chloroform (20 mL) and a solution of zinc acatate (200 mg) in methanol (5 mL) was added. The solution was refluxed for 30 min and evaporated to dryness. The crude zinc benzochlorin was rapidly chromatographed on silica, eluting with 2% methanol/dichloromethane and the major green fraction collected, evaporated and dried. The hydroxypropylsulfonylamide zinc octaethylbenzochlorin (34) (200 mg) was stirred with CDl (400 mg) in CH₂Cl₂ (50 ml) and in the presence of DMAP (70 mg) at room temperature until the reaction was complete. 3-Amino-1-propanol (1.5 ml) was then added to the solution and stirred overnight at room temperature. The reaction mixture was washed with water (2×50 ml), dried and evaporated to dryness. The residue was purified by column chromatography on silica gel. The product was isolated using 5% MeOH/CH₂Cl₂ and crystallized from CH₂Cl₂ /MeOH. Yield of compound (36)=220 mg.

### Example 35

### Compound (37)

Sulfonyl chloride octaethylbenzochlorin (150 mg) was dissolved in dichloromethane (20 mL) and diethanolamine (0.3 mL) was added. The solution was stirred for 1 hr and methanol (20 mL) added. The dichloromethane was removed by rotary evaporation and the precipitated benzochlorin filtered. The solid was dissolved in chloroform (20 mL) and a solution of zinc acetate (200 mg) in methanol (5 mL) was added. The solution was refluxed for 30 min and evaporated to dryness. The crude zinc benzochlorin was rapidly chromatographed on silica, eluting with 2% methanol/dichloromethane and the major green fraction collected, evaporated and dried. The diethanolsulfonylamide zinc octaethylbenzochlorin (35) (204 mg) was stirred with CDl (400 mg) in CH₂Cl₂ (50 ml) and in the presence of DMAP (70 mg) at room temperature until the reaction was complete. 1,1′,3,3′-tetramethylguanidine (0.5 g) was then added to the solution and stirred overnight at room temperature. The reaction mixture was washed with water (2×50 ml), dried and evaporated to dryness. The residue was purified by column chromatography on silica gel. The product was isolated using 5% MeOH/CH₂Cl₂ and crystallized from CH₂Cl₂/MeOH. Yield of compound (37)=218 mg.

### Example 36

### Compound (38)

2-Desvinyl-1-hydroxyethyl purpurin 18 hexylimide methyl ester (Pim, R═CH₃) (100 mg) was stirred with CDl (100 mg) in CH₂Cl₂ (25 ml) and in the presence of DMAP (25 mg) at room temperature until the reaction was complete (3 h). Hexylamine (0.5 ml) was then added to the solution and stirred for 6 h at room temperature. The reaction mixture was diluted with CH₂Cl₂ (25 ml) and washed with 1N HCl (1×50 ml) followed by 10% aq. NaHCO₃ (1×50 ml) and water (1×50 ml), dried and evaporated to dryness. The residue was purified by column chromatography on silica gel. The product was eluted using 5% acetone/CH₂Cl₂ and then crystallized from CH₂Cl₂/hexane. Yield of compound (38)=92 mg.

### Example 37

### Compound (39)

2-Desvinyl-2-(1-hydroxymethyl) purpurin 18 hexylimide methyl ester (Pim, R═CH₃) (150 mg) was stirred with CDl (150 mg) in CH₂Cl₂ (50 ml) and in the presence of DMAP (25 mg) at room temperature until the reaction was complete (3 h). 3-Amino-1-propanol (1.5 ml) was then added to the solution and stirred overnight at room temperature. The reaction mixture was washed with water (2×50 ml), dried and evaporated to dryness. The residue was purified by column chromatography on silica gel. The product was isolated using 5% MeOH/CH₂Cl₂ and crystallized from CH₂Cl₂/MeOH/ether. Yield of compound (39)=147 mg.

### Example 38

### Compound (40)

2-Desvinyl-2-(1-hydroxymethyl) purpurin 18 hexylimide methyl ester (Pim, R═CH₃) (150 mg) was stirred with CDl (150 mg) in CH₂Cl₂ (50 ml) and in the presence of DMAP (25 mg) at room temperature until the reaction was complete (3 h). 2-(2-Aminoethoxy)ethanol (1.0 ml) was then added to the solution and stirred for 6 h at room temperature. The reaction mixture was washed with water (2×50 ml), dried and evaporated to dryness. The residue was purified by column chromatography on silica gel. The product was isolated using 10% MeOH/CH₂Cl₂ and crystallized from CH₂Cl₂/hexane. Yield of compound (40)=143 mg.

### Example 39

### Compound (41)

2-Desvinyl-2-(1-hydroxymethyl) purpurin 18 hexylimide methyl ester (Pim, R═H) (150 mg) was stirred with CDl (150 mg) in CH₂Cl₂ (50 ml) and in the presence of DMAP (25 mg) at room temperature until the reaction was complete (3 h). 3-Aminopropanol (1.0 ml) was then added to the solution and stirred for 6 h at room temperature. The reaction mixture was washed with water (2×50 ml), dried and evaporated to dryness. The residue was purified by column chromatography on silica gel. The product was isolated using 10% MeOH/CH₂Cl₂ and crystallized from CH₂Cl₂/hexane. Yield of compound (41)=139 mg.

### Example 40

### Compound (42)

2-Desvinyl-2-(1-hydroxymethyl) purpurin 18 hexylimide methyl ester (Pim, R═CH₃) (150 mg) was stirred with CDl (150 mg) in CH₂Cl₂ (50 ml) and in the presence of DMAP (25 mg) at room temperature until the reaction was complete (3 h). N,N-Dimethylaminoethylamine (1.0 ml) was then added to the solution and stirred for 6 h at room temperature. The reaction mixture was washed with water (2×50 ml), dried and evaporated to dryness. The residue was purified by column chromatography on silica gel. The product was isolated using 10% MeOH/CH₂Cl₂ and crystallized from CH₂Cl₂/hexane. Yield of compound (42)=147 mg.

### Example 41

### Compound (43)

Purpurin 18 hexylimide methyl ester (300 mg) was dissolved in THF (100 mL) and a solution of KOH (500 mg) in water (10 mL) added dropwise. The solution was stirred for 3 hours at room temperature after which time the ester had hydrolysed. The solution was rotary evaporated to remove the THF and water (5 mL) was added. Acetic acid was added dropwise until a thick precipitate occurred. This was filtered and dried overnight in a vacuum oven at 60° C. The purpurin 18 hexylimide propionic acid (230 mg) was dissolved in dichloromethane (50 mL) and tetrahydrofuran (50 mL) and triethylamine was added (0.3 mL). The solution was cooled to O° C. in an ice bath. Ethyl chloroformate (0.3 mL) was added and the solution stirred for 1 hr at room temperature. 3-Aminopropanol (0.5 mL) was added and the reaction closely monitored by TLC (5% acetone/dichloromethane). When deemed complete the reaction was poured into water (100 mL) and the organic phase separated and rotoevaporated. The residue was chromatographed on silica using 2% methanol dichloromethane as eluent and the major brown fraction collected. The organic layer was removed by rotoevaporation and the product (PimA) was precipitated from dichloromethane/hexane. Yield=230 mg. PimA (230 mg) was dissolved in dichloromethane (50 mL) and CDl (150 mg) and DMAP (25 mg) added at room temperature. The solution was stirred until the reaction was complete (3 h). 2-(2-Aminoethoxy)ethanol (1.0 ml) was then added to the solution and stirred for 6 h at room temperature. The reaction mixture was washed with water (2×50 ml), dried and evaporated to dryness. The residue was purified by column chromatography on silica gel. The product was isolated using 10% MeOH/CH₂Cl₂ and crystallized from CH₂Cl₂/hexane. Yield of compound (43)=180 mg.

### Example 42

### Compound (44)

PimA (230 mg) produced as described in the synthesis of compound (43) was dissolved in dichloromethane (50 mL) and CDl (150 mg) and DMAP (25 mg) added at room temperature. The solution was stirred until the reaction was complete (3 h). 3-Aminopropanol (1.0 ml) was then added to the solution and stirred for 6 h at room temperature. The reaction mixture was washed with water (2×50 ml), dried and evaporated to dryness. The residue was purified by column chromatography on silica gel. The product was isolated using 7% MeOH/CH₂Cl₂ and crystallized from CH₂Cl₂/hexane. Yield of compound (44)=190 mg.

### Example 43

### Compound (45)

PimA (230 mg) produced as described in the synthesis of compound (43) was dissolved in dichloromethane (50 mL) and CDl (150 mg) and DMAP (25 mg) added at room temperature. The solution was stirred until the reaction was complete (3 h). N,N-dimethylaminoethylamine (1.0 ml) was then added to the solution and stirred for 6 h at room temperature. The reaction mixture was washed with water (2×50 ml), dried and evaporated to dryness. The residue was purified by column chromatography on silica gel. The product was isolated using 11% MeOH/CH₂Cl₂ and crystallized from CH₂Cl₂/hexane. Yield of compound (45)=192 mg.

### Example 44

### Compound (46)

Hematoporphyrin dimethyl ester (200 mg) was dissolved in dichloromethane (50 mL) and CDl (200 mg) and DMAP (25 mg) added at room temperature. The solution was stirred until the reaction was complete (3 h). 2-(2-Aminoethoxy)ethanol (1.0 ml) was then added to the solution and stirred for 6 h at room temperature. The reaction mixture was washed with water (2×50 ml), dried and evaporated to dryness. The residue was purified by column chromatography on silica gel. The product was isolated using 7% MeOH/CH₂Cl₂ and crystallized from CH₂Cl₂/hexane. Yield of compound (46)=190 mg.

### Example 45

### Compound (47)

Hematoporphyrin dimethyl ester (200 mg) was dissolved in dichloromethane (50 mL) and CDl (200 mg) and DMAP (25 mg) added at room temperature. The solution was stirred until the reaction was complete (3 h). 3-Aminopropanol (1.0 ml) was then added to the solution and stirred for 6 h at room temperature. The reaction mixture was washed with water (2×50 ml), dried and evaporated to dryness. The residue was purified by column chromatography on silica gel. The product was isolated using 7% MeOH/CH₂Cl₂ and crystallized from CH₂Cl₂/hexane. Yield of compound (47)=199 mg.

### Example 46

### Compound (48)

Hematoporphyrin dimethyl ester (200 mg) was dissolved in dichloromethane (50 mL) and CDl (200 mg) and DMAP (25 mg) added at room temperature. The solution was stirred until the reaction was complete (3 h). N,N-dimethylaminoethylamine (1.0 ml) was then added to the solution and stirred for 6 h at room temperature. The reaction mixture was washed with water (2×50 ml), dried and evaporated to dryness. The residue was purified by column chromatography on silica gel. The product was isolated using 11% MeOH/CH₂Cl₂/0.5% triethylamine and crystallized from CH₂Cl₂/hexane. Yield of compound (48)=155 mg.

### Example 47

### Compound (49)

2,4-Diethyl-1,3,5,8-tetraethyl-6,7-bis(3-hydroxypropan-1-yl)porphine (100 mg) was dissolved in dichloromethane (50 mL) and CDl (200 mg) and DMAP (25 mg) added at room temperature. The solution was stirred until the reaction was complete (3 h). 2-(2-Aminoethoxy)ethanol (1.0 ml) was then added to the solution and stirred for 6 h at room temperature. The reaction mixture was washed with water (2×50 ml), dried and evaporated to dryness. The residue was purified by column chromatography on silica gel. The product was isolated using 7% MeOH/CH₂Cl₂ and crystallized from CH₂Cl₂/hexane. Yield of compound (49)=107 mg.

### Example 48

### Compound (50)

2,4-Diethyl-1,3,5,8-tetraethyl-6,7-bis(3-hydroxypropan-1-yl)porphine (100 mg) was dissolved in dichloromethane (50 mL) and CDl (200 mg) and DMAP (25 mg) added at room temperature. The solution was stirred until the reaction was complete (3 h). 3-Aminopropanol (1.0 ml) was then added to the solution and stirred for 6 h at room temperature. The reaction mixture was washed with water (2×50 ml), dried and evaporated to dryness. The residue was purified by column chromatography on silica gel. The required product was isolated using 7% MeOH/CH₂Cl₂ and crystallized from CH₂Cl₂/hexane. Yield of compound (50)=100 mg.

### In Vivo Biological Response

### Example 49

### Tumor Treatment

The carbamate compounds were formulated in egg yolk phosphatidyl choline (EYP) and phosphate buffered saline (PBS) (pH 7.4). These were sterilized by filtration through a 0.2-micron nylon filter and determined to be stable for at least several weeks following formulation by HPLC. Five Sprague-Dawley rats with subcutaneous chondrosarcoma tumors in the flank of a certain volume (150-250 mm³) were injected intravenously with various drugs at various doses. Three hours after the injection the tumors were exposed to 664-nm light at light doses of 125 J/cm² or 200 J/cm². The end point of the study was the observation of tumor regrowth (averaged over the animals) following the treatment.

Table 2 illustrates the results for the best drug and light doses that were tested in the above system and are compared with the well known photosensitizer SnET2 under optimal conditions (24 hrs post drug administration).

TABLE 2
Chondrosarcoma tumor growth delay for the carbamate
macrocycles.

| | Drug Dose | | |
|---|---|---|---|
| Drug tested | (μmol/kg) | Light Dose (J/cm2) | Days (regrowth) |
| (13) | 1.5 | 125 | 14 |
| (3) | 1.5 | 125 | 10 |
| (6) | 0.75 | 125 | 11 |
| (7) | 1.0 | 125 | 23 |
| (4) | 1.0 | 125 | 4/5 cured |
| SnET2 | 2.0 | 125 | 13 |

Cured = no regrowth of tumor

The data clearly demonstrates that in the above tumor model the compounds of the present invention at comparable drug doses are equivalent or more effective than SnET2 in delaying tumor growth in rats.

### Example 50

### In Vivo Corneal Neovascular Shut Down Experimentally Induced Corneal Neovascularization

Corneal neovessels were experimentally induced in Sprague Dawley rats with an N-heptanol chemical scrub. The chemical scrub was used to remove the corneal epithelium and stem cells, allowing the neovessels to grow across the entire cornea. PDT was performed at approximately 3 weeks after the chemical scrub when the neovessels formed a uniform network across the cornea. The PDT treatment was applied to the corneal surface with a laser wavelength that was optimized for the given absorption spectrum.

The laser energy was coupled through a slit lamp biomicroscope with a slit lamp adapter. A 3.0 mm spot size was used (Area=7.07 mm²). The light dose delivered was varied from 5-25 J/cm². The efficacy of neovessel closure was evaluated by measuring the area of treated cornea that remained neovessel-free out to 28 days following PDT. Accurate area measurements were taken using fluorescein angiography and measuring the area of neovessel-free cornea. Absence of fluorescein leakage in the treatment area demonstrated closure of the neovessels. The dosimetry and results of selected carbamate molecules in this model are summarized in Table 3.

TABLE 3
A summary of the optimal drug dose and time interval for PDT
treatment of corneal neovessels induced by an n-heptanol scrub. The light
dose was 20 J/cm² at the corresponding wavelength for optimal excitation of
each photosensitizer.

| | | | | Extent of | | | | |
|---|---|---|---|---|---|---|---|---|
| | | | Time | neovessel | | | | Extent of |
| | | | Interval of | closure at days | | | | neovessel |
| | Excitation | | treatment | 1-21 after | | | | closure at 28 |
| | Wavelength | Drug Dose | post dose | treatment | | | | days after |
| Molecule | (nm) | (μmoles/kg) | (min) | 1 | 7 | 14 | 21 | treatment |
| Visudyne | 689 | 1.4 | 15 | 4 | 1 | 0 | 0 | 0 |
| (2) | 664 | 1.0 | 10 | 5 | 5 | 2 | 1 | 0 |
| (3) | 664 | 1.5 | 15 | 5 | 4 | 3 | 2 | 2 |
| (6) | 664 | 1.0 | 10 | 5 | 4 | 4 | 4 | 4 |
| (7) | 664 | 1.0 | 10 | 5 | 3 | 2 | 2 | 1 |
| (5) | 664 | 0.5 | 10 | 5 | 3 | 2 | 2 | 2 |
| (1) | 664 | 1.0 | 10 | 4 | 1 | 1 | 1 | 0 |

⁼-Grading scale based on corneal fluorescein angiography: 0 = 0-0.5 mm, 1 = 0.51-1.0 mm, 2 = 1.1-1.5 mm, 3 = 1.6-2.0 mm, 4 = 2.1-3.0 mm, 5 = >3.0 mm

The data demonstrates that several of the compounds of the present invention are more effective at sustaining neovessel shut down in the eye of rats compared to Visudyne (Vertoporfin), which is the current treatment for age related macular degeneration in photodynamic therapy.

### Example 51

### Normal Choriocapillaris Rabbit Model

Selected carbamate molecules were also evaluated in a normal choriocapillaris model in the pigmented rabbit. This model used the choriocapillaris as a surrogate for neovasculature to demonstrate PDT efficacy and longevity of vessel closure in the posterior segment of the eye (G. A. Peyman, D. M. Moshfeghi, A. M. Moshfeghi, B. Khoobehi, D. R. Doiron, G. B. Primbs, D. H. Crean, “Photodynamic Therapy for Choriocapillaris Using Tin Ethyletiopurpurin (SnET2)”, Ophthalmic Surg Lasers, 1997, 28:409417).

The selected photosensitizers were administered intravenously at varying drug doses, the light dose was set constant at 20 J/cm², and the time interval was varied from 5-30 minutes between drug and light administration. Two PDT treatment areas were placed on the fundus of each eye in each rabbit. Fluorescein angiography was used to evaluate vessel closure following PDT out to 28 days. The dosimetry and efficacy results of these molecules are summarized in Table 4.

TABLE 4
Optimal dosimetry and results summarizing the closure of the
choriocapillaris at 28 days following PDT. The light dose for all
treatments was 20 J/cm². The data is an average for five rabbits.

| | Drug Dose | Time Interval | Closure at 28 |
|---|---|---|---|
| Molecule | (μmoles/kg) | (min) | Days⁼ |
| Visudyne | 1.4 | 5-10 | 4 |
| (3) | 2.5 | 5-30 | 4 |
| (6) | 1.0 | 5-30 | 4 |
| (7) | 1.5 | 5-30 | 3 |
| (5) | 0.75 | 5-30 | 3 |

⁼Grading scale based on fluorescein angiography: 1 = 0-25%, 2 = 26-50%, 3 = 51-75%, 4 > 75%

### Example 52

### Experimentally Induced Choroidal Neovascularization

Three of the carbamate molecules, (3), (6), and (7), were evaluated in a laser-induced choroidal neovascularization model in rats. Laser photocoagulation was used to stimulate choroidal neovessel growth on the fundus of the rat (E. T. Dobi, C. Puliafito, M. A. Destro, “A new model of experimental choroidal neovascularization in the rat”, Arch. Ophthalmol. 1989; 107: 264-269). The PDT treatments were performed approximately 3 weeks after the laser photocoagulation, which was when the choroidal neovasculariztion lesioris were fully developed. The lesions were PDT treated using a 0.5 mm spot that covered the entire CNV lesion. Fluorescein angiography and histopathology were used to evaluate the CNV closure. Initial flush of the fluorescein angiography showed that molecules (3) and (6) (2.0 μmoles/kg, 10-20 minutes post injection) closed the CNV lesion at 7 days after PDT. Molecule (7) (1.5 & 3.0 μmoles/kg, 10-20 minutes post injection) also demonstrated CNV closure at 7 days post PDT based on fluorescein angiography. Fluorescein angiography of (7) at 28 days following PDT showed closure of the CNV at 10-40 minute intervals for 3.0 μmoles/kg. In comparison, Visudyne also showed CNV closure at 7 days post treatment at a drug dose of 1.4 μmoles/kg, with light treatment 10-20 minutes post injection.

In summary, the pharmacological properties of the novel compounds according to the invention are substantially different from those of existing photosensitizers described to date in the literature. In particular, the compounds investigated possess the following properties.

(I) They are distributed and localized in ophthalmic neovessels and other diseased tissues following injection.

(II) They are activated at wavelengths of 300-900 nm to cause selective biological effects in the target tissue.

(III) Following light activation, they cause significant sustained neovessel closure of occular neovessels.

(IV) They demonstrate short periods of normal skin photosensitivity in rats (1-6 hrs)

(V) They are metabolized rapidly in vivo to less photoactive compounds.

(VI) Metabolism of peripheral ester groups is enhanced by the addition of the carbamate moiety.

(VII) They are stable in formulations for at least several weeks, which lends itself well to lyophilization technology if required.

(VIII) They are effective at causing therapeutically significant neovessel closure in advanced ophthalmic animal model systems with efficacy equal to or greater than the currently approved ophthalmic photosensitizer.

What is claimed is:

1. Compounds of formula I: 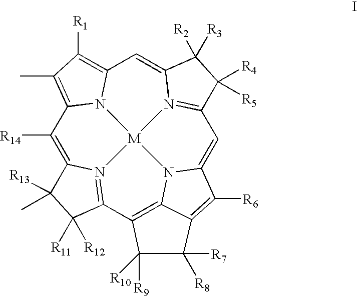
wherein:
R₁, R₂, R₃, R₄, R₅, R₆, R₇, R₈, R₉, R₁₀, R₁₁, R₁₂, R₁₃, and R₁₄ are independently selected from the group consisting of:
H, halogen, substituted or unsubstituted C1-C20 alkyl, heteroalkyl, haloalkyl, heterohaloalkyl, cycloalkyl, aryl, substituted aryl, alkenyl, substituted alkenyl, alkynyl, substituted alkynyl, amide, ester, ether, polyether, alkoxy, aryloxy, haloalkoxy, amino, alkylcarbonyloxy, alkoxycarbonyl, aryloxycarbonyl, azo, arylcarbonyloxy, alkoxycarbonyloxy, aryloxycarbonyloxy, sulfinyl, sulfonyl, silil, carbamoyl, heterocyclic, nitro, nitroso, formyloxy, isocyano, cyanate, isocyanate, thiocyanate, isothiocyanate, N(alkyl)₂, N(aryl)₂, CH═CH(aryl), CH═CHCH₂N(CH₃)₂, CH═CHCH₂N⁺(CH₃)₃A, CH═N(alkyl)₂⁺A, N(alkyl)₃⁺A, CN, OH, CHO, COCH₃, CO(alkyl), CO₂H, CO₂Na, CO₂K, CH(CH₃)OH, CH(CH₃)O-alkyl, CH(CH₃)O-alkoxy, CH(CH₃)O-aryl, CH(CH₃)NH-alkyl, CH(CH₃)NH-cycloalkyl, CH(CH₃)NH-heteroalkyl, CH(CH₃)NH-heteroalkoxy, CH(CH₃)-(amino acid), CH(CH₃)-(amino acid ester), CH(CH₃)-(amino acid amide), C(X)₂C(X)₃, and CH═NR₁₅, where X is selected from H and halogen, R₁₅ is selected from OH, O-alkyl, O-ether, O-alkylamino, NHCOCH₂N(CH₃)₂, NHCOCH₂N(CH₃)₃⁺A, NHCOCH₂-(pyridinium)⁺A, (CH₂)ₙO-alkoxy, and
CO₂R₁₆, where R₁₆ is selected from H, a physiologically acceptable counter ion, a C1-C20 straight or branched chain alkyl, haloalkyl, heteroalkyl, haloheteroalkyl, aryl, heteroaryl, heterocycle, a mono-, di-, or polyhydroxyalkyl residue, a mono-, di-, or polyhydroxyaryl residue, and a functional group of less than about 100,000 daltons;
(CH₂)ₙOH and (CH₂)ₙOR₁₇, where R₁₇ is selected from alkyl, haloalkyl, heteroalkyl, haloheteroalkyl, aryl, heteroaryl, heterocycle, a protecting group, a mono-, di-, or polyhydroxyalkyl residue, a mono-, di-, or polyhydroxyaryl residue, and a functional group of less than about 100,000 daltons, and n is an integer ranging from 0 to 4;
(CH₂)ₙCO₂R₁₈, (CHX)ₙCO₂R₁₈, and (CX₂)ₙCO₂R₁₈, where X is selected from OH, OR₁₉, and a halogen, and R₁₈ and R₁₉ are independently selected from H, a physiologically acceptable counter ion, acetyl, a straight or branched chain C1-C20 alkyl, haloalkyl, heteroalkyl, haloheteroalkyl, aryl, heteroaryl, heterocycle, a mono-, di-, or polyhydroxyalkyl residue, a mono-, di-, or polyhydroxyaryl residue, and a functional group of less than about 100,000 daltons, and n is an integer ranging from 0 to 4;
CONH(R₂₀), CONHNH(R₂₀), CO(R₂₀), CON(R₂₀)₂, CON(R₂₀)(R₂₁) (CH₂)ₙCONH(R₂₀), (CH₂)ₙCON(R₂₀)₂, (CH₂)ₙCOR₂₀, (CH₂)ₙCON(R₂₀)(R₂₁), (CX₂)ₙCONH(R₂₀), (CX₂)ₙCON(R₂₀)₂, (CX₂)ₙCON(R₂₀)(R₂₁), (CX₂)ₙCOR₂₀, (CH₂)ₙCONHNH(R₂₀), (CX₂)ₙCONHNH(R₂₀), (CHX)ₙCONH(R₂₀), (CHX)ₙCONHNH(R₂₀), (CHX)ₙCO(R₂O), (CHX)ₙCON(R₂₀)₂, and (CHX)ₙCON(R₂₀)(R₂₁), where X is selected from OH, OR₂₂, SR₂₂, and a halogen, and R₂₀, R₂₁ and R₂₂ are independently selected from H, NH₂, acetyl, a straight or branched chain C1-C20 alkyl, haloalkyl, haloheteroalkyl, heteroalkyl, aryl, heteroaryl, heterocycle, a mono-, di-, or polyhydroxyalkyl residue, a mono-, di-, or polyhydroxyaryl residue, an amino acid ester, an amino acid amide, a mono-, di-, or polyetheralkyl residue, a mono-, di-, or polyetheraryl residue, and a functional group of less than about 100,000 daltons, and n is an integer ranging from 1 to 4;
S(R₂₃), CH(CH₃)S(R₂₃), (CH₂)ₙS(R₂₃), (CH₂)ₙNH(R₂₃), (CH₂)ₙNHNH(R₂₃), (CH₂)ₙN(R₂₃)₂, (CH₂)ₙN(R₂₃)(R₂₄), (CH₂)ₙN(R₂₃)(R₂₄)(R₂₅)⁺A, CH═N(R₂₃), CH═NN(R₂₃)(R₂₄), and amino acids containing —NH(R₂₃) or —N(R₂₃)(R₂₄), where R₂₃, R₂₄ and R₂₅ are independently selected from H, OH, O-alkyl, NH₂, acetyl, a straight or branched chain C1-C20 alkyl, haloalkyl, heteroalkyl, haloheteroalkyl, aryl, heteroaryl, heterocycle, a mono-, di-, or polyhydroxyalkyl residue, a mono-, di-, or polyhydroxyaryl residue, a mono-, di-, or polyetheralkyl residue, a mono-, di-, or polyetheraryl residue, and a functional group of less than about 109,000 daltons, where R₂₃, R₂₄ and R₂₅ together may possess the atoms necessary to constitute an aromatic ring system, n is an integer ranging from 0 to 4, and A is a physiologically acceptable counter ion;
(CH₂)ₙOPO(OR₂₆)₂ and (CH₂)ₙPO(OR₂₆)₂, where R₂₆ is selected from H, a physiologically acceptable counter ion, a straight or branched chain C1-C20 alkyl, haloalkyl, heteroalkyl, haloheteroalkyl, aryl, heteroaryl, heterocycle, a mono-, di-, or polyhydroxyalkyl residue, a mono-, di-, or polyhydroxyaryl residue, a mono-, di-, or polyetheralkyl residue, a mono-, di-, or polyetheraryl residue, and a functional group of less than about 100,000 daltons, and n is an integer ranging from 0 to 4;
(CH₂)ₙNHCOR₂₇ and (CH₂)ₙNHNHCOR₂₇, where R₂₇ is selected from a straight or branched chain C1-C20 alkyl, haloalkyl, heteroalkyl, haloheteroalkyl, aryl, heteroaryl, heterocycle, and a functional group of less than about 100,000 daltons, and n is an integer ranging from between 0 to 4;
SO₃R₂₈, SO₂NHR₂₈, SO₂N(R₂₈)₂, SO₂NHNHR₂₈, SO₂R₂₈, SO₃R₂₈, (CH₂)ₙSO₂NHR₂₈, (CH₂)ₙSO₂N(R₂₈)₂, (CH₂)ₙSO₂NHNHR₂₈, and (CH₂)ₙSO₂R₂₈, where R₂₈ is selected from H, OH, a physiologically acceptable counter ion, a straight or branched chain C1-C20 alkyl, haloalkyl, heteroalkyl, haloheteroalkyl, aryl, heteroaryl, heterocycle, a mono-, di-, or polyhydroxyalkyl residue, a mono-, di-, or polyhydroxyaryl residue, a mono-, di-, or polyetheralkyl residue, a mono-, di-, or polyetheraryl residue, and a functional group of less than about 100,000 daltons, where NHR₂₈ can be an amino acid, an amino acid salt, an amino acid ester residue, or an amino acid amide residue, and n is an integer ranging from 0 to 4;
aryl and substituted aryl, which may bear one or more substituents with a molecular weight of less than or equal to about 100,000 daltons;
wherein:
R₃ and R₄ may form a bond;
R₁₂ and R₁₃ may form a bond;
R₇ and R₈ may form a ═O; and
R₉ and R₁₀ may form a ═O;
with the proviso that at least one of R₁ through R₂₈ is a functional group that comprises a carbamate of the formulae —OCON(R₂₉)₂, —OCON═C(R₂₉)₂, —OCONR₂₉R₃₀, or —OCON═C(R₂₉)(R₃₀), where R₂₉ and R₃₀ are independently selected from H, C1-C20 alkyl, C1-C20 cycloalkyl, aryl, NH₂, N(CH₃)₂, (CH₂)ₙOH, (CH₂)ₙO-alkyl, (CH₂)ₙOCOCH₃, (CH₂)ₙO(CH₂)ₘOH, (CH₂)ₙO(CH₂)ₘOCOCH₃, (CH₂)ₙO(CH₂)ₘO-alkyl, (CH₂)ₙN((CH₂)ₘOH)₂, (CH₂)ₙN((CH₂)ₘO-alkyl)₂, (CH₂)ₙN((CH₂)ₘO-alkylether)₂, ((CH₂)ₙO)ₘ((CH₂)_{Q})OH, (CH₂)ₙO(CH₂)ₘNH₂, (CH₂)ₙO(CH₂)ₘN(CH₃)₂, (CH₂)ₙO(CH₂)ₘN(CH₃)₃⁺A, (CH₂)ₙN((CH₂)ₘNH₂)₂, (CH₂)ₙN(CH₂)ₘN(CH₃)₂, (CH₂)ₙO-haloalkyl, (CH₂)ₙN(CH₂)ₘN(CH₃)₃⁺A)₂, ((CH₂)ₙO)ₘ(CH₂O)_{Q}COCH₃, an alkylphosphate residue, an alkylsulfonic acid residue, an alkylsulfonic ester residue, alkylsulfonic amide residue, an alkylmorpholino residue, an alkylheterocyclic residue, an alkylthiol residue, a mono-, di-, or polyhydroxyalkyl residue, a mono-, di-, or polyhydroxyaryl residue, a mono-, di-, or polyetheralkyl residue, and a mono-, di-, or polyetheraryl residue, where Q, n and m are integers ranging from 0 to 10,000, and A is a physiologically acceptable counter ion; and
M is selected from 2H, a metal cation, and photoactive metal ions selected from Ga³⁺, Pt²⁺, Pd²⁺, Sn⁴⁺, In³⁺, Ge⁴⁺, Si⁴⁺, Al³⁺, Zn²⁺, and Mg²⁺;
or a pharmaceutically acceptable salt, prodrug, solvate, or metabolite thereof.

2. A pharmaceutical composition comprising an effective diagnostic or therapeutic amount of the compound of claim 1, together with at least one pharmaceutically acceptable carrier or excipient.

3. The pharmaceutical composition according to claim 2 used to treat ophthalmic diseases.

4. The pharmaceutical composition of claim 3 wherein said ophthalmic diseases are selected from proliferative retinopathies, macular edema, corneal neovascularization, conjunctival neovascularization, ocular tumors, viral retinitis adjunct to glaucoma filtration surgery and cyclodestruction, posterior capsule opacification, and age related macular degeneration.

5. The pharmaceutical composition according to claim 2 used to treat cardiovascular diseases.

6. The pharmaceutical composition according to claim 2 used to treat skin diseases.

7. Compounds of formula II: 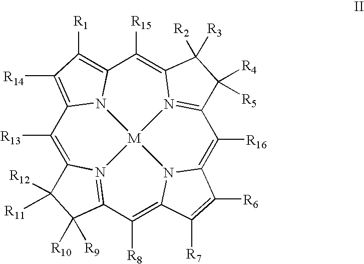
wherein:
R₁, R₂, R₃, R₄, R₅, R₆, R₇, R₈, R₉, R₁₀, R₁₁, R₁₂, R₁₃, R₁₄, R₁₅, and R₁₆ are independently selected from the group consisting of:
H, halogen, substituted or unsubstituted C1-C20 alkyl, heteroalkyl, haloalkyl, heterohaloalkyl, cycloalkyl, aryl, substituted aryl, alkenyl, substituted alkenyl, alkynyl, substituted alkynyl, amide, ester, ether, polyether, alkoxy, aryloxy, haloalkoxy, amino, alkylcarbonyloxy, alkoxycarbonyl, aryloxycarbonyl, azo, arylcarbonyloxy, alkoxycarbonyloxy, aryloxycarbonyloxy, sulfinyl, sulfonyl, silil, carbamoyl, heterocyclic, nitro, nitroso, formyloxy, isocyano, cyanate, isocyanate, thiocyanate, isothiocyanate, N(alkyl)₂, N(aryl)₂, CH═CH(aryl), CH═CHCH₂N(CH₃)₂, CH═CHCH₂N⁺(CH₃)₃A, CH═N(alkyl)₂⁺A, N(alkyl)₃⁺A, CN, OH, CHO, COCH₃, CO(alkyl), CO₂H, CO₂Na, CO₂K, CH(CH₃)OH, CH(CH₃)O-alkyl, CH(CH₃)O-alkoxy, CH(CH₃)O-aryl, CH(CH₃)NH-alkyl, CH(CH₃)NH-cycloalkyl, CH(CH₃)NH-heteroalkyl, CH(CH₃)NH-heteroalkoxy, CH(CH₃)-(amino acid), CH(CH₃)-(amino acid ester), CH(CH₃)-(amino acid amide), C(X)₂C(X)₃, and CH═NR₁₇, where X is selected from H and halogen, R₁₇ is selected from OH, O-alkyl, O-ether, O-alkylamino, NHCOCH₂N(CH₃)₂, NHCOCH₂N(CH₃)₃⁺A, NHCOCH₂-(pyridinium)⁺A, (CH₂)ₙO-alkoxy, and (CH₂)ₙO-alkyl, n is an integer ranging from 0 to 8, and A is a physiologically acceptable charge balancing ion;
CO₂R₁₈, where R₁₆ is selected from H, a physiologically acceptable counter ion, a C1-C20 straight or branched chain alkyl, haloalkyl, heteroalkyl, haloheteroalkyl, aryl, heteroaryl, heterocycle, a mono-, di-, or polyhydroxyalkyl residue, a mono-, di-, or polyhydroxyaryl residue, and a functional group of less than about 100,000 daltons;
(CH₂)ₙOH and (CH₂)ₙOR₁₉, where R₁₉ is selected from alkyl, haloalkyl, heteroalkyl, haloheteroalkyl, aryl, heteroaryl, heterocycle, a protecting group, a mono-, di-, or polyhydroxyalkyl residue, a mono-, di-, or polyhydroxyaryl residue, and a functional group of less than about 100,000 daltons, and n is an integer ranging from 0 to 4;
(CH₂)ₙCO₂R₂₀, (CHX)ₙCO₂R₂₀, and (CX₂)ₙCO₂R₂₀, where X is selected from OH, OR₂₁, and a halogen, and R₂₀ and R₂₁, are independently selected from H, a physiologically acceptable counter ion, acetyl, a straight or branched chain C1-C20 alkyl, haloalkyl, heteroalkyl, haloheteroalkyl, aryl, heteroaryl, heterocycle, a mono-, di-, or polyhydroxyalkyl residue, a mono-, di-, or polyhydroxyaryl residue, and a functional group of less than about 100,000 daltons, and n is an integer ranging from 0 to 4;
CONH(R₂₂), CONHNH(R₂₂), CO(R₂₂), CON(R₂₂)₂, CON(R₂₂)(R₂₃), (CH₂)ₙCONH(R₂₂), (CH₂)ₙCON(R₂₂)₂, (CH₂)ₙCOR₂₂, (CH₂)ₙCON(R₂₂)(R₂₃), (CX₂)ₙCONH(R₂₂), (CX₂)ₙCON(R₂₂)₂, (CX₂)ₙCO N(R₂₂)(R₂₃), (CX₂)ₙCOR₂₂, (CH₂)ₙCONHNH(R₂₂), (CX₂)ₙCONHNH(R₂₂), (CHX)ₙCONH(R₂₂), (CHX)ₙCONHNH(R₂₂), (CHX)ₙCO(R₂₂), (CHX)ₙCON(R₂₂)₂, and (CHX)ₙCON(R₂₂)(R₂₃), where X is selected from OH, OR₂₄, SR₂₄, and a halogen, and R₂₂, R₂₃ and R₂₄ are independently selected from H, NH₂, acetyl, a straight or branched chain C1-C20 alkyl, haloalkyl, haloheteroalkyl, heteroalkyl, aryl, heteroaryl, heterocycle, a mono-, di-, or polyhydroxyalkyl residue, a mono-, di-, or polyhydroxyaryl residue, an amino acid ester, an amino acid amide, a mono-, di-, or polyetheralkyl residue, a mono-, di-, or polyetheraryl residue, and a functional group of less than about 100,000 daltons, and n is an integer ranging from 1 to 4;
S(R₂₅), CH(CH₃)S(R₂₅), (CH₂)ₙS(R₂₅), (CH₂)ₙNH(R₂₅), (CH₂)ₙNHNH(R₂₅), (CH₂)ₙN(R₂₅)₂, (CH₂)ₙN(R₂₅)(R₂₆), (CH₂)ₙN(R₂₅)(R₂₆)(R₂₇)⁺A, CH═N(R₂₅), CH═NN(R₂₅)(R₂₆), and amino acids containing —NH(R₂₅) or —N(R₂₅)(R₂₆), where R₂₄, R₂₆ and R₂₇ are independently selected from H, OH, O-alkyl, NH₂, acetyl, a straight or branched chain C1-C20 alkyl, haloalkyl, heteroalkyl, haloheteroalkyl, aryl, heteroaryl, heterocycle, a mono-, di-, or polyhydroxyalkyl residue, a mono-, di-, or polyhydroxyaryl residue, a mono-, di-, or polyetheralkyl residue, a mono-, di-, or polyetheraryl residue, and a functional group of less than about 100,000 daltons, where R₂₅, R₂₆ and R₂₇ together may possess the atoms necessary to constitute an aromatic ring system, n is an integer ranging from 0 to 4, and A is a physiologically acceptable counter ion;
(CH₂)ₙOPO(OR₂₈)₂ and (CH₂)ₙPO(OR₂₈)₂, where R₂₈ is selected from H, a physiologically acceptable counter ion, a straight or branched chain C1-C20 alkyl, haloalkyl, heteroalkyl, haloheteroalkyl, aryl, heteroaryl, heterocycle, a mono-, di-, or polyhydroxyalkyl residue, a mono-, di-, or polyhydroxyaryl residue, a mono-, di-, or polyetheralkyl residue, a mono-, di-, or polyetheraryl residue, and a functional group of less than about 100,000 daltons, and n is an integer ranging from 0 to 4;
(CH₂)ₙNHCOR₂₉ and (CH₂)ₙNHNHCOR₂₉, where R₂₉ is selected from a straight or branched chain C1-C20 alkyl, haloalkyl, heteroalkyl, haloheteroalkyl, aryl, heteroaryl, heterocycle, and a functional group of less than about 100,000 daltons, and n is an integer ranging from 0 to 4;
SO₃R₃₀, SO₂NHR₃₀, SO₂N(R₃₀)₂, SO₂NHNHR₃₀, SO₂R₃₀, SO₃R₃₀, (CH₂)ₙSO₂NHR₃₀, (CH₂)ₙSO₂N(R₃₀)₂, (CH₂)ₙSO₂NHNHR₃₀, and (CH₂)ₙSO₂R₃₀, where R₃₀ is selected from H, OH, a physiologically acceptable counter ion, a straight or branched chain C1-C20 alkyl, haloalkyl, heteroalkyl, haloheteroalkyl, aryl, heteroaryl, heterocycle, a mono-, di-, or polyhydroxyalkyl residue, a mono-, di-, or polyhydroxyaryl residue, a mono-, di-, or polyetheralkyl residue, a mono-, di-, or polyetheraryl residue, and a functional group of less than about 100,000 daltons, where NHR₃₀ can be an amino acid, an amino acid salt, an amino acid ester residue or an amino acid amide residue, and n is an integer ranging from 0 to 4; and
aryl or substituted aryl, which may bear one or more substituents with a molecular weight of less than or equal to about 100,000 daltons;
wherein:
R₃ and R₄ may form a bond; and
R₁₀and R₁₁ may form a bond;
with the proviso that at least one of R₁ through R₃₀ is a functional group comprising a carbamate of the formulae —OCON(R₂₉)₂, —OCON═C(R₂₉)₂, —OCONR₂₉R₃₀, or —OCON═C(R₂₉)(R₃₀), where R₂₉ and R₃₀ are independently selected from H, C1-C20 alkyl, C1-C20 cycloalkyl, aryl, NH₂, N(CH₃)₂, (CH₂)ₙOH, (CH₂)ₙO-alkyl, (CH₂)ₙOCOCH₃, (CH₂)ₙO(CH₂)ₘOH, (CH₂)ₙO(CH₂)ₘOCOCH₃, (CH₂)ₙO(CH₂)ₘO-alkyl, (CH₂)ₙN((CH₂)ₘOH)₂, (CH₂)ₙN((CH₂)ₘO-alkyl)₂, (CH₂)ₙN((CH₂)ₘO-alkylether)₂, ((CH₂)ₙO)ₘ(CH₂)_{Q}OH, (CH₂)ₙO(CH₂)ₘNH₂, (CH₂)ₙO(CH₂)ₘN(CH₃)₂, (CH₂)ₙO(CH₂)ₘN(CH₃)₃⁺A, (CH₂)ₙN((CH₂)ₘNH₂)₂, (CH₂)ₙN(CH₂)ₘN(CH₃)₂, (CH₂)ₙO-haloalkyl, (CH₂)ₙN((CH₂)ₘN(CH₃)₃⁺A)₂, ((CH₂)ₙO)ₘ(CH₂O)_{Q}COCH₃, an alkylphosphate residue, an alkylsulfonic acid residue, an alkylsulfonic ester or alkylsulfonic amide reside, an alkylmorpholino residue, an alkylheterocyclic residue, an alkylthiol residue, a mono-, di-, or polyhydroxyalkyl residue, a mono-, di-, or polyhydroxyaryl residue, a mono-, di-, or polyetheralkyl residue, and a mono-, di-, or polyetheraryl residue, where Q, n, and m are integers ranging from 0 to 10,000, and A is a physiologically acceptable counter ion; and
M is selected from 2H, a metal cation, and photoactive metal ions selected from Ga³⁺, Pt²⁺, Pd²⁺, Sn⁴⁺, In³⁺, Ge⁴⁺, Si⁴⁺, Al³⁺, Zn²⁺, and Mg²⁺;
or a pharmaceutically acceptable salt, prodrug, solvate, or metabolite thereof.

8. A pharmaceutical composition comprising an effective diagnostic or therapeutic amount of the compound of claim 7, together with at least one pharmaceutically acceptable carrier or excipient.

9. The pharmaceutical composition according to claim 8 used to treat ophthalmic diseases.

10. The pharmaceutical composition of claim 9 wherein said ophthalmic diseases are selected from proliferative retinopathies, macular edema, corneal neovascularization, conjunctival neovascularization, ocular tumors, viral retinitis adjunct to glaucoma filtration surgery and cyclodestruction, posterior capsule opacification, and age related macular degeneration.

11. The pharmaceutical composition according to claim 8 used to treat cardiovascular diseases.

12. The pharmaceutical composition according to claim 8 used to treat skin diseases.

13. Compounds of formula IIIA and IIIB: 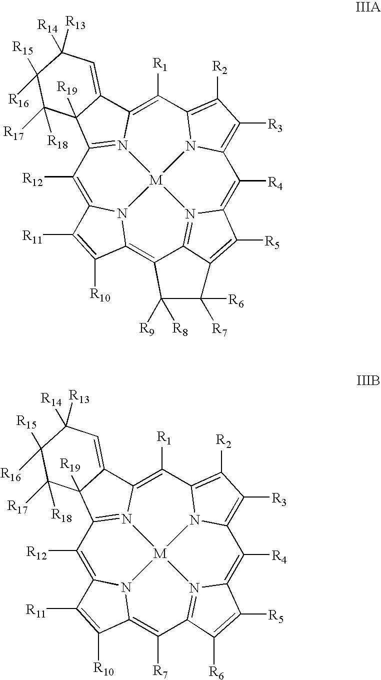
wherein:
R₁, R₂, R₃, R₄, R₅, R₆, R₇, R₈, R₉, R₁₀, R₁₁, R₁₂, R₁₃, R₁₄, R₁₅, R₁₆, R₁₇, R₁₈, and R₁₉, are independently selected from the group consisting of:
H, halogen, substituted or unsubstituted C1-C20 alkyl, heteroalkyl, haloalkyl, heterohaloalkyl, cycloalkyl, aryl, substituted aryl, alkenyl, substituted alkenyl, alkynyl, substituted alkynyl, amide, ester, ether, polyether, alkoxy, aryloxy, haloalkoxy, amino, alkylcarbonyloxy, alkoxycarbonyl, aryloxycarbonyl, azo, arylcarbonyloxy, alkoxycarbonyloxy, aryloxycarbonyloxy, sulfinyl, sulfonyl, silil, carbamoyl, heterocyclic, nitro, nitroso, formyloxy, isocyano, cyanate, isocyanate, thiocyanate, isothiocyanate, N(alkyl)₂, N(aryl)₂, CH═CH(aryl), CH═CHCH₂N(CH₃)₂, CH═CHCH₂N⁺(CH₃)₃A, CH═N(alkyl)₂⁺A, N(alkyl)₃⁺A, CN, OH, CHO, COCH₃, CO(alkyl), CO₂H, CO₂Na, CO₂K, CH(CH₃)OH, CH(CH₃)O-alkyl, CH(CH₃)O-alkoxy, CH(CH₃)O-aryl, CH(CH₃)NH-alkyl, CH(CH₃)NH-cycloalkyl, CH(CH₃)NH-heteroalkyl, CH(CH₃)NH-heteroalkoxy, CH(CH₃)-(amino acid), CH(CH₃)-(amino acid ester), CH(CH₃)-(amino acid amide), C(X)₂C(X)₃, and CH═NR₂₀, where X is selected from H and halogen, R₂₀ is selected from OH, O-alkyl, O-ether, O-alkylamino, NHCOCH₂N(CH₃)₂, NHCOCH₂N(CH₃)₃⁺A, NHCOCH₂-(pyridinium)⁺A, (CH₂)ₙO-alkoxy, and (CH₂)ₙO-alkyl, n is an integer ranging from 0 to 8, and A is a physiologically acceptable charge balancing ion;
CO₂R₂₁, where R₂₁ is selected from H, a physiologically acceptable counter ion, a C1-C20 straight or branched chain alkyl, haloalkyl, heteroalkyl, haloheteroalkyl, aryl, heteroaryl, heterocycle, a mono-, di-, or polyhydroxyalkyl residue, a mono-, di-, or polyhydroxyaryl residue, and a functional group of less than about 100,000 daltons;
(CH₂)ₙOH and (CH₂)ₙOR₂₂, where R₂₂ is selected from alkyl, haloalkyl, heteroalkyl, haloheteroalkyl, aryl, heteroaryl, heterocycle, a protecting group, a mono-, di-, or polyhydroxyalkyl residue, a mono-, di-, or polyhydroxyaryl residue, and a functional group of less than about 100,000 daltons, and n is an integer ranging from 0 to 4;
(CH₂)ₙCO₂R₂₃, (CHX)ₙCO₂R₂₃, and (CX₂)ₙCO₂R₂₃, where X is selected from OH, OR₂₄, and a halogen, and R₂₃ and R₂₄ are independently selected from H, a physiologically acceptable counter ion, acetyl, a straight or branched chain C1-C20 alkyl, haloalkyl, heteroalkyl, haloheteroalkyl, aryl, heteroaryl, heterocycle, a mono-, di-, or polyhydroxyalkyl residue, a mono-, di-, or polyhydroxyaryl residue, and a functional group of less than about 100,000 daltons, and n is an integer ranging from 0 to 4;
CONH(R₂₅), CONHNH(R₂₅), CO(R₂₅), CON(R₂₅)₂, CON(R₂₅)(R₂₆), (CH₂)ₙCONH(R₂₅), (CH₂)ₙCON(R₂₅)₂, (CH₂)ₙCOR₂₅, (CH₂)ₙCON(R₂₅)(R₂₆), (CX₂)ₙCONH(R₂₅), (CX₂)ₙCON(R₂₅)₂, (CX₂)ₙCON(R₂₅)(R₂₆), (CX₂)ₙCOR₂₅, (CH₂)ₙCONHNH(R₂₅), (CX₂)ₙCONHNH(R₂₅), (CHX)ₙCONH(R₂₅), (CHX)ₙCONHNH(R₂₅), (CHX)ₙCO(R₂₅), (CHX)ₙCON(R₂₅)₂, and (CHX)ₙCON(R₂₅)(R₂₆), where X is selected from OH, OR₂₇, SR₂₇, and a halogen, and R₂₅, R₂₆ and R₂₇ are independently selected from H, NH₂, acetyl, a straight or branched chain C1-C20 alkyl, haloalkyl, haloheteroalkyl, heteroalkyl, aryl, heteroaryl, heterocycle, a mono-, di-, or polyhydroxyalkyl residue, a mono-, di-, or polyhydroxyaryl residue, an amino acid ester, an amino acid amide, a mono-, di-, or polyetheralkyl residue, a mono-, di-, or polyetheraryl residue, and a functional group of less than about 100,000 daltons, and n is an integer ranging from 1 to 4; S(R₂₈), CH(CH₃)S(R₂₈), (CH₂)ₙS(R₂8), (CH₂)ₙNH(R₂₈), (CH₂)ₙNHNH(R₂₈), (CH₂)ₙN(R₂₈)₂, (CH₂)ₙN(R₂₈)(R₂₉), (CH₂)ₙN(R₂₈)(R₂₉)(R₃₀)⁺A, CH═N(R₂₈), CH═NN(R₂₈)(R₂₉), and amino acids containing-NH(R₂₈) or —N(R₂₈)(R₂₉), where R₂₈, R₂₉ and R₃₀ are independently selected from H, OH, O-alkyl, NH₂, acetyl, a straight or branched chain C1-C20 alkyl, haloalkyl, heteroalkyl, haloheteroalkyl, aryl, heteroaryl, heterocycle, a mono-, di-, or polyhydroxyalkyl residue, a mono-, di-, or polyhydroxyaryl residue, a mono-, di-, or polyetheralkyl residue, a mono-, di-, or polyetheraryl residue, and a functional group of less than about 10,000 daltons, where R₂₈, R₂₉ and R₃₀ together may possess the atoms necessary to constitute an aromatic ring system, n is an integer ranging from 0 to 4, and A is a physiologically acceptable counter ion;
(CH₂)ₙOPO(OR₃₁)₂ and (CH₂)ₙPO(OR₃₁)₂, where R₃₁ is selected from H, a physiologically acceptable counter ion, a straight or branched chain C1-C20 alkyl, haloalkyl, heteroalkyl, haloheteroalkyl, aryl, heteroaryl, heterocycle, a mono-, di-, or polyhydroxyalkyl residue, a mono-, di-, or polyhydroxyaryl residue, a mono-, di-, or polyetheralkyl residue, a mono-, di-, or polyetheraryl residue, and a functional group of less than about 100,000 daltons, and n is an integer ranging from 0 to 4;
(CH₂)ₙNHCOR₃₂ and (CH₂)ₙNHNHCOR₃₂, where R₃₂ is selected from a straight or branched chain C1-C20 alkyl, haloalkyl, heteroalkyl, haloheteroalkyl, aryl, heteroaryl, heterocycle, and a functional group of less than about 100,000 daltons, and n is an integer ranging from 0 to 4;
SO₃R₃₄, SO₂NHR₃₄, SO₂N(R₃₄)₂, SO₂NHNHR₃₄, SO₂R₃₄, SO₃R₃₄, (CH₂)ₙSO₂NHR₃₄, (CH₂)ₙSO₂N(R₃₄)₂, (CH₂)ₙSO₂NHNHR₃₄, and (CH₂)ₙSO₂R₃₄, where R₃₄ is selected from H, OH, a physiologically acceptable counter ion, a straight or branched chain C1-C20 alkyl, haloalkyl, heteroalkyl, haloheteroalkyl, aryl, heteroaryl, heterocycle, a mono-, di-, or polyhydroxyalkyl residue, a mono-, di-, or polyhydroxyaryl residue, a mono-, di-, or polyetheralkyl residue, a mono-, di-, or polyetheraryl residue, and a functional group of less than about 100,000 daltons, where NHR₃₄ can be an amino acid, an amino acid salt, an amino acid ester residue, or an amino acid amide residue, and n is an integer ranging from 1 to 4; and
aryl or substituted aryl, which may bear one or more substituents with a molecular weight of less than or equal to about 100,000 daltons;
wherein:
R₁₄ and R₁₅ may form a bond; and
R₆ and R₇ may form a ═O;
with the proviso that at least one of R₁ through R₃₄ is a functional group comprising a carbamate of the formulae —OCON(R₃₅)₂, —OCON═C(R₃₅)₂, —OCONR₃₅R₃₆, or —OCON═C(R₃₅)(R₃₆), where R₃₅ and R₃₆ are independently selected from H, C1-C20 alkyl, C1-C20 cycloalkyl, aryl, NH₂, N(CH₃)₂, (CH₂)ₙOH, (CH₂)ₙO-alkyl, (CH₂)ₙOCOCH₃, (CH₂)ₙO(CH₂)ₘOH, (CH₂)ₙO(CH₂)ₘOCOCH₃, (CH₂)ₙO(CH₂)ₘO-alkyl, (CH₂)ₙN((CH₂)ₘOH)₂, (CH₂)ₙN((CH₂)ₘO-alkyl)₂, (CH₂)ₙN((CH₂)ₘO-alkylether)₂, ((CH₂)ₙO)ₘ(CH₂)_{Q}OH, (CH₂)ₙO(CH₂)ₘNH₂, (CH₂)ₙO(CH₂)ₘN(CH₃)₂, (CH₂)ₙO(CH₂)ₘN(CH₃)₃⁺A, (CH₂)ₙN((CH₂)ₘNH₂)₂, (CH₂)ₙN(CH₂)ₘN(CH₃)₂, (CH₂)ₙO-haloalkyl, (CH₂)ₙN((CH₂)ₘN(CH₃)₃⁺A)₂, ((CH₂)ₙO)ₘ((CH₂O)_{Q}COCH₃, an alkylphosphate residue, an alkylsulfonic acid residue, an alkylsulfonic ester or alkylsulfonic amide reside, an alkylmorpholino residue, an alkylheterocyclic residue, an alkylthiol residue, a mono-, di-, or polyhydroxyalkyl residue, a mono-, di-, or polyhydroxyaryl residue, a mono-, di-, or polyetheralkyl residue, and a mono-, di-, or polyetheraryl residue, wherein Q, n and m are integers ranging from 0 to 10,000, and A is a physiologically acceptable counter ion; and
M is selected from 2H, a metal cation, and photoactive metal ions selected from Ga³⁺, Pt²⁺, Pd²⁺, Sn⁴⁺, In³⁺, Ge⁴⁺, Si⁴⁺, Al³⁺, Zn²⁺, and Mg²⁺;
or a pharmaceutically acceptable salt, prodrug, solvate, or metabolite thereof.

14. A pharmaceutical composition comprising an effective diagnostic or therapeutic amount of the compound of claim 13, together with at least one pharmaceutically acceptable carrier or excipient.

15. The pharmaceutical composition according to claim 14 used to treat ophthalmic diseases.

16. The pharmaceutical composition of claim 15 wherein said ophthalmic diseases are selected from proliferative retinopathies, macular edema, corneal neovascularization, conjunctival neovascularization, ocular tumors, viral retinitis adjunct to glaucoma filtration surgery and cyclodestruction, posterior capsule opacification, and age related macular degeneration.

17. The pharmaceutical composition according to claim 14 used to treat cardiovascular diseases.

18. The pharmaceutical composition according to claim 14 used to treat skin diseases.

19. Compounds of formulas IVA and IVB: 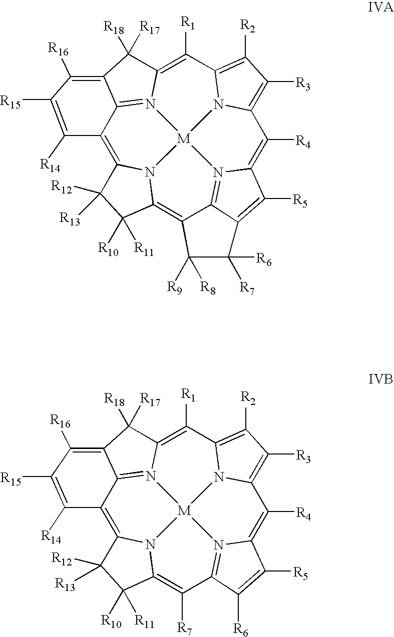
wherein:
R₁, R₂, R₃, R₄, R₅ , R₆, R₇, R₈, R₉, R₁₀, R₁₁, R₁₂, R₁₃, R₁₄, R₁₅, R₁₆, R₁₇, and R₁₈, are independently selected from the group consisting of:
H, halogen, substituted or unsubstituted C1-C20 alkyl, heteroalkyl, haloalkyl, heterohaloalkyl, cycloalkyl, aryl, substituted aryl, alkenyl, substituted alkenyl, alkynyl, substituted alkynyl, amide, ester, ether, polyether, alkoxy, aryloxy, haloalkoxy, amino, alkylcarbonyloxy, alkoxycarbonyl, aryloxycarbonyl, azo, arylcarbonyloxy, alkoxycarbonyloxy, aryloxycarbonyloxy, sulfinyl, sulfonyl, silil, carbamoyl, heterocyclic, nitro, nitroso, formyloxy, isocyano, cyanate, isocyanate, thiocyanate, isothiocyanate, N(alkyl)₂, N(aryl)₂, CH═CH(aryl), CH═CHCH₂N(CH₃)₂, CH═CHCH₂N⁺(CH₃)₃A, CH═N(alkyl)₂₊A, N(alkyl)₃₊A, CN, OH, CHO, COCH₃, CO(alkyl), CO₂H, CO₂Na, CO₂K, CH(CH₃)OH, CH(CH₃)O-alkyl, CH(CH₃)O-alkoxy, CH(CH₃)O-aryl, CH(CH₃)NH-alkyl, CH(CH₃)NH-cycloalkyl, CH(CH₃)NH-heteroalkyl, CH(CH₃)NH-heteroalkoxy, CH(CH₃)-(amino acid), CH(CH₃)-(amino acid ester), CH(CH₃)-(amino acid amide), C(X)₂C(X)₃, and CH═NR₁₉, where X is selected from H and halogen, R₁₉ is selected from OH, O-alkyl, O-ether, O-alkylamino, NHCOCH₂N(CH₃)₂, NHCOCH₂N(CH₃)₃⁺A, NHCOCH₂-(pyridinium)⁺A, (CH₂)ₙO-alkoxy, and (CH₂)ₙO-alkyl, n is an integer ranging from 0 to 8, and A is a physiologically acceptable charge balancing ion;
CO₂R₂₀, where R₂₀ is selected from H, a physiologically acceptable counter ion, a C1-C20 straight or branched chain alkyl, haloalkyl, heteroalkyl, haloheteroalkyl, aryl, heteroaryl, heterocycle, a mono-, di-, or polyhydroxyalkyl residue, a mono-, di-, or polyhydroxyaryl residue, and a functional group of less than about 100,000 daltons;
(CH₂)ₙOH and (CH₂)ₙOR₂₁, where R₂₁ is selected from alkyl, haloalkyl, heteroalkyl, haloheteroalkyl, aryl, heteroaryl, heterocycle, a protecting group, a mono-, di-, or polyhydroxyalkyl residue, a mono-, di-, or polyhydroxyaryl residue, and a functional group of less than about 100,000 daltons, and n is an integer ranging from 0 to 4;
(CH₂)ₙCO₂R₂₂, (CHX)ₙCO₂R₂₂, and (CX₂)ₙCO₂R₂₂, where X is selected from OH, OR₂₃, and a halogen, and R₂₂ and R₂₃ are independently selected from H, a physiologically acceptable counter ion, acetyl, a straight or branched chain C1-C20 alkyl, haloalkyl, heteroalkyl, haloheteroalkyl, aryl, heteroaryl, heterocycle, a mono-, di-, or polyhydroxyalkyl residue, a mono-, di-, or polyhydroxyaryl residue, and a functional group of less than about 100,000 daltons, and n is an integer ranging from 0 to 4;
CONH(R₂₄), CONHNH(R₂₄), CO(R₂₄), CON(R₂₄)₂, CON(R₂₄)(R₂₅), (CH₂)ₙnCONH(R₂₄), (CH₂)ₙCON(R₂₄)₂, (CH₂)ₙCOR₂₄, (CH₂)ₙCON(R₂₄)(R₂₅), (CX₂)ₙCONH(R₂₄), (CX₂)ₙCON(R₂₄)₂, (CX₂)ₙCON(R₂₄)(R₂₅), (CX₂)ₙCOR₂₄, (CH₂)ₙCONHNH(R₂₄), (CX₂)ₙCONHNH(R₂₄), (CHX)ₙCONH(R₂₄), (CHX)ₙCONHNH(R₂₄), (CHX)ₙCO(R₂₄), (CHX)ₙCON(R₂₄)₂, and (CHX)ₙCON(R₂₄)(R₂₅), where X is selected from OH, OR₂₆, SR₂₆, and a halogen, and R₂₄, R₂₅ and R₂₆ are independently selected from H, NH₂, acetyl, a straight or branched chain C1-C20 alkyl, haloalkyl, haloheteroalkyl, heteroalkyl, aryl, heteroaryl, heterocycle, a mono-, di-, or polyhydroxyalkyl residue, a mono-, di-, or polyhydroxyaryl residue, an amino acid ester, an amino acid amide, a mono-, di-, or polyetheralkyl residue, a mono-, di-, or polyetheraryl residue, and a functional group of less than about 100,000 daltons, and n is an integer ranging from 1 to 4;
S(R₂₇), CH(CH₃)S(R₂₇), (CH₂)ₙS(R₂₇), (CH₂)ₙNH(R₂₇), (CH₂)ₙNHNH(R₂₇), (CH₂)ₙN(R₂₇)₂, (CH₂)ₙN(R₂₇)(R₂₈), (CH₂)ₙN(R₂₇)(R₂₈)(R₂₉)⁺A, CH═N(R₂₇), CH═NN(R₂₇)(R₂₈), and amino acids containing —NH(R₂₇) or —N(R₂₇)(R₂₈), where R₂₇, R₂₈ and R₂₉ are independently selected from H, OH, O-alkyl, NH₂, acetyl, a straight or branched chain C1-C20 alkyl, haloalkyl, heteroalkyl, haloheteroalkyl, aryl, heteroaryl, heterocycle, a mono-, di-, or polyhydroxyalkyl residue, a mono-, di-, or polyhydroxyaryl residue, a mono-, di-, or polyetheralkyl residue, a mono-, di-, or polyetheraryl residue, and a functional group of less than about 100,000 daltons, where R₂₇, R₂₈ and R₂₉ together may possess the atoms necessary to constitute an aromatic ring system, n is an integer ranging from 0 to 4, and A is a physiologically acceptable counter ion;
(CH₂)ₙOPO(OR₃₀)₂ and (CH₂)ₙPO(OR₃₀)₂, where R₃₀ is selected from H, a physiologically acceptable counter ion, a straight or branched chain C1-C20 alkyl, haloalkyl, heteroalkyl, haloheteroalkyl, aryl, heteroaryl, heterocycle, a mono-, di-, or polyhydroxyalkyl residue, a mono-, di-, or polyhydroxyaryl residue, a mono-, di-, or polyetheralkyl residue, a mono-, di-, or polyetheraryl residue, and a functional group of less than about 100,000 daltons, and n is an integer ranging from 0 to 4;
(CH₂)ₙNHCOR₃₁ and (CH₂)ₙNHNHCOR₃₁, where R₃₁ is selected from a straight or branched chain C1-C20 alkyl, haloalkyl, heteroalkyl, haloheteroalkyl, aryl, heteroaryl, heterocycle, and a functional group of less than about 100,000 daltons, and n is an integer ranging from 0 to 4;
SO₃R₃₂, SO₂NHR₃₂, SO₂N(R₃₂)₂, SO₂NHNHR₃₃, SO₂R₃₃, SO₃₃R₃₃, (CH₂)ₙSO₂NHR₃₃, (CH₂)ₙSO₂N(R₃₃)₂, (CH₂)ₙSO₂NHNHR₃₃, and (CH₂)ₙSO₂R₃₃, where R₃₃ is selected from H, OH, a physiologically acceptable counter ion, a straight or branched chain C1-C20 alkyl, haloalkyl, heteroalkyl, haloheteroalkyl, aryl, heteroaryl, heterocycle, a mono-, di-, or polyhydroxyalkyl residue, a mono-, di-, or polyhydroxyaryl residue, a mono-, di-, or polyetheralkyl residue, a mono-, di-, or polyetheraryl residue, and a functional group of less than about 100,000 daltons, where NHR₃₃ can be an amino acid, an amino acid salt, an amino acid ester residue, or an amino acid amide residue, and n is an integer ranging from 1 to 4; and
aryl and substituted aryl, which may bear one or more substituents with a molecular weight of less than or equal to about 100,000 daltons;
wherein:
R₁₀ and R₁₃ may form a bond;
R₆ and R₇ may form a ═O; and
R₈ and R₉ may form a ═O;
with the proviso that at least one of R₁ through R₃₃ is a functional group that comprises a carbamate of the formulae —OCON(R₃₄)₂, —OCON═C(R₃₄)₂, —OCONR₃₄R₃₅ or —OCON═C(R₃₄)(R₃₅), where R₃₄ and R₃₅ are independently selected from H, C1-C20 alkyl, C1-C20 cycloalkyl, aryl, NH₂, N(CH₃)₂, (CH₂)ₙOH, (CH₂)ₙO-alkyl, (CH₂)ₙOCOCH₃, (CH₂)ₙO(CH₂)ₘOH, (CH₂)ₙO(CH₂)ₘOCOCH₃, (CH₂)ₙO(CH₂)ₘO-alkyl, (CH₂)ₙN((CH₂)ₘOH)₂, (CH₂)ₙN((CH₂)ₘO-alkyl)₂, (CH₂)ₙN((CH₂)ₘO-alkylether)₂, ((CH₂)ₙO)ₘ(CH₂)_{Q}OH, (CH₂)ₙO(CH₂)ₘNH₂, (CH₂)ₙO(CH₂)ₘN(CH₃)₂, (CH₂)ₙO(CH₂)ₘN(CH₃)₃⁺A, (CH₂)ₙN((CH₂)ₘNH₂)₂, (CH₂)ₙN(CH₂)ₘN(CH₃)₂, (CH₂)ₙO-haloalkyl, (CH₂)ₙN((CH₂)ₘN(CH₃)₃⁺A)₂, ((CH₂)ₙO)ₘ(CH₂O)_{Q}COCH₃, an alkylphosphate residue, an alkylsulfonic acid residue, an alkylsulfonic ester or alkylsulfonic amide reside, an alkylmorpholino residue, an alkylheterocyclic residue, an alkylthiol residue, a mono-, di-, or polyhydroxyalkyl residue, a mono-, di-, or polyhydroxyaryl residue, a mono-, di-, or polyetheralkyl residue, and a mono-, di-, or polyetheraryl residue, where Q, n, and m are integers between 0 and 10,000, and A is physiologically acceptable counter ion; and
M is selected from 2H, a metal cation, and photoactive metal ions selected from Ga³⁺, Pt²⁺, Pd²⁺, Sn⁴⁺, In³⁺, Ge⁴⁺, Si⁴⁺, Al³⁺, Zn²⁺, and Mg²⁺;
or a pharmaceutically acceptable salt, prodrug, solvate, or metabolite thereof.

20. A pharmaceutical composition comprising an effective diagnostic or therapeutic amount of the compound of claim 19, together with at least one pharmaceutically acceptable carrier or excipient.

21. The pharmaceutical composition according to claim 20 used to treat ophthalmic diseases.

22. The pharmaceutical composition of claim 21 wherein said ophthalmic diseases are selected from proliferative retinopathies, macular edema, corneal neovascularization, conjunctival neovascularization, ocular tumors, viral retinitis adjunct to glaucoma filtration surgery and cyclodestruction, posterior capsule opacification, and age related macular degeneration.

23. The pharmaceutical composition according to claim 20 used to treat cardiovascular diseases.

24. The pharmaceutical composition according to claim 20 used to treat skin diseases.

25. Compounds of formula V: 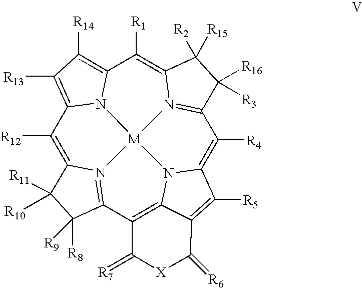
wherein:
R₁, R₂, R₃, R₄, R₅, R₆, R₇, R₈, R₉, R₁₀, R₁₁, R₁₂, R₁₃, R₁₄, R₁₅, and R₁₆ are independently selected from the group consisting of:
H, halogen, substituted or unsubstituted C1-C20 alkyl, heteroalkyl, haloalkyl, heterohaloalkyl, cycloalkyl, aryl, substituted aryl, alkenyl, substituted alkenyl, alkynyl, substituted alkynyl, amide, ester, ether, polyether, alkoxy, aryloxy, haloalkoxy, amino, alkylcarbonyloxy, alkoxycarbonyl, aryloxycarbonyl, azo, arylcarbonyloxy, alkoxycarbonyloxy, aryloxycarbonyloxy, sulfinyl, sulfonyl, silil, carbamoyl, heterocyclic, nitro, nitroso, formyloxy, isocyano, cyanate, isocyanate, thiocyanate, isothiocyanate, N(alkyl)₂, N(aryl)₂, CH═CH(aryl), CH═CHCH₂N(CH₃)₂, CH═CHCH₂N⁺(CH₃)₃A, CH═N(alkyl)₂⁺A, N(alkyl)₃⁺A, CN, OH, CHO, COCH₃, CO(alkyl), CO₂H, CO₂Na, CO₂K, CH(CH₃)OH, CH(CH₃)O-alkyl, CH(CH₃)O-alkoxy, CH(CH₃)O-aryl, CH(CH₃)NH-alkyl, CH(CH₃)NH-cycloalkyl, CH(CH₃)NH-heteroalkyl, CH(CH₃)NH-heteroalkoxy, CH(CH₃)-(amino acid), CH(CH₃)-(amino acid ester), CH(CH₃)-(amino acid amide), C(X)₂C(X)₃, and CH═NR₁₇, where X is selected from H and halogen, R₁₇ is selected from OH, O-alkyl, O-ether, O-alkylamino, NHCOCH₂N(CH₃)₂, NHCOCH₂N(CH₃)₃⁺A, NHCOCH₂-(pyridinium)⁺A, (CH₂)ₙO-alkoxy, and (CH₂)ₙO-alkyl, n is an integer ranging from 0 to 8, and A is a physiologically acceptable charge balancing ion;
CO₂R₁₈, where R₁₈ is selected from H, a physiologically acceptable counter ion, a C1-C20 straight or branched chain alkyl, haloalkyl, heteroalkyl, haloheteroalkyl, aryl, heteroaryl, heterocycle, a mono-, di-, or polyhydroxyalkyl residue, a mono-, di-, or polyhydroxyaryl residue, and a functional group of less than about 100,000 daltons;
(CH₂)ₙOH and (CH₂)ₙOR₁₉, where R₁₉ is selected from alkyl, haloalkyl, heteroalkyl, haloheteroalkyl, aryl, heteroaryl, heterocycle, a protecting group, a mono-, di-, or polyhydroxyalkyl residue, a mono-, di-, or polyhydroxyaryl residue, and a functional group of less than about 100,000 daltons, and n is an integer ranging from 0 to 4;
(CH₂)ₙCO₂R₂₀, (CHX)ₙCO₂R₂₀, and (CX₂)ₙCO₂R₂₀, where X is selected from OH, OR₂₁, and a halogen, and R₂₀ and R₂₁ are independently selected from H, a physiologically acceptable counter ion, acetyl, a straight or branched chain C1-C20 alkyl, haloalkyl, heteroalkyl, haloheteroalkyl, aryl, heteroaryl, heterocycle, a mono-, di-, or polyhydroxyalkyl residue, a mono-, di-, or polyhydroxyaryl residue, and a functional group of less than about 100,000 daltons, and n is an integer ranging from 1 to 4;
CONH(R₂₂), CONHNH(R₂₂), CO(R₂₂), CON(R₂₂)₂, CON(R₂₂)(R₂₃), (CH₂)ₙCONH(R₂₂), (CH₂)ₙCON(R₂₂)₂, (CH₂)ₙCOR₂₂, (CH₂)ₙCON(R₂₂)(R₂₃), (CX₂)ₙCONH(R₂₂), (CX₂)ₙCON(R₂₂)₂, (CX₂)ₙCON(R₂₂)(R₂₃), (CX₂)ₙCOR₂₂, (CH₂)ₙCONHNH(R₂₂), (CX₂)ₙCONHNH(R₂₂), (CHX)ₙCONH(R₂₂), (CHX)ₙCONHNH(R₂₂), (CHX)ₙCO(R₂₂), (CHX)ₙCON(R₂₂)₂, and (CHX)ₙCON(R₂₂)(R₂₃), where X is selected from OH, OR₂₄, SR₂₄, and a halogen, and R₂₂, R₂₃ and R₂₄ are independently selected from H, NH₂, acetyl, a straight or branched chain C1-C20 alkyl, haloalkyl, haloheteroalkyl, heteroalkyl, aryl, heteroaryl, heterocycle, a mono-, di-, or polyhydroxyalkyl residue, a mono-, di-, or polyhydroxyaryl residue, an amino acid ester, an amino acid amide, a mono-, di-, or polyetheralkyl residue, a mono-, di-, or polyetheraryl residue, and a functional group of less than about 100,000 daltons, and n is an integer ranging from 1 to 4;
S(R₂₅), CH(CH₃)S(R₂₅), (CH₂)ₙS(R₂₅), (CH₂)ₙNH(R₂₅) (CH₂)ₙNHNH(R₂₅), (CH₂)ₙN(R₂₅)₂, (CH₂)ₙN(R₂₅)(R₂₆), (CH₂)ₙN(R₂₅)(R₂₆)(R₂₇)⁺A, CH═N(R₂₅), CH═NN(R₂₅)(R₂₆), and amino acids containing —NH(R₂₅) or —N(R₂₅)(R₂₆), where R₂₅, R₂₆ and R₂₇ are independently selected from H, OH, O-alkyl, NH₂, acetyl, a straight or branched chain C1-C20 alkyl, haloalkyl, heteroalkyl, haloheteroalkyl, aryl, heteroaryl, heterocycle, a mono-, di-, or polyhydroxyalkyl residue, a mono-, di-, or polyhydroxyaryl residue, a mono-, di-, or polyetheralkyl residue, a mono-, di-, or pplyetheraryl residue, and a functional group of less than about 100,000 daltons, where R₂₅, R₂₆ and R₂₇ may together possess the atoms necessary to constitute an aromatic ring system, n is an integer ranging from 0 to 4, and A is a physiologically acceptable counter ion;
(CH₂)ₙOPO(OR₂₈)₂ and (CH₂)ₙPO(OR₂₈)₂, where R₂₈ is selected from H, a physiologically acceptable counter ion, a straight or branched chain C1-C20 alkyl, haloalkyl, heteroalkyl, haloheteroalkyl, aryl, heteroaryl, heterocycle, a mono-, di-, or polyhydroxyalkyl residue, a mono-, di-, or polyhydroxyaryl residue, a mono-, di-, or polyetheralkyl residue, a mono-, di-, or polyetheraryl residue, and a functional group of less than about 100,000 daltons, and n is an integer ranging from 0 to 4;
(CH₂)ₙNHCOR₂₉ and (CH₂)ₙNHNHCOR₂₉, where R₂₉ is selected from a straight or branched chain C1-C20 alkyl, haloalkyl, heteroalkyl, haloheteroalkyl, aryl, heteroaryl, heterocycle, and a functional group of less than about 100,000 daltons, and n is an integer ranging from 0 to 4;
SO₃R₃₀, SO₂NHR₃₀, SO₂N(R₃₀)₂, SO₂NHNHR₃₀, SO₂R₃₀, SO₃R₃₀,
(CH₂)ₙSO₂NHR₃₀, (CH₂)ₙSO₂N(R₃₀)₂, (CH₂)ₙSO₂NHNHR₃₀, and (CH₂)ₙSO₂R₃₀, where R₃₀ is selected from H, OH, a physiologically acceptable counter ion, a straight or branched chain C1-C20 alkyl, haloalkyl, heteroalkyl, haloheteroalkyl, aryl, heteroaryl, heterocycle, a mono-, di-, or polyhydroxyalkyl residue, a mono-, di-, or polyhydroxyaryl residue, a mono-, di-, or polyetheralkyl residue, a mono-, di-, or polyetheraryl residue, and a functional group of less than about 100,000 daltons, where NHR₃₀ can be an amino acid, an amino acid salt, an amino acid ester residue, or an amino acid amide residue, and n is an integer ranging from 0 to 4;
aryl and substituted aryl, which may bear one or more substituents with a molecular weight of less than or equal to about 100,000 daltons;
wherein:
R₁₅ and R₁₆ may form a bond;
R₉ and R₁₀ may form a bond;
R₂ and R₆ may independently be O or N(R₃₁), where R₃₁ is an alkyl;
X is selected from O and N(R₃₂), where R₃₂ is selected from alkyl, an amino acid, an amino acid ester, an amino acid amide, (CH₂)ₙOH, (CH₂)ₙO-alkyl, (CH₂)ₙOCOCH₃, (CH₂)ₙO(CH₂)ₘOH, (CH₂)ₙO(CH₂)ₘOCOCH₃, (CH₂)ₙO(CH₂)ₘO-alkyl, (CH₂)ₙN((CH₂)ₘOH)₂, (CH₂)ₙN((CH₂)ₘO-alkyl)₂, (CH₂)ₙN((CH₂)ₘO-alkylether)₂, ((CH₂)ₙO)ₘ(CH₂)_{Q}OH, (CH₂)ₙO(CH₂)ₘNH₂, (CH₂)ₙO(CH₂)ₘN(CH₃)₂, (CH₂)ₙO(CH₂)ₘN(CH₃)₃⁺A, (CH₂)ₙN((CH₂)ₘNH₂)₂, (CH₂)ₙN(CH₂)ₘN(CH₃)₂, (CH₂)ₙO-haloalkyl, (CH₂)ₙN(CH₂)ₘN(CH₃)₃⁺A, ((CH₂)ₙO)ₘ(CH₂O)_{Q}COCH₃, a mono-, di-, or polyhydroxyalkyl residue, a mono-, di-, or polyhydroxyaryl residue, a mono-, di-, or polyetheralkyl residue, a mono-, di-, or polyetheraryl residue, a functional group that possesses a carbamate moiety of the formulae —OCON(R₃₃)₂, —OCON═C(R₃₃)₂, —OCONR₃₃R₃₄ or —OCON═C(R₃₃)(R₃₄), and a functional group having a molecular weight less than or equal to 100,000 daltons, where R₃₃ and R₃₄ are independently selected from H, C1-C20 alkyl, C1-C20 cycloalkyl, aryl, NH₂, N(CH₃)₂, (CH₂)ₙOH, (CH₂)ₙO-alkyl, (CH₂)ₙOCOCH₃, (CH₂)ₙO(CH₂)ₘOH, (CH₂)ₙO(CH₂)ₘOCOCH₃, (CH₂)ₙO(CH₂)ₘO-alkyl, (CH₂)ₙN((CH₂)ₘOH)₂, (CH₂)ₙN((CH₂)ₘO-alkyl)₂, (CH₂)ₙN((CH₂)ₘO-alkylether)₂, ((CH₂)ₙO)ₘ(CH₂)_{Q}OH, (CH₂)ₙO(CH₂)ₘNH₂, (CH₂)ₙO(CH₂)ₘN(CH₃)₂, (CH₂)ₙO(CH₂)ₘN(CH₃)₃⁺A, (CH₂)ₙN((CH₂)ₘNH₂)₂, (CH₂)ₙN(CH₂)ₘN(CH₃)₂, (CH₂)ₙO-haloalkyl, (CH₂)ₙN((CH₂)ₘN(CH₃)₃⁺A)₂, ((CH₂)ₙO)ₘ(CH₂O)_{Q}COCH₃, an alkylphosphate residue, an alkylsulfonic acid residue, an alkylsulfonic ester, an alkylsulfonic amide reside, an alkylmorpholino residue, an alkylheterocyclic residue, an alkylthiol residue, a mono-, di-, or polyhydroxyalkyl residue, a mono-, di-, or polyhydroxyaryl residue, a mono-, di-, or polyetheralkyl residue, and a mono-, di-, or polyetheraryl residue, where A is a physiologically acceptable counter ion, and Q, n, and m are integers ranging from 0 to 10,000;
with the proviso that at least one of R₁ through R₃₀ is a functional group that comprises a carbamate of the formulae —OCON(R₃₃)₂, —OCON═C(R₃₃)₂, —OCONR₃₃R₃₄ or —OCON═C(R₃₃)(R₃₄); and
M is selected from 2H, a metal cation, and photoactive metal ions selected from Ga ³⁺, Pt²⁺, Pd²⁺, Sn⁴⁺, In³⁺, Ge⁴⁺, Si⁴⁺, Al³⁺, Zn²⁺, and Mg²⁺;
or a pharmaceutically acceptable salt, prodrug, solvate, or metabolite thereof.

26. A pharmaceutical composition comprising an effective diagnostic or therapeutic amount of the compound of claim 25, together with at least one pharmaceutically acceptable carrier or excipient.

27. The pharmaceutical composition according to claim 26 used to treat ophthalmic diseases.

28. The pharmaceutical composition of claim 27 wherein said ophthalmic diseases are selected from proliferative retinopathies, macular edema, corneal neovascularization, conjunctival neovascularization, ocular tumors, viral retinitis adjunct to glaucoma filtration surgery and cyclodestruction, posterior capsule opacification, and age related macular degeneration.

29. The pharmaceutical composition according to claim 25 used to treat cardiovascular diseases.

30. The pharmaceutical composition according to claim 25 used to treat skin diseases.

31. Compounds of the following formula: 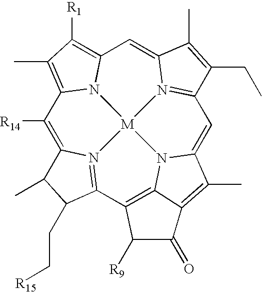
wherein:
R₁ is selected from (CH₂)ₙOCON(R₂₉)₂, (CH₂)ₙOCON═C(R₂₉)₂, (CH₂)ₙOCONR₂₉R₃₀, (CH₂)ₙOCON═C(R₂₉)(R₃₀), CH(OCON(R₂₉)₂)CH₃, CH(OCON═C(R₂₉)₂)CH₃, CH(OCONR₂₉R₃₀)CH₃, and CH(OCON═C(R₂₉)(R₃₀))CH₃, where R₂₉ and R₃₀ are independently selected from H, C1-C20 alkyl, C1-C20 cycloalkyl, aryl, NH₂, N(CH₃)₂, (CH₂)ₙOH, (CH₂)ₙO-alkyl, (CH₂)ₙOCOCH₃, (CH₂)ₙO(CH₂)ₘOH, (CH₂)ₙO(CH₂)MOCOCH₃, (CH₂)ₙO(CH₂)ₘO-alkyl, (CH₂)ₙN((CH₂)ₘOH)₂, (CH₂)ₙN((CH₂)ₘO-alkyl)₂, (CH₂)ₙN((CH₂)ₘO-alkylether)₂, ((CH₂)ₙO)ₘ((CH₂)_{Q})OH, (CH₂)ₙO(CH₂)ₘNH₂, (CH₂)ₙO(CH₂)ₘN(CH₃)₂, (CH₂)ₙO(CH₂)ₘN(CH₃)₃⁺A, (CH₂)ₙN((CH₂)ₘNH₂)₂, (CH₂)ₙN(CH₂)ₘN(CH₃)₂, (CH₂)ₙO-haloalkyl, (CH₂)ₙN(CH₂)ₙN(CH₃)₃⁺A)₂, ((CH₂)ₙO)ₘ(CH₂O)_{Q}COCH₃, an alkylphosphate residue, an alkylsulfonic acid residue, an alkylsulfonic ester residue, alkylsulfonic amide residue, an alkylmorpholino residue, an alkylheterocyclic residue, an alkylthiol residue, a mono-, di-, or polyhydroxyalkyl residue, a mono-, di-, or polyhydroxyaryl residue, a mono-, di-, or polyetheralkyl residue, and a mono-, di-, or polyetheraryl residue, where Q, n, and m are integers ranging from 0 to 10,000, and A is a physiologically acceptable counter ion;
R₉ and R14 are selected from H, methyl, and a halogen;
R₁₅ is selected from NH₂, NH₃⁺A, N(alkyl)₂, N(alkyl₃)₃⁺A, CO₂R₁₆, CONR₁₆R₁₇, an amino acid containing NR₁₆R₁₇, an amino acid ester containing NR₁₆R₁₇, and an amino acid amide containing NR₁₆R₁₇, where R₁₆ and R₁₇ are independently selected from H, a physiologically acceptable counter ion, a C1-C20 straight or branched chain alkyl, haloalkyl, heteroalkyl, haloheteroalkyl, aryl, heteroaryl, heterocycle, a mono-, di-, or polyhydroxyalkyl residue, and a mono-, di-, or polyhydroxyaryl residue, and A is a physiologically acceptable counter ion; and
M is selected from 2H, a metal cation, and photoactive metal ions selected from Ga³⁺, Pt²⁺, Pd²⁺, Sn⁴⁺, In³⁺, Ge⁴⁺, Si⁴⁺, Al³⁺, Zn²⁺, and Mg²⁺;
or a pharmaceutically acceptable salt, prodrug, solvate, or metabolite thereof.

32. A pharmaceutical composition comprising an effective, diagnostic or therapeutic amount of the compound of claim 31, together,with at least one pharmaceutically acceptable carrier or excipient.

33. The pharmaceutical composition according to claim 32 used to treat ophthalmic diseases.

34. The pharmaceutical composition of claim 33 wherein said ophthalmic diseases are selected from proliferative retinopathies, macular edema, corneal neovascularization, conjunctival neovascularization, ocular tumors, viral retinitis adjunct to glaucoma filtration surgery and cyclodestruction, posterior capsule opacification, and age related macular degeneration.

35. The pharmaceutical composition according to claim 32 used to treat cardiovascular diseases.

36. The pharmaceutical composition according to claim 32 used to treat skin diseases.

37. Compounds of the following formula: 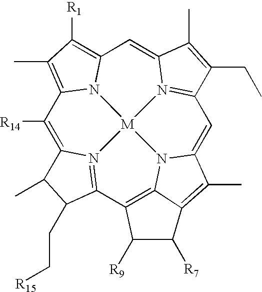
wherein:
R₁ is selected from H, CH₃, CH₂CH₃, CH═CH₂, CH₂OH, CH₂OAc, CH₂O-alkyl, CH₂O-alkoxy, CH═CHCH₂N(CH₃)₂, CH═CHCH₂N(CH₃)₃⁺A⁻, COCH₃, CHO, CH(OH)CH₃, CH(O-alkyl)CH₃, CH(O-alkoxy)CH₃, CH₂CH₂O-alkyl, CH₂CH₂O-alkoxy, and CH₂CH₂OAc;
R₇ is selected from OCON(R₂₉)₂, OCON═C(R₂₉)₂, OCONR₂₉R₃₀, and OCON═C(R₂₉)(R₃₀), where R₂₉and R₃₀ are independently selected from H, C1-C20 alkyl, C1-C20 cycloalkyl, aryl, NH₂, N(CH₃)₂, (CH₂)ₙOH, (CH₂)ₙO-alkyl, (CH₂)ₙOCOCH₃, (CH₂)ₙO(CH₂)ₘOH, (CH₂)ₙO(CH₂)ₘOCOCH₃, (CH₂)ₙO(CH₂)ₘO-alkyl, (CH₂)ₙN((CH₂)ₘOH)₂, (CH₂)ₙN((CH₂)ₘO-alkyl)₂, (CH₂)ₙN((CH₂)ₘO-alkylether)₂, ((CH₂)ₙO)ₘ((CH₂)_{Q})OH, (CH₂)ₙO(CH₂)ₘNH₂, (CH₂)ₙO(CH₂)ₘN(CH₃)₂, (CH₂)ₙO(CH₂)ₘN(CH₃)₃⁺A, (CH₂)ₙN((CH₂)ₘNH₂)₂, (CH₂)ₙN(CH₂)ₘN(CH₃)₂, (CH₂)ₙO-haloalkyl, (CH₂)ₙN(CH₂)ₘN(CH₃)₃ A)₂, ((CH₂)ₙO)ₘ(CH₂O)_{Q}COCH₃, an alkylphosphate residue, an alkylsulfonic acid residue, an alkylsulfonic ester residue, alkylsulfonic amide residue, an alkylmorpholino residue, an alkylheterocyclic residue, an alkylthiol residue, a mono-, di-, or polyhydroxyalkyl residue, a mono-, di-, or polyhydroxyaryl residue, a mono-, di-, or polyetheralkyl residue, and a mono-, di-, or polyetheraryl residue, where Q, n, and m are integers ranging from 0 to 10,000, and A is a physiologically acceptable counter ion;
R₁₄ is selected from H, methyl, and a halogen;
R₉ and R₁₅ are independently selected from NH₂, NH₃⁺A, N(alkyl)₂, N(alkyl₃)₃⁺A, CO₂R₁₆, CONR₁₆R₁₇, an amino acid containing NR₁₆R₁₇, an amino acid ester containing NR₁₆R₁₇, and an amino acid amide containing NR₁₆R₁₇, where R₁₆ and R₁₇ are independently selected from H, a physiologically acceptable counter ion, a C1-C20 straight or branched chain alkyl, haloalkyl, heteroalkyl, haloheteroalkyl, aryl, heteroaryl, heterocycle, a mono-, di-, or polyhydroxyalkyl residue, and a mono-, di-, or polyhydroxyaryl residue, and A is a physiologically acceptable counter ion; and
M is selected from 2H, a metal cation, and photoactive metal ions selected from Ga³⁺, Pt²⁺, Pd²⁺, Sn⁴⁺, In³⁺, Ge⁴⁺, Si⁴⁺, Al³⁺, Zn²⁺, and Mg²⁺;
or a pharmaceutically acceptable salt, prodrug, solvate, or metabolite thereof.

38. A pharmaceutical composition comprising an effective diagnostic or therapeutic amount of the compound of claim 37, together with at least one pharmaceutically acceptable carrier or excipient.

39. The pharmaceutical composition according to claim 38 used to treat ophthalmic diseases.

40. The pharmaceutical composition of claim 39 wherein said ophthalmic diseases are selected from proliferative retinopathies, macular edema, corneal neovascularization, conjunctival neovascularization, ocular tumors, viral retinitis adjunct to glaucoma filtration surgery and cyclodestruction, posterior capsule opacification, and age related macular degeneration.

41. The pharmaceutical composition according to claim 38 used to treat cardiovascular diseases.

42. The pharmaceutical composition according to claim 38 used to treat skin diseases.

43. Compounds of the following formula: 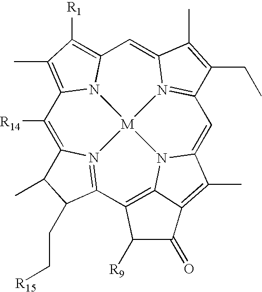
wherein:
R₁ is selected from H, CH₃, CH₂CH₃, CH═CH₂, CH₂OH, CH₂OAc, CH₂O-alkyl, CH₂O-alkoxy, CH═CHCH₂N(CH₃)₂, CH═CHCH₂N(CH₃)₃⁺A⁻, COCH₃, CHO, CH(OH)CH₃, CH(O-alkyl)CH₃, CH(O-alkoxy)CH₃, CH₂CH₂O-alkyl, CH₂CH₂O-alkoxy, and CH₂CH₂OAc, and A is a physiologically acceptable counter ion;
R₉ and R₁₅ are independently selected from NH₂, NH₃⁺A, N(alkyl)₂, N(alkyl₃)₃⁺A, CO₂R₁₆, CONR₁₆R₁₇, CO₂(CH₂)ₙOCON(R₂₉)₂, CO₂(CH₂)ₙOCON═C(R₂₉)₂, CO₂(CH₂)ₙOCONR₂₉R₃₀, CO₂(CH₂)ₙOCON═C(R₂₉)(R₃₀), CONH(CH₂)ₙOCON(R₂₉)₂, CONH(CH₂)ₙOCON═C(R₂₉)₂, CONH(CH₂)ₙOCONR₂₉R₃₀, CONH(CH₂)ₙOCON═C(R₂₉)(R₃₀), an amino acid containing NR₁₆R₁₇, an amino acid ester containing NR₁₆R₁₇, and an amino acid amide containing NR₁₆R₁₇, where R₁₆ and R₁₇ are independently selected from H, a physiologically acceptable counter ion, a C1-C20 straight or branched chain alkyl, haloalkyl, heteroalkyl, haloheteroalkyl, aryl, heteroaryl, heterocycle, a mono-, di-, or polyhydroxyalkyl residue, and a mono-, di-, or polyhydroxyaryl residue, and where R₂₉ and R₃₀ are independently selected from H, C1-C20 alkyl, C1-C20 cycloalkyl, aryl, NH₂, N(CH₃)₂, (CH₂)ₙOH, (CH₂)ₙO-alkyl, (CH₂)ₙOCOCH₃, (CH₂)ₙO(CH₂)ₘOH, (CH₂)ₙO(CH₂)ₘOCOCH₃, (CH₂)ₙO(CH₂)ₘO-alkyl, (CH₂)ₙN((CH₂)ₘOH)₂, (CH₂)ₙN((CH₂)ₘO-alkyl)₂, (CH₂)ₙN((CH₂)ₘO-alkylether)₂, ((CH₂)ₙO)ₘ((CH₂)_{Q})OH, (CH₂)ₙO(CH₂)ₘNH₂, (CH₂)ₙO(CH₂)ₘN(CH₃)₂, (CH₂)ₙO(CH₂)ₘN(CH₃)₃⁺A, (CH₂)ₙN((CH₂)ₘNH₂)₂, (CH₂)ₙN(CH₂)ₘN(CH₃)₂, (CH₂)ₙO-haloalkyl, (CH₂)ₙN(CH₂)ₘN(CH₃)₃⁺A)₂, ((CH₂)ₙO)ₘ(CH₂O)_{Q}COCH₃, an alkylphosphate residue, an alkylsulfonic acid residue, an alkylsulfonic ester residue, alkylsulfonic amide residue, an alkylmorpholino residue, an alkylheterocyclic residue, an alkylthiol residue, a mono-, di-, or polyhydroxyalkyl residue, a mono-, di-, or polyhydroxyaryl residue, a mono-, di-, or polyetheralkyl residue, and a mono-, di-, or polyetheraryl residue, where Q, n, and m are integers ranging from 0 to 10,000, and A is a physiologically acceptable counter ion, wherein at least one of R₉ and R₁₅ comprises a carbamate group;
R₁₄ is selected from H, methyl, and a halogen; and
M is selected from 2H, a metal cation, and photoactive metal ions selected from Ga³⁺, Pt²⁺, Pd²⁺, Sn⁴⁺, In³⁺, Ge⁴⁺, Si⁴⁺, Al³⁺, Zn²⁺, and Mg²⁺;
or a pharmaceutically acceptable salt, prodrug, solvate, or metabolite thereof.

44. A pharmaceutical composition comprising an effective diagnostic or therapeutic amount of the compound of claim 43, together with at least one pharmaceutically acceptable carrier or excipient.

45. The pharmaceutical composition according to claim 44 used to treat ophthalmic diseases.

46. The pharmaceutical composition of claim 45 wherein said ophthalmic diseases are selected from proliferative retinopathies, macular edema, corneal neovascularization, conjunctival neovascularization, ocular tumors, viral retinitis adjunct to glaucoma filtration surgery and cyclodestruction posterior capsule opacification and age related macular degeneration.

47. The pharmaceutical composition according to claim 44 used to treat cardiovascular diseases.

48. The pharmaceutical composition according to claim 44 used to treat skin diseases.

49. Compounds of the following formula: 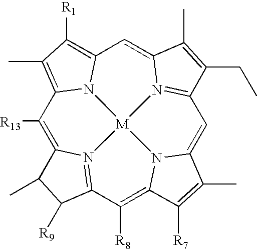
wherein:
R₁ is selected from (CH₂)ₙOCON(R₂₉)₂, (CH₂)ₙOCON═C(R₂₉)₂, (CH₂)ₙOCONR₂₉R₃₀, (CH₂)ₙOCON═C(R₂₉)(R₃₀), CH(OCON(R₂₉)₂)CH₃, CH(OCON═C(R₂₉)₂)CH₃, CH(OCONR₂₉R₃₀)CH₃, and CH(OCON═C(R₂₉)(R₃₀))CH₃, where R₂₉ and R₃₀ are independently selected from H, C1-C20 alkyl, C1-C20 cycloalkyl, aryl, NH₂, N(CH₃)₂, (CH₂)ₙOH, (CH₂)ₙO-alkyl, (CH₂)ₙOCOCH₃, (CH₂)ₙO(CH₂)ₘOH, (CH₂)ₙO(CH₂)ₘOCOCH₃, (CH₂)ₙO(CH₂)ₘO-alkyl, (CH₂)ₙN((CH₂)ₘOH)₂, (CH₂)ₙN((CH₂)ₘO-alkyl)₂, (CH₂)ₙN((CH₂)ₘO-alkylether)₂, ((CH₂)ₙO)ₘ((CH₂)_{Q})OH, (CH₂)ₙO(CH₂)ₘNH₂, (CH₂)ₙO(CH₂)ₘN(CH₃)₂, (CH₂)ₙO(CH₂)ₘN(CH₃)₃⁺A, (CH₂)ₙN((CH₂)ₘNH₂)₂, (CH₂)ₙN(CH₂)ₘN(CH₃)₂, (CH₂)ₙO-haloalkyl, (CH₂)ₙN(CH₂)ₘN(CH₃)₃⁺A)₂, ((CH₂)ₙO)ₘ(CH₂O)_{Q}COCH₃, an alkylphosphate residue, an alkylsulfonic acid residue, an alkylsulfonic ester residue, alkylsulfonic amide residue, an alkylmorpholino residue, an alkylheterocyclic residue, an alkylthiol residue, a mono-, di-, or polyhydroxyalkyl residue, a mono-, di-, or polyhydroxyaryl residue, a mono-, di-, or polyetheralkyl residue, and a mono-, di-, or polyetheraryl residue, where Q, n, and m are integers ranging from 0 to 10,000, and A is a physiologically acceptable counter ion;
R₁₃ is selected from H, methyl, and a halogen;
R₇, R₈ and R₉ are independently selected from NH₂, NH₃^{A}, N(alkyl)₂, N(alkyl₃)₃⁺A, CO₂R₁₆, CONR₁₆R₁₇, (CH₂)ₙCO₂R₁₆, (CH₂)ₙCONR₁₆R₁₇, an amino acid containing NR₁₆R₁₇, an amino acid ester containing NR₁₆R₁₇, and an amino acid amide containing NR₁₆R₁₇, where R₁₆ and R₁₇ are independently selected from H, a physiologically acceptable counter ion, a C1-C20 straight or branched chain alkyl, haloalkyl, heteroalkyl, haloheteroalkyl, aryl, heteroaryl, heterocycle, a mono-, di-, or polyhydroxyalkyl residue, and a mono-, di-, or polyhydroxyaryl residue, where A is a physiologically acceptable counter ion and n is an integer from 1 to 4; and
M is selected from 2H, a metal cation, and photoactive metal ions selected from Ga³⁺, Pt²⁺, Pd²⁺, Sn⁴⁺, In³⁺, Ge⁴⁺, Si⁴⁺, Al³⁺, Zn²⁺, and Mg²⁺;
or a pharmaceutically acceptable salt, prodrug, solvate, ometabolite thereof.

50. A pharmaceutical composition comprising an effective diagnostic or therapeutic amount of the compound of claim 49, together with at least one pharmaceutically acceptable carrier or excipient.

51. The pharmaceutical composition according to claim 50 used to treat ophthalmic diseases.

52. The pharmaceutical composition of claim 51 wherein said ophthalmic diseases are selected from proliferative retinopathies, macular edema, corneal neovascularization, conjunctival neovascularization, ocular tumors, viral retinitis adjunct to glaucoma filtration surgery and cyclodestruction, posterior capsule opacification, and age related macular degeneration.

53. The pharmaceutical composition according to claim 50 used to treat cardiovascular diseases.

54. The pharmaceutical composition according to claim 51 used to treat skin diseases.

55. Compounds of the following formula: 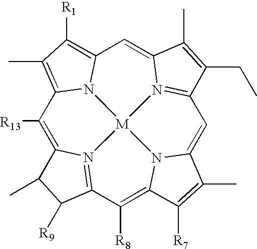
wherein:
R₁ is selected from H, CH₃, CH₂CH₃, CH═CH₂, CH₂OH, CH₂OAc, CH₂O-alkyl, CH₂O-alkoxy, CH═CHCH₂N(CH₃)₂, CH═CHCH₂N(CH₃)₃⁺A⁻, COCH₃, CHO, CH(OH)CH₃, CH(O-alkyl)CH₃, CH(O-alkoxy)CH3, CH₂CH₂O-alkyl, CH₂CH₂O-alkoxy, and CH₂CH₂OAc, where A is a physiologically acceptable counter ion;
R₁₃ is selected from H, methyl, and halogen;
R₇, R₈, and R₉ are independently selected from (CH₂)ₙNH₂, (CH₂)ₙNH₃⁺A, (CH₂)ₙN(alkyl)₂, (CH₂)ₙN(alkyl₃)₃⁺A, (CH₂)ₙCO₂R₁₆, (CH₂)ₙCONR₁₆R₁₇, (CH₂)ₙOCON(R₂₉)₂, (CH₂)ₙOCON═C(R₂₉)₂, (CH₂)ₙOCONR₂₉R₃₀, (CH₂)ₙOCON═C(R₂₉)(R₃₀), (CH₂)ₙCO₂(CH₂)ₙOCON(R₂₉)₂, (CH₂)ₙCO₂(CH₂)ₙOCON═C(R₂₉)₂, (CH₂)ₙCO₂(CH₂)ₙOCONR₂₉R₃₀, (CH₂)ₙCO₂(CH₂)ₙOCON═C(R₂₉)(R₃₀), (CH₂)ₙCONH(CH₂)ₙOCON(R₂₉)₂, (CH₂)ₙCONH(CH₂)ₙOCON═C(R₂₉)₂, (CH₂)ₙCONH(CH₂)ₙOCONR₂₉R₃₀, (CH₂)ₙCONH(CH₂)ₙOCON═C(R₂₉)(R₃₀), an amino acid containing NR₁₆R₁₇, an amino acid ester containing NR₁₆R₁₇, and an amino acid amide containing NR₁₆R₁₇, where R₁₆ and R₁₇ are independently selected from H, a physiologically acceptable counter ion, a C1-C20 straight or branched chain alkyl, haloalkyl, heteroalkyl, haloheteroalkyl, aryl, heteroaryl, heterocycle, a mono-, di-, or polyhydroxyalkyl residue, and a mono-, di-, or polyhydroxyaryl residue, where R₂₉ and R₃₀ are independently selected from H, C1-C20 alkyl, C1-C20 cycloalkyl, aryl, NH₂, N(CH₃)₂, (CH₂)ₙOH, (CH₂)ₙO-alkyl, (CH₂)ₙOCOCH₃, (CH₂)ₙO(CH₂)ₘOH, (CH₂)ₙO(CH₂)ₘOCOCH₃, (CH₂)ₙO(CH₂)ₘO-alkyl, (CH₂)ₙN((CH₂)ₘOH)₂, (CH₂)ₙN((CH₂)ₘO-alkyl)₂, (CH₂)ₙN((CH₂)ₘO-alkylether)₂, ((CH₂)ₙO)ₘ((CH₂)_{Q})OH, (CH₂)ₙO(CH₂)ₘNH₂, (CH₂)ₙO(CH₂)ₘN(CH₃)₂, (CH₂)ₙO(CH₂)ₘN(CH₃)₃⁺A, (CH₂)ₙN((CH₂)ₘNH₂)₂, (CH₂)ₙN(CH₂)ₘN(CH₃)₂, (CH₂)ₙO-haloalkyl, (CH₂)ₙN(CH₂)ₘN(CH₃)₃⁺A)₂, ((CH₂)ₙO)ₘ(CH₂O)_{Q}COCH₃, an alkylphosphate residue, an alkylsulfonic acid residue, an alkylsulfonic ester residue, alkylsulfonic amide residue, an alkylmorpholino residue, an alkylheterocyclic residue, an alkylthiol residue, a mono-, di-, or polyhydroxyalkyl residue, a mono-, di-, or polyhydroxyaryl residue, a mono-, di-, or polyetheralkyl residue, and a mono-, di-, or polyetheraryl residue, where Q, n and m are integers ranging from 0 to 10,000, and A is a physiologically acceptable counter ion, wherein at least one of R₇, R₈ or R₉ possesses a carbamate moiety in its structure;
R₁₃ is selected from H, methyl, and a halogen; and
M is selected from 2H, a metal cation, and photoactive metal ions selected from Ga³⁺, Pt²⁺, Pd²⁺, Sn⁴⁺, In³⁺, Ge⁴⁺, Si⁴⁺, Al³⁺, Zn²⁺, and Mg²⁺;
or a pharmaceutically acceptable salt, prodrug, solvate, or metabolite thereof.

56. A pharmaceutical composition comprising an effective diagnostic or therapeutic amount of the compound of claim 55, together with at least one pharrnaceutically acceptable carrier or excipient.

57. The pharmaceutical composition according to claim 56 used to treat ophthalmic diseases.

58. The pharmaceutical composition of claim 57 wherein said ophthalmic diseases are selected from proliferative retinopathies, macular edema, corneal neovascularization, conjunctival neovascularization, ocular tumors, viral retinitis adjunct to glaucoma filtration surgery and cyclodestruction, posterior capsule opacification, and age related macular degeneration.

59. The pharmaceutical composition according to claim 56 used to treat cardiovascular diseases.

60. The pharmaceutical composition according to claim 56 used to treat skin diseases.

61. Compounds of the following formula: 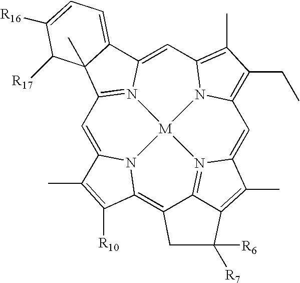
wherein:
R₆, R₇, R₁₀, R₁₆, and R₁₇ are independently selected from H, OH, (CH₂)ₙNH₂, (CH₂)ₙNH₃⁺A, (CH₂)ₙN(alkyl)₂, (CH₂)ₙN(alkyl₃)₃⁺A, (CH₂)ₙCO₁₂R₁₉, (CH₂)ₙCONR₁₉R₂₀, (CH₂)ₙOCON(R₂₉)₂, (CH₂)ₙOCON═C(R₂₉)₂, (CH₂)ₙOCONR₂₉R₃₀, (CH₂)ₙOCON═C(R₂₉)(R₃₀), (CH₂)ₙCO₂(CH₂)ₙOCON(R₂₉)₂, (CH₂)ₙCO₂(CH₂)ₙOCON═C(R₂₉)₂, (CH₂)ₙCO₂(CH₂)ₙOCONR₂₉R₃₀, (CH₂)ₙCO₂(CH₂)ₙOCON═C(R₂₉)(R₃₀), (CH₂)ₙCONH(CH₂)ₙOCON(R₂₉)₂, (CH₂)ₙCONH(CH₂)ₙOCON═C(R₂₉)₂, (CH₂)ₙCONH(CH₂)ₙOCONR₂₉R₃₀, (CH₂)ₙCONH(CH₂)ₙOCON═C(R₂₉)(R₃₀), an amino acid containing NR₁₆R₁₇, an amino acid ester containing NR₁₆R₁₇, and an amino acid amide containing NR₁₆R₁₇, where R₁₉ and R₂₀ are independently selected from H, a physiologically acceptable counter ion, a C1-C20 straight or branched chain alkyl, haloalkyl, heteroalkyl, haloheteroalkyl, aryl, heteroaryl, heterocycle, a mono-, di-, or polyhydroxyalkyl residue, and a mono-, di-, or polyhydroxyaryl residue, and where R₂₉ and R₃₀ are independently selected from H, C1-C20 alkyl, C1-C20 cycloalkyl, aryl, NH₂, N(CH₃)₂, (CH₂)ₙOH, (CH₂)ₙO-alkyl, (CH₂)ₙOCOCH₃, (CH₂)ₙO(CH₂)ₘOH, (CH₂)ₙO(CH₂)ₘOCOCH₃, (CH₂)ₙO(CH₂)ₘO-alkyl, (CH₂)ₙN((CH₂)ₘOH)₂, (CH₂)ₙN((CH₂)ₘO-alkyl)₂, (CH₂)ₙN((CH₂)ₘO-alkylether)₂, ((CH₂)ₙO)ₘ((CH₂)_{Q})OH, (CH₂)ₙO(CH₂)ₘNH₂, (CH₂)ₙO(CH₂)ₘN(CH₃)₂, (CH₂)ₙO(CH₂)ₘN(CH₃)₃⁺A, (CH₂)ₙN((CH₂)ₘNH₂)₂, (CH₂)ₙN(CH₂)ₘN(CH₃)₂, (CH₂)ₙO-haloalkyl, (CH₂)ₙN(CH₂)ₘN(CH₃)₃⁺A)₂, ((CH₂)ₙO)ₘ(CH₂O)_{Q}COCH₃, an alkylphosphate residue, an alkylsulfonic acid residue, an alkylsulfonic ester residue, alkylsulfonic amide residue, an alkylmorpholino residue, an alkylheterocyclic residue, an alkylthiol residue, a mono-, di-, or polyhydroxyalkyl residue, a mono-, di-, or polyhydroxyaryl residue, a mono-, di-, or polyetheralkyl residue, and a mono-, di-, or polyetheraryl residue, where Q, n, and m are integers ranging from 0 to 10,000, and A is a physiologically acceptable counter ion, wherein at least one of R₆, R₇, R₁₀, R₁₆, and R₁₇ possesses a carbamate moiety in its structure, and R₆ and R₇ may form a ═O; and
M is selected from 2H, a metal cation, and photoactive metal ions selected from Ga³⁺, Pt²⁺, Pd²⁺, Sn⁴⁺, In³⁺, Ge⁴⁺, Si⁴⁺, Al³⁺, Zn²⁺, and Mg²⁺; or a pharmaceutically acceptable salt, prodrug, solvate, or metabolite thereof.

62. A pharmaceutical composition comprising an effective diagnostic or therapeutic amount of the compound of claim 61, together with at least one pharmaceutically acceptable carrier or excipient.

63. The pharmaceutical composition according to claim 62 used to treat ophthalmic diseases.

64. The pharmaceutical composition of claim 63 wherein said ophthalmic diseases are selected from proliferative retinopathies, macular edema, corneal neovascularization, conjunctival neovascularization, ocular tumors, viral retinitis adjunct to glaucoma filtration surgery and cyclodestruction, posterior capsule opacification, and age related macular degeneration.

65. The pharmaceutical composition according to claim 62 used to treat cardiovascular diseases.

66. The pharmaceutical composition according to claim 62 used to treat skin diseases.

67. Compounds of the following formula: 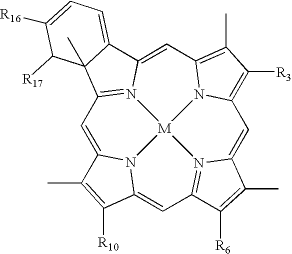
wherein:
R₃, R₆, R₁₀, R₁₆, and R₁₇ are independently selected from CH═CH₂, (CH₂)ₙCO₂R₁₉, (CH₂)ₙCONR₁₉R₂₀, (CH₂)ₙOCON(R₂₉)₂, (CH₂)ₙOCON═C(R₂₉)₂, (CH₂)ₙOCONR₂₉R₃₀, (CH₂)ₙOCON═C(R₂₉)(R₃₀), (CH₂)ₙCO₂(CH₂)ₙOCON(R₂₉)₂, (CH₂)ₙCO₂(CH₂)ₙOCON═C(R₂₉)₂, (CH₂)ₙCO₂(CH₂)ₙOCONR₂₉R₃₀, (CH₂)ₙCO₂(CH₂)ₙOCON═C(R₂₉)(R₃₀), (CH₂)ₙCONH(CH₂)ₙOCON(R₂₉)₂, (CH₂)ₙCONH(CH₂)ₙOCON═C(R₂₉)₂, (CH₂)ₙCONH(CH₂)ₙOCONR₂₉R₃₀, (CH₂)ₙCONH(CH₂)ₙOCON═C(R₂₉)(R₃₀), CH(OCON(R₂₉)₂)CH₃, CH(OCON═C(R₂₉)₂)CH₃, CH(OCONR₂₉R₃₀)CH₃, CH(OCON═C(R₂₉)(R₃₀))CH₃, an amino acid containing NR₁₉R₂₀, an amino acid ester containing NR₁₉R₂₀, and an amino acid amide containing NR₁₉R₂₀, where R₁₉ and R₂₀ are independently selected from H, a physiologically acceptable counter ion, a C1-C20 straight or branched chain alkyl, haloalkyl, heteroalkyl, haloheteroalkyl, aryl, heteroaryl, heterocycle, a mono-, di-, or polyhydroxyalkyl residue, and a mono-, di-, or polyhydroxyaryl residue, where R₂₉ and R₃₀ are independently selected from H, C1-C20 alkyl, C1-C20 cycloalkyl, aryl, NH₂, N(CH₃)₂, (CH₂)ₙOH, (CH₂)ₙO-alkyl, (CH₂)ₙOCOCH₃, (CH₂)ₙO(CH₂)ₘOH, (CH₂)ₙO(CH₂)ₘOCOCH₃, (CH₂)ₙO(CH₂)ₘO-alkyl, (CH₂)ₙN((CH₂)ₘOH)₂, (CH₂)ₙN((CH₂)ₘO-alkyl)₂, (CH₂)ₙN((CH₂)ₘO-alkylether)₂, ((C H₂)ₙO)ₘ((CH₂)_{Q})OH, (CH₂)ₙO(CH₂)ₘNH₂, (CH₂)ₙO(CH₂)ₘN(CH₃)₂, (CH₂)ₙO(CH₂)ₘN(CH₃)₃⁺A, (CH₂)ₙN((CH₂)ₘNH₂)₂, (CH₂)ₙN(CH₂)ₘN(CH₃(CH₂)ₙO-haloalkyl, (CH₂)ₙN(CH₂)ₘN(CH₃)₃⁺A)₂, ((CH₂)ₙO)ₙ(CH₂O)_{Q}COCH₃, an alkylphosphate residue, an alkylsulfonic acid residue, an alkylsulfonic ester residue, alkylsulfonic amide residue, an alkylmorpholino residue, an alkylheterocyclic residue, an alkylthiol residue, a mono-, di-, or polyhydroxyalkyl residue, a mono-, di-, or polyhydroxyaryl residue, a mono-, di-, or polyetheralkyl residue, and a mono-, di-, or polyetheraryl residue, where Q, n, and m are integers ranging from 0 to 10,000, and A is a physiologically acceptable counter ion, and wherein at least one of R₆, R₇, R₁₀, R₁₆, and R₁₇ possesses a carbamate moiety in its structure; and
M is selected from 2H, a metal cation, and photoactive metal ions selected from Ga³⁺, Pt²⁺, Pd²⁺, Sn⁴⁺, In³⁺, Ge⁴⁺, Si⁴⁺, Al³⁺, Zn²⁺, and Mg²⁺; or a pharmaceutically acceptable salt, prodrug, solvate, or metabolite thereof.

68. A pharmaceutical composition comprising an effective diagnostic or therapeutic amount of the compound of claim 67, together with at least one pharmaceutically acceptable carrier or excipient.

69. The pharmaceutical composition according to claim 68 used to treat ophthalmic diseases.

70. The pharmaceutical composition of claim 69 wherein said ophthalmic diseases are selected from proliferative retinopathies, macular edema, corneal neovascularization, conjunctival neovascularization, ocular tumors, viral retinitis adjunct to glaucoma filtration surgery and cyclodestruction, posterior capsule opacification, and age related macular degeneration.

71. The pharmaceutical composition according to claim 68 used to treat cardiovascular diseases.

72. The pharmaceutical composition according to claim 68 used to treat skin diseases.

73. Compounds of the following formula: 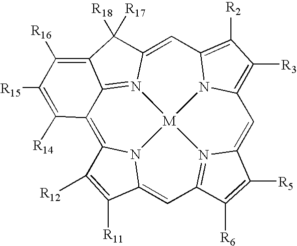
wherein:
R₂, R₃, R₅, R₆, R₁₁, R₁₂, R₁₄, R₁₅, R₁₆, R₁₇, and R₁₈ are indepently selected from H, a C1-C20 straight or branched chain alkyl, haloalkyl, heteroalkyl, haloheteroalkyl, aryl, heteroaryl, heterocycle, (CH₂)ₙCO₂R₁₉, (CH₂)ₙCONR₁₉R₂₀, (CH₂)ₙOCON(R₂₉)₂, (CH₂)ₙOCON═C(R₂₉)₂, (CH₂)ₙOCONR₂₉R₃₀, (CH₂)ₙOCON═C(R₂₉)(R₃₀), (CH₂)ₙCO₂(CH₂)ₙOCON(R₂₉)₂, (CH₂)ₙCO₂(CH₂)ₙOCON═C(R₂₉)₂, (CH₂)ₙCO₂(CH₂)ₙOCONR₂₉R₃₀, (CH₂)ₙCO₂(CH₂)ₙOCON═C(R₂₉)(R₃₀), (CH₂)ₙCONH(CH₂)ₙOCON(R₂₉)₂, (CH₂)ₙCONH(CH₂)ₙOCON═C(R₂₉)₂, (CH₂)ₙCONH(CH₂)ₙOCONR₂₉R₃₀, (CH₂)ₙCONH(CH₂)ₙOCON═C(R₂₉)(R₃₀), CH(OCON(R₂₉)₂)CH₃, CH(OCON═C(R₂₉)₂)CH₃, CH(OCONR₂₉R₃₀)CH₃, CH(OCON═C(R₂₉)(R₃₀))CH₃, SO₂NH(CH₂)ₙOCON(R₂₉)₂, SO₂NH(CH₂)ₙOCON═C(R₂₉)₂, SO₂NH(CH₂)ₙOCONR₂₉R₃₀, SO₂NH(CH₂)ₙOCON═C(R₂₉)(R₃₀), SO₂N((CH₂)ₙOCON(R₂₉)₂)₂, SO₂N((CH₂)ₙOCON═C(R₂₉)₂)₂, SO₂N((CH₂)ₙOCONR₂₉R₃)₂, SO₂N((CH₂)ₙOCON═C(R₂₉)(R₃₀))₂, an amino acid containing NR₁₉R₂₀, an amino acid ester containing NR₁₉R₂₀, and an amino acid amide containing NR₁₉R₂₀, where R₁₉ and R₂₀ are independently selected from H, a physiologically acceptable counter ion, a C1-C20 straight or branched chain alkyl, haloalkyl, heteroalkyl, haloheteroalkyl, aryl, heteroaryl, heterocycle, a mono-, di-, or polyhydroxyalkyl residue, and a mono-, di-, or polyhydroxyaryl residue, where R₂₉ and R₃₀ are independently selected from H, C1-C20 alkyl, C1-C20 cycloalkyl, aryl, NH₂, N(CH₃)₂, (CH₂)ₙOH, (CH₂)ₙO-alkyl, (CH₂)ₙOCOCH₃, (CH₂)ₙO(CH₂)ₘOH, (CH₂)ₙO(CH₂)ₘOCOCH₃, (CH₂)ₙO(CH₂)ₘO-alkyl, (CH₂)ₙN((CH₂)ₘOH)₂, (CH₂)ₙN((CH₂)ₙO-alkyl)₂, (CH₂)ₙN((CH₂)ₘO-alkylether)₂, ((CH₂)ₙO)ₘ((CH₂)_{Q})OH, (CH₂)ₙO(CH₂)ₘNH₂, (CH₂)ₙO(CH₂)ₘN(CH₃)₂, (CH₂)ₙO(CH₂)ₘN(CH₃)₃⁺A, (CH₂)ₙN((CH₂)ₘNH₂)₂, (CH₂)ₙN(CH₂)ₘN(CH₃)₂, (CH₂)ₙO-haloalkyl, (CH₂)ₙN(CH₂)ₙN(CH₃)₃⁺A)₂, ((CH₂)ₙO)ₘ(CH₂O)_{Q}COCH₃, an alkylphosphate residue, an alkylsulfonic acid residue, an alkylsulfonic ester residue, alkylsulfonic amide residue, an alkylmorpholino residue, an alkylheterocyclic residue, an alkylthiol residue, a mono-, di-, or polyhydroxyalkyl residue, a mono-, di-, or polyhydroxyaryl residue, a mono-, di-, or polyetheralkyl residue, and a mo o-, di-, or polyetheraryl residue, where Q, n, and m are integers ranging from 0 to 10,000, and A is a physiologically acceptable counter ion, wherein at least one of R₂, R₃, R₅, R₆, R₁₁, R₁₂, R₁₄, R₁₅, R₁₆, R₁₇, and R₁₈ possesses a carbamate moiety in its structure;
M is selected from 2H, a metal cation, and photoactive metal ions selected from Ga³⁺, Pt²⁺, Pd²⁺, Sn⁴⁺, In³⁺, Ge⁴⁺, Si⁴⁺, Al³⁺, Zn²+, and Mg²⁺;
or a pharmaceutically acceptable salt, prodrug, solvate, or: metabolite thereof.

74. A pharmaceutical composition comprising an effective diagnostic or therapeutic amount of the compound of claim 73, together with at least one pharmaceutically acceptable carrier or excipient.

75. The pharmaceutical composition according to claim 74 used to treat ophthalmic diseases.

76. The pharmaceutical composition of claim 75 wherein said ophthalmic diseases are selected from proliferative retinopathies, macular edema, corneal neovascularization, conjunctival neovascularization, ocular tumors, viral retinitis adjunct to glaucoma filtration surgery and cyclodestruction, posterior capsule opacification, and age related macular degeneration.

77. The pharmaceutical composition according to claim 74 used to treat cardiovascular diseases.

78. The pharmaceutical composition according to claim 74 used to treat skin diseases.

79. Compounds of the following formula: 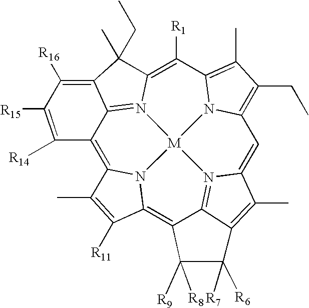
wherein:
R₁, R₆, R₇, R₈, R₉, R₁₁, R₁₄, R₁₅, and R₁₆are independently selected from H, OH, O-alkyl, CHO, methyl, halogen, (CH₂)ₙCO₂R₁₉, (CH₂)ₙCONR₁₉R₂₀, —OCON(R₂₉)₂, —OCON═C(R₂₉)₂, —OCONR₂₉R₃₀, OCON═C(R₂₉)(R₃₀), (CH₂)ₙOCON(R₂₉)₂, (CH₂)ₙOCON═C(R₂₉)₂, (CH₂)ₙOCONR₂₉R₃₀, (CH₂)ₙOCON═C(R₂₉)(R₃₀), (CH₂)ₙCO₂(CH₂)ₙOCON(R₂₉)₂, (CH₂)ₙCO₂(CH₂)ₙOCON═C(R₂₉)₂, (CH₂)ₙCO₂(CH₂)ₙOCONR₂₉R₃₀, (CH₂)ₙCO₂(CH₂)ₙOCON═C(R₂₉)(R₃₀), (CH₂)ₙCONH(CH₂)ₙOCON(R₂₉)₂, (CH₂)ₙCONH(CH₂)ₙOCON═C(R₂₉)₂, (CH₂)ₙCONH(CH₂)ₙOCONR₂₉R₃₀, (CH₂)ₙCONH(CH₂)ₙOCON═C(R₂₉)(R₃₀), CH(OCON(R₂₉)₂)CH₃, CH(OCON═C(R₂₉)₂)CH₃, CH(OCONR₂₉R₃₀)CH₃, CH(OCON═C(R₂₉)(R₃₀))CH₃, SO₂NH(CH₂)ₙOCON(R₂₉)₂, SO₂NH(CH₂)ₙOCON═C(R₂₉)₂, SO₂NH(CH₂)ₙOCONR₂₉R₃₀, SO₂NH(CH₂)ₙOCON═C(R₂₉)(R₃₀), SO₂N((CH₂)ₙOCON(R₂₉)₂)₂, SO₂N((CH₂)ₙOCON═C(R₂₉)₂)₂, SO₂N((CH₂)ₙOCONR₂₉R₃₀)₂, SO₂N((CH₂)ₙOCON═C(R₂₉)(R₃₀))₂, an amino acid containing NR₁₉R₂₀, an amino acid ester containing NR₁₉R₂₀, and an amino acid amide containing NR₁₉R₂₀, where R₁₉ and R₂₀ can be independently selected from H, a physiologically acceptable counter ion, a Cl1-C20 straight or branched chain alkyl, haloalkyl, heteroalkyl, haloheteroalkyl, aryl, heteroaryl, heterocycle, a mono-, di-, or polyhydroxyalkyl residue, and a mono-, di-, or polyhydroxyaryl residue, and where R₂₉ and R₃₀ are independently selected from H, C1-C20 alkyl, C1-C20 cycloalkyl, aryl, NH₂, N(CH₃)₂, (CH₂)ₙOH, (CH₂)ₙO-alkyl, (CH₂)ₙOCOCH₃, (CH₂)ₙO(CH₂)ₘOH, (CH₂)ₙO(CH₂)ₘOCOCH₃, (CH₂)ₙO(CH₂)ₘO-alkyl, (CH₂)ₙN((CH₂)ₘOH)₂, (CH₂)ₙN((CH₂)ₘO-alkyl)₂, (CH₂)ₙN((CH₂)ₘO-alkylether)₂, ((CH₂)ₙO)ₘ((CH₂)_{Q})OH, (CH₂)ₙO(CH₂)ₘNH₂, (CH₂)ₙO(CH₂)ₘN(CH₃)₂, (CH₂)ₙO(CH₂)ₘN(CH₃)₃⁺A, (CH₂)ₙN((CH₂)ₘNH₂)₂, (CH₂)ₙN(CH₂)ₘN(CH₃)₂, (CH₂)ₙO-haloalkyl, (CH₂)ₙN(CH₂)ₘN(CH₃)₃⁺A)₂, ((CH₂)ₙO)ₘ(CH₂O)_{Q}COCH₃, an alkylphosphate residue, an alkylsulfonic acid residue, an alkylsulfonic ester residue, alkylsulfonic amide residue, an alkylmorpholino residue, an alkylheterocyclic residue, an alkylthiol residue, a mono-, di-, or polyhydroxyalkyl residue, a mono-, di-, or polyhydroxyaryl residue, a mono-, di-, or polyetheralkyl residue, and a mono-, di-, or polyetheraryl residue, where Q, n, and m are integers ranging from 0 to 10,000, and A is a physiologically acceptable counter ion, wherein at least one of R₆, R₇, R₈, R₉, R₁₁, R₁₄, R₁₅, R₁₆ possesses a carbamate moiety in its structure; and
M is selected from 2H, a metal cation, and photoactive metal ions selected from Ga³⁺, Pt²⁺, Pd²⁺, Sn⁴⁺, In³⁺, Ge⁴⁺, Si⁴⁺, Al³⁺, Zn²+, and Mg²⁺;
or a pharmaceutically acceptable salt, prodrug, solvate, or metabolite thereof.

80. A pharmaceutical composition comprising an effective diagnostic or therapeutic amount of the compound of claim 79, together with at least one pharmaceutically acceptable carrier or excipient.

81. The pharmaceutical composition according to claim 79 used to treat ophthalmic diseases.

82. The pharmaceutical composition of claim 81 wherein said ophthalmic diseases are selected from proliferative retinopathies, macular edema, corneal neovascularization, conjunctival neovascularization, ocular tumors, viral retinitis adjunct to glaucoma filtration surgery and cyclodestruction, posterior capsule opacification, and age related macular degeneration.

83. The pharmaceutical composition according to claim 79 used to treat cardiovascular diseases.

84. The pharmaceutical composition according to claim 79 used to treat skin diseases.

85. Compounds of the following formula: 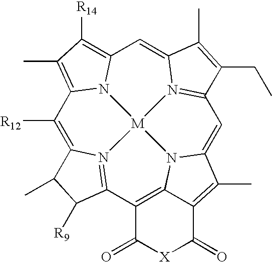
wherein:
R₉, R₁₂, and R₁₄ are independently selected from H, halogen, CH₃, CH₂CH₃, CH═CH₂, CH₂OH, CH₂OAc, CH₂O-alkyl, CH₂O-alkoxy, CH═CHCH₂N(CH₃)₂, CH═CHCH₂N(CH₃)₃⁺A⁻, COCH₃, CHO, CH(OH)CH₃, CH(O-alkyl)CH₃, CH(O-alkoxy)CH₃, CH₂CH₂O-alkyl, CH₂CH₂O-alkoxy, CH₂CH₂OAc, (CH₂)ₙCO₂R₁₉, (CH₂)ₙCONR₁₉R₂₀, (CH₂)ₙOCON(R₂₉)₂, (CH₂)ₙOCON═C(R₂₉)₂, (CH₂)ₙOCONR₂₉R₃₀, (CH₂)ₙOCON═C(R₂₉)(R₃₀), (CH₂)ₙCO₂(CH₂)ₙOCON(R₂₉)₂, (CH₂)ₙCO₂(CH₂)ₙOCON═C(R₂₉)₂, (CH₂)ₙCO₂(CH₂)ₙOCONR₂₉R₃₀, (CH₂)ₙCO₂(CH₂)ₙOCON═C(R₂₉)(R₃₀), (CH₂)ₙCONH(CH₂)ₙOCON(R₂₉)₂, (CH₂)ₙCONH(CH₂)ₙOCON═C(R₂₉)₂, (CH₂)ₙCONH(CH₂)ₙOCONR₂₉R₃₀, (CH₂)ₙCONH(CH₂)ₙOCON═C(R₂₉)(R₃₀), CH(OCON(R₂₉)₂)CH₃, CH(OCON═C(R₂₉)₂)CH₃, CH(OCONR₂₉R₃₀)CH₃, CH(OCON═C(R₂₉)(R₃₀))CH₃, an amino acid containing NR₁₉R₂₀, an amino acid ester containing NR₁₉R₂₀, and an amino acid amide containing NR₁₉R₂₀, where R₁₉ and R₂₀ can be independently selected from H, a physiologically acceptable counter ion, a C1-C20 straight or branched chain alkyl, haloalkyl, heteroalkyl, haloheteroalkyl, aryl, heteroaryl, heterocycle, a mono-, di-, or polyhydroxyalkyl residue, and a mono-, di-, or polyhydroxyaryl residue, and where R₂₉ and R₃₀ are independently selected from H, C1-C20 alkyl, C1-C20 cycloalkyl, aryl, NH₂, N(CH₃)₂, (CH₂)ₙOH, (CH₂)ₙO-alkyl, (CH₂)ₙOCOCH₃, (CH₂)ₙO(CH₂)ₘOH, (CH₂)ₙO(CH₂)ₘOCOCH₃, (CH₂)ₙO(CH₂)ₘO-alkyl, (CH₂)ₙN((CH₂)ₘOH)₂, (CH₂)ₙN((CH₂)ₘO-alkyl)₂, (CH₂)ₙN((CH₂)ₘO-alkylether)₂, ((CH₂)ₙO)ₘ((CH₂)_{Q})OH, (CH₂)ₙO(CH₂)ₘNH₂, (CH₂)ₙO(CH₂)ₘN(CH₃)₂, (CH₂)ₙO(CH₂)ₘN(CH₃)₃⁺A, (CH₂)ₙN((CH₂)ₘNH₂)₂, (CH₂)ₙN(CH₂)ₘN(CH₃)₂, (CH₂)ₙO-haloalkyl, (CH₂)ₙN(CH₂)ₘN(CH₃)₃⁺A)₂, ((CH₂)ₙO)ₘ(CH₂O)_{Q}COCH₃, an alkylphosphate residue, an alkylsulfonic acid residue, an alkylsulfonic ester residue, alkylsulfonic amide residue, an alkylmorpholino residue, an alkylheterocyclic residue, an alkylthiol residue, a mono-, di-, or polyhydroxyalkyl residue, a mono-, di-, or polyhydroxyaryl residue, a mono-, di-, or polyetheralkyl residue, and a mono-, di-, or polyetheraryl residue, where Q, n, and m are integers ranging from 0 to 10,000, and A is a physiologically acceptable counter ion;
X is selected from 0 and N(R₃₂), where R₃₂ is selected from alkyl, an amino acid, an amino acid ester, an amino acid amide, (CH₂)ₙOH, (CH₂)ₙO-alkyl, (CH₂)ₙOCOCH₃, (CH₂)ₙO(CH₂)ₘOH, (CH₂)ₙO(CH₂)ₘOCOCH₃, (CH₂)ₙO(CH₂)ₙO-alkyl, (CH₂)ₙN((CH₂)ₘOH)₂, (CH₂)ₙN((CH₂)ₘO-alkyl)₂, (CH₂)ₙN((CH₂)ₘO-alkylether)₂, ((CH₂)ₙO)ₘ(CH₂)_{Q}OH, (CH₂)ₙO(CH₂)ₘNH₂, (CH₂)ₙO(CH₂)ₘN(CH₃)₂, (CH₂)ₙO(CH₂)ₘN(CH₃)₃⁺A, (CH₂)ₙN((CH₂)ₘNH₂)₂, (CH₂)ₙN(CH₂)ₘN(CH₃)₂, (CH₂)ₙO-haloalkyl, (CH₂)ₙN(CH₂)ₘN(CH₃)₃⁺A, ((CH₂)ₙO)ₘ(CH₂O)_{Q}COCH₃, a mono-, di-, or polyhydroxyalkyl residue, a mono-, di-, or polyhydroxyaryl residue, a mono-, di-, or polyetheralkyl residue, a mono-, di-, or polyetheraryl residue, and a functional group that possesses a carbamate moiety of the formulae —OCON(R₂₉)₂, —OCON═C(R₂₉)₂, —OCONR₂₉R₃₀ or —OCON═C(R₂₉)(R₃₀), where Q, n, and m are integers ranging from 0 to 10,000;
wherein at least one of R₉, R₁₂, R₁₄, or X possesses a carbamate moiety in its structure; and
M is selected from 2H, a metal cation, and photoactive metal ions selected from Ga³⁺, Pt²⁺, Pd²⁺, Sn⁴⁺, In³⁺, Ge⁴⁺, Si⁴⁺, Al³⁺, Zn²⁺, and Mg²⁺;
or a pharmaceutically acceptable salt, prodrug, solvate, or metabolite thereof.

86. A pharmaceutical composition comprising an effective diagnostic or therapeutic amount of the compound of claim 85, together with at least one pharmaceutically acceptable carrier or excipient.

87. The pharmaceutical composition according to claim 86 used to treat ophthalmic diseases.

88. The pharmaceutical composition of claim 87 wherein said ophthalmic diseases are selected from proliferative retinopathies, macular edema, corneal neovascularization, conjunctival neovascularization, ocular tumors, viral retinitis adjunct to glaucoma filtration surgery and cyclodestruction, posterior capsule opacification, and age related macular degeneration.

89. The pharmaceutical composition according to claim 86 used to treat cardiovascular diseases.

90. The pharmaceutical composition according to claim 86 used to treat skin diseases.

91. A method for reducing the biological activity in vivo ofia biologically active carbamate photosensitizer comprising: providing a biologically active carbamate photosensitizer; enzymatically cleaving the biologically active carbamate photosensitizer in vivo to produce metabolites that are less biologically active than the biologically active carbamate photosensitizer.

92. The method of claim 91 wherein the metabolites produced are biologically inactive.

93. The method of claim 91 wherein the biologically active carbamate photosensitizer is produced from a hydroxyl-containing photosensitizer that displays poor photodynamic biological activity in vivo.

94. The method of claim 91 wherein enzymatic cleavage of the carbamate photosensitizer in vivo results in a therapeutically useful reduction in skin phototoxicity.

95. The method according to claim 91 wherein enzymatic cleavage of the carbamate photosensitizer in vivo results in a therapeutically useful reduction in occular phototoxicity.
